(19) United States
(12) Patent Application Publication
(10) Pub. No.: US 2025/0354117 A1
(43) Pub. Date: Nov. 20, 2025

(54) METHODS OF GENERATING NATURAL KILLER CELLS FROM PLURIPOTENT STEM CELLS AND COMPOSITIONS THEREOF

(71) Applicant: THE CHILDREN'S MEDICAL CENTER CORPORATION, Boston, MA (US)

(72) Inventors: George Q. DALEY, Cambridge, MA (US); Deepak K. JHA, Boston, MA (US); Mohammad Ali Toufic NAJIA, Cambridge, MA (US)

(21) Appl. No.: 18/867,655

(22) PCT Filed: May 19, 2023
(86) PCT No.: PCT/US23/67215

§ 371 Date: Nov. 20, 2024

Related U.S. Application Data
(60) Provisional application No. 63/344,057, filed on May 20, 2022.

(51) Int. Cl. C12N 5/0783; A61K 40/15; A61K 40/31; A61K 40/42

(52) U.S. Cl. CPC C12N 5/0646; A61K 40/15; A61K 40/31; A61K 40/42; C12N 2506/45; C12N 2510/00

(57) ABSTRACT

Methods provided herein describe a way to generate NK cells from pluripotent stem cells by disrupting the expression of a gene.

### CROSS REFERENCE TO RELATED APPLICATIONS

This application claims benefit under 35 U.S.C. § 119(e) of U.S. Provisional Application No. 63/344,057, filed May 20, 2022, the contents of which is incorporated herein by reference in its entirety.

### GOVERNMENT SUPPORT

This invention was made with government support under grant numbers HL134812 and DK120535, awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on May 19, 2023 is named 701039-191580WOPT_SL2.xml and is 40,037 bytes in size.

### TECHNICAL FIELD

The field of the invention relates to immune cell differentiation methods and compositions for use thereof.

### BACKGROUND

Cancer immunotherapy harnesses the immune system in order to target and destroy tumor cells. One type of cell utilized in cancer immunotherapy includes natural-killer (NK) cells, which destroys tumor cells by identifying surface markers associated with oncogenic transformation. Because cancer immunotherapy is coupled with other forms of treatment, it can result in the suppression of the patient's immune system. Pluripotent stem cells (including donor-derived and induced pluripotent stem cells (iPSCs or iPS cells)), are able to provide an ample supply of immune cells that can be utilized in different conditions.

### SUMMARY

The methods and compositions described herein were discovered, in part, by the observation that cells of different lineages comprise different patterns of transposable elements, which can aid in identification of cells in a particular lineage. The studies described in the working examples also demonstrate that modulation of transposable element repression by way of inhibiting TRIM28 can induce cells to preferentially generate natural killer T cells as compared to other lymphoid lineage or myeloid lineage cells.

Accordingly, provided herein are methods for generating a natural-killer (NK) cell from a pluripotent stem cell engineered to lack TRIM28 and/or EHMT1 expression and/activity.

One aspect provided herein relates to a method for generating a natural killer (NK) cell comprising: differentiating a pluripotent stem cell engineered to lack TRIM28 expression and/or activity for a sufficient time to promote differentiation to a CD56+ NK cell.

In one embodiment of this aspect and all other aspects described herein, the pluripotent stem cell comprises an induced pluripotent stem (iPS) cell, an embryonic stem cell, a donor-derived stem cell, a bone marrow cell, or a cord blood cell.

In another embodiment of this aspect and all other aspects described herein, the cord blood cell and/or bone marrow cell comprises a CD34+ hemogenic endothelial cell.

In another embodiment of this aspect and all other aspects described herein, the pluripotent stem cell engineered to lack TRIM28 expression and/or activity is generated using a CRISPR-Cas9 system.

In another embodiment of this aspect and all other aspects described herein, the pluripotent stem cell is engineered to delete or mutate a gene and/or protein encoding TRIM28, thereby reducing expression and/or activity of TRIM28.

In another embodiment of this aspect and all other aspects described herein, the method further comprises treatment with at least one additional inhibitor of EHMT1 and/or SETDB1.

In another embodiment of this aspect and all other aspects described herein, the NK cell generated is a CD56+, CD56+CD3−, CD56+CD3−CD8+, or CD56+CD3−CD8− NK cell.

In another embodiment of this aspect and all other aspects described herein, the cell engineered to lack TRIM28 expression comprises a pattern of transposable elements that is substantially similar to the pattern of transposable elements in a cell committed to the lymphoid lineage.

Another aspect provided herein relates to a method for generating an NK cell comprising contacting a pluripotent stem cell with an inhibitor of TRIM28 expression and/or activity and culturing under conditions and for a sufficient time to promote differentiation to an NK cell.

In one embodiment of this aspect and all other aspects described herein, the NK cell is CD56+, CD56+CD3−, CD56+CD3−CD8+, or CD5+6CD3−CD8− NK cell.

In another embodiment of this aspect and all other aspects described herein, the inhibitor of TRIM28 expression and/or activity comprises an inhibitory nucleic acid, a small molecule, or a peptide.

In another embodiment of this aspect and all other aspects described herein, the inhibitory nucleic acid is selected from the group consisting of: an siRNA, an shRNA, a miRNA, an antisense oligonucleotide, an aptamer, a ribozyme, and a triplex forming oligonucleotide.

In another embodiment of this aspect and all other aspects described herein, the method further comprises a step of administering or contacting with at least one inhibitor that modulates methylation of DNA.

In another embodiment of this aspect and all other aspects described herein, at least one inhibitor that inhibits methylation of DNA inhibits the expression and/or activity of one or more of: DNMT; MBD; DNA demethylase; HMT; methyl-histone binding protein; histone demethylase; HAT; acetyl-binding protein; or HDAC.

In another embodiment of this aspect and all other aspects described herein, the method further comprises administering or contacting with at least one inhibitor that targets sumoylation.

In another embodiment of this aspect and all other aspects described herein, at least one inhibitor that targets sumoylation is an E3 ligase inhibitor.

Another aspect provided herein relates to a method for generating an NK cell, the method comprising: contacting a pluripotent stem cell treated with an inhibitor that disrupts TRIM28 binding with one or more binding partners.

In one embodiment of this aspect and all other aspects described herein, one or more binding partners are selected from the group comprising of: KRAB-ZNF transcription factors, MDM2, p53, the NuRD complex (comprising of NuRD, Mi2α, and an HDAC), SETDB1, CBF-A, and HP1.

In another embodiment of this aspect and all other aspects described herein, the cell engineered to lack TRIM28 expression comprises a pattern of transposable elements that is substantially similar to the pattern of transposable elements in a cell committed to the lymphoid lineage.

In another embodiment of this aspect and all other aspects described herein, the NK cell is a CD56+, CD56+CD3−, CD56+CD3−CD8+, or CD56+CD3−CD8− NK cell.

Another aspect provided herein relates to an engineered NK cell generated using the method of any one the embodiments described herein, wherein the NK cell lacks TRIM28 expression or activity.

Another aspect provided herein relates to an engineered NK cell generated using the method of any one of the embodiments as described herein.

Another aspect provided herein describes a therapeutic cell composition comprising an NK cell of any one of the embodiments described herein or a population thereof, and a pharmaceutically acceptable carrier.

In one embodiment of this aspect and all other aspects described herein, the therapeutic composition is for use in cellular replacement therapy in a patient.

Another aspect provided herein relates to a therapeutic CAR-NK cell composition comprising an NK cell that lacks TRIM28 expression and/or activity, wherein the NK cell expresses a chimeric antigen receptor (CAR).

In one embodiment of this aspect and all other aspects described herein, the NK cell is an CD56+, CD56+CD3−, CD56+CD3−CD8+, or CD56+CD3−CD8− NK cell.

In another embodiment of this aspect and all other aspects described herein, the NK cell is generated by in vitro differentiation of a pluripotent stem cell engineered to lack TRIM28 expression and/or activity.

In another embodiment of this aspect and all other aspects described herein, the composition further comprises a pharmaceutically acceptable carrier.

In another embodiment of this aspect and all other aspects described herein, the cell is autologous to the subject to be treated.

In another embodiment of this aspect and all other aspects described herein, the composition further comprises a pharmaceutically acceptable carrier.

Another aspect provided herein relates to a method of treating a subject in need thereof, the method comprising: administering an NK cell of any embodiment described herein in combination with a NK cell engager (NKCE), bispecific killer cell engager (BiKE), or trispecific killer cell engager (TRiKE) to a subject in need thereof.

Another aspect provided herein relates to a method of treating a subject in need thereof, comprising administering a therapeutic cell composition of any one of the embodiments described herein to a subject in need thereof.

In one embodiment of this aspect and all other aspects described herein, the subject in need thereof has or is at risk of having cancer.

In another embodiment of this aspect and all other aspects described herein, the subject in need thereof has or is undergoing chemotherapy and/or irradiation.

In another embodiment of this aspect and all other aspects described herein, the cancer comprises a leukemia or a lymphoma.

In another embodiment of this aspect and all other aspects described herein, the cancer is of a B-cell lymphoma; a low grade/follicular non-Hodgkin's lymphoma (NHL); a small lymphocytic (SL) NHL; an intermediate grade/follicular NHL; an intermediate grade diffuse NHL; a high grade immunoblastic NHL; a high grade lymphoblastic NHL; a high grade small non-cleaved cell NHL; a bulky disease NHL; a mantle cell lymphoma; an AIDS-related lymphoma; a Waldenstrom's Macroglobulinemia); a chronic lymphocytic leukemia (CLL); an acute lymphoblastic leukemia (ALL); a Hairy cell leukemia; or a chronic myeloblastic leukemia.

In another embodiment of this aspect and all other aspects described herein, the subject in need thereof is human.

Another aspect provided herein relates to a method for identifying or selecting a lymphoid progenitor cell, the method comprising detecting an increase in the number of upregulated transposable elements in a progenitor cell as compared to a reference, thereby identifying a lymphoid progenitor cell.

Another aspect provided herein relates to a method for comparing the pattern of transposable elements in a progenitor cell to the pattern of transposable elements in a progenitor cell committed to the lymphoid progenitor cell, and wherein the presence of a substantially similar pattern of transposable elements as compared to a lymphoid progenitor cell is detected, the cell is identified as a lymphoid progenitor cell.

In one embodiment of this aspect and all other aspects described herein, the reference comprises a reference cell or population or a reference value.

In another embodiment of this aspect and all other aspects described herein, the method further comprises a step of isolating the lymphoid progenitor cell.

In another embodiment of this aspect and all other aspects described herein, the transposable elements are selected from the group comprising: endogenous retroviruses (ERVs), long interspersed elements (LINEs) and short interspersed elements (SINEs).

### BRIEF DESCRIPTION OF THE DRAWINGS

FIG. 1 is the quantification of frameshifting indels in CD34+ cord blood from Cas9 ribonucleoproteins (RNPs).

FIG. 2 is data showing the knockout of EHMT1, TRIM28 or SETDB1 resulting in CD56+ NK lineage skewing.

FIG. 3 is data showing the knockout of EHMT1, TRIM28 or SETDB1 results in CD56+ NK lineage skewing.

FIG. 4 depicts multiple exemplary strategies to generate NK cells.

FIG. 5 shows EHMT1 and TRIM28 knockout (KO) NK cells can exhibit different cytotoxic properties.

FIG. 6 depicts knockout of TRIM28 reduces clonogenic erythroid potential of pluripotent stem cells.

FIG. 7 shows that the knockout of EHMT1, TRIM28, or SETDB1 concordantly attenuates differentiation to CD14+ monocytes.

FIG. 8 depicts that the knockout of TRIM28 affects T-cell progenitor specification.

FIG. 9 shows that ABC enhancers are sufficient to cluster hematopoietic cell types.

FIG. 10 shows ABC enhancers mark hematopoietic cell-types and enrich for lineage-specific transcription factors.

FIG. 11 examines how ABC enhancers mark hematopoietic cell-types, enrich for lineage-specific transcription factors, and uncovers functionally relevant enhancer-gene linkages.

FIG. 12 determines that transposable element expression is sufficient to classify hematopoietic cells using gene clustering.

FIG. 13 depicts that transposable element expression in hematopoiesis is skewed in a lineage-specific manner.

FIG. 14 determines the loss of SETDB1 attenuates lymphoid progenitor commitment.

FIG. 15 examines the loss of T-cell fates in SETDB1 knockouts.

FIG. 16 shows the knockout of EHMT1 markedly enhances NK fates at the expense of T-cells.

FIG. 17 depicts the over-arching model.

FIGS. 18A-18C shows expression of transposable elements alone was sufficient to classify distinct hematopoietic cell types and resulted in lineage clustering comparable to clustering derived from expressed genes (FIGS. 18A-18B). TEs were progressively up-regulated during lymphoid differentiation, initiating at common lymphoid progenitors (CLPs) (FIG. 18C).

FIGS. 18D-18K investigates dynamically expressed TEs during differentiation by identifying differentially expressed TEs in progenitors and mature progeny relative to hematopoietic stem cells (HSCs) (FIGS. 18D-18H). B cells exhibited the largest number of up-regulated TEs compared to HSCs, which comprised ERV, LINE and SINE TE families (FIG. 18E). TEs also exhibited cell type specificity amongst closely related cell types such NK, CD4 T and CD8 T cells (FIG. 18F-18H). Gene module scores were calculated for genes involved in H3K9 methylation across all single cells and identified that mature cell types exhibited reduced expression of H3K9 methylation genes compared to HSPCs, consistent with the up-regulation of TE expression (FIG. 18I). The nucleosome remodeling and deacetylation (NuRD) complex, which operates on chromatin marked by H3K9 methylation to mediate epigenetic silencing, demonstrated similar expression dynamics as the H3K9 methylation gene program (FIG. 18J). H3K9 demethylation expression was elevated in mature hematopoietic cell types compared to HSPCs (FIG. 18K).

FIGS. 19A-19D depicts a gene-centric loss-of-function approach to components of TE epigenetic machinery (FIG. 19A), focusing on H3K9 methyltransferases that were dynamically expressed between HSPCs and mature hematopoietic cell types (FIG. 19B), including EHMT1, SUV39H2, SETDB1, and TRIM28. TRIM28 expression attenuates in mature hematopoietic progeny compared to HSPCs (FIG. 19B). CRISPR/Cas9 was utilized to generate arrayed knockouts with two distinct gRNAs per gene in primary HSPCs then assessed differentiation to myeloid-erythroid, T cell and B cell fates (FIG. 19A). Following six weeks of in vitro differentiation, knockout of EHMT1 resulted in a reproducibly pronounced reduction of CD56−CD3+ T cells (gRNA01: 13.9±1.8%, gRNA02: 6.6±0.69%) and a coupled increase of CD56+CD3− natural killer (NK) cells (gRNA01: 35.4±5.7%, gRNA02: 58.7±4.9%) compared to an AAVS1-targeting gRNA control (T cells: 38.7±12.5%, NK cells: 1.2±0.45%) (FIG. 19C). In line with the hypothesis, knockout of TRIM28 phenocopied the T-to-NK cell lineage shift observed in EHMT1 knockouts (FIG. 19D).

FIGS. 19E-19J shows NK cells produced from EHMT1 (FIG. 19E) or TRIM28 (FIG. 19F) knockouts also expressed canonical NK markers such as CD7 (FIGS. 19G-19H) and CD8 (FIGS. 19I-19J), supportive of their NK identity. Loss of EHMT1 or TRIM28 resulted in differing degrees of CD8 expression, potentially suggesting the generation of distinct NK subtypes.

FIGS. 19K-19M determines knockout of EHMT1 resulted in an NK lineage shift at the attenuation of CD19+ B cells and CD14+ monocytes following five weeks of differentiation (FIG. 19K-19M). While a statistically significant decrease in CD19+ B cells was not observed (FIG. 19L), knockout of TRIM28 also enhanced the proportion of NK cell fates at the expense of CD14+ monocytes (FIGS. 19K, 19M). Additionally, in this assay, knockout of SETDB1 phenocopied loss of EHMT1 (FIG. 19K). Finally, knockout of EHMT1, TRIM28 or SETDB1 resulted in an overall increase in NK cell numbers, underscoring the role of heterochromatin regulation in NK cell fate commitment.

FIGS. 20A-20H shows B cells exhibited the largest number of up-regulated TEs compared to HSCs, which comprised ERV, LINE and SINE TE families (FIG. 20A). TEs are examined in monocytes compared to HSCs (FIG. 20B). TEs of the ERV1 subfamily were preferentially expressed in CD8 T cells and repressed in CD4 T cells (FIG. 20C-20D).

FIGS. 20E-20H examines scRNA-Seq profiles on 35,882 cells covering the full spectrum of the human hematopoietic system (Granja et al 2019 Nature Biotech) to analyze the expression H3K9 methylation-related genes during differentiation (FIG. 20E). Gene module scores were calculated for genes involved in H3K9 methylation across all single cells and identified that mature cell types exhibited reduced expression of H3K9 methylation genes compared to HSPCs, consistent with the up-regulation of TE expression (FIG. 20F). The nucleosome remodeling and deacetylation (NuRD) complex, which operates on chromatin marked by H3K9 methylation to mediate epigenetic silencing, demonstrated similar expression dynamics as the H3K9 methylation gene program (FIG. 20G). H3K9 demethylation expression was elevated in mature hematopoietic cell types compared to HSPCs (FIG. 20H).

FIGS. 21A-21D examines the delivery of Cas9 ribonucleoproteins (RNPs) resulted in highly efficient formation of frameshifting indels for each gRNA in CD34+ umbilical cord blood, enabling experiments directly on the knockout pool of cells (FIGS. 21A-21B). The generation of differentiated cell types was examined after knockout of TRIM28 (FIGS. 21C-21D). T cell differentiation was first initiated on CD34+ HSPCs 48 hours post-delivery of Cas9 RNPs using a Notch-based, stroma-free assay.

FIGS. 21E-21G determines knockout of EHMT1 or TRIM28 resulted in a reduction of T cell associated antigens, such as CD5, two weeks into in vitro differentiation without affecting overall cell viability, suggesting an early manifestation of the T-to-NK fate choice within lymphoid progenitors (FIGS. 21E-21G).

FIGS. 21H-21K shows after following six weeks of in vitro differentiation, knockout of EHMT1 resulted in a reproducibly pronounced reduction of CD56−CD3+ T cells (gRNA01: 13.9±1.8%, gRNA02: 6.6±0.69%) and a coupled increase of CD56+CD3− natural killer (NK) cells (gRNA01: 35.4±5.7%, gRNA02: 58.7±4.9%) compared to an AAVS1-targeting gRNA control (T cells: 38.7±12.5%, NK cells: 1.2±0.45%) (FIG. 21H). Knockout of TRIM28 phenocopied the T-to-NK cell lineage shift observed in EHMT1 knockouts (FIG. 21I). Additionally, in this assay, knockout of SETDB1 phenocopied loss of EHMT1 (FIG. 21J). Finally, knockout of EHMT1, TRIM28 or SETDB1 resulted in an overall increase in NK cell numbers (FIG. 21K), underscoring the role of heterochromatin regulation in NK cell fate commitment.

FIGS. 22A-22G show a comprehensive atlas of enhancer-gene regulation throughout the human hematopoietic system. FIG. 22A shows a schematic overview of the approach to generate enhancer-gene maps of the human hematopoietic system to dissect TE contributions to gene regulation. HSC=hematopoietic stem cell; MPP=multipotent progenitor; CMP=common myeloid progenitor; LMPP=lymphoid-primed multipotent progenitor; MEP=megakaryocyte-erythrocyte progenitor; GMP=granulocyte-monocyte progenitor; CLP=common lymphoid progenitor; CFU-E=erythroid colony forming unit; ProE=proerythroblast; BasoE=basophilic erythroblast; PolyE=polychromatic erythroblast; OrthoE=orthochromatic erythroblast; Mega=megakaryocyte; Mono=monocyte; iMΦ=inflammatory macrophage; sMΦ=suppressor macrophage; mDC=myeloid dendritic cell; pDC=plasmacytoid dendritic cell; CD8n=naive CD8+ T cell; CD8 cm=central memory CD8+ T cell; CD8em=effector memory CD8+ T cell; Treg=regulatory T cell; Teff=CD4+ T effector cell; Th1=CD4+ T helper 1 cell; Th2=CD4+ T helper 2 cell; Th17=CD4+ T helper 17 cell; Tfh=CD4+ T follicular helper cell; gdT=γδT cell. FIG. 22B examines unsupervised t-SNE on the top 50 principal components for the 120,000 most variably accessible ABC enhancers across all hematopoietic cell types. Each dot represents a primary hematopoietic sample and the colors represent clusters identified by density clustering. FIG. 22C exhibits a cluster residence heatmap showing the percent of each FACS-identified hematopoietic cell type that resides within each of the 16 annotated clusters. FIG. 22D shows a heatmap visualizing the proportion of enhancer-gene connections shared across all profiled hematopoietic cell types. An enhancer-gene link is considered to be shared between two cell types if the predicted gene is the same and the ABC enhancers overlap. The rows and columns are hierarchically ordered the same, and the lineage identity of the cell types is noted atop. FIG. 22E examines the number of enhancer connections per gene for all genes with ABC predictions in HSCs (top) and CD19+ B cells (bottom). Cell-type specific regulators are noted within the plot. FIGS. 22F-22G analyzes transcription factor footprinting specifically within ABC enhancers of the noted cell types (FIG. 22F) or lineages (FIG. 22G). The sequence logo and JASPAR identifier of the transcription factor motif utilized for the footprinting analysis is noted as an inset on each plot. The Tn5 insertion bias track for each motif is shown below each footprint plot.

FIGS. 23A-23E. TE families contribute to cell-type specific hematopoietic gene regulation FIG. 23A examines enrichment of transposable element families (rows) within ABC enhancers of human hematopoietic cell types (columns). Rows and columns are hierarchy clustered based on the enrichment score. Enrichment was determined using the GIGGLE framework (Layer et al. 2018) and a significantly enriched TE was defined if at least 20 elements of the TE family overlapped ABC enhancers of a cell type, had an odds ratio >2.5 and a Fisher's two-tailed p-value <0.01. FIGS. 23B and 23D shows a heatmap of ATAC-Seq signal over the set of LTR10A/F elements (n=56) (FIG. 23B) or LTR2B elements (n=26) (FIG. 23D) overlapping ABC enhancers. Each column is the aggregate ATAC-Seq signal across all donors and replicates. The average, normalized ATAC-Seq signal within a +/−1 kb window of the elements is displayed on the bottom of each heatmap. FIG. 23C analyses binding predictions for FOSL2 (JASPAR motif ID: MA0478.1) in LTR10A/F ABC enhancers using the TOBIAS transcription factor occupancy framework. The footprint fold change column represents the matched change in FOSL2 footprint scores between the resting and activated T cell states. Log 2(FC) is calculated as log 2(activated/resting). The binding prediction columns depict whether individual FOSL2 binding sites were predicted by TOBIAS to be bound/unbound in resting and activated T cell states. FIG. 23E analyses binding predictions for SPI1 (HOCOMOCOv11) in LTR2B ABC enhancers, analogous to FIG. 23C.

FIG. 24A-24H examines the modulation of TE regulatory machinery influences lineage output from hematopoietic stem and progenitor cells. FIG. 24A shows an experimental scheme to genetically knockout genes involved in TE regulation via heterochromatin formation within CD34+ umbilical cord blood, and to assess in vitro lymphoid differentiation. FIGS. 24B-24C analyze the fractional abundance of CD56−CD3+ T and CD56+CD3− NK cells within the CD45+DAPI− population quantified by flow cytometry following six weeks of in vitro T cell differentiation (n=3 independent differentiation experiments per gRNA condition). *=p<0.05 in comparison to the AAVS1 gRNA control evaluated with a two-sided Wilcoxon rank-sum test. FIG. 24D examines flow cytometry profiling of CD5 and CD7 on lymphoid progenitors following 14 days of in vitro T cell differentiation. The plots are pregated on CD45+DAPI− cells. FIG. 24E examines flow cytometry profiling of CD56+CD3− NK and CD56−CD3+ T cell populations following 28 days and 42 days of in vitro T cell differentiation. The plots are pregated on CD45+DAPI− cells. FIG. 24F shows the Fractional abundance of CD19+ B cells, CD56+ NK cells and CD14+ monocytes within the CD45+DAPI− population quantified by flow cytometry following 35 days of differentiation in an MS5 co-culture assay (n=3 independent differentiation experiments per knockout condition). *=p<0.05 in comparison to the AAVS1 gRNA control evaluated with a two-sided Wilcoxon rank-sum test. FIGS. 24G-24H shows representative flow cytometry plots of CD19+ B cells, CD56+ NK cells and CD14+ monocytes across gRNA conditions targeting the AAVS1 locus, EHMT1 and TRIM28. The plots are pregated on CD45+DAPI− cells.

FIGS. 25A-25G analyze knockout of EHMT1 or TRIM28 generate distinct hematopoietic progenitors with early NK characteristics. FIG. 25A shows scATAC-Seq latent semantic indexing UMAP projection and clustering of 23,593 cells encompassing AAVS1, EHMT1 gRNA01, EHMT1 gRNA02, TRIM28 gRNA01 and TRIM28 gRNA02 conditions at D4+14 of in vitro lymphoid differentiation. FIG. 25B examine scRNA-Seq UMAP projection and clustering of 25,288 cells encompassing AAVS1, EHMT1 gRNA01, EHMT1 gRNA02, TRIM28 gRNA01 and TRIM28 gRNA02 conditions at D4+14 of in vitro lymphoid differentiation. FIGS. 25C-25D examine scATAC-Seq UMAP plot (FIG. 25C) and scRNA-Seq UMAP plot (FIG. 25D) colored by the embedding density of cells from the indicated gRNA conditions. FIG. 25E shows the visualization of the expression of T and NK cell associated genes overlaid on the scRNA-Seq UMAP. FIG. 25F examine select marker genes that are differentially expressed (FDR<0.05, two-tailed Wilcoxon rank-sum test) in each transcriptional cluster from scRNA-Seq. Color scale corresponds to z-scored, log-transformed mean gene-expression counts for each cluster. FIG. 25G analyses the UMAP projection of scATAC-Seq data colored by chromVAR TF motif bias-corrected deviations for the indicated factors. TF motifs represent a clustered archetype derived from (Vierstra et al. 2020).

FIG. 26A-26I. examines how EHMT1 and TRIM28 knockout NK cells exhibit unique phenotypic and molecular states. FIG. 26A depicts an experimental scheme to genetically knockout EHMT1 or TRIM28 within CD34+ umbilical cord blood and perform NK-supportive differentiation. FIG. 26B shows the expression of CD56 and CD3 assayed by flow cytometry on D4+28 of in vitro NK differentiation. Plots are pre-gated on viable CD45+ cells. FIG. 26C examines the quantification of the number of CD56+CD3− NK cells on D4+28 of in vitro differentiation for all gRNA conditions. n=3 independent differentiation replicates per gRNA. *=p<0.05 in comparison to the AAVS1 gRNA control evaluated with a two-sided Wilcoxon rank-sum test. FIG. 26D shows the expression of CD56 and CD16 assayed by flow cytometry on D4+28 of in vitro NK differentiation. Plots are pre-gated on viable CD45+CD56+ cells. FIG. 26E examines the quantification of the number of CD56+CD16+ NK cells on D4+28 of in vitro differentiation for all gRNA conditions. n=3 independent differentiation replicates per gRNA. *=p<0.05 in comparison to the AAVS1 gRNA control evaluated with a two-sided Wilcoxon rank-sum test. FIG. 26F shows the heatmap of changes in ATAC-Seq chromatin accessibility for the top TFs with the greatest accessibility variability between gRNA conditions. The color scale corresponds to chromVAR accessibility deviation z-scores of a TF across conditions. n=3 independent differentiation replicates. FIG. 26G examines an expression heatmap of select genes exhibiting differential expression (log 2(fold change)>1 and FDR<0.05, DESeq2) in at least one gRNA condition compared to the AAVS1 gRNA control. The color scale corresponds to z-scored, log-transformed mean gene-expression counts. FIG. 26H analyses GSEA on RNA-Seq data comparing TRIM28 gRNA versus AAVS1 gRNA demonstrating over-representation of genes related to type I interferon production (GO:0032481) within TRIM28 gRNA cells. FIG. 26I examines the proportion of IFN-γ+NK cells measured by intracellular flow cytometry across unstimulated and stimulated conditions. Each dot represents an independent differentiation replicate. *=p<0.05 in comparison to the AAVS1 gRNA control evaluated with a two-sided Wilcoxon rank-sum test.

FIG. 27A-27C examines precision-recall curves comparing ABC enhancer-gene predictions to experimental CRISPR data in K562s (FIGS. 27A-27B) or an expanded compendium of hematopoietic cell lines: GM12878, THP1+/−stimulation, Jurkat+/−stimulation, and BJAB+/−stimulation (FIG. 27C). CRISPRi-FlowFISH screen data was derived from (Fulco et al. 2019) for analysis in FIGS. 27A-27B and from (Nasser et al. 2021) for analysis in FIG. 27C. The full ABC model in FIG. 27A refers to measurements of enhancer activity with H3K27ac ChIP-Seq and chromatin accessibility, and contact frequency measurements from a 10-cell type averaged HiC dataset, as originally reported in (Fulco et al. 2019). The modified ABC model in FIG. 27B-27C utilizes only chromatin accessibility for enhancer activity and a power-law of genomic distance to approximate HiC.

FIG. 27D shows a heatmap of the enrichment of predicted enhancers from the modified ABC model within ChIP-Seq-defined chromHMM states from the Roadmap Epigenomics Project. Enrichment was determined with a binomial test. The chromHMM epigenomes used for the enrichment analysis are noted along the top and the ATAC-Seq data used to make the ABC predictions was derived from (Nasser et al. 2021).

FIGS. 27E-27I examines normalized ATAC-Seq sequencing tracks depicting ABC enhancer-gene linkages across various hematopoietic cell types. Known and experimentally verified enhancers are highlighted with blue shading. The thickness of the enhancer-gene link is scaled by the ABC score. The genomic regions visualized are noted in hg38 coordinates below each sequencing track.

FIG. 28A examines box and whisker plots of TSS enrichment scores for all samples within each hematopoietic cell type. Each dot represents an individual ATAC-Seq sample. The hinges represent the 25th to 75th percentile.

FIG. 28B shows box and whisker plots of the fraction of ATAC-Seq peaks within a sample identified as ABC enhancers for each hematopoietic cell type. Each dot represents an individual ATAC-Seq sample. The hinges represent the 25th to 75th percentile.

FIG. 28C shows box and whisker plots of the Pearson correlations of chromatin accessibility in the pan-hematopoiesis ABC enhancer peakset between all samples (technical replicates and different donors) in each hematopoietic cell type. The hinges represent the 25th to 75th percentile.

FIG. 28D examine the decision metrics used for the density clustering methodology, where rho is the number of points that are closer than the cutoff distance to a given point and delta is the distance between a given and any other point with higher density. Clusters were determined with rho=0.8 and delta=0.08.

FIG. 28E shows dot plot of enrichment of GO Biological Processes within ABC-linked genes for each hematopoietic cell type noted. Enrichment p-values, which were determined by a binomial test were FDR corrected and only terms with an FDR<0.01 are plotted.

FIGS. 29A-29B shows quantification of indels by TIDE analysis for each CRISPR/Cas9 RNP-mediated knockout in CD34+ umbilical cord blood HSPCs. An aliquot of the population of CD34+ cord blood cells were collected for gDNA extraction two days following nucleofection of CRISPR/Cas9 RNPs. The indel locus was PCR dialed-out from the genome and Sanger sequenced. Replicates represent TIDE quantification from forward and reverse Sanger sequencing traces of the indel amplicon.

FIGS. 29C-29D examines quantification of the absolute numbers of CD56+CD3− NK cells on D4+46 of in vitro T cell differentiation across all gRNA conditions (n=3 replicate differentiations per gRNA). *=p<0.05 in comparison to the AAVS1 gRNA using a two-tailed Wilcoxon rank-sum test.

FIG. 29E shows quantification of indels in an analogous manner as described in (A-B) on CD56+CD3− NK cells on D4+28 of in vitro T cell differentiation. Replicates represent n=3 independent differentiations per gRNA.

FIG. 29F analyses the concordance of frameshifting indel frequencies quantified in the starting CD34+ HSPC population versus frameshifting indel frequencies within D4+28 NK cells.

FIG. 29G-29H examines quantification of total viable cells, the proportion of CD5+CD7+ lymphoid progenitors (FIG. 29G) or CD56+CD3− NK cells (FIG. 29H) as determined by flow cytometry, and the absolute cell counts of each population across all gRNA conditions from in vitro T cell differentiation. Replicates represent n=3 independent differentiations per gRNA. *=p<0.05 in comparison to the AAVS1 gRNA using a two-tailed Wilcoxon rank-sum test.

FIG. 29I shows the proportion of CD56+CD3− NK and CD56−CD3+ T cells within the CD45+DAPI− population, quantified on D28 of in vitro T cell differentiation starting from CD34+ umbilical cord blood HSPCs treated continuously with UNC0642. Error bars represent the standard deviation across n=3 independent differentiations per dose of UNC0642. *=p<0.05 compared to the DMSO control using a two-tailed Wilcoxon sum-rank test.

FIG. 29J examines representative flow cytometry plots of data summarized in (I). The plots are pre-gated on CD45+DAPI− cells.

FIG. 29K-29L examines the quantification of the absolute numbers of CD56+CD3− NK cells (FIG. 29K) and total viable cells (FIG. 29L) on D4+35 of the MS5 co-culture assay across all gRNA conditions (n=3 replicate differentiations per gRNA). *=p<0.05 in comparison to the AAVS1 gRNA using a two-tailed Wilcoxon rank-sum test.

FIG. 29M shows the quantification of the proportion (top) and absolute cell counts (bottom) of CD19+ B cell progenitors on D4+35 of the MS5 co-culture assay. Replicates represent n=2 independent differentiations per gRNA. *=p<0.05 in comparison to the AAVS1 gRNA using a two-tailed Wilcoxon rank-sum test.

FIG. 30A examines aggregated scATAC-Seq fragment size distributions for each gRNA library demonstrating sub-, mono- and multi nucleosome spanning fragments.

FIG. 30B shows enrichment of ATAC-Seq accessibility +/−2 kb of transcription start sites for each gRNA library.

FIG. 30C depicts violin and box-whisker plot of the normalized TSS enrichment for each single cell passing quality control filters per gRNA library.

FIG. 30D depicts violin and box-whisker plot of the number of total aligned fragments for each single cell passing quality control filters per gRNA library.

FIG. 30E shows the proportion of cells from a gRNA condition residing within scATAC-Seq (top) or scRNA-Seq (bottom) clusters.

FIG. 30F examines UMAP projections of scATAC-Seq (top) or scRNA-Seq (bottom) and cells belonging to each gRNA condition are highlighted.

FIG. 30G depicts RNA Velocity analysis (steady-state model) projected onto the scRNA-Seq UMAP projection.

FIG. 30H shows pseudotime trajectory representation of the divergence of EHMT1 gRNAs from AAVS1 gRNA control cells using scATAC-Seq data and overlaid on the UMAP projection of single cells.

FIG. 31A examines ATAC-Seq chromatin accessibility of TE families that are significantly derepressed in either EHMT1 or TRIM28 gRNA conditions compared to the AAVS1 gRNA control at D4+28 of NK differentiation. The color scale corresponds to a scaled chromVAR deviation.

FIG. 31B shows quantification of the number of significantly derepressed TE families per gRNA in comparison to the AAVS1 gRNA control (FDR<0.1) at D4+28 of NK differentiation. n=3 replicate differentiations per gRNA.

FIG. 31C analyses the gene expression heatmap of zinc finger proteins that are differentially expressed in EHMT1 or TRIM28 gRNA conditions compared to the AAVS1 gRNA control. Differentially expressed genes were determined with DESeq2 and defined as log 2(fold-change)>1 and FDR<0.05. The (*) designates whether the zinc finger is annotated to contain a KRAB domain in the Uniprot database. n=3 differentiation replicates per gRNA.

FIG. 31D shows the number of transcription factor binding sites mapped on each consensus position of the TE. The x-axis indicates nucleotide positions of the TE family consensus sequence, and the y-axis indicates number of TE copies harboring the transcription factor binding sites at each position.

FIG. 32A examines the expression of NK surface markers assayed by flow cytometry on D4+28 of in vitro NK differentiation. Plots are pre-gated on viable CD45+CD56+ cells.

FIG. 32B shows a scaled expression of EHMT1 and TRIM28 across all gRNA conditions demonstrating that the knockouts result in transcriptional attenuation of their respective target gene.

FIG. 32C analyses the expression of a core NK signature gene set, derived from (Crinier et al. 2018) across all gRNA conditions. n=3 replicate differentiations per gRNA.

FIG. 32D shows a K562 killing assay using 0:1, 1:1, 3:1 and 10:1 mixtures of in vitro derived NK cells from all gRNA conditions to K562 cells. Co-cultures were incubated for 4 hours and then Annexin V+7AAD+ cells were assessed by flow cytometry.

FIG. 32E-32F examines the principal-component analysis (PCA) plots of RNA-Seq gene expression (left) and ATAC-Seq chromatin accessibility profiles (right) for all gRNA conditions on D4+28 of in vitro NK differentiation and n=3 independent differentiation replicates per gRNA.

FIG. 33 shows a schematic of the in vitro T cell differentiation experiment starting from CD34+ umbilical cord blood cells (top) and quantification of the proportion of CD56+CD3− NK cells and CD56−CD3+ T cells following 28 days of differentiation (bottom left) treated continuously with UNC0642. Error bars represent standard deviation. N=3 independent differentiation replicates per dose of UNC0642. Representative flow cytometry plots (bottom right) of CD56 and CD3 expression after 28 days of differentiation across various doses of UNC0642, demonstrating the dose-dependent acquisition of NK cells. *=p<0.05.

FIG. 34 demonstrates human iPS cells were differentiated to NK cells using a protocol described by Zhu, Kaufman (2019). Methods in Molecular Biology (doi.org/10.1007/978-1-4939-9728-2_12). Following embryoid body formation, cells were treated with 50 nM UNC0642 or DMSO for 28 days of NK differentiation. Flow cytometry profiling was performed on the Day 28 timepoint for CD56 and CD122 (IL2RB) (left), NKp46 (center) and CD16 (right).

FIG. 35A-35F examines rapid and selective degradation of endogenous TRIM28 in human iPS cells. FIG. 35A shows an experimental approach to generate a human iPS cell line that encodes degradation-sensitive TRIM28-FKBP alleles using the dTAG system. An mNeon-FKBP12^{F36V} open reading frame DNA sequence was knocked into the TRIM28 locus (SEQ ID NO: 32) to tag the N-terminus of TRIM28 and thus create an mNeon-FKBP12^{F36}V-TRIM28 fusion protein. The knockin was achieved through CRISPR/Cas9-mediated HDR (gRNA, SEQ ID: 31) where the knockin cassette was delivered to cells in an rAAV6 vector. FIG. 35B examines bulk knockin efficiency was assessed with flow cytometry for mNeon five days after simultaneous CRISPR/Cas9 RNP nucleofection and rAAV6 infection of iPS cells (MOI: 50,000 viral genomes/cells). FIG. 35C shows how iPS knockin clones were derived by single cell FACS on the mNeon+ population from FIG. 35B and genotyped by PCR using primers that flank the knockin locus. The wild-type, unedited locus results in an amplicon of 1,204 bp and the knockin locus generates an amplicon of 2,371 bp. (*) denotes biallelic knockin clones. FIG. 35D analyzes microscopy for DAPI and mNeon on a biallelic knockin iPS clone to visualize nuclear localization of mNeon signal, consistent with the known nuclear localization of endogenous TRIM28 proteins. FIG. 35E examines titration of degrader molecules (dTAG-13 and dTAG^{V}-1) on a biallelic knockin iPS clone, assayed by flow cytometry for mNeon as a quantitative proxy for TRIM28 protein expression. FIG. 35F shows a western-blot for TRIM28 on four iPS clones treated with DMSO or 500 nM dTAG^{V}-1 for 24 hours. FKBP12^{F36V} tagged TRIM28 experienced ligand-dependent proteolysis with near-complete degradation after 24 hours of exposure to the dTAG^{V}-1 ligand.

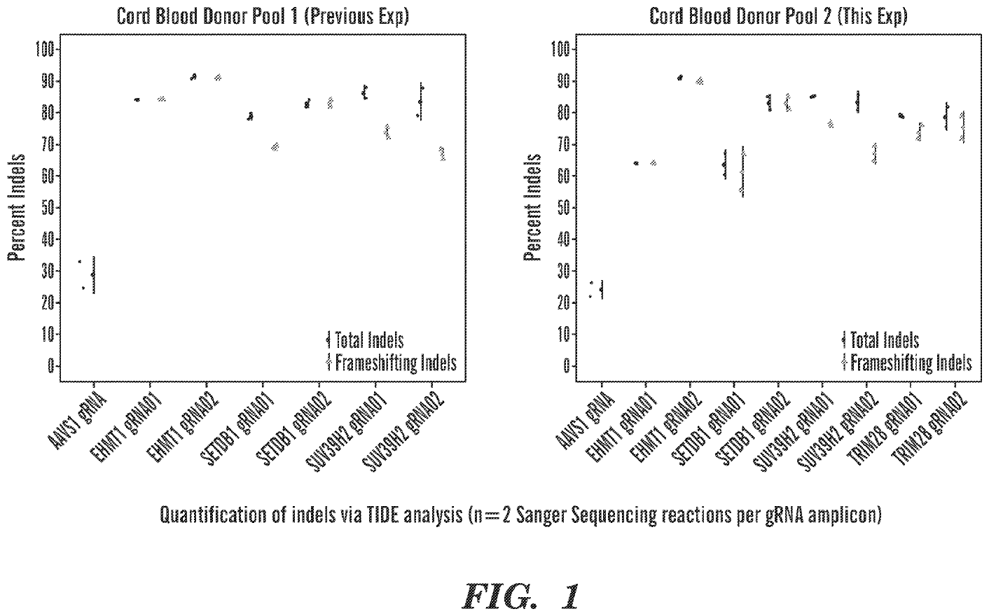

### DETAILED DESCRIPTION

The various aspects described herein are based, in part, on the inventors' discovery of a method for generating a natural killer (NK) cell by differentiating a pluripotent stem cell engineered to lack TRIM28 expression and/or activity for a sufficient time to promote differentiation to a CD56+NK cell.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed embodiments, because the scope of the invention is limited only by the claims. Unless otherwise defined, all technical and scientific terms used herein have the same meaning as commonly understood by one of ordinary skill in the art to which this invention(s) belongs. If there is an apparent discrepancy between the usage of a term in the art and its definition provided herein, the definition provided within the specification shall prevail.

Unless otherwise defined herein, scientific and technical terms used in connection with the present application shall have the meanings that are commonly understood by those of ordinary skill in the art to which this disclosure belongs. It should be understood that embodiments of the invention are not limited to the particular methodology, protocols, and reagents, etc., described herein and as such can vary. The terminology used herein is for the purpose of describing particular embodiments only, and is not intended to limit the scope of the methods and compositions described herein, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “decrease”, “reduced”, “reduction”, or “inhibit” are all used herein to mean a decrease by a statistically significant amount. In some embodiments of any of the aspects described herein, the terms “reduce,” “reduction” or “decrease” or “inhibit” typically mean a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments of any of the aspects described herein, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein “culturing under conditions and for a sufficient time to promote differentiation to an NK cell,” refers to the conditions needed to promote differentiation of a pluripotent stem cell that lacks TRIM28 expression and/or activity to an NK cell (e.g., a CD56+ NK cell). It is shown herein that inhibition or deletion of TRIM28 in pluripotent stem cells results in induction of NK cells under a variety of culture conditions. For example, the pluripotent stem cell can be grown using a method that does or does not comprise co-culturing with stromal cells or any other type of supporting cell, such as a mouse embryonic fibroblast (MEF). The pluripotent stem cell can be grown in NK-cell-differentiation media or CD3+-T-cell differentiation media. Alternatively, the pluripotent stem cell can be grown in single-positive-T-cell-differentiation media. The pluripotent stem cell can be cultured in the differentiation medium for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, at least 56 days, at least 57 days, at least 58 days, at least 59 days, at least 60 days, at least 61 days, at least 62 days, at least 63 days, at least 64 days, at least 65 days, at least 66 days, at least 67 days, at least 68 days, at least 69 days, at least 70 days, at least 71 days, at least 72 days, at least 73 days, at least 74 days, at least 75 days, at least 76 days, at least 77 days, at least 78 days, at least 79 days, at least 80 days, at least 81 days, at least 82 days, at least 83 days, at least 84 days, at least 85 days, at least 86 days, at least 87 days, at least 88 days, at least 89 days, at least 90, at least 91 days, at least 92 days, at least 93 days, at least 94 days, at least 95 days, at least 96 days, at least 97 days, at least 98 days, at least 99 days, at least 100 days, at least 101 days, at least 102 days, at least 103 days, at least 104 days, at least 105 days, at least 106 days, at least 107 days, at least 108 days, at least 109 days, at least 110 days, at least 111 days, at least 112 days, at least 113 days, at least 114 days, at least 115 days, at least 116 days, at least 117 days, at least 118 days, at least 119 days, at least 120 days, at least 121 days, at least 122 days, at least 123 days, at least 124 days, at least 125 days, at least 126 days, at least 127 days, at least 128 days, at least 129 days, at least 130 days, at least 131 days, at least 132 days, at least 133 days, at least 134 days, at least 135 days, at least 136 days, at least 137 days, at least 138 days, at least 139 days, at least 140 days, at least 141 days, at least 142 days, at least 143 days, at least 144 days, at least 145 days, at least 146 days, at least 147 days, at least 148 days, at least 149 days, at least 150 days, at least 151 days, at least 152 days, at least 153 days, at least 154 days, at least 155 days, at least 156 days, at least 157 days, at least 158 days, at least 159 days, at least 160 days, at least 161 days, at least 162 days, at least 163 days, at least 164 days, at least 165 days, at least 166 days, at least 167 days, at least 168 days, at least 169 days, at least 170 days, at least 171 days, at least 172 days, at least 173 days, at least 174 days, at least 175 days, at least 176 days, at least 177 days, at least 178 days, at least 179 days, at least 180 days, at least 181 days, at least 182 days, at least 183 days, at least 184 days, at least 185 days, at least 186 days, at least 187 days, at least 188 days, at least 189 days, at least 190 days, at least 191 days, at least 192 days, at least 193 days, at least 194 days, at least 195 days, at least 196 days, at least 197 days, at least 198 days, at least 199 days, at least 200 days, at least 201 days, at least 202 days, at least 203 days, at least 204 days, at least 205 days, at least 206 days, at least 207 days, at least 208 days, at least 209 days, at least 210 days, at least 211 days, at least 212 days, at least 213 days, at least 214 days, at least 215 days, at least 216 days, at least 217 days, at least 218 days, at least 219 days, at least 220 days, at least 221 days, at least 222 days, at least 223 days, at least 224 days, at least 225 days, at least 226 days, at least 227 days, at least 228 days, at least 229 days, at least 230 days, at least 231 days, at least 232 days, at least 233 days, at least 234 days, at least 235 days, at least 236 days, at least 237 days, at least 238 days, at least 239 days, at least 240 days, at least 241 days, at least 242 days, at least 243 days, at least 244 days, at least 245 days, at least 246 days, at least 247 days, at least 248 days, at least 249 days, at least 250 days, at least 251 days, at least 252 days, at least 253 days, at least 254 days, at least 255 days, at least 256 days, at least 257 days, at least 258 days, at least 259 days, at least 260 days, at least 261 days, at least 262 days, at least 263 days, at least 264 days, at least 265 days, at least 266 days, at least 267 days, at least 268 days, at least 269 days, at least 270 days, at least 271 days, at least 272 days, at least 273 days, at least 274 days, at least 275 days, at least 276 days, at least 277 days, at least 278 days, at least 279 days, at least 280 days, at least 281 days, at least 282 days, at least 283 days, at least 284 days, at least 285 days, at least 286 days, at least 287 days, at least 288 days, at least 289 days, at least 290 days, at least 291 days, at least 292 days, at least 293 days, at least 294 days, at least 295 days, at least 296 days, at least 297 days, at least 298 days, at least 299 days, at least 300 days, at least 301 days, at least 302 days, at least 303 days, at least 304 days, at least 305 days, at least 306 days, at least 307 days, at least 308 days, at least 309 days, at least 310 days, at least 311 days, at least 312 days, at least 313 days, at least 314 days, at least 315 days, at least 316 days, at least 317 days, at least 318 days, at least 319 days, at least 320 days, at least 321 days, at least 322 days, at least 323 days, at least 324 days, at least 325 days, at least 326 days, at least 327 days, at least 328 days, at least 329 days, at least 330 days, at least 331 days, at least 332 days, at least 333 days, at least 334 days, at least 335 days, at least 336 days, at least 337 days, at least 338 days, at least 339 days, at least 340 days, at least 341 days, at least 342 days, at least 343 days, at least 344 days, at least 345 days, at least 346 days, at least 347 days, at least 348 days, at least 349 days, at least 350 days, at least 351 days, at least 352 days, at least 353 days, at least 354 days, at least 355 days, at least 356 days, at least 357 days, at least 358 days, at least 359 days, at least 360 days, at least 361 days, at least 362 days, at least 363 days, at least 364 days, at least 365 days in the same medium or medium that has been replaced over time. In some embodiments, the cells as described herein can be grown in a medium that promotes lymphoid progenitor expansion and differentiation into T cells, optionally in a tissue culture plate that is coated with a coating material to promote lymphoid cells to adhere to tissue culture plates. The medium can be serum free or can comprise serum. The media can include, but should not be limited to the following: Iscove's Modified Dulbecco's Medium (MDM), Bovine Serum Albumin, recombinant human insulin, human transferrin (iron-saturated), 2-mercaptoethanol, and additional supplements. The medium can contain additional growth factors and/or supplements. In some embodiments, a medium that promotes lymphoid progenitor expansion and differentiation into NK cells, preferably coated with a coating material to promote lymphoid cells to adhere to tissue culture plates. The medium can be serum free. The medium can include, but is not limited to the following: Iscove's Modified Dulbecco's Medium (MDM), Bovine Serum Albumin, recombinant human insulin, human transferrin (iron-saturated), 2-mercaptoethanol, and additional supplements.

As used herein, the term “contacting a cell with an inhibitor of TRIM28,” refers to the placement or introduction of, for example, an inhibitor of TRIM28 on or into a cell(s) by a method or route which results in at least partial inhibition of expression and/or activity of TRIM28 in the cell. In some embodiments, TRIM28 expression or activity is decreased in a cell by at least 10% following a step of contacting the cell with a TRIM28 inhibitor as compared to a substantially similar cell that is not treated with the TRIM28 inhibitor. In other embodiments, TRIM28 expression or activity is decreased in a cell by at least 20%, at least 30%, at least 40%, at least 50%, at least 60%, at least 70%, at least 80%, at least 90%, at least 95%, at least 99%, or even 100% (e.g., TRIM28 expression or activity is below detectable levels or absent in the cell) as compared to a substantially similar cell that is not treated with the TRIM28 inhibitor. Contacting of cells can be ex vivo or in vitro.

As used herein, the term “engineered to lack TRIM28” refers to a process that alters or deletes one or more genes or gene expression products that encode TRIM28, such that the cell lacks TRIM28 expression and/or activity. In some embodiments, the term “engineered to lack TRIM28” can also be applied to progeny of a parental cell engineered to lack TRIM28. In some embodiments, the pluripotent stem cell has been engineered to lack TRIM28 using a genomic modification system such as a CRISPR/Cas9 genomic modification system. Cells that are engineered to lack TRIM28 can be verified using techniques known in the art such as fluorescence activated cell sorting (FACS), ELISA (enzyme linked immunosorbent assay), western blot, immunoprecipitation, and immunofluorescence which are described herein.

As used herein the term “CAR NK cell” refers to an NK cell made by the methods described herein that is further modified to express a chimeric antigen receptor (CAR) and which can be used as an anti-cancer therapy. These receptors can be both antigen binding and NK-cell activating receptors and can bind to a cell-surface ligand (e.g., a tumor antigen). A CAR NK cell is an exemplary therapeutic cell composition that can be used in the treatment of cancer or other states of immunodeficiency in a subject.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but not limited to, siRNA, shRNA, microRNA, a double stranded RNA (dsRNA), and the like. RNA interference is known to those of skill in the art and as such is not described in detail herein.

In some embodiments of any of the aspects, RNAi is mediated by an inhibitory RNA (iRNA). The iRNA can be single stranded or double stranded. The iRNA can be dsRNA, siRNA, shRNA, endogenous microRNA (miRNA), or artificial miRNA. In some embodiments, the iRNA mediates the targeted cleavage of an RNA transcript via an RNA-induced silencing complex (RISC) pathway. An iRNA as described herein effects inhibition of the expression and/or activity of a target, e.g., TRIM28. In some embodiments of any of the aspects described herein, contacting a cell with the inhibitor nucleic acid (e.g., an iRNA) results in a decrease in the TRIM28 mRNA level in a cell by at least about 5%, about 10%, about 20%, about 30%, about 40%, about 50%, about 60%, about 70%, about 80%, about 90%, about 95%, about 99%, up to and including 100% (e.g., below detectable levels) of the TRIM28 mRNA level found in the cell without the presence of the iRNA.

As used herein, the term “histone methyltransferase inhibitor” is any molecule that inhibits expression of a histone methyltransferase (e.g., SETDB1, HP1), or inhibits the catalytic activity of the enzyme to methylate the lysine resides on the substrate histone protein. TRIM28 is known to interact with histone methyltransferases including, but not limited to, SETDB1 and HP1. In one embodiment, the histone methyltransferase inhibitor reduces the expression and/or activity of a histone methyltransferase in a cell by at least 10% as compared to a substantially similar cell that is not contacted with the inhibitor. A histone methyltransferase inhibitor can be an siRNA or dsRNA that inhibits expression of SETDB1 and/or HP lin the inhibited cell, or a guide RNA-mediated system that promotes the degradation of the mRNA of SETDB1 and/or HP1in the inhibited cell. In one embodiment, a histone methyltransferase inhibitor can be a small molecule that antagonizes the enzyme activity. Examples include, but are not limited to, small molecules AMI-1, A-366, BIX-01294, BIX01338, BRD4770, chaetocin, UNC0224, UNC0631, UNC0638, UNC0642, UNC0646, EPZ5676, EPZ005687, GSK343, EPZ-6438, 3-deazaneplanocin A (DZNeP) HCl, UNC1999, MM-102, SGC 0946, Entacapone, EPZ015666, UNC0379, Ell, MI-2 (Menin-MLL Inhibitor), MI-3 (Menin-MLL Inhibitor), PFI-2, GSK126, EPZ004777, BRD4770, and EPZ-6438 as described herein.

As used herein, the term “TRIM28 binding partner” refers to a polypeptide (e.g., an endogenous polypeptide) that interacts directly or indirectly with TRIM28 and promotes a function in the cell (e.g., transcription). In one embodiment, the TRIM28 binding partner includes, but is not limited to, KRAB-ZNF transcription factors, MDM2, p53, the NuRD complex (comprising of NuRD, Mi2α, and an HDAC), SETDB1, CBF-A, and HP1. TRIM28 binding partners can be stable or transient interactions. TRIM28 binding partners can be strong or weak interactions. TRIM28 binding partners can be a part of a multi-subunit complex and the subunits of these complexes can be identical or different (e.g., SETDB1-TRIM28 complex). TRIM28 binding partners can be stimulated by environmental conditions such as post-translational modifications, conformational changes in TRIM28 and/or its binding partner, or localization to a distinct area of the cell. TRIM28 binding partners can be involved in cellular processes (e.g., histone methylation). TRIM28 binding partners can be disrupted by targeting their bonds (e.g., hydrophobic bonding, van der Waals forces, salt bridges) at binding domains. TRIM28 binding partners can be examined through a variety of techniques known in the art such as ELISA (enzyme linked immunosorbent assay), western blot, immunoprecipitation, and immunofluorescence which are described herein. Blocking peptides specific to TRIM28 can be found commercially and exemplary examples include, but are not limited to TRIM28/KAP1 antibody blocking peptide (Cat. No. LS-E29986, LS Bio, Seattle, WA), KAP1 blocking peptide (Cat. No. GTX31274-PEP, GeneTex, Irvine, CA), TRIM28 (extracellular) blocking peptide (Cat. No. BLP-NR018, Alomone Labs, Jerusalem, Israel).

As used herein, “inhibitor of TRIM28” refers to a molecule or compound which can decrease the expression and/or activity of TRIM28, e.g., by at least 10% as compared to TRIM28 expression and/or activity in a substantially similar cell that is not treated with the TRIM28 inhibitor, e.g., at least 20%, at least 30%, at least 40%, at least 50%, at least 60%, at least 70%, at least 80%, at least 90%, at least 95%, at least 95%, at least 99% or even 100% (i.e., the absence of TRIM28 activity or reduction of TRIM28 activity below detectable levels using an RT-PCR assay). Exemplary methods of contacting include inhibitory molecules preventing contact between TRIM28 and one of its binding partners.

As used herein, the term “molecular glue” refers to a small molecule that interacts with a protein surface on a first and second protein to induce or enhance affinity of these two proteins (i.e., the first and second protein) to each other. Molecular glues can be used to induce or maintain protein-protein interactions, specifically with proteins that are a TRIM28 binding partner. Molecular glues can act on TRIM28 along with an accessory protein, such as a substrate adaptor protein, presenter protein, or effector protein. Molecular glues can enhance protein surfaces on TRIM28 that otherwise would be inaccessible by other small molecules and/or proteins. Exemplary examples of molecular glues include, but are not limited to, cyclosporine A, cyclophilin A glues, voclosporine, sanglifehrin, FK506, and FK520. In some embodiments, molecular glues can be used to disrupt TRIM28 expression and/or activity.

The term “degrader” or “dTAG” refers to a degradation tag system that utilizes chemical conjugation with derivatized phthalimides that harness the function of the Cereblon E3 ubiquitin ligase complex (dTAG) for target-specific protein degradation. By expressing a FKBP12 functional mutant in frame with a protein of interest (e.g., TRIM28), the dTAG will degrade FKBP12 and the protein of interest. This system works for targeting a specific protein of interest (e.g., TRIM28) and is a rapid and reversible form of degradation, which is beneficial in systems where the permanent loss of the protein is not permissible. Examples of dTAGs include, but are not limited to, dTAG-12, dTAG-7, dTAG-13, and dTAG-48 (Nabet et al, 2018. Nat Chem Biol. 14(5): 431-441).

The term “cellular replacement therapy” refers to administration of a cell (e.g., an NK cell or a CAR NK cell) to a subject in need thereof. In some embodiments, an NK cell is administered to replace NK cells that are lost, for example, due to chemotherapy treatment or another treatment that induces immunodeficiency. NK cell replacement can not only restore the original function of the NK cells lost during treatment, but they can also be enhanced to target specific antigens (e.g., the addition of CAR-NK cells and/or natural-killer cell engager (NKCEs) to target tumor cells). In some embodiments, NK cells generated from pluripotent stem cells that lack TRIM28 expression and/or activity can be used to replace or augment NK cells in a subject. It is also specifically contemplated herein that an NK cell generated as described herein can comprise further genetic modifications as desired.

The term “knockin” as used herein refers to a gene sequence is inserted at a particular locus. Knockins can be used in any research field. Different types of knockins can include, but not be limited to, constitutive knockins, humanization knockins, reporter/tag knockins, and targeting transgenics that utilize a particular locus which provides full control of the gene expression. This targeted insertion can result in a genetic mutation. In some embodiments, a knockin can be used to visualize TRIM28 expression and/or activity. In other embodiments, a knockin can be used to disrupt TRIM28 expression and/or activity.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example.”

All references provided herein are incorporated by reference in their entirety.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### TRIM28

Tripartite motif-containing 28 (TRIM28) mediates transcriptional control by interaction with the Krüppel-associated box repression domain found in transcription factors. It localizes to the nucleus and associates with specific chromatin groups. The protein contains three zinc binding domains, a RING domain, a B-box type 1, a B-box type 2, and a coiled-coil region. TRIM28 is ubiquitously expressed and its functions include transcriptional regulation, cellular differentiation and proliferation, DNA damage repair, viral suppression, and apoptosis. Most of these functions are dependent on post-translational modifications.

TRIM28 can repress transcription by binding directly to the genome (both with and without the need for binding partners) or through the induction of heterochromatin formation via the Mi2α-SETDB1-HP1 macromolecular structure. It can also interact with histone methyltransferases and deacetylases via its C-terminal plant homeodomain (PHD) and bromodomain in order to control transcription epigenetically. Thus, where TRIM28 is a transcriptional repressor, it follows that inhibition of TRIM28 can enhance transcription in a cell.

TRIM28 referred to in this aspect, and all aspects and embodiments described herein in this application, can comprise the nucleotide sequences of: SEQ ID NO. 1, SEQ ID NO. 2, and SEQ ID NO. 3, or any fragment or portion thereof.

### Pluripotent and Multipotent Stem Cells

Any pluripotent stem cell can be engineered or treated to lack TRIM28 expression and/or activity as part of a method to generate NK cells as described herein. Pluripotent stem cells retain the capacity, under different conditions, to differentiate to cell types characteristic of all three germ cell layers (endoderm, mesoderm and ectoderm) as assessed using for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers. Exemplary pluripotent stem cells that can be used with the methods described herein comprise embryonic stem cells, cord blood cells, bone marrow cells, and induced pluripotent stem cells.

As used herein, the terms “induced pluripotent stem cell,” “iPSC,” “hiPSC,” and “human induced pluripotent stem cell” are used interchangeably to refer to a pluripotent cell artificially derived from a differentiated somatic cell (e.g., a fibroblast). iPSCs are capable of self-renewal and differentiation into cell hematopoietic stem cells, including cells of the lymphoid lineages, as well as various types of mature cells. To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. Methods for detecting the expression of such markers can include, for example, RT-PCR and immunological methods that detect the presence of the encoded polypeptides, such as western blot, which is described herein.

In one embodiment, the stem cell or progenitor cell engineered to lack TRIM28 or treated to inhibit TRIM28 is a CD34+ hemogenic endothelial cell. As used herein, a CD34+ hemogenic endothelial cell refers to a transient, specialized endothelial cell with the capacity to generate hematopoietic and progenitor cells. These cells then have the potential to generate into different lineages (including, but not limited to, lymphoid and myeloid). These cells can originate from the human umbilical blood cord.

In some embodiments, a multipotent stem cell can be engineered or treated to lack TRIM28 expression and/or activity as part of a method to generate NK cells as described herein. Some non-limiting examples of multipotent stem cells include hematopoietic stem cells, and mesenchymal stem cells.

As used herein, the term “hematopoietic stem cell” or “HSC” refers to a stem cell that can give rise to all the blood cell types of the three hematopoietic lineages, erythroid, lymphoid, and myeloid. These cell types include the myeloid lineages (monocytes and macrophages, neutrophils, basophils, eosinophils, erythrocytes, megakaryocytes/platelets, dendritic cells), and the lymphoid lineages (T-cells, B-cells, NK-cells).

In one embodiment, natural killer cells derived as described herein are differentiated from pluripotent or multipotent stem cells that are obtained directly from the subject to whom they will be administered (i.e., autologous transplantation). As used herein, “autologous” refers to deriving cells from the same sample or subject. Thus, in some embodiments, the NK cells used can be derived from the subject to be treated.

In another embodiment, the therapeutic NK cells generated as described herein can be non-autologous or allogeneic. As used herein, “allogeneic” refers to cells (e.g., pluripotent stem cells or multipotent stem cells) obtained from one or more different donors of the same species, where the genes at one or more loci are not identical. For example, a therapeutic NK cell composition being administered to a subject can be derived from umbilical cord blood obtained from one or more unrelated donor subjects, or from one or more non-identical siblings. Another example of a therapeutic NK cell composition being administered to a subject can be derived from bone marrow obtained from one or more unrelated donor subjects, or from one or more non-identical siblings. In some embodiments, syngeneic stem cell populations can be used to generate therapeutic NK cells, such as those obtained from genetically identical animals, or from identical twins. For non-autologous transplantation, the recipient can be treated with an immunosuppressive drug to reduce the risk of rejection of the transplanted cell, if necessary.

### Engineering Cells to Lack TRIM28

Methods of generating NK cells as described herein can include a step of engineering a pluripotent or multipotent stem cell to lack TRIM28 prior to differentiation of such cells to NK cells. The term “engineered” can be used interchangeably with “genetic manipulation” or “genetic modification” to refer to a change in the genetic and/or epigenetic makeup of a cell introduced by the hand of man, and includes, for example, gene editing, which changes the chromosomal DNA of the cell. While any site-directed mutagenesis approach for the modification of chromosomal DNA can be used, non-limiting examples of gene editing include CRISPR-Cas mediated chromosomal cleavage, with or without the use of a homologous recombination template, inheritable epigenetic silencing (so-called “CRISPRoff”), base editing, prime editing, and zinc-finger nuclease or TALEN-mediated cleavage of a target sequence or sequences, also with or without the use of a homologous recombination or replacement template, as well as other gene editing systems as described herein. Such methods can be employed herein to inactivate TRIM28 expression by modified the chromosomal DNA such that the target gene is not expressed.

Such inactivation can include deletion of all or a portion of TRIM28 or its coding sequence, insertion of a sequence that disrupts expression of TRIM29, or replacement of a coding sequence with that encoding another polypeptide, among others. For example, genetic modification can refer to alterations, additions, and/or deletion of genes or portions of genes. A genetically modified cell can also refer to a cell with an added, deleted and/or altered gene or portion of a gene (e.g., TRIM28). A genetically modified cell can also refer to a cell with an added nucleic acid sequence that is not a gene or gene portion.

In one embodiment, a genomic modification system (e.g., CRISPR/Cas) can be used to reduce or eliminate expression of TRIM28 in a cell. One of skill in the art can readily apply the principles of CRISPR/Cas mediated genomic modification to modulate expression and/or activity of TRIM28. Any CRISPR-associated nuclease can be used with the methods and compositions described herein. CRISPR nuclease systems are known to those of skill in the art, and include but are not limited to, Cas9, Cas12, Cas12a, or the like, see e.g., patents/applications 8,993,233, US 2015/0291965, US 2016/0175462, US 2015/0020223, US 2014/0179770, 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; 8,865,406; 8,871,445; each of which are incorporated by reference in their entirety. The nuclease can also be a phage Cas nuclease, e.g., CasΦ (e.g., Pausch et al. Science 369:333-7 (2020); which is incorporated by reference herein in its entirety).

Typically, the term “CRISPR/Cas system” refers collectively to transcripts and other elements involved in the expression of or direction of the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is/are derived from a type I, type II, or type III CRISPR system. In some embodiments, one or more elements of a CRISPR system is derived from a particular organism comprising an endogenous CRISPR system, such as Streptococcus pyogenes. In some embodiments, the CRISPR/Cas system involves a ‘base editing system’ or a ‘prime editing system’ using modified conventional Cas endonucleases to change specific bases without cutting both strands of DNA.

A CRISPR system is typically characterized by elements that promote the formation of a CRISPR complex at the site of a target sequence. In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides.

As used herein, the terms “guide RNA” or “gRNA” refer to a nucleic acid comprising a sequence that determines the specificity of an enzyme, e.g., the Cas DNA binding protein of a CRISPR/Cas system, to a polynucleotide target. The gRNA (e.g., guide RNA) can comprise a polynucleotide sequence with at least partial complementarity with a TRIM28 nucleic acid sequence, sufficient to hybridize with the TRIM28 nucleic acid sequence and to direct sequence-specific binding of an enzyme, e.g, a nuclease, to the TRIM28 nucleic acid sequence, to induce insertions, deletions, indels, and/or mutations of a target, thereby reducing the expression of TRIM28. Exemplary gRNAs for TRIM28 include, but are not limited to, CCAGCGGGTGAAGTACACCA and CTTCCCAGGCAGTACCACTG.

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off_Spotter™; and Synthego™; which are available on the world wide web).

Alternatively, genomic modification can be performed using one or more of meganucleases, Zinc finger nucleases (ZFNs), and/or transcription-activator like effector nucleases (TALENs).

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29(18): 3757-3774). The LAGLIDADG meganucleases with a single copy of the LAGLIDADG motif form homodimers, whereas members with two copies of the LAGLIDADG are found as monomers. Similarly, the GlY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). In the case of the NHN family, the members are defined by motifs containing two pairs of conserved histidines surrounded by asparagine residues (see Chevalier et al. (2001), Nucleic Acids Res. 29(18): 3757-3774). The four families of meganucleases are widely separated from one another with respect to conserved structural elements and, consequently, DNA recognition sequence specificity and catalytic activity.

Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867, the contents of which are incorporated herein by reference in its entirety). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision BioSciences' Directed Nuclease Editor™ genome editing technology.

ZFN and TALEN restriction endonuclease technology utilizes a non-specific DNA cutting enzyme, which is linked to a specific DNA sequence recognizing peptide(s) such as zinc fingers and transcription activator-like effectors (TALEs). Typically, an endonuclease whose DNA recognition site and cleaving site are separate from each other is selected and its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be used to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, CA).

Alternatively, genome editing can be performed using recombinant adeno-associated virus (rAAV) based genome engineering, which is a genome-editing platform centered around the use of rAAV vectors and that enables insertion, deletion or substitution of DNA sequences into the genomes of live mammalian cells. The rAAV genome is a single-stranded deoxyribonucleic acid (ssDNA) molecule, either positive- or negative-sensed, which is about 4.7 kilobase long. These single-stranded DNA viral vectors have high transduction rates and can stimulate endogenous homologous recombination in the absence of causing double strand DNA breaks in the genome. One of skill in the art can design a rAAV vector to target a desired genomic locus and perform both gross and/or subtle endogenous gene alterations in a cell, such as a deletion. rAAV genome editing has the advantage in that it targets a single allele and does not result in any off-target genomic alterations. rAAV genome editing technology is commercially available, for example, the rAAV GENESIS™ system from Horizon™ (Cambridge, UK).

### Inhibiting TRIM28 Expression and or Activity

In some embodiments, TRIM28 expression and/or activity is modulated transiently at either the RNA or protein level. That is, a TRIM28 inhibitor can be used to reduce or inhibit TRIM28 expression and/or activity and does not involve alteration of the TRIM28 at the genomic level. Exemplary inhibitors of TRIM28 that can transiently modulate TRIM28 expression and/or activity include, for example, small molecules, peptides, or RNA interference (RNAi) molecules, a class of genetic control approaches involving double- or single-stranded RNAs including, but not limited to siRNA, shRNA, miRNA, that function through the RNA-induced silencing complex (RISC) to inhibit expression of target genes.

In some embodiments, inhibition of TRIM28 expression and/or activity need not be permanent. For example, it can be beneficial to knock down expression of TRIM28 with e.g., RNA-specific Cas nuclease, antisense expression, etc. RNAi or other inhibitory molecules can be administered to the pluripotent or multipotent stem cell (e.g., in any of a number of different lipid complexes, among other delivery options), or can alternatively be expressed from a construct that is administered to or contacted with the pluripotent or multipotent stem cell. In one embodiment, such cells can be transiently transfected with one or more constructs encoding an RNAi molecule (e.g., encoding expression of an shRNA); in such instances, it is anticipated that over time, and absent active selection for the construct, the transfected construct would be lost, providing transient expression of the inhibitor.

In some embodiments of any of the aspects described herein, the agent that inhibits TRIM28 is an inhibitory nucleic acid. Exemplary inhibitor nucleic acids include, but are not limited to, double-stranded RNAs (dsRNAs), inhibitory RNAs (iRNAs), a small interfering RNA (siRNA), microRNA (miRNA), or short hairpin RNA (shRNA). Double-stranded RNA molecules (dsRNA) have been shown to block gene expression in a highly conserved regulatory mechanism known as RNA interference (RNAi). The use of these iRNAs enables the targeted degradation of mRNA transcripts, resulting in decreased expression and/or activity of TRIM28. One skilled in the art can design a further siRNA, shRNA, or miRNA to target the nucleic acid sequence of TRIM28 (e.g., SEQ ID NO: 1), e.g., using publicly available design tools. siRNA, shRNA, or miRNA is commonly made using companies such as Dharmacon (Layfayette, CO) or Sigma Aldrich (St. Louis, MO).

In some embodiments of any of the aspects described herein, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., TRIM28, it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive. Similarly, the region of complementarity to the TRIM28 sequence is between 15 and 30 base pairs in length inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193).

In some embodiments, a TRIM28 inhibitor for use as described herein comprises a small molecule. As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the small molecule is a heterorganic compound or an organometallic compound.

Where TRIM28 is an E3 ligase, small molecule inhibitors of E3 ligases can be also be used to inhibit TRIM28 activity and/or expression. E3 ligase inhibitors prevent the transfer or the placement of ubiquitin onto E1 activating enzymes, E2 conjugating enzymes, E3 ubiquitin ligases, or their downstream targets or contributes to the removal of ubiquitin from E1 activating enzymes, E2 conjugating enzymes, E3 ubiquitin ligases, or their downstream targets. E3 ligase inhibitors are able to decrease the activity of activating, conjugating, and/or forming ubiquitin chains by at least 10%, at least 20%, at least 30%, at least 40%, at least 50%, at least 60%, at least 70%, at least 80%, at least 90%, at least 100% of the endogenous E3 ligase (e.g., TRIM28). The activation, conjugation, and/or forming ubiquitin chains can be analyzed using western blot or other immunological assays described herein. In one embodiment, the E3 ligase inhibitor targets TRIM28 expression and/or activity. Exemplary E3 ligase inhibitors include, but are not limited to, thalidomide, proTAME, NSC 66811, Nutlin 3, HLI 373, JNJ 26854165, SMER 3, heclin, A01, Apcin, CSN5i-3, GS 143, Idasanutlin, Nimbolide, and PRT 4165.

Another method of inhibiting the expression and/or activity of a protein (e.g., TRIM28) is through the use of proteolysis-targeting chimeras (PROTACs). PROTACs engage both an E3 ubiquitin ligase and a target protein meant for degradation. PROTACs can degrade at least 10%, at least 20%, at least 30%, at least 40%, at least 50%, at least 60%, at least 70%, at least 80%, at least 90%, at least 100% of their target protein. Degradation of a target can be measured using western blot or other immunological assays described herein. PROTACs bind to their targets with high selectivity rather than inhibit the target protein's enzymatic activity. A PROTAC comprises a E3 ligase and a linker to target the protein of interest (e.g., TRIM28). In one embodiment, a PROTAC can disrupt TRIM28 expression and/or activity. Exemplary PROTACs include, but are not limited to, CRBN, DCAF15, IAP, MDM2, VHL (as E3 ligases), PEGs, alkyl-chain, and alkyl/ether (as linkers).

In some embodiments, the methods described herein further comprise contacting a cell with one or more additional inhibitors (e.g., an inhibitor that targets sumoylation). As used herein, the term “inhibitor that targets sumoylation” refers to any molecule that inhibits the transfer or the placement of small ubiquitin-like modifiers (SUMOs) including SUMO1, SUMO2, and/or SUMO3 onto E2 conjugating enzymes, E3 ligases, or their downstream targets or contributes to the removal of SUMO1, SUMO2, and/or SUMO3 from E2 conjugating enzymes, E3 ligases, or their downstream targets. At a minimum, the “inhibitor that targets sumoylation” will reduce at least one of these activities in a cell contacted with such an inhibitor by at least 10% as compared to the level of the same activity in a substantially similar cell that is not contacted with an inhibitor that targets sumoylation.

Small molecule inhibitors for sumoylation include, but are not limited to TAK-981, Ginkgolic acid, Kerriamycin B, Compound 21, Davidiin, Tannic acid, ML-792, GSK145A, 2-D08, Spectomycin B1, and Compound 2, N-[1-butyl-3-(4-methylphenyl)sulfonylpyrrolo[3,2-b]quinoxalin-2-yl]-4-methoxybenzamide, SCHEMBL17445226, SCHEMBL17445231, MLS-0437119.0001, SCHEMBL17445215, HMS2174I04, 2-amino-1-butyl-1H-pyrrolo[2,3-b]quinoxaline-3-carbonitrile, MLS-0437282.0001, MLS-0437281.0001, MLS-0437278.0001, MLS-0437122.0001, Oprea1_342954, SMR000060157, MLS000054842, MLS000068755, SCHEMBL17445220, SCHEMBL17445221, MLS-0437115.0001, MLS-0437112.0001, MLS-0437315.0001, SCHEMBL17445217, MLS-0437322.0001, MLS-0437321.0001, MLS-0437319.0001, MLS-0437317.0001, MLS-0437313.0001, SCHEMBL21663654, MLS-0437318.0001, MLS-0437316.0001, SCHEMBL17445218, MLS-0437314.0001, MLS-0437129.0001, MLS-0437128.0001, SCHEMBL17445233, SCHEMBL17445232, MLS-0437125.0001, MLS-0437124.0001, MLS-0437123.0001, CHEMBL1727666, N-(1-butyl-3-cyano-1H-pyrrolo[2,3-b]quinoxalin-2-yl)-4-chlorobenzamide, Oprea1_774457, SCHEMBL17445214, MLS-0435771.0001, STK876314, STK876313, MLS-0435709.0001, AKOS022131277, CHEMBL1519689, MLS000555532, MLS000595294, SMR000194740, MLS000588660, MLS000079917, MLS000054842, ZINC2318750, SCHEMBL17445213, SCHEMBL17445219, SCHEMBL17445228, MLS-0437107.0001, MLS-0437105.0001, MLS-0437104.0001, ZINC12868093, ZINC2308212, Oprea1_124958, AKOS005156744, and SCHEMBL15256902.

### Measuring TRIM28 Expression and or Activity

Methods to measure TRIM28-specific gene expression products are known to a skilled artisan and can include, but are not limited to: flow cytometry, fluorescence activated cell sorting, live ELISA (enzyme linked immunosorbent assay), western blot, immunoprecipitation, and immunofluorescence using detection reagents such as an antibody or protein binding agents. In some embodiments, an anti-TRIM28 antibody is optionally labeled with a detectable marker for ease of detection and/or measurement of TRIM28 expression.

In some embodiments, fluorescence activated cell sorting or “FACS” can be used in combination with an antibody or antigen binding fragment thereof to detect cells that have been engineered to lack TRIM28 expression.

For example, anti-TRIM28 antibodies described herein are commercially available and can be used with the methods and compositions described herein to measure protein expression levels of TRIM28 (Cat. No. MA1-2023; Invitrogen, Carlsbad, CA), anti-KAP1 (Cat. No. 1B9G12, Proteintech, Rosemont, IL), anti-KAP1 (TRIM28) (Cat. No. OTI2H10, OriGene, Rockville, MD), anti-KAP-1 (Cat. No. BL-248-2G6, Bethyl Laboratories, Montgomery, TX). Alternatively, since the amino acid sequences for TRIM28 described herein are known and publicly available at the NCBI website, one of skill in the art can raise their own antibodies against these polypeptides of interest for the purpose of the methods described herein.

The amino acid sequences of the polypeptides described herein have been assigned NCBI accession numbers for different species such as human, mouse and rat. In particular, the NCBI accession numbers for the amino acid sequence of human TRIM28 is included herein, e.g., SEQ ID NO. 2.

In some embodiments of any of the aspects, immunohistochemistry (“IHC”) and immunocytochemistry (“ICC”) techniques can be used to assess the expression, or lack thereof, of TRIM28 in a population of cells treated as described herein. IHC is the application of immunochemistry to tissue sections, whereas ICC is the application of immunochemistry to cells or tissue imprints after they have undergone specific cytological preparations such as, for example, liquid-based preparations. Both IHC and ICC typically use antibodies directed against a desired target molecule (e.g., TRIM28) inside or on the surface of cells.

While methods that can be performed in intact cells are preferred, in some embodiments of any of the aspects described herein, the assay can be a western blot analysis, for example, of a portion of a cell population engineered or treated to lack TRIM28 expression. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as radioimmunoassay (RIA), ELISA (enzyme linked immunosorbent assay), “sandwich” immunoassays, immunoprecipitation assays, immunodiffusion assays, agglutination assays, e.g., latex agglutination, complement-fixation assays, immunoradiometric assays, fluorescent immunoassays, e.g., FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay.

In some embodiments, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, one or more of the reagents (e.g., an antibody reagent and/or nucleic acid probe) described herein can comprise a detectable label and/or comprise the ability to generate a detectable signal (e.g., by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g., antibodies and nucleic acid probes) are well known in the art. In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means.

In other embodiments, the detection reagent is label with a fluorescent compound. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of any of the aspects, a detectable label can be a radiolabel including, but not limited to 3H, 125I, 35S, 14C, 32P, and 33P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system.

### Cell Differentiation to NK Cells

Provided herein are methods and compositions for generating NK cells (e.g., therapeutic NK cells or CAR NK cells) by way of differentiation of a pluripotent or multipotent stem cell that has been engineered or treated to lack TRIM28 expression and/or activity. Where generation of a CAR NK cell is desired, the pluripotent or multipotent stem cell can also be engineered to express at least one chimeric antigen receptor (CAR).

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell (e.g., an induced pluripotent stem cell) or another pluripotent or multipotent stem cell can be cultured under conditions that allow differentiation to lineage-restricted precursor cells (e.g., a cell “committed to a particular lineage” such as a mesodermal stem cell or a endodermal stem cell), which in turn can differentiate into other types of precursor cells further down the pathway (such as an tissue specific precursor, for example, a lymphocyte precursor), and then to an end-stage differentiated cell, which plays a characteristic role in a certain tissue type, and may or may not retain the capacity to proliferate further.

Inhibition of TRIM28 in a pluripotent or multipotent stem cell induces the generation of NK cells preferentially over other cell types under a variety of conditions, as shown in the working Examples. Thus, one of skill in the art will recognize that the conditions for differentiating NK cells can widely vary while still producing NK cells from cells engineered or treated to lack TRIM28 expression and/or activity.

One of skill in the art can determine the emergence of NK cells during a differentiation protocol by detecting the presence of NK cell-specific markers or detecting the loss or absence of pluripotent stem cell markers. In one embodiment, the emergence of CD56 indicates the generation of NK cells.

As used herein, a “cell-surface marker” refers to any molecule that is expressed on the surface of a cell. Cell-surface markers often provide antigenic determinants to which antibodies can bind to.

As used herein, the term “positive for” when referring to a cell positive for a marker (e.g., an NK cell marker means that a cell surface marker is detectable above background levels on the cell using immunofluorescence microscopy or flow cytometry methods, such as fluorescence activated cell sorting (FACS). Alternatively, the terms “positive for” or “expresses a marker” means that expression of mRNA encoding a cell surface or intracellular marker is detectable above background levels using RT-PCR. The expression level of a cell surface marker or intracellular marker can be compared to the expression level obtained from a negative control (i.e., cells known to lack the marker) or by isotype controls (i.e., a control antibody that has no relevant specificity and only binds non-specifically to cell proteins, lipids or carbohydrates). Thus, a cell that “expresses” a marker (or is “positive for a marker”) has an expression level detectable above the expression level determined for the negative control for that marker. Exemplary markers useful for identifying NK cells include CD56, CD8, NKG2A, NKG2D, NKp30, NKp44, NKp46, CD161, 2B4, NTB-A, CRACC, DNAM-1, CD69, CD25, NKp44 and/or KIRs.

As used herein, the term “negative for” when referring to a cell negative for a marker (or the term “does not express”) means that a cell surface marker cannot be detected above background levels on the cell using immunofluorescence microscopy or flow cytometry methods, such as fluorescence activated cell sorting (FACS). Alternatively, the terms “negative” or “does not express” means that expression of the mRNA for an intracellular marker or cell surface marker cannot be detected above background levels using RT-PCR. The expression level of a cell surface marker or intracellular marker can be compared to the expression level obtained from a negative control (i.e., cells known to lack the marker) or by isotype controls (i.e., a control antibody that has no relevant specificity and only binds non-specifically to cell proteins, lipids or carbohydrates). Thus, a cell that “does not express” a marker appears similar to the negative control for that marker. In some embodiments, it is advantageous to detect the loss of stem cell markers during differentiation of pluripotent or multipotent stem cells to NK cells as described herein. In some embodiments, the natural killer cells described herein can be selected based on the lack of expression of CD3, and/or CD8, as well the lack of expression of gene markers of pluripotency.

Undifferentiated ES cells express genes that can be used as markers to detect the presence of undifferentiated cells. The polypeptide products of such genes can be used as markers for negative selection. For example, see U.S.S.N. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including, but not limited to, stage-specific embryonic antigen (SSEA)-3, SSEA-4, TRA-I-60, TRA-1-81, and alkaline phosphatase.

In some embodiments, particularly where the NK cells are to be used therapeutically, it is desirable to test for NK cell activity. For example, a chromium-51 release assay can be used for the quantification of NK-mediated cytotoxicity. A typical chromium-51 release assay comprises a target cell that is labeled with 51-chromium and the sample of NK cells to be tested. The 51-chromium is released from the target cells by NK-mediated cytolysis and can be detected using standard means for measuring gamma radiation (e.g., gamma counter, in a liquid scintillation counter or in a microplate). Effective NK cell activity results in a higher release of 51-chromium and is thus indicative that the NK cells derived using the methods described herein will have therapeutic efficacy (Elsner et al. 2020. Methods Enzymol. 631: 497-512.)

In some embodiments, flow cytometry methods can be employed to enrich NK cells based on their cell characteristics. This technique can be used to separate NK cells from undifferentiated cells in a population, for cell counting, cell sorting, biomarker detection, and the like.

### Transposable Elements

“Transposons”, “transposable elements”, or “TEs” include a short piece of nucleic acid bounded by repeat sequences. In some embodiments, transposons can include endogenous retroviruses (ERVs), long interspersed elements (LINEs), and short interspersed elements, (SINEs). Active transposons encode enzymes that facilitate the insertion of the nucleic acid into DNA sequences. These transposable elements transpose through a cut-and-paste mechanism; the element-encoded transposase catalyzes the excision of the transposon from its original location and promotes its reintegration elsewhere in the genome. Autonomous members of a transposon family can express an active transposase, the trans-acting factor for transposition, and thus are capable of transposing on their own. Nonautonomous elements have mutated transposase genes but may retain cis-acting DNA sequences. These cis-acting DNA sequences are also referred to as inverted terminal repeats. Some inverted repeat sequences include one or more direct repeat sequences. These sequences usually are embedded in the terminal inverted repeats (IRs) of the elements, which are required for mobilization in the presence of a complementary transposase from another element.

The working examples described herein show that cells can be identified as being committed to the lymphoid lineage by way of a pattern of transposable elements. A pattern of transposable elements can be determined by measuring the expression of a plurality of transposable elements (e.g., enhancers). Cells committed to the lymphoid lineage have a pattern of transposable elements that is increased in expression as compared to e.g., cells of a myeloid lineage, thus the emergence of such a pattern of transposable elements permits identification of a cell as being committed to the lymphoid lineage. In such embodiments, it is desirable to compare the pattern of transposable elements to a known reference or reference control (e.g., a cell that is committed to the lymphoid lineage).

A cell is said to be “substantially similar” to another cell if the pattern of transposable elements is substantially similar to the pattern of transposable elements in a reference cell (e.g., a cell committed to the lymphoid lineage (i.e., they are at least 90% similar in the pattern of transposable elements as determined by gene and transposable element clustering analysis). In some embodiments, such cells will also be substantially similar in at least one relevant function (e.g., NK cell activity).

The pattern of transposable elements utilizes activity and contact (ABC) enhancers in order to cluster hematopoietic cell types into different groups, such as HSPC, myeloid, lymphoid, and erythroid cells. As shown in the working examples, a pattern that was upregulated in transposable element activity indicated that the lineage of the cell was lymphoid relative to the pluripotent stem cell and a pattern that was downregulated in transposable element activity indicated that the lineage of the cell was myeloid relative to the pluripotent stem cell. In some embodiments, transposable elements also exhibited cell type specificity amongst closely related cell types such NK, CD4+ T and CD8+ T cells, for example transposable elements of the ERV1 subfamily were preferentially expressed in CD8+ T cells and repressed in CD4+ T cells.

As used herein, an “ABC enhancer” is an enhancer that is characterized by its activity and contact with a transposable element. An enhancer is a nucleotide sequence that increases the rate of genetic transcription by preferentially increasing the activity of the nearest promoter on the same DNA molecule. ABC enhancers can be clustered using an ABC model that predicts enhancer-gene linkages in hematopoietic cells.

### Pharmaceutical Compositions

Provided herein are therapeutic NK cell compositions comprising NK cells generated as described herein. Therapeutic compositions contain a physiologically tolerable carrier together with NK cells and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. At a minimum, the pharmaceutically acceptable carrier will comprise an osmolarity that permits retention of cell viability.

In general, the NK cells described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the NK cells as described herein using routine experimentation.

A cell composition comprising NK cells generated as described herein can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

In some embodiments, such a pharmaceutical composition can comprise components that can enhance viability of the cells (e.g., growth factors, osmotic regulators, carbohydrates etc.) or inhibit microbial growth (e.g., antibacterial, antifungals) etc.

### Cancer

In some embodiments, the NK cells generated as described herein can be used in methods of cell-based cancer immunotherapy. Cancer immunotherapy allows for tumor-specific treatment by targeting antigens present on tumor cell surfaces through the use of the immune system.

As used herein, the term “cancer immunotherapy” or “cell-based cancer immunotherapy” refers to a treatment that uses a person's own immune system to fight tumor cells, and in particular refers to the administration of therapeutic NK cells or CAR NK cells for the treatment of cancer.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

Examples of cancer that can be treated with the therapeutic NK cells described herein include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

### Therapeutic Applications

Natural Killer Cells (NK cells) are cells of the innate immune system that generally function to kill virally-infected cells and to control early signs of cancer. Such cells have anti-tumor, antiviral and antimicrobial activity. The successful use of NK cells for the treatment of cancer has been previously reported. [Ruggeri et al (2005) Curr Opin Immunol 17: 211-7; Ren et al (2007) Cancer Biother Radiopharm 22: 223-34; Koehl et al (2004) Blood Cells Mol Dis 33: 261-6.176 Passweg et al (2004) Leukaemia 18:1835-8]. One advantage to the use of NK cells in cancer immunotherapy is that NK cells are generally well tolerated and the risk of graft-vs-host disease is low.

One obstacle to the use of NK cells as therapeutics is that only relatively small numbers of NK cells can be isolated from regular leukapheresis products. The methods and compositions described herein permit the generation of NK cells in vitro in quantities that are sufficient for the use of such cells in therapies for the treatment of cancer.

In some embodiments, the NK cells generated as described herein are used to generate chimeric antigen receptor NK (CAR NK) cells. Chimeric Antigen Receptors (CARs) are recombinant receptors that recognize a specific protein or antigen expressed on a target cell, such as a tumor cell. When an NK cell expresses a CAR, it is referred to as a “CAR NK cell.” CARs are able to direct a specific immune response against cells that express the antigen they bind to. CAR NK cells can be particularly useful in the treatment of cancer by employing a CAR that recognizes a cancer or tumor antigen.

A typical CAR comprises an antigen-specific binding domain, commonly derived from a single chain variable fragment (scFv), a spacer region, a transmembrane domain, and an intracellular signaling domain that transmits activation and costimulatory signals to the cells (e.g., the NK cells) in which they are expressed. Depending on the number of signaling domains, CARs are classified into 1st generation (one), 2nd generation (two), or 3rd generation (three) CARs (Dotti et al., Immunol Rev. 2014 January; 257(1)).

The “spacer” or “hinge” region, is the connecting sequence between the antigen-binding domain and the transmembrane domain. The most common sequence used as spacer is the constant immunoglobulin IgG1 hinge-CH2-CH3 Fc domain. WO 2016/042461 discloses CARs comprising spacer regions deriving from the extracellular domain of the human low affinity nerve growth factor receptor (LNGFR).

The extracellular domain of an exemplary CAR comprises an antigen-specific targeting domain that has the function of binding to the target antigen of interest. The antigen-specific targeting domain can be any naturally occurring, synthetic, semi-synthetic, or a molecule produced recombinant technology, protein, peptide or oligo peptide that specifically binds to the target antigen.

Examples of possible antigen-specific targeting domains include antibodies or antibody fragments or derivatives, synthetic or naturally occurring ligands of the targeted receptor including molecules, binding or extracellular domains of receptors or binding proteins.

In a preferred embodiment, the antigen-specific targeting domain is, or is derived from, an antibody. An antibody is a protein, or a polypeptide sequence derived from an immunoglobulin able to bind with an antigen. Antibody as herein used includes polyclonal or monoclonal, multiple or single chain antibodies as well as immunoglobulins, whether deriving from natural or recombinant source.

An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

In a preferred embodiment, the binding domain is a single chain antibody (scFv). The scFv may be murine, human or humanized scFv.

In a preferred embodiment the target antigen is a tumor antigen.

As used herein the term tumor antigen includes antigens expressed on tumor cells including biomarkers or cell surface markers that are found on tumor cells and are not substantially found on normal tissues or restricted in their expression in non-vital normal tissues.

In some embodiments, the spacer of the CAR comprises a fragment derived from the extracellular domain of human low affinity nerve growth factor (LNGFR) as disclosed in WO 2016/042461, the contents of which are incorporated herein by reference in its entirety.

Where LNGFR is not expressed on the majority of human hematopoietic cells, spacer units derived from LNGFR can be used to facilitate selection of cells genetically engineered to express CARs and quantitative analysis of transduced gene expression by immunofluorescence.

Any method known in the art can be employed to express a CAR in an NK cell as described herein. In one embodiment, the CAR is expressed from a lentiviral vector or is inserted into the genome of the pluripotent or multipotent stem cell used to derive the NK cells as described herein using any desired genomic modification method.

In some embodiments, a therapeutic composition can comprise an NK cell as described herein in combination with a natural killer cell engager, a bi-functional natural killer cell engager or a tri-functional natural killer cell engager. As used herein, a “NKCE”, a “BiKE”, and a “TRiKE” are known as natural killer cell engagers ((NKCE), a bi-functional natural killer cell engager (BiKE), and/or a tri-functional natural killer cell engager (TRiKE)). These molecules are built from fragments of monoclonal antibodies and are designed to interact with multiple different binding targets on both NK cells and tumor cells in order to achieve an immune response. In some embodiments, the composition can be co-administered with a NKCE, a BiKE, and/or a TRiKE.

### Administration of Cells and Efficacy

As used herein, the terms “administering,” “introducing” and “transplanting” are used interchangeably in the context of the placement of cells, e.g., NK cells derived from pluripotent stem cells that lack TRIM28 as described herein into a subject, by a method or route which results in at least partial localization of the introduced cells at a desired site, such as a site of tumor cell growth, such that a desired effect(s) is produced. The NK cells can be administered through injection into a vein or tissue or alternatively be administered by any appropriate route which results in delivery to a desired location in the subject where at least a portion of the injected cells or components of the cells remain viable. The period of viability of the cells after administration to a subject can be as short as a few hours, e.g., twenty-four hours, to a few days, to as long as 2-6 (e.g., the lifespan of a typical NK cell).

In some embodiments, the NK cells described herein can be administered to the vein and/or tissue in an effective amount for the treatment of a cancer (e.g., leukemia or a lymphoma). The term “effective amount” as used herein refers to the amount of a population of NK cells, needed to alleviate at least one or more symptoms of a disease or disorder, including but not limited to a cancer such as a leukemia or a lymphoma. An “effective amount” relates to a sufficient amount of a composition to provide the desired effect, e.g., treat a subject having a cancer such as a leukemia or a lymphoma, decrease tumor growth, increase the presence of immune cells following chemotherapy, etc. The term “therapeutically effective amount” therefore refers to an amount of NK cells, or a composition comprising such cells that is sufficient to promote a particular effect when administered to a typical subject, such as one who has, or is at risk for, a cancer comprising of cancer. An effective amount as used herein would also include an amount sufficient to prevent or delay the development of a symptom of the disease, alter the course of a disease symptom (for example but not limited to, slow the progression of a symptom of the disease), or reverse a symptom of the disease. Exemplary symptoms of cancer can include, but are not limited to, fatigue, fever, pain, loss of appetite, weight loss, headaches, shortness of breath, a lump or thickening of the skin, anemia, and the like. It is understood that for any given case, an appropriate “effective amount” can be determined by one of ordinary skill in the art using routine experimentation.

In some embodiments, the subject is first diagnosed as having cancer or a disease or disorder affecting either the blood, bone marrow, and/or lymph nodes prior to administering the cells according to the methods described herein. In some embodiments, the subject is first diagnosed as being at risk of developing cancer (e.g., genetic mutation or family history of the disease) prior to administering the cells.

For use in the various aspects described herein, an effective amount of NK cells comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more NK cells. In the alternative embodiment, an effective amount of NK cells comprises at least 1×10³ to 5×10¹⁰, at least 1×10³ to 1×10¹⁰, at least 1×10³ to 5×10⁹, at least 1×10³ to 1×10⁹, at least 1×10³ to 5×10⁸, at least 1×10³ to 1×10⁸, at least 1×10³ to 5×10¹¹, at least 1×10⁵ to 1×10¹⁰, at least 1×10⁶ to 5×10¹⁰, at least 1×10⁷ to 1×10¹⁰ at least 1×10⁸ to 1×10¹⁰, at least 1×10⁹ to 1×10¹⁰, at least 1×10⁹ to 1×10¹¹, at least 1×10¹⁰ to 1×10¹², at least 1×10¹¹ to 1×10¹³ NK cells.

An effective amount of NK cells can be administered to a subject at least once a day, at least twice a day, at least three times a day, or more in order to sufficiently prevent or delay the development of a symptom of the disease and/or alter the course of a disease symptom. An effective amount of NK cells can be administered over the time span of 1 day, span of 2 days, span of 3 days, span of 4 days, span of 5 days, span of 6 days, span of 7 days, span of 7 days, span of 8 days, span of 9 days, span of 10 days, span of 11 days, span of 12 days, span of 13 days, span of 14 days, span of 15 days, span of 16 days, span of 17 days, span of 18 days, span of 19 days, span of 20 days, span of 21 days, span of 22 days, span of 23 days, span of 24 days, span of 25 days, span of 26 days, span of 27 days, span of 28 days, span of 29 days, span of 30 days or more. An effective amount of NK cells can be administered consecutively or intermittently during the course of treatment.

The NK cells can be derived from one or more donors, or can be obtained from an autologous source. In some embodiments of the aspects described herein, the NK cells are expanded or differentiated from pluripotent stem cells lacking TRIM28 cells in culture prior to administration to a subject in need thereof.

Exemplary modes of administration for use in the methods described herein include, but are not limited to, injection and systemic administration. “Injection” includes, without limitation, intravenous, intratumor, or intraarterial delivery.

In some embodiments, a therapeutically effective amount of NK cells is administered using direct injection into the vein, artery, or directly into the tumor either alone or in combination with an infusion product and/or an additional treatment for cancer. These methods are particularly aimed at therapeutic treatments of human subjects having, or at risk of having a cancer such as leukemia or lymphoma. The NK cells described herein can be administered to a subject having cancer by any appropriate route which results in an effective treatment in the subject. In some embodiments of the aspects described herein, a subject having cancer is first selected prior to administration of the cells.

The choice of formulation for a cell composition will depend upon the specific composition used and the number of NK cells to be administered; such formulations can be adjusted by the skilled practitioner. However, as an example, where the composition includes NK cells in a pharmaceutically acceptable carrier, the composition can include a suspension of the cells in an appropriate buffer at an effective concentration of cells per mL of solution. The formulation can also include cell nutrients, a simple sugar (e.g., for osmotic pressure regulation) or other components to maintain the viability and/or assist in delivery and establishment of the cells at the site of the tumor.

Efficacy testing can be performed during the course of treatment using the methods described herein. Measurements of the degree of severity of a number of symptoms associated with a particular ailment are noted prior to the start of a treatment and then at a later specific time period after the start of the treatment. In some embodiments, a pharmaceutical composition comprising an NK cell or CAR NK cell as described herein or a population thereof can be used for cellular replacement therapy or cell-based cancer immunotherapy in a subject.

The efficacy of a treatment comprising a composition as described herein for the treatment of a e.g., cancer can be determined by the skilled clinician. However, a treatment is considered “effective treatment,” as the term is used herein, if any one or all of the signs or symptoms of e.g., cancer are altered in a beneficial manner, other clinically accepted symptoms or markers of disease are improved or ameliorated, e.g., by at least 10% following treatment with NK cells or CAR NK cells as described herein. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of cancer; or (2) relieving the disease, e.g., causing regression of symptoms; and (3) preventing or reducing the likelihood of the development of infection or sepsis.

In some embodiments, efficacy is assessed by measuring a beneficial clinical effect. Such beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, efficacious treatment can refer to prolonging survival as compared to expected survival if not receiving treatment. Thus, one of skill in the art realizes that a treatment can improve the disease condition, but may not be a complete cure for the disease. Successful treatment can also be assessed by a reduction in the need for medical interventions (e.g., blood transfusions), reduction in hospital or emergency room visits, or other markers of an improved quality of life. In some embodiments, treatment can include prophylaxis. However, in alternative embodiments, treatment does not include prophylaxis.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

The technology may be as described in any one of the following numbered paragraphs:

Paragraph 1: A method for generating a natural killer (NK) cell comprising: differentiating a pluripotent stem cell engineered to lack TRIM28 expression and/or activity for a sufficient time to promote differentiation to a CD56⁺ NK cell.

Paragraph 2: The method of paragraph 1, wherein the pluripotent stem cell comprises an induced pluripotent stem (iPS) cell, an embryonic stem cell, a cord blood cell, and/or a bone marrow cell.

Paragraph 3: The method of paragraph 2, wherein the cord blood cell and/or the bone marrow cell comprises a CD34+ hemogenic endothelial cell.

Paragraph 4: The method of paragraph 1, wherein the pluripotent stem cell engineered to lack TRIM28 expression and/or activity is generated using a CRISPR-Cas9 system.

Paragraph 5: The method of paragraph 2, wherein the pluripotent stem cell is engineered to delete or mutate a gene and/or protein encoding TRIM28, thereby reducing expression and/or activity of TRIM28.

Paragraph 6: The method of paragraph 4, further comprising treatment with at least one additional inhibitor of EHMT1 and/or SETDB1.

Paragraph 7: The method of paragraph 1, wherein the NK cell generated is a CD56+, CD56+CD3−, CD56+CD3−CD8+, or CD56CD3−CD8− NK cell.

Paragraph 8: The method of claim 1, wherein the cell engineered to lack TRIM28 expression comprises a pattern of transposable elements that is substantially similar to the pattern of transposable elements in a cell committed to the lymphoid lineage.

Paragraph 9: A method for generating an NK cell comprising contacting a pluripotent stem cell with an inhibitor of TRIM28 expression and/or activity and culturing under conditions and for a sufficient time to promote differentiation to an NK cell.

Paragraph 10: The method of paragraph 9, wherein the NK cell is CD56+, CD56+CD3−, CD56+CD3−CD8+, or CD56CD3−CD8− NK cell, etc.

Paragraph 11: The method of paragraph 9, wherein the inhibitor of TRIM28 expression and/or activity comprises an inhibitory nucleic acid, a small molecule, or a peptide.

Paragraph 12: The method of paragraph 11, wherein the inhibitory nucleic acid is selected from the group consisting of an siRNA, an shRNA, a miRNA, an antisense oligonucleotide, an aptamer, a ribozyme, and a triplex forming oligonucleotide.

Paragraph 13: The method of paragraph 12, further comprising a step of administering or contacting with at least one inhibitor that modulates methylation of DNA.

Paragraph 14: The method of paragraph 13, wherein at least one inhibitor that inhibits methylation of DNA inhibits the expression and/or activity of one or more of: DNMT; MBD; DNA demethylase; HMT; methyl-histone binding protein; histone demethylase; HAT; acetyl-binding protein; or HDAC.

Paragraph 15: The method of paragraph 11, wherein further comprising administering or contacting with at least one inhibitor that targets sumoylation.

Paragraph 16: The method of paragraph 15, wherein at least one inhibitor that targets sumoylation is an E3 ligase inhibitor.

Paragraph 17: A method for generating an NK cell, the method comprising: contacting a pluripotent stem cell treated with an inhibitor that disrupts TRIM28 binding with one or more binding partners.

Paragraph 18: The method of paragraph 17 wherein the one or more binding partners is selected from the group consisting of KRAB-ZNF transcription factors, MDM2, p53, the NuRD complex (comprising of NuRD, Mi2α, and an HDAC), SETDB1, CBF-A, and HP1.

Paragraph 19: The method of paragraph 17, wherein the cell engineered to lack TRIM28 expression comprises a pattern of transposable elements that is substantially similar to the pattern of transposable elements in a cell committed to the lymphoid lineage.

Paragraph 20: The method of paragraph 1, wherein the NK cell is a CD56+, CD56+CD3−, CD56+CD3−CD8+, or CD56CD3−CD8− NK cell.

Paragraph 21: An engineered NK cell generated using the method of any one of claims 1-20, wherein the NK cell lacks TRIM28 expression or activity.

Paragraph 22: An engineered NK cell generated using the method of any one of paragraphs 17-21.

Paragraph 23: A therapeutic cell composition comprising an NK cell of paragraph 22 or a population thereof, and a pharmaceutically acceptable carrier.

Paragraph 24: The therapeutic composition of paragraph 23, for use in cellular replacement therapy in a patient.

Paragraph 25: A therapeutic CAR-NK cell composition comprising an NK cell that lacks TRIM28 expression and/or activity, wherein the NK cell expresses a chimeric antigen receptor (CAR).

Paragraph 26: The therapeutic CAR-NK cell of paragraph 25, wherein the NK cell is an CD56+, CD56+CD3−, CD56+CD3−CD8+, or CD56CD3−CD8− NK cell.

Paragraph 27: The therapeutic CAR-NK cell of paragraph 25, wherein the NK cell is generated by in vitro differentiation of a pluripotent stem cell engineered to lack TRIM28 expression and/or activity.

Paragraph 28: The therapeutic CAR-NK cell composition of paragraph 25, wherein the composition further comprises a pharmaceutically acceptable carrier.

Paragraph 29: The therapeutic CAR-NK cell composition of paragraph 25, wherein the cell is autologous to the subject to be treated.

Paragraph 30: The therapeutic CAR-NK composition of paragraph 29, further comprising a pharmaceutically acceptable carrier.

Paragraph 31: A method of treating a subject in need thereof, the method comprising: administering an NK cell of paragraph 21 in combination with a NK cell engager (NKCE), bispecific killer cell engager (BiKE), or trispecific killer cell engager (TRiKE) to a subject in need thereof.

Paragraph 32: A method of treating a subject in need thereof, comprising administering a therapeutic cell composition of paragraph 25 or 31 to a subject in need thereof.

Paragraph 33: The method of paragraph 32, wherein the subject in need thereof has or is at risk of having cancer.

Paragraph 34: The method of paragraph 31 or 32, wherein the subject in need thereof has or is undergoing chemotherapy and/or irradiation.

Paragraph 35: The method of paragraph 33, wherein the cancer comprises a leukemia or a lymphoma.

Paragraph 36: The method of paragraph 35, wherein the cancer is of a B-cell lymphoma; a low grade/follicular non-Hodgkin's lymphoma (NHL); a small lymphocytic (SL) NHL; an intermediate grade/follicular NHL; an intermediate grade diffuse NHL; a high grade immunoblastic NHL; a high grade lymphoblastic NHL; a high grade small non-cleaved cell NHL; a bulky disease NHL; a mantle cell lymphoma; an AIDS-related lymphoma; a Waldenstrom's Macroglobulinemia); a chronic lymphocytic leukemia (CLL); an acute lymphoblastic leukemia (ALL); a Hairy cell leukemia; or a chronic myeloblastic leukemia.

Paragraph 37: The method of paragraphs 32-34, wherein the subject in need thereof is human.

Paragraph 38: A method for comparing the pattern of transposable elements in a progenitor cell to the pattern of transposable elements in a progenitor cell committed to the lymphoid progenitor cell, and wherein the presence of a substantially similar pattern of transposable elements as compared to a lymphoid progenitor cell is detected, the cell is identified as a lymphoid progenitor cell.

Paragraph 39: The method of paragraph 38, wherein the reference comprises a reference cell or population or a reference value.

Paragraph 40: The method of paragraph 39, wherein the reference cell or population comprises a hematopoietic stem cell or a myeloid progenitor cell.

Paragraph 41: The method of paragraph 40, further comprising a step of isolating the lymphoid progenitor cell.

Paragraph 42: The method of paragraph 38, wherein the transposable elements are selected from the group comprising: endogenous retroviruses (ERVs), long interspersed elements (LINEs), and short interspersed elements (SINEs).

### EXAMPLES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Example 1

Transposable Elements and their Regulatory Machinery are Dynamically Expressed During Hematopoietic Differentiation

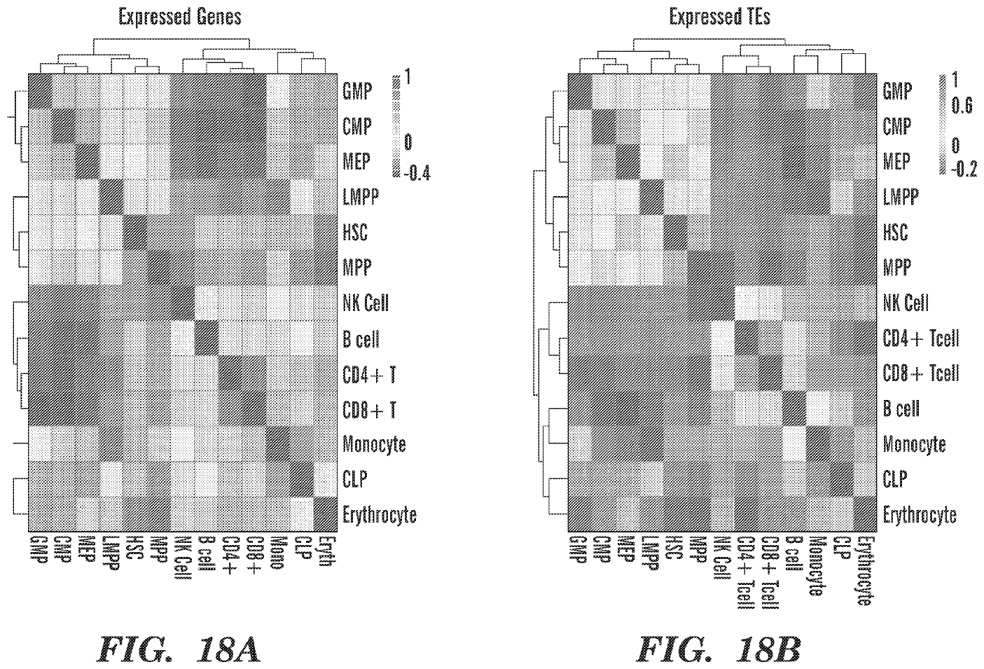

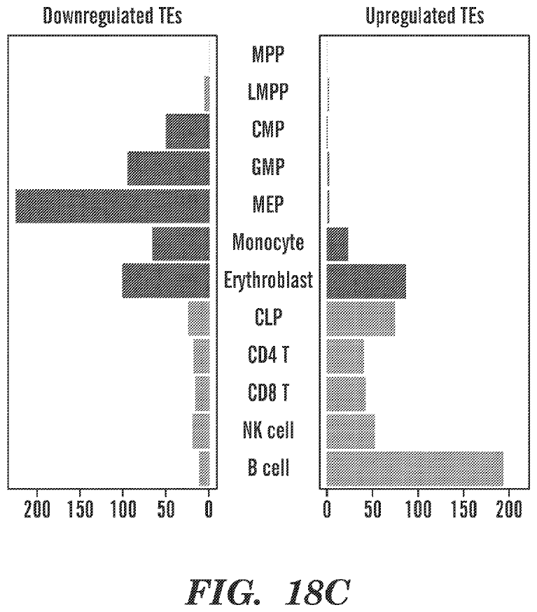

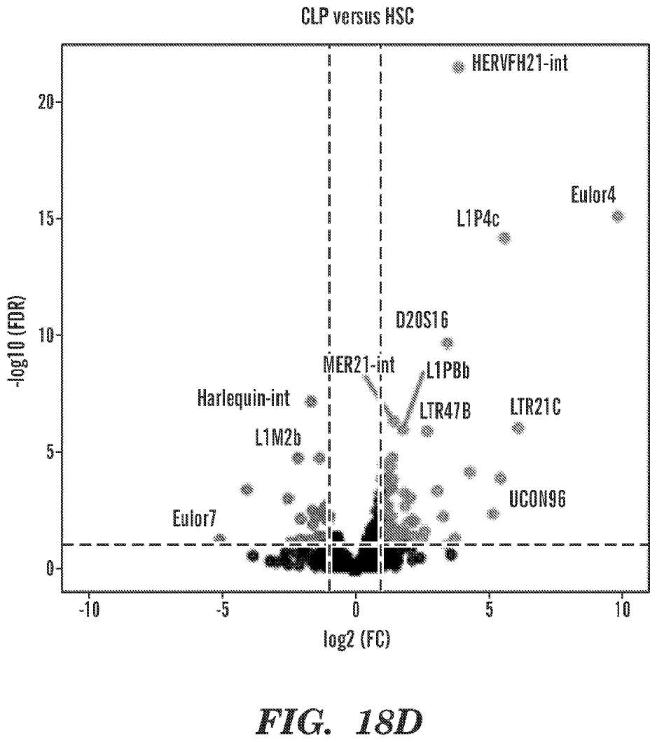

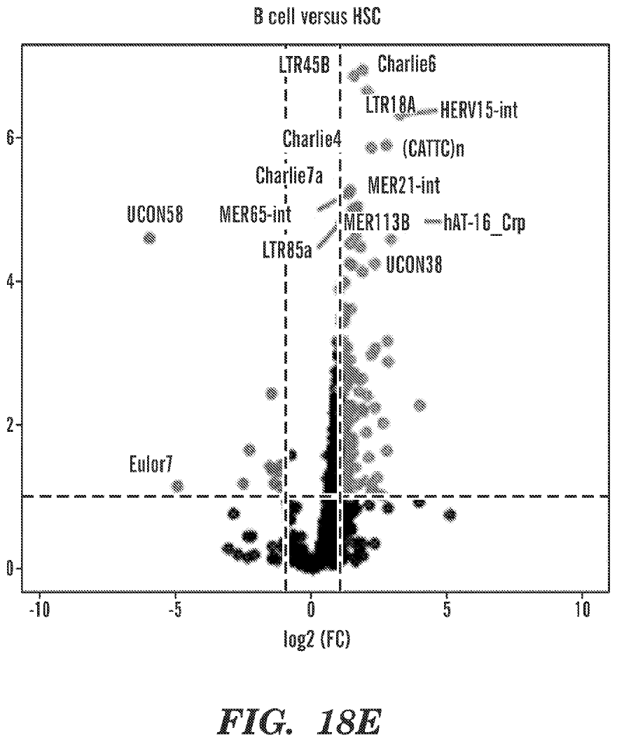

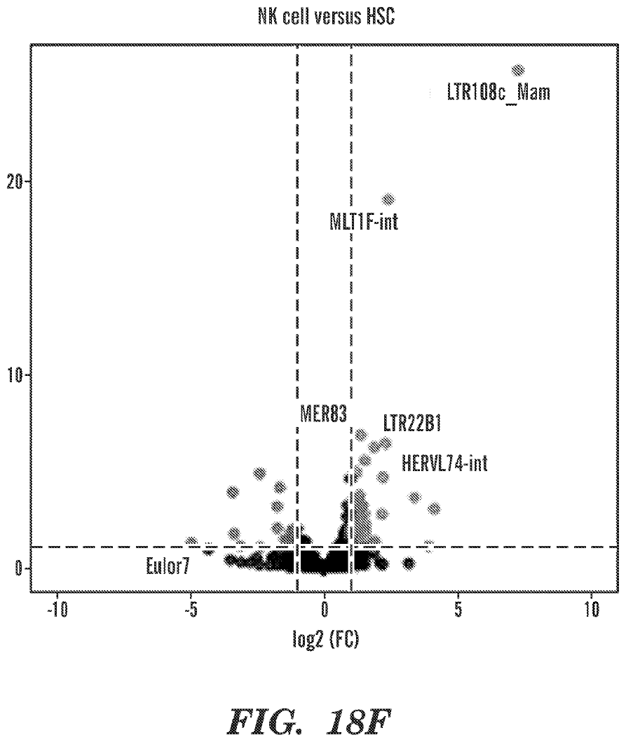

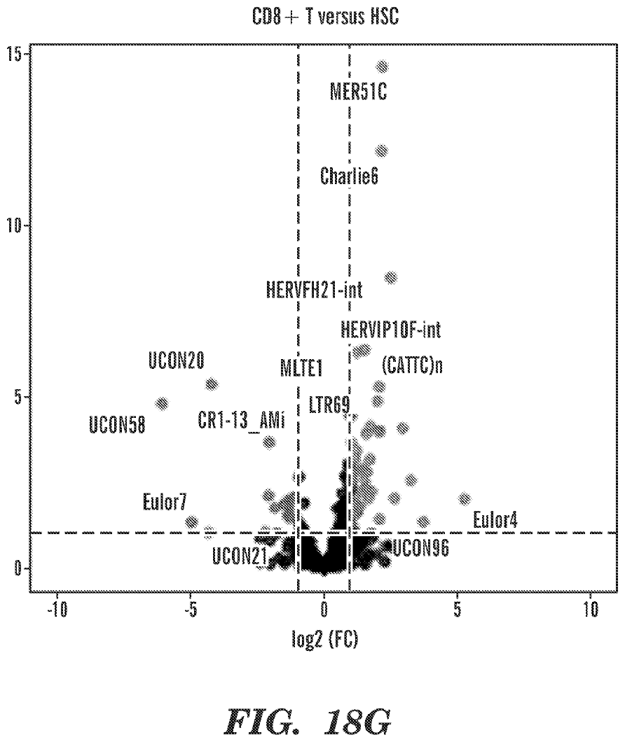

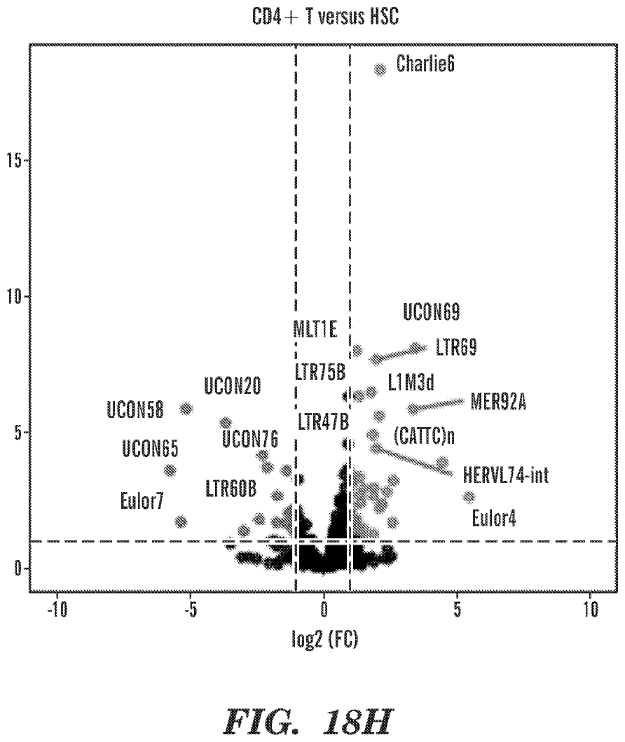

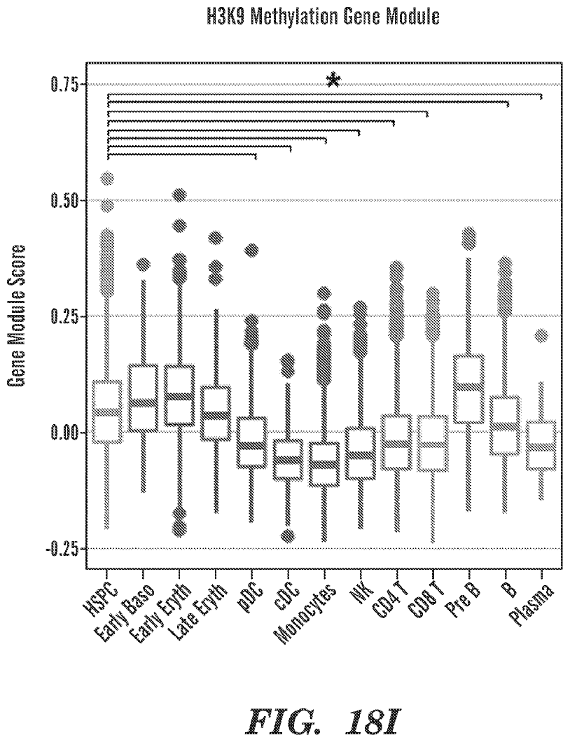

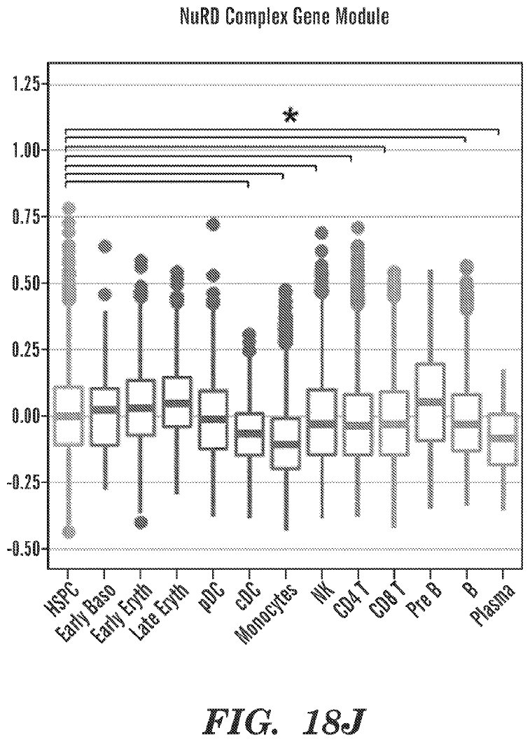

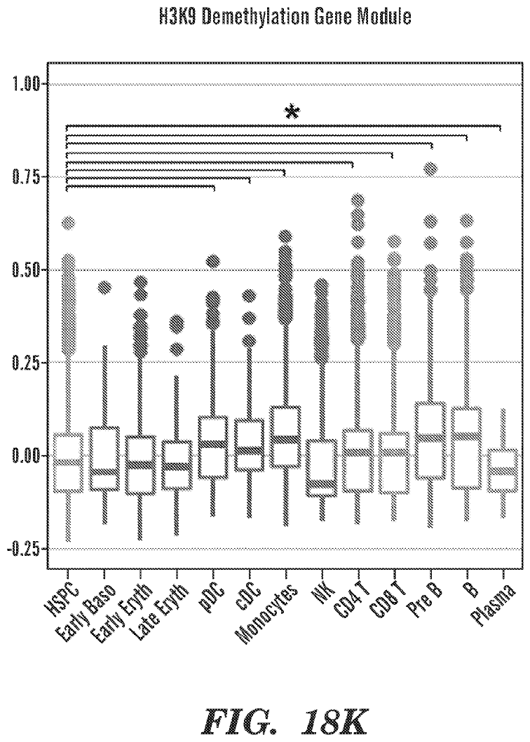

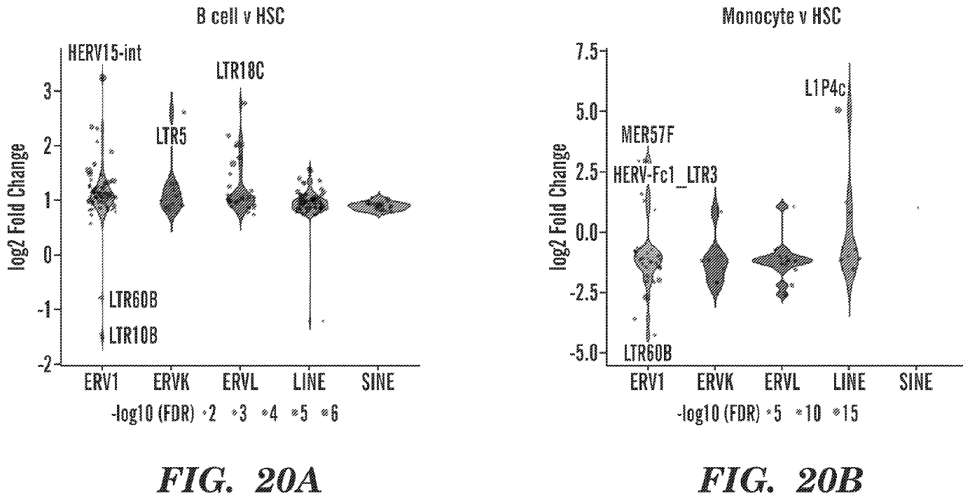

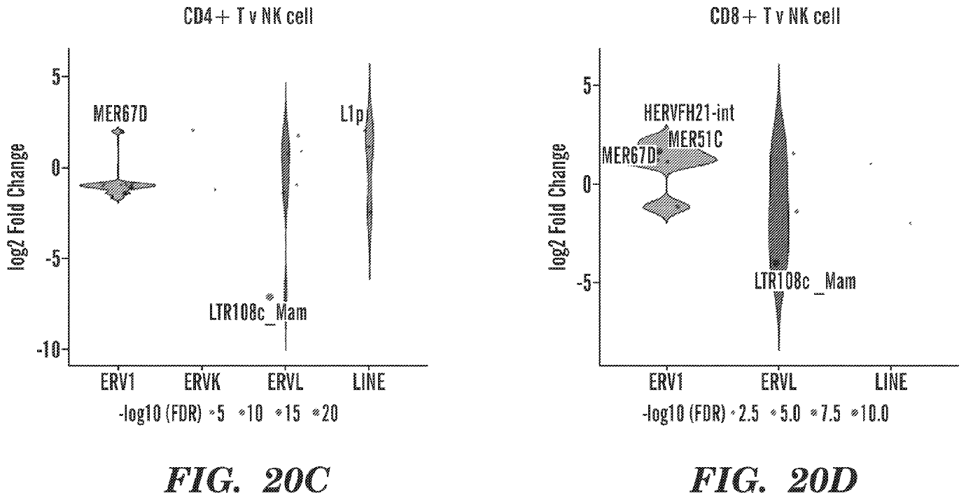

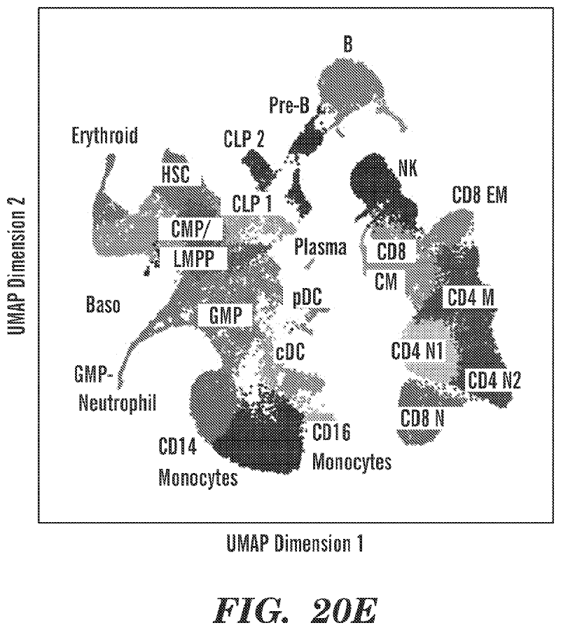

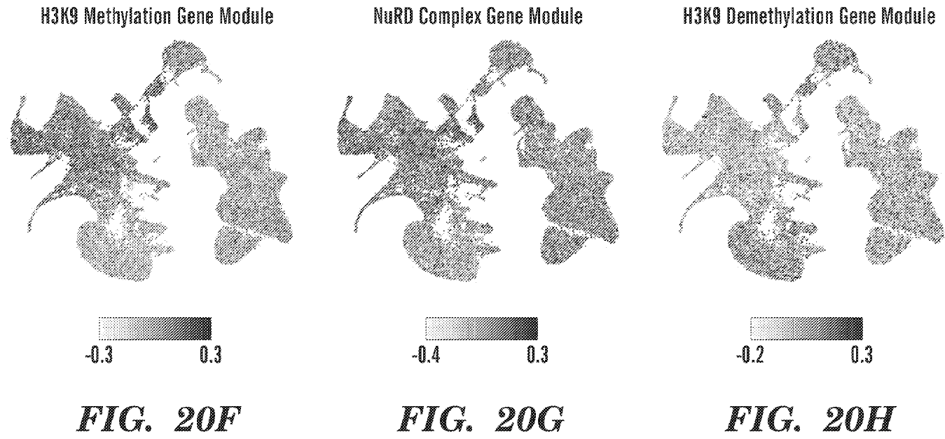

Prior analysis by the inventors indicated that specific transposable element (TE) families are enriched in ABC enhancers in a cell-type dependent manner and contribute to hematopoietic gene regulatory networks. Thus, it was then asked whether the expression of TEs exhibit cell type specificity. The inventors utilized RNA-Seq data of FACS-isolated hematopoietic stem and progenitor populations from human bone marrow and mature cell types from peripheral blood (Corces et al 2016) to quantify transposable element expression in the human hematopoietic system. Expression of transposable elements alone was sufficient to classify distinct hematopoietic cell types and resulted in lineage clustering comparable to clustering derived from expressed genes (FIGS. 18A-18B). These observations indicate that TE expression is non-random in the hematopoietic system and are reflective of cell identity. Dynamically expressed TEs were then investigated during differentiation by identifying differentially expressed TEs in progenitors and mature progeny relative to hematopoietic stem cells (HSCs) (FIGS. 18C-18H). Notably, TEs were progressively up-regulated during lymphoid differentiation, initiating at common lymphoid progenitors (CLPs) (FIG. 18C). Conversely, more TEs were down-regulated in various myeloid/erythroid progenitors and mature progeny compared to lymphoid cells. B cells exhibited the largest number of up-regulated TEs compared to HSCs, which comprised ERV, LINE and SINE TE families (FIGS. 18E, 20A). TEs also exhibited cell type specificity amongst closely related cell types such NK, CD4 T and CD8 T cells (FIGS. 18F-18H), where TEs of the ERV1 subfamily were preferentially expressed in CD8 T cells and repressed in CD4 T cells (FIGS. 20C-20D). Collectively these expression data reinforce the inventors epigenomic observations that TEs exhibit cell type specificity in the hematopoietic system.

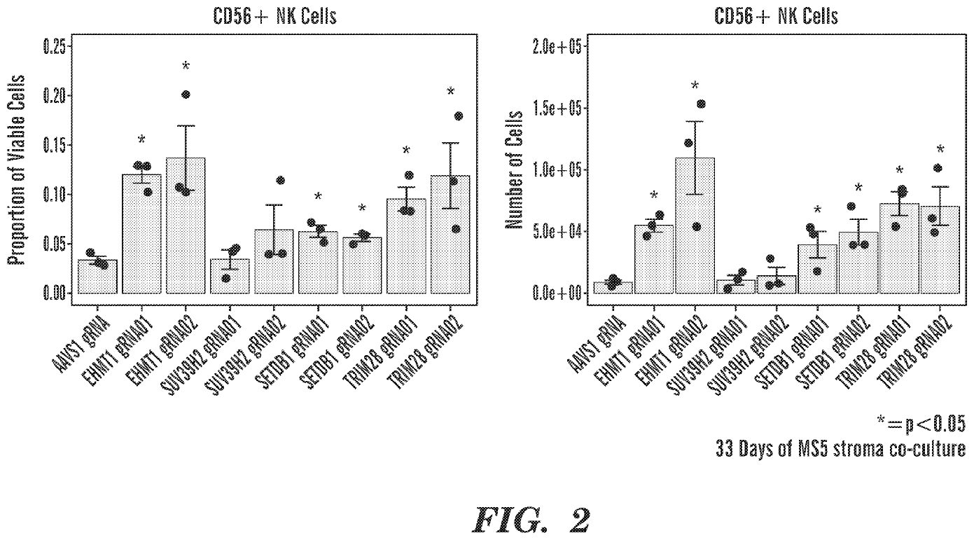

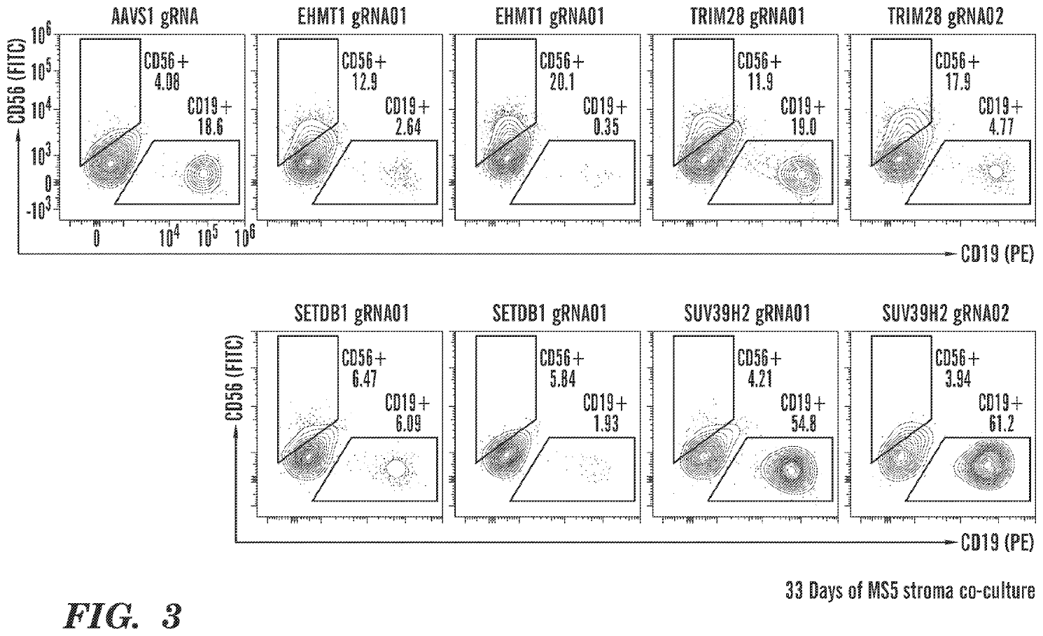

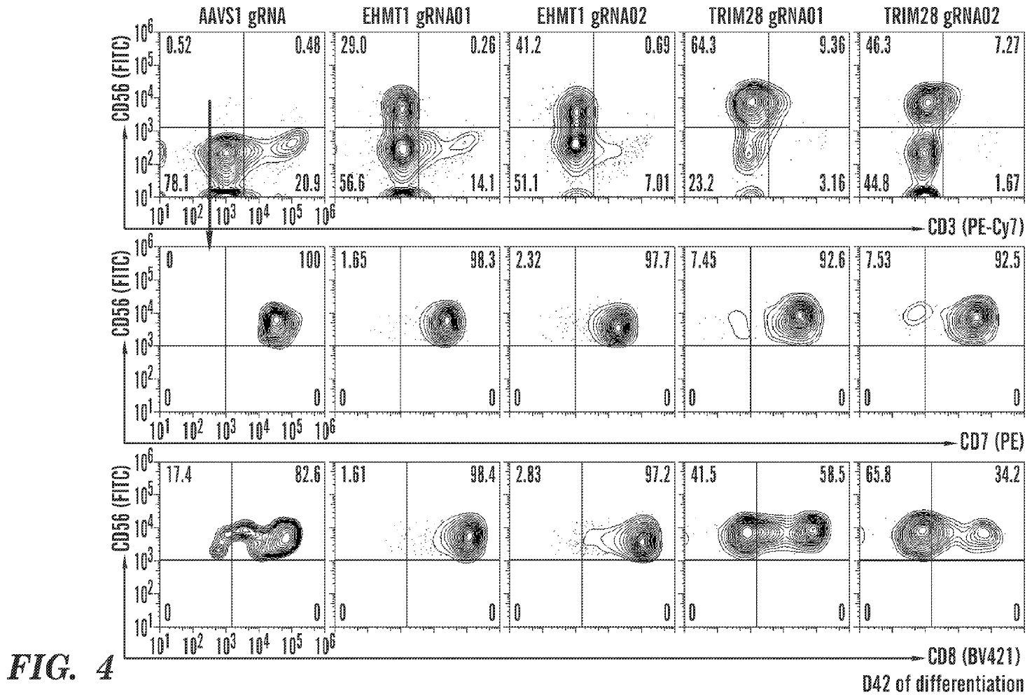

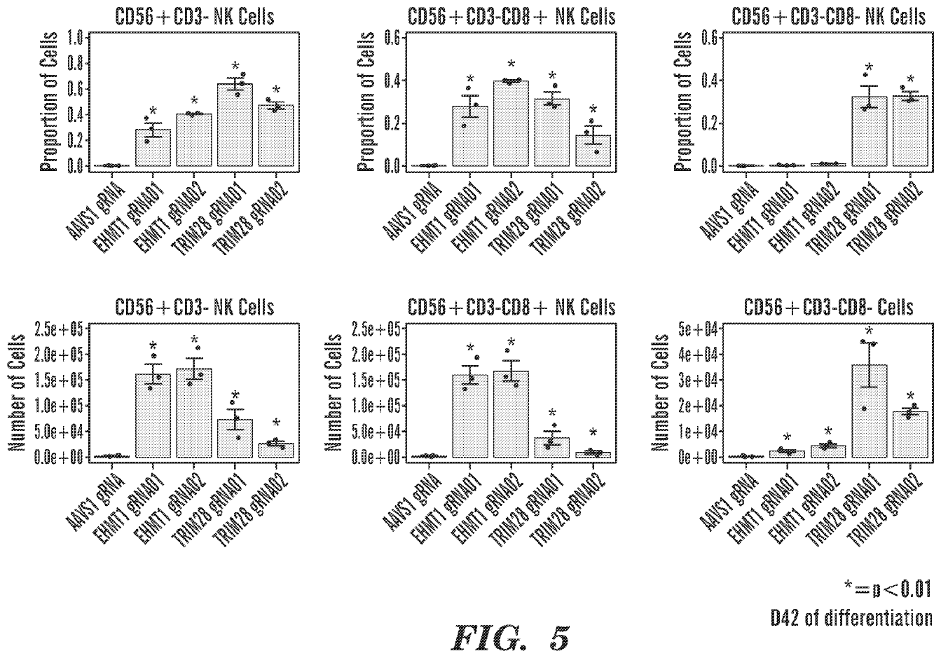

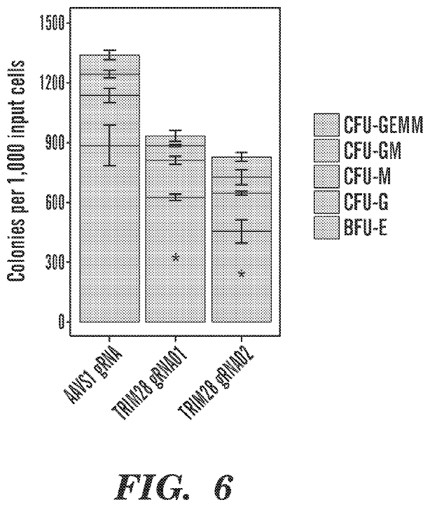

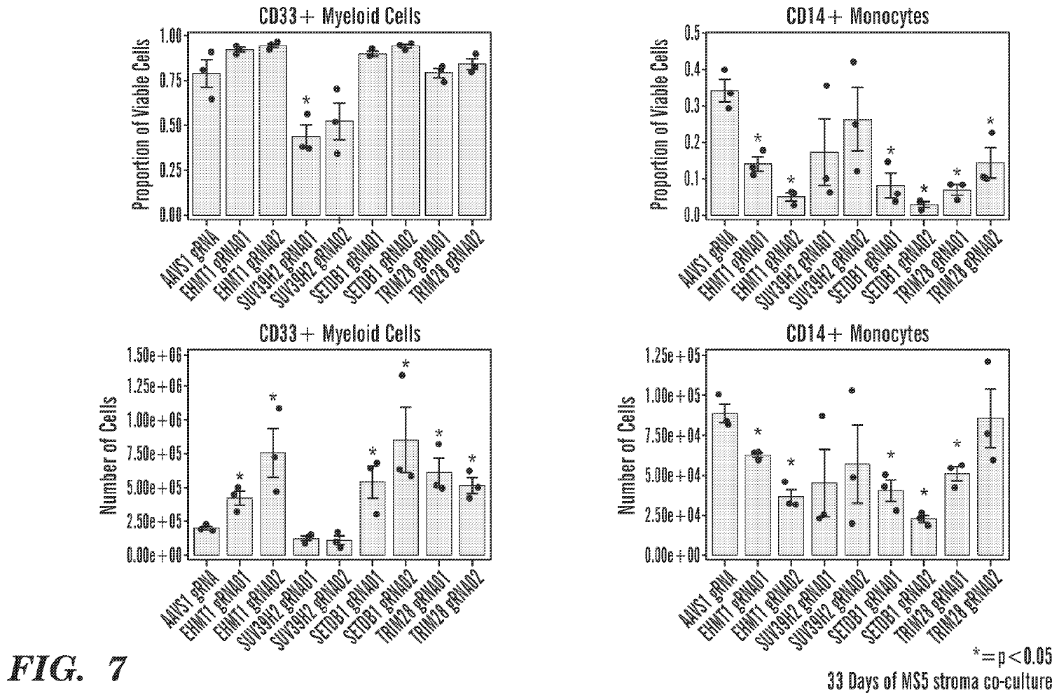

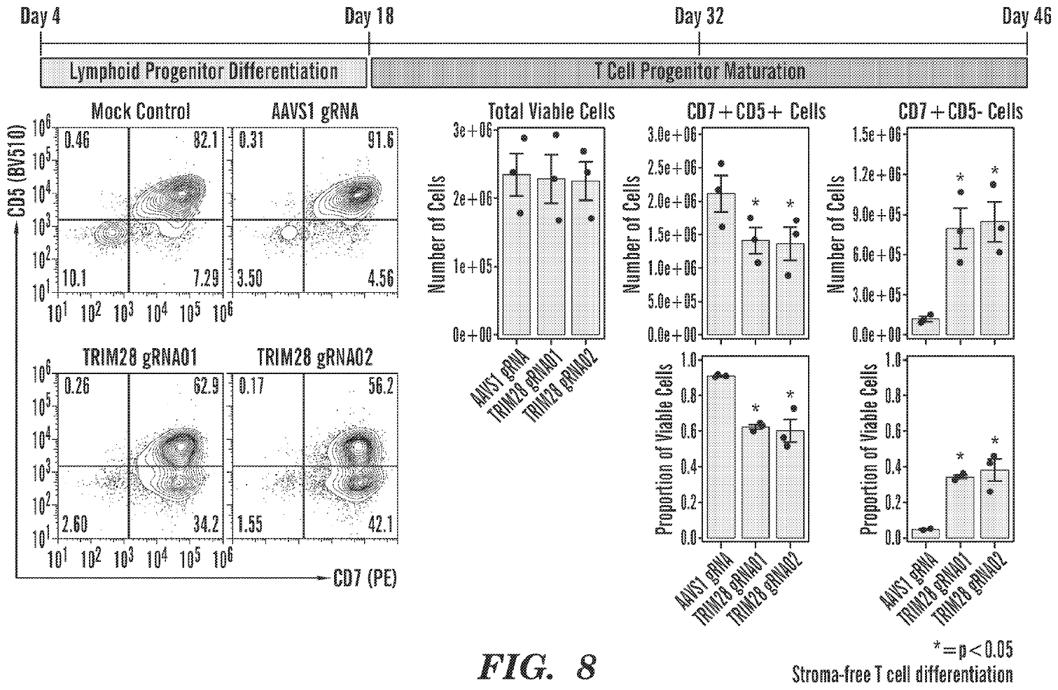

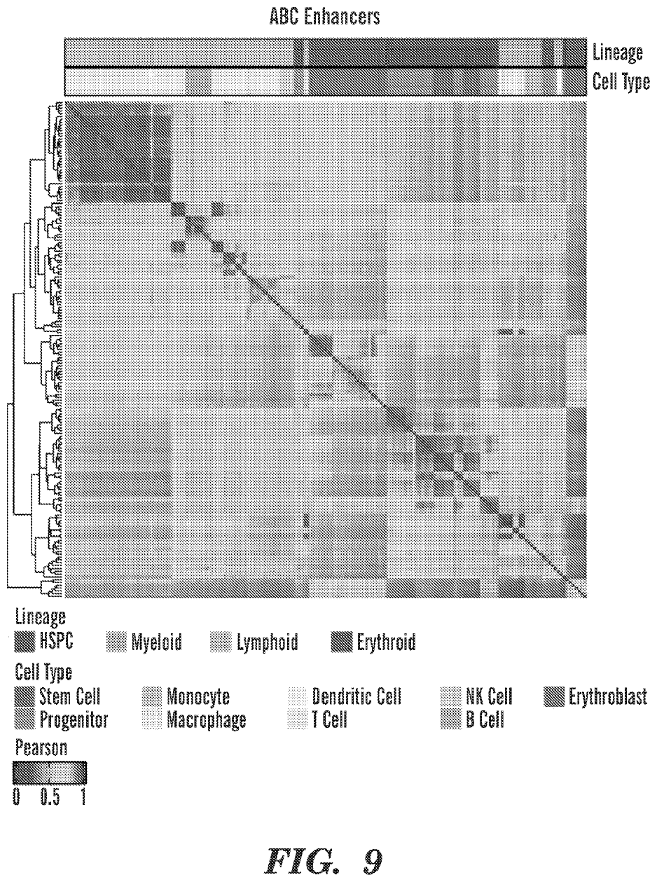

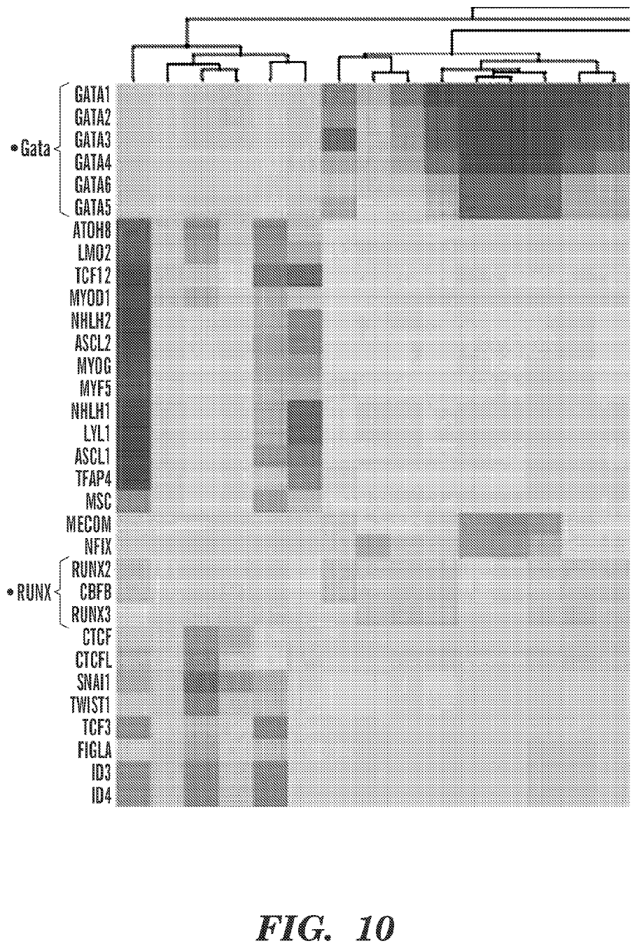

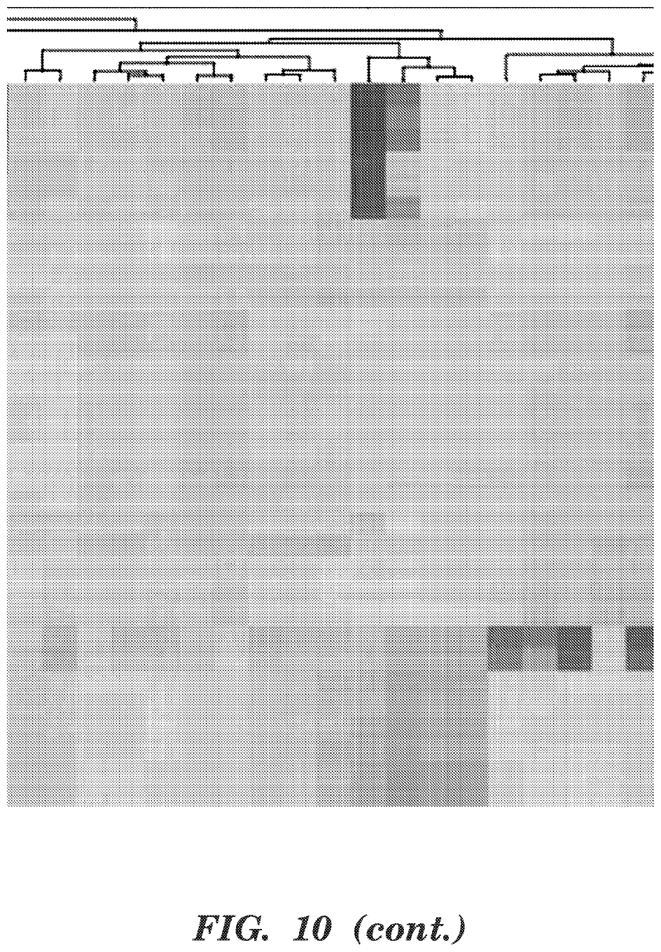

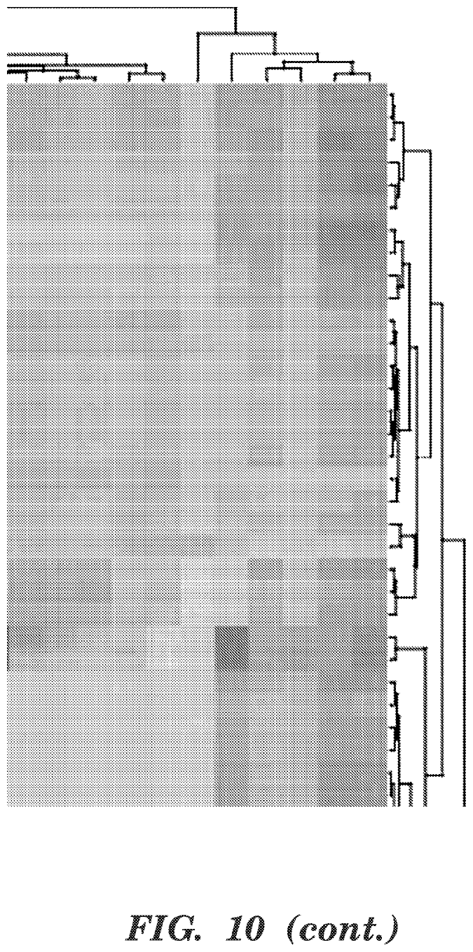

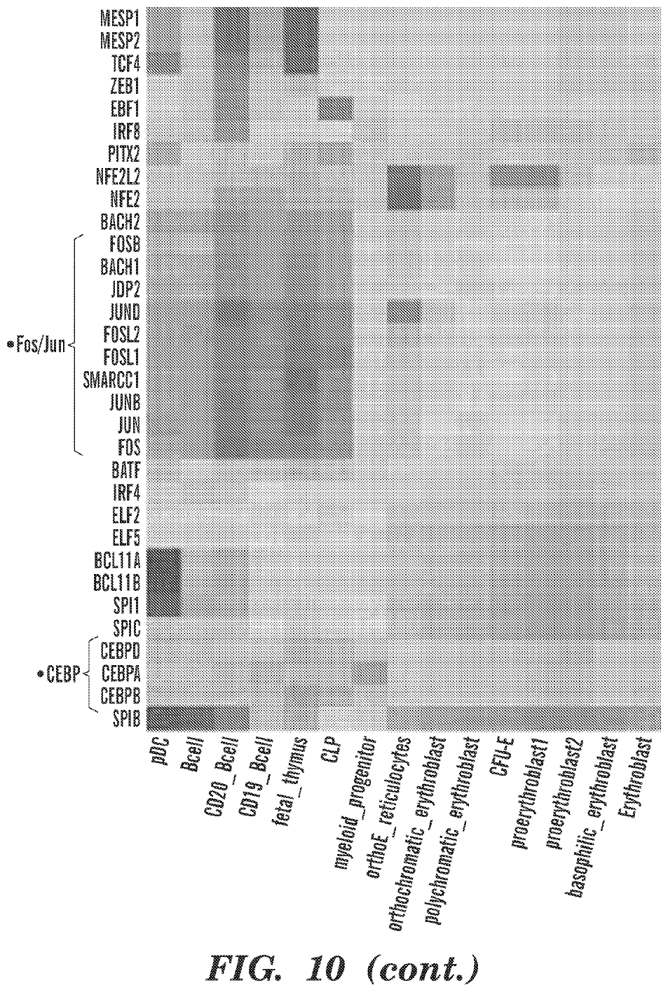

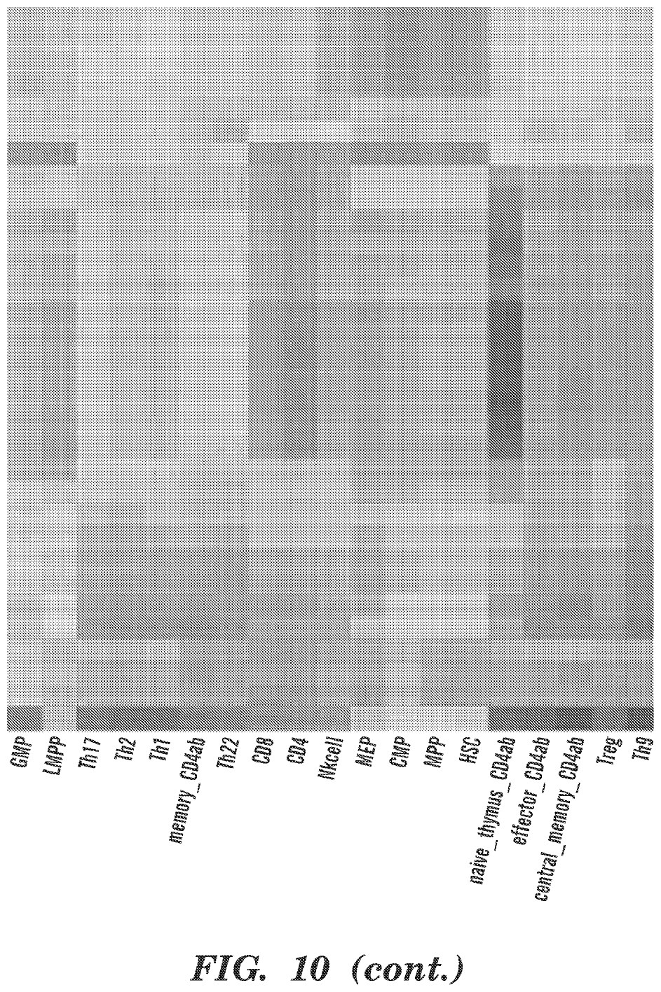

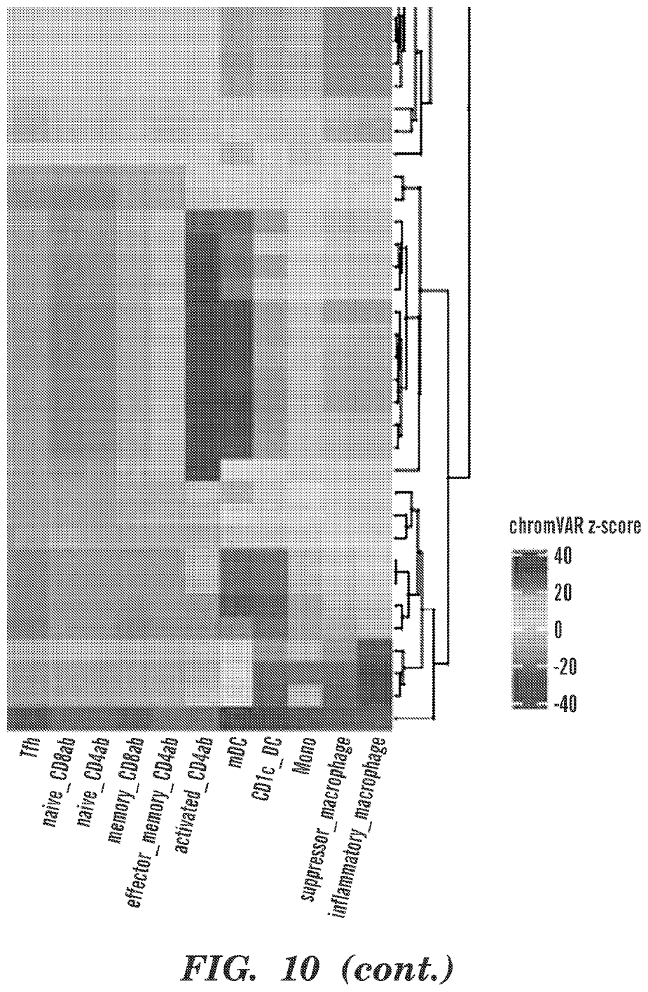

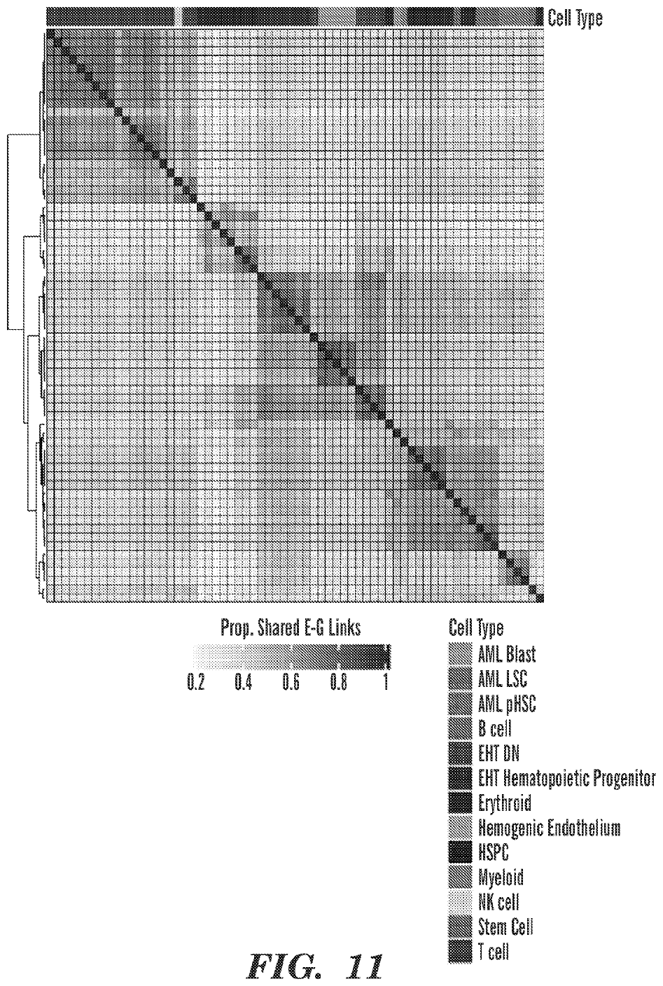

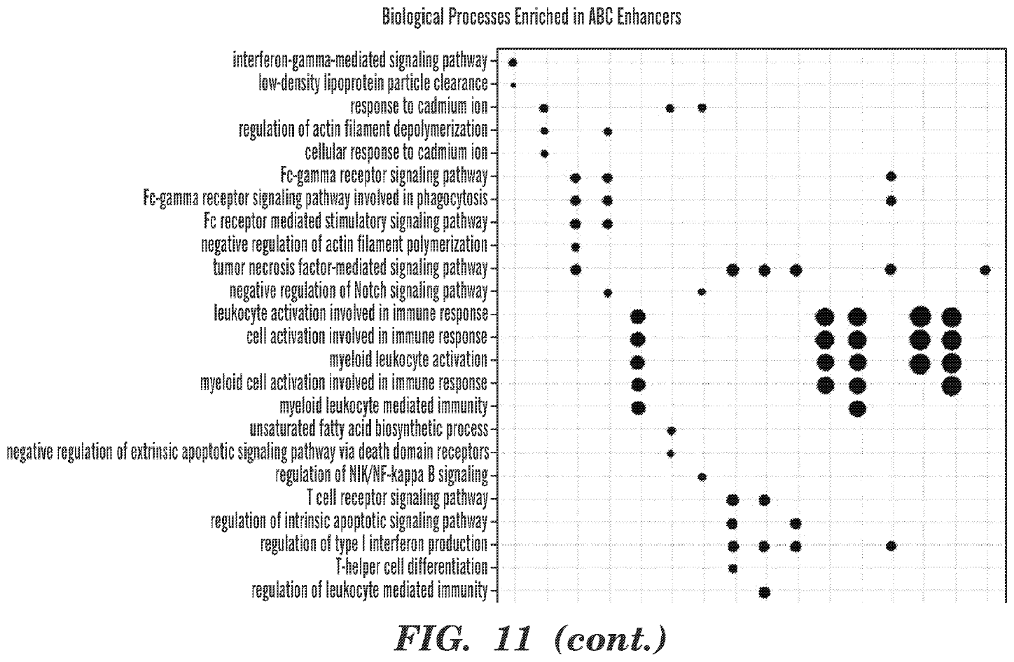

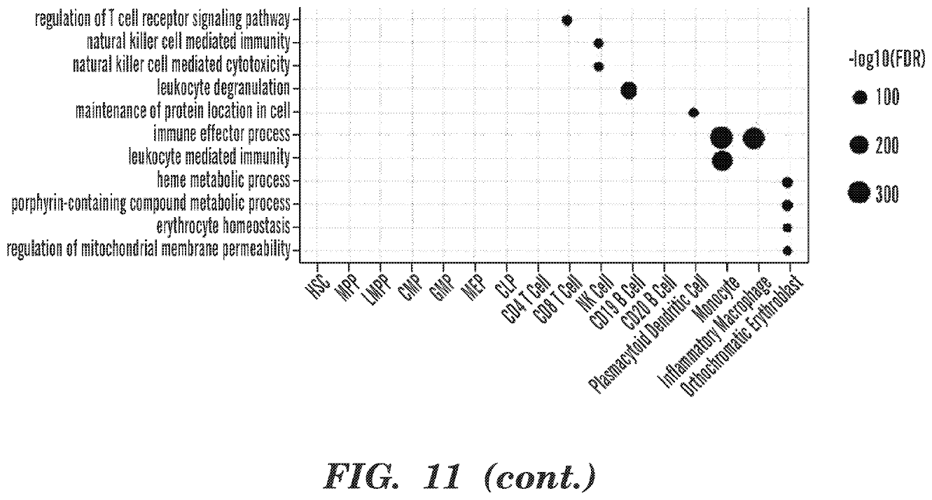

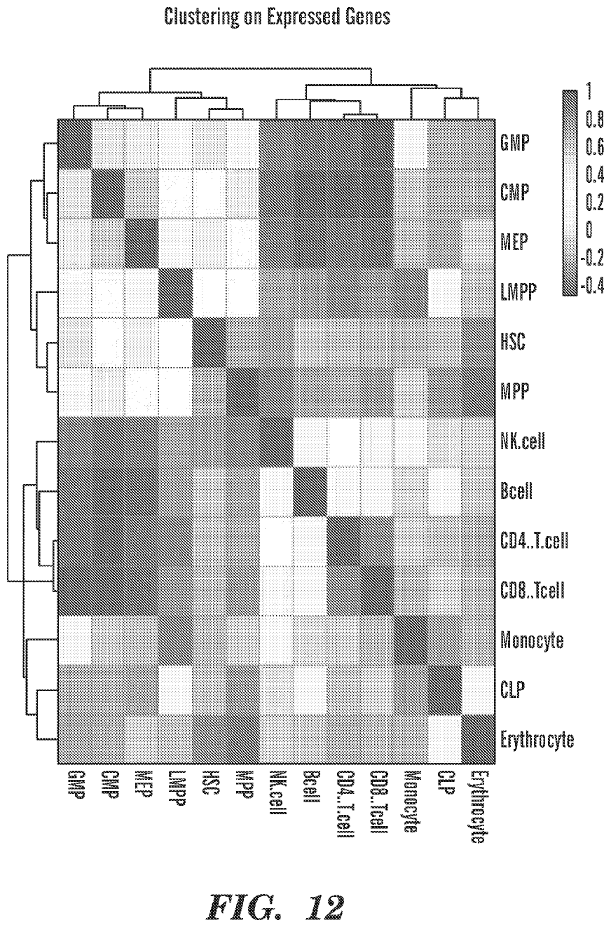

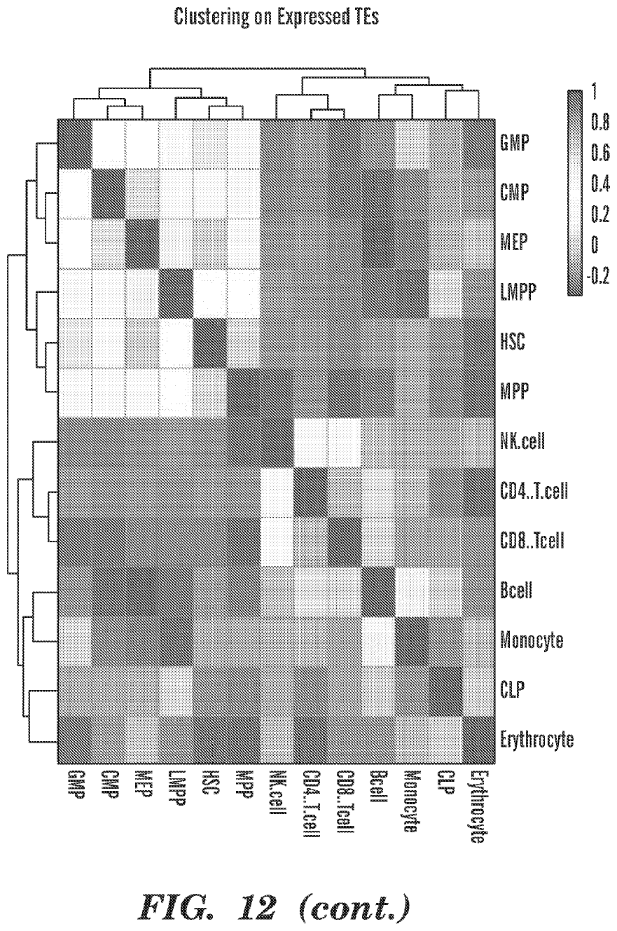

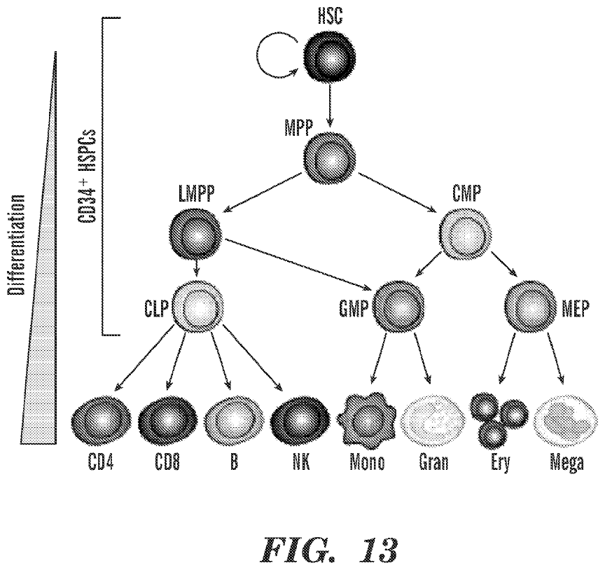

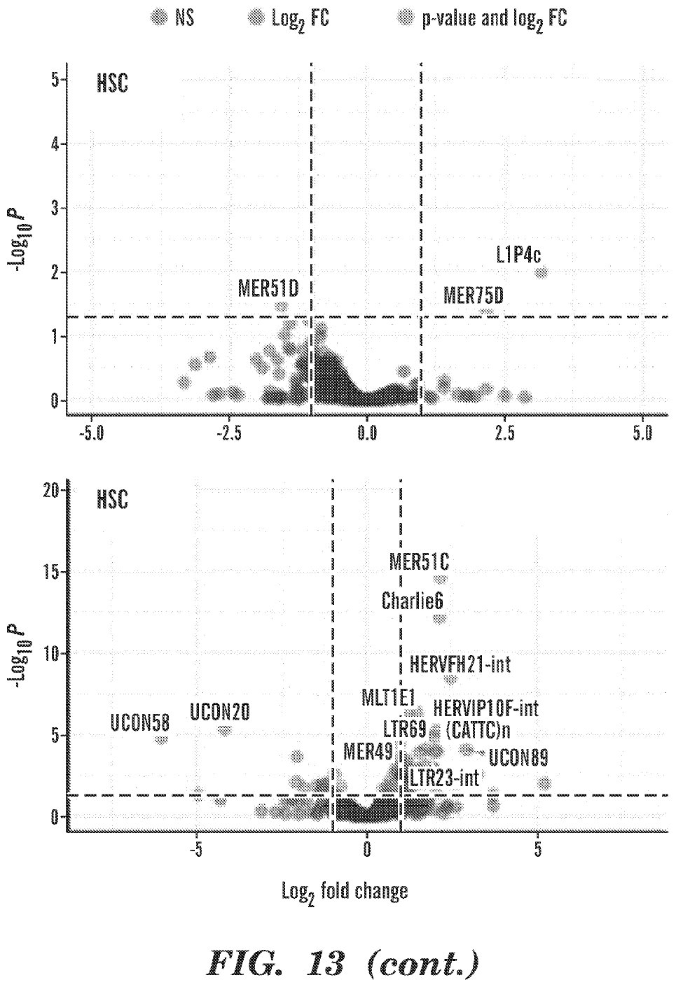

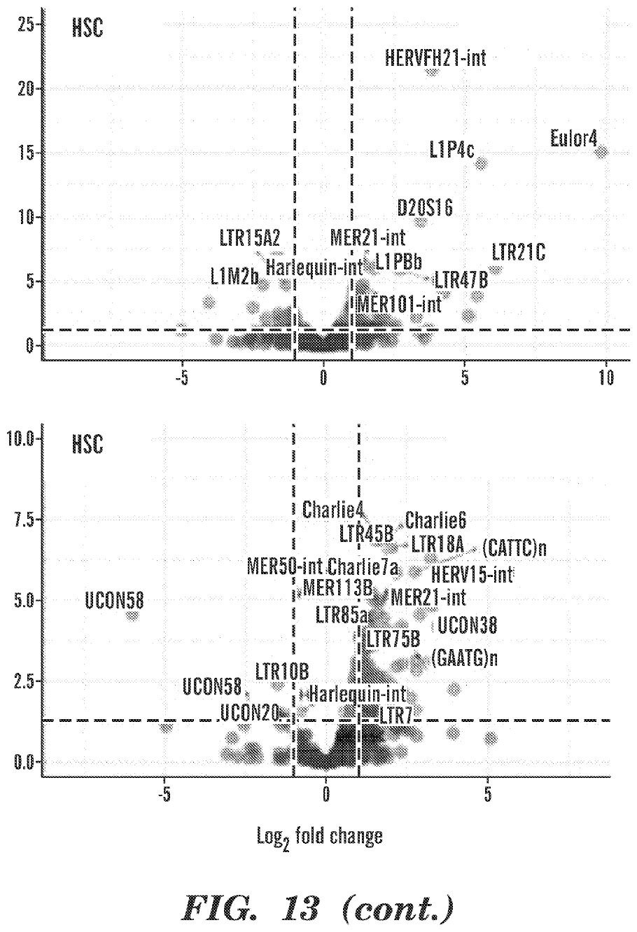

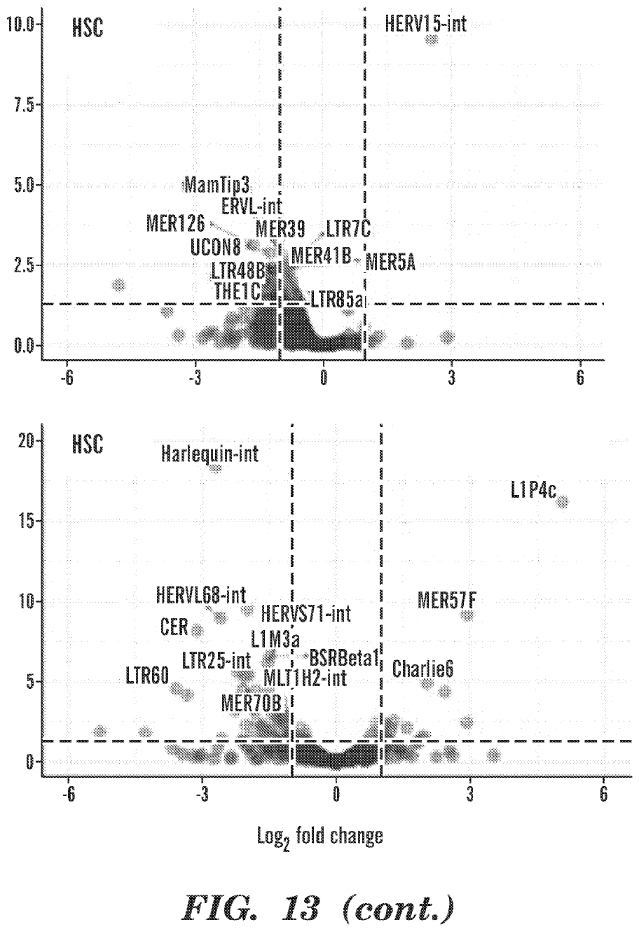

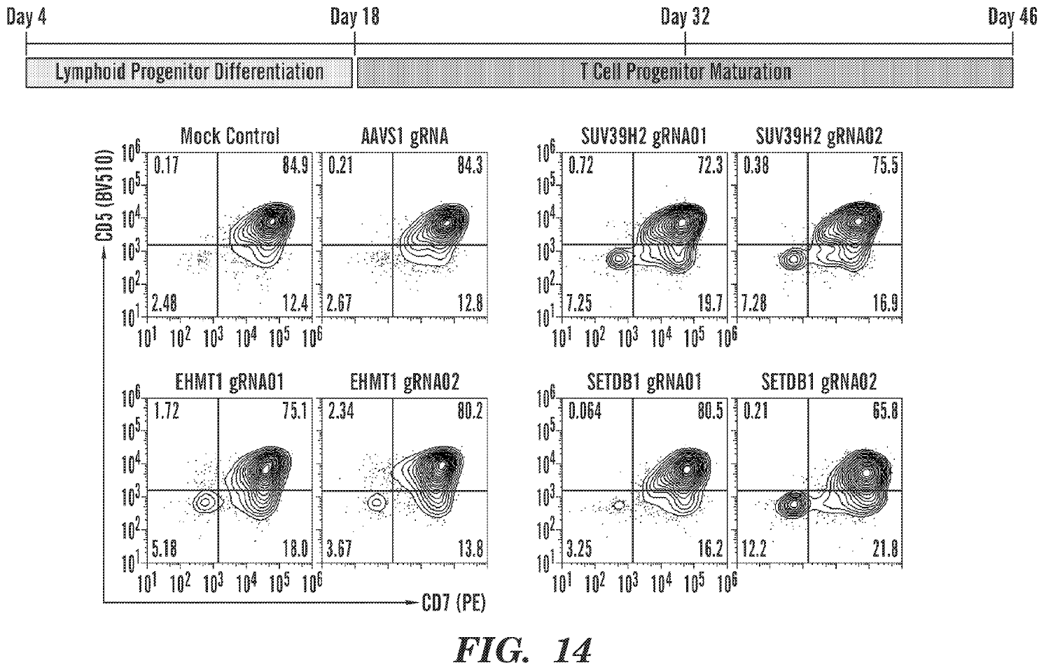

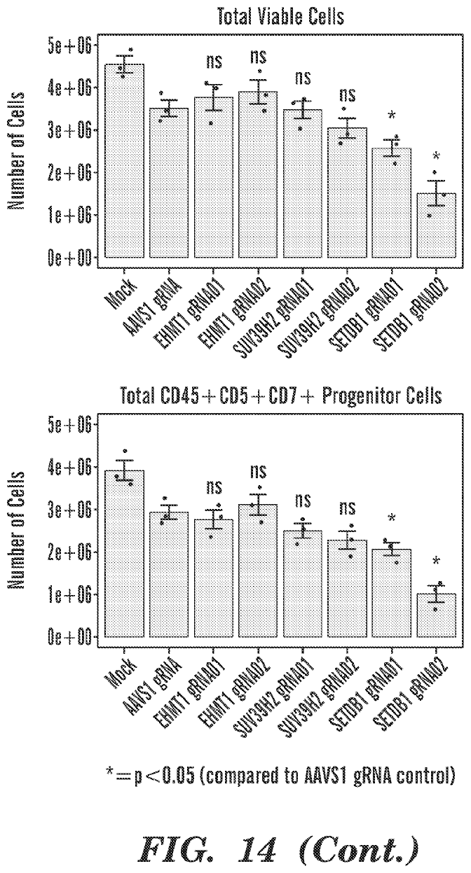

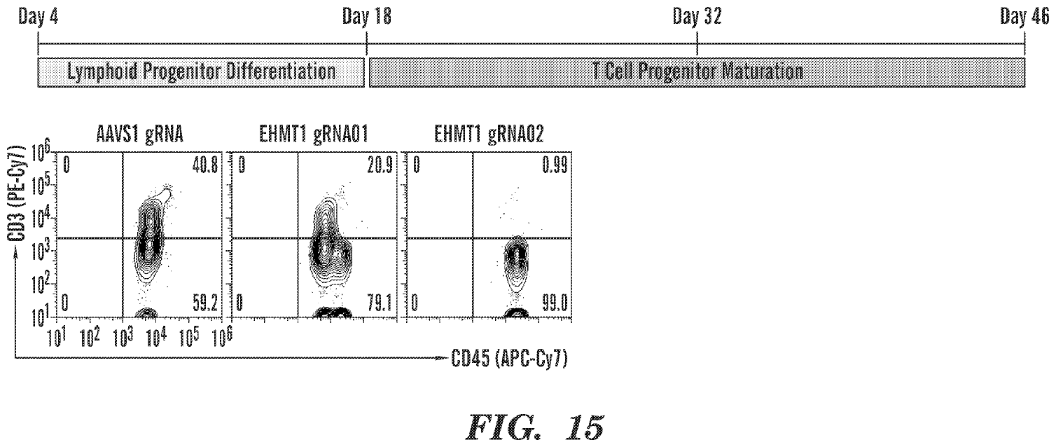

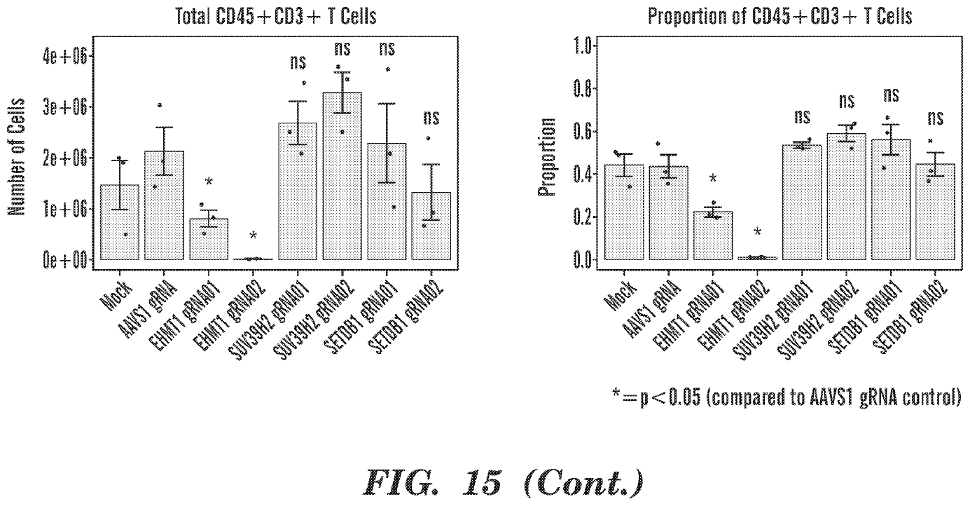

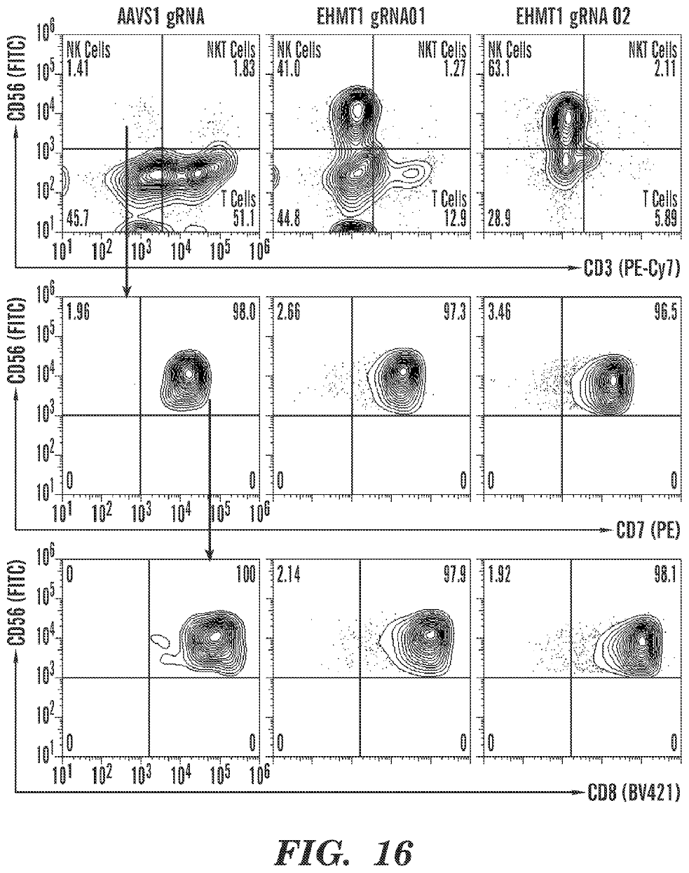

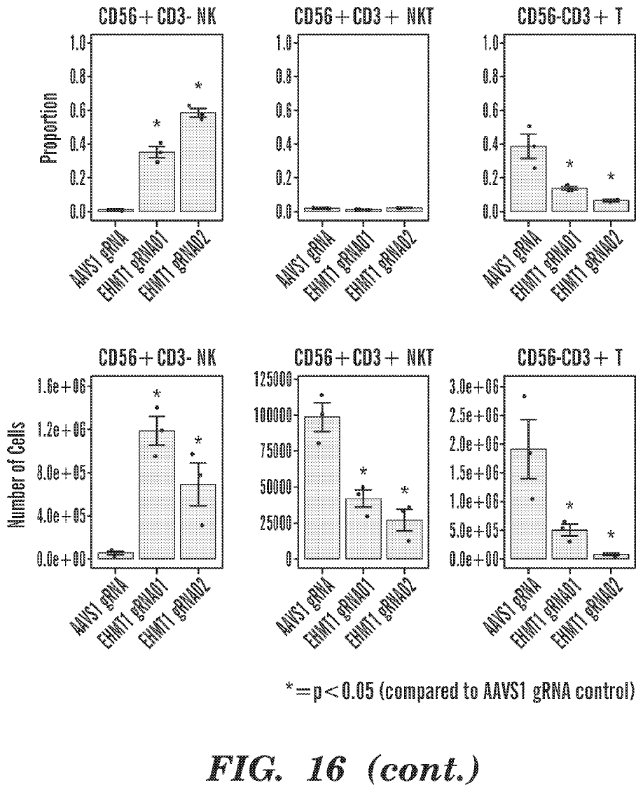

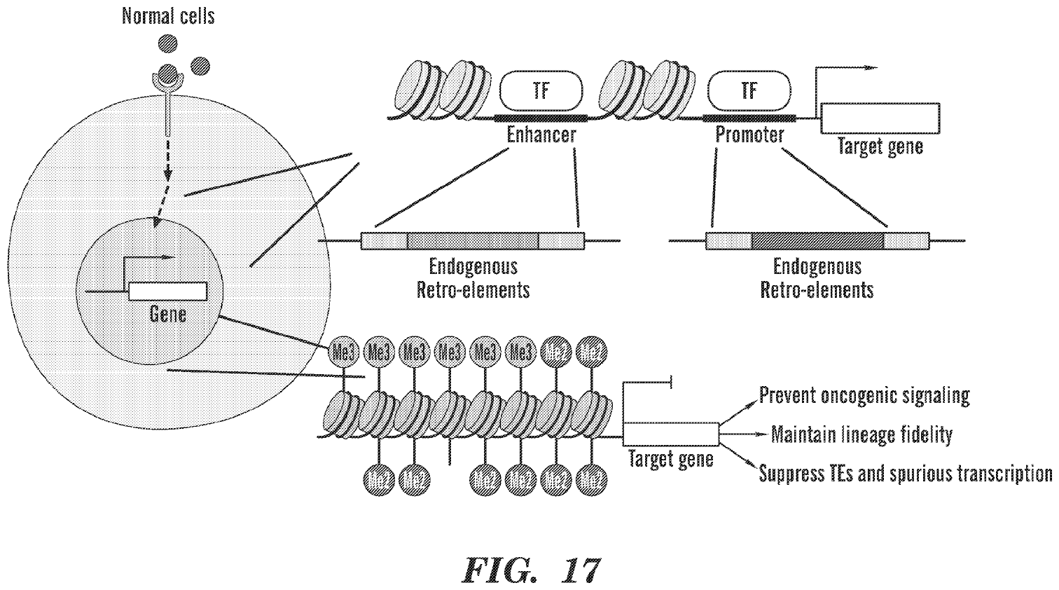

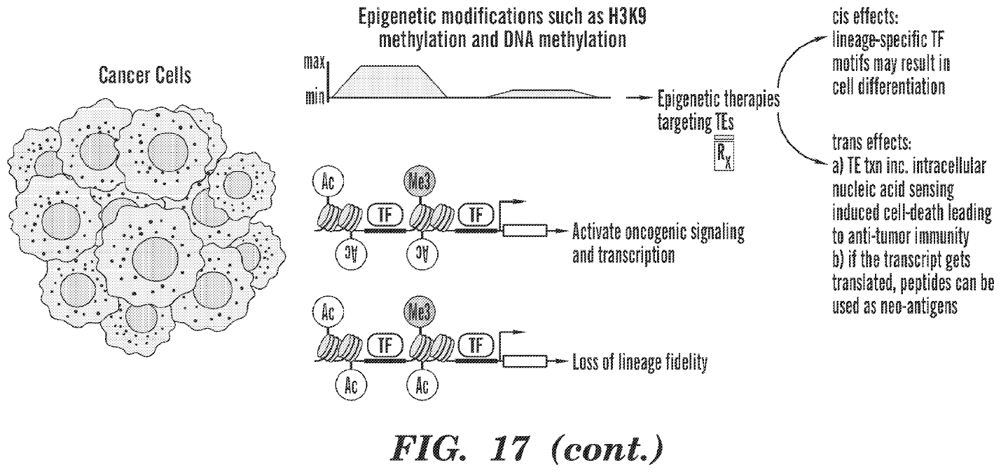

The dynamic and cell-type specific expression of TEs in the hematopoietic system prompted the inventors to hypothesize whether TEs are dynamically regulated during differentiation. In particular, heterochromatin conferred by H3K9 methylation is the main barrier to the expression of retroelements and endogenous retroviruses. H3K9 methylation is deposited and maintained by an array of H3K9 methyltransferases, which is recognized by epigenetic repressors to elicit and transcriptional silencing.scRNA-Seq profiles on 35,882 cells covering the full spectrum of the human hematopoietic system (Granja et al 2019 Nature Biotech) were utilized to analyze the expression of H3K9 methylation-related genes during differentiation (FIG. 2E). Gene module scores were calculated for genes involved in H3K9 methylation across all single cells and it was determined that mature cell types exhibited reduced expression of H3K9 methylation genes compared to HSPCs, consistent with the up-regulation of TE expression (FIGS. 18I, 20F). The nucleosome remodeling and deacetylation (NuRD) complex, which operates on chromatin marked by H3K9 methylation to mediate epigenetic silencing, demonstrated similar expression dynamics as the H3K9 methylation gene program (FIGS. 18J, 20G), further supporting the observations that the expression of TE regulatory machinery is anti-correlated to TE expression. Consistent with these observations, H3K9 demethylation expression was elevated in mature hematopoietic cell types compared to HSPCs (FIGS. 18K, 20H). Overall, these data indicate that TEs are upregulated during lymphoid differentiation with associated downregulation of TE regulatory machinery, and indicate that epigenetic regulation of TEs is concomitant with lymphoid fate specification.

### TE Regulatory Machinery Directs Lymphoid Lineage Decisions

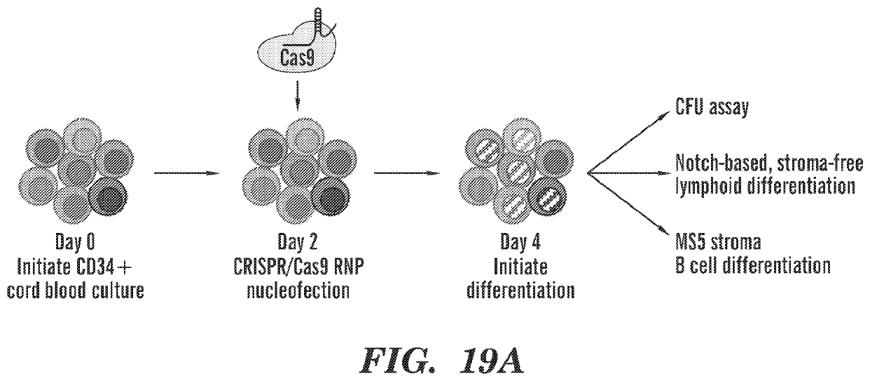

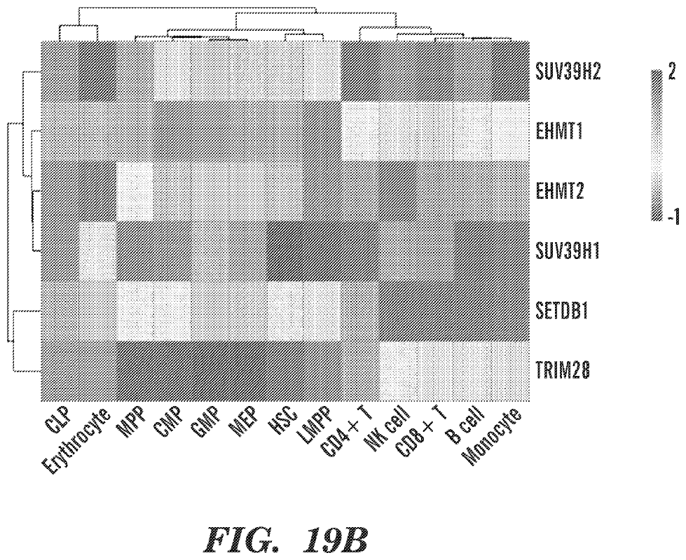

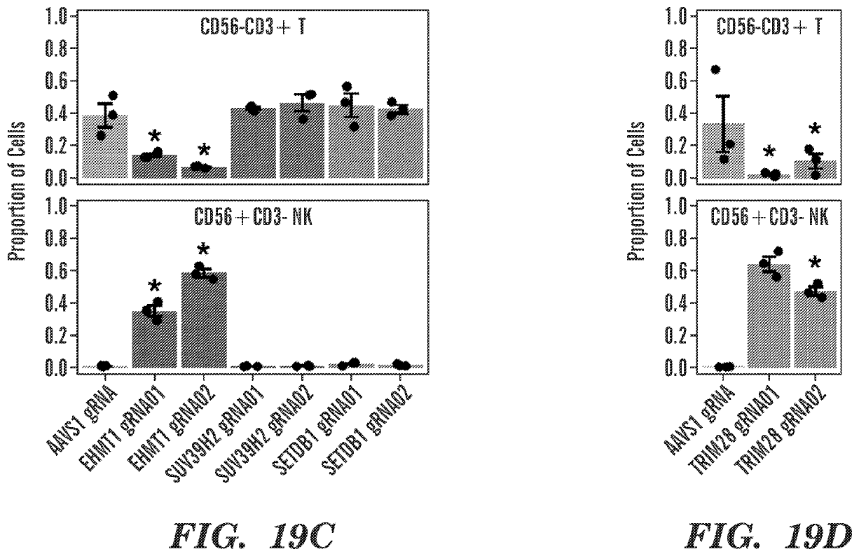

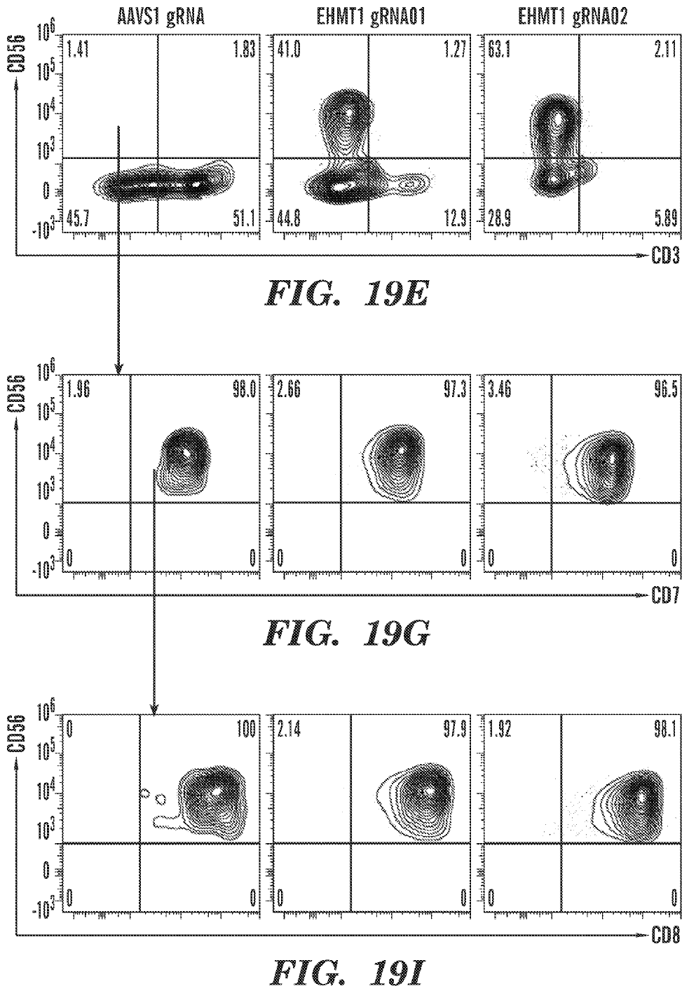

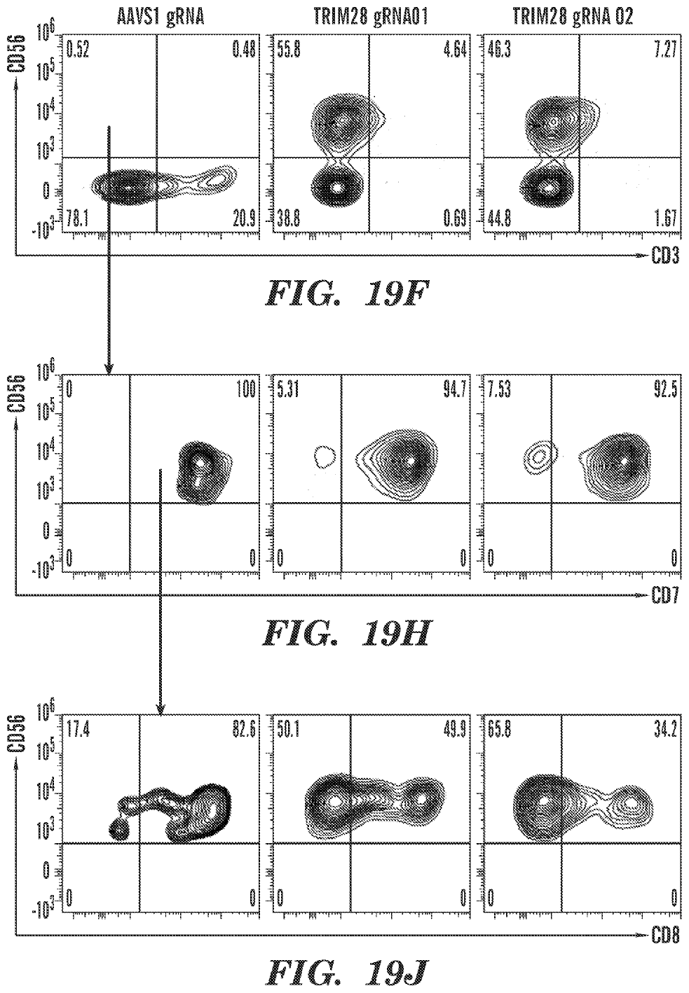

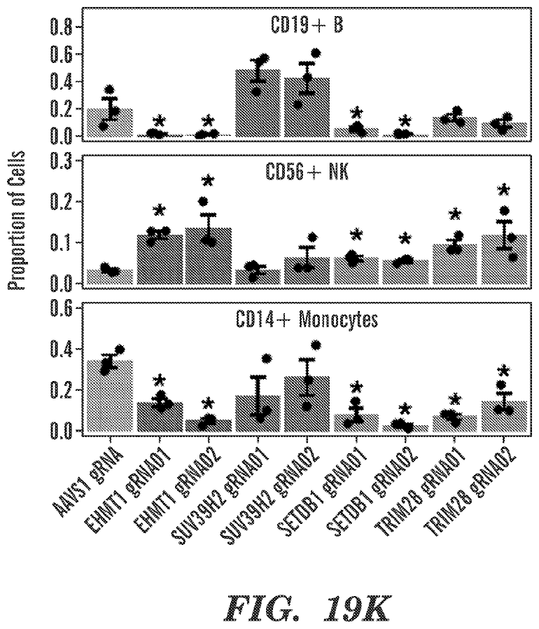

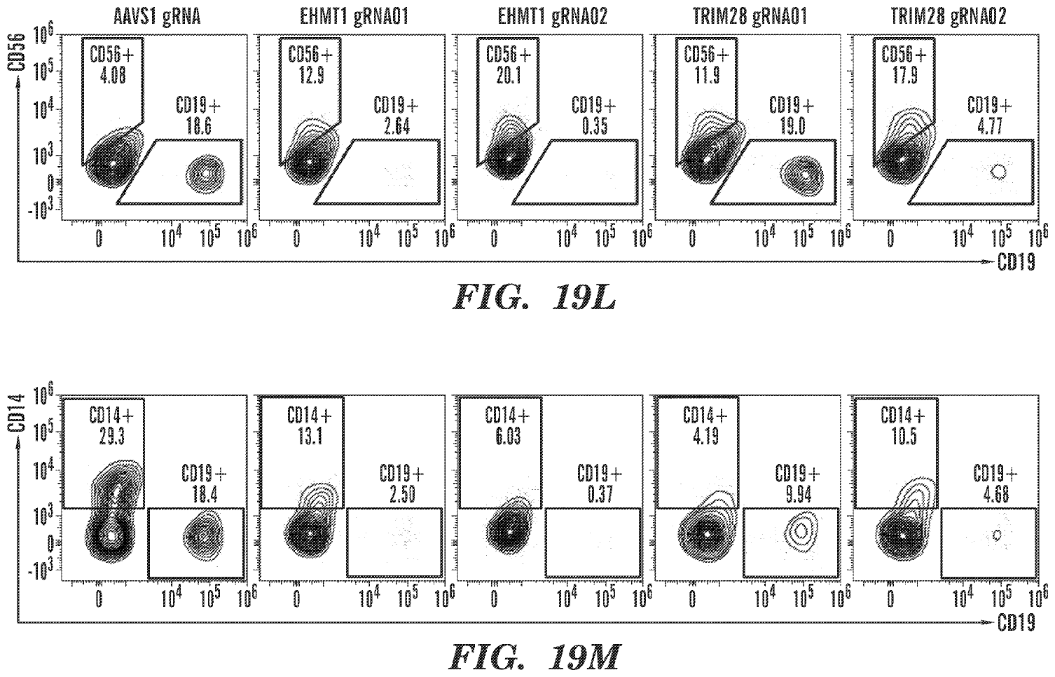

It was then asked whether perturbation of TE regulation influences lymphoid differentiation, and a gene-centric loss-of-function approach was applied to components of TE epigenetic machinery (FIG. 19A). Based on the scRNA-Seq analysis, the inventors prioritized H3K9 methyltransferases that were dynamically expressed between HSPCs and mature hematopoietic cell types (FIG. 19B), which included EHMT1, SUV39H2, and SETDB1. Furthermore, the inventors also analyzed expression of TRIM28, a well-documented master regulator of TEs that binds KRAB zinc finger proteins (KZFPs) to recruit H3K9 methyltransferases and NuRD complexes to epigenetically silence TE loci. Consistent with the observations that TEs are upregulated in lymphoid cells during differentiation, TRIM28 expression attenuates in mature hematopoietic progeny compared to HSPCs (FIG. 19B).

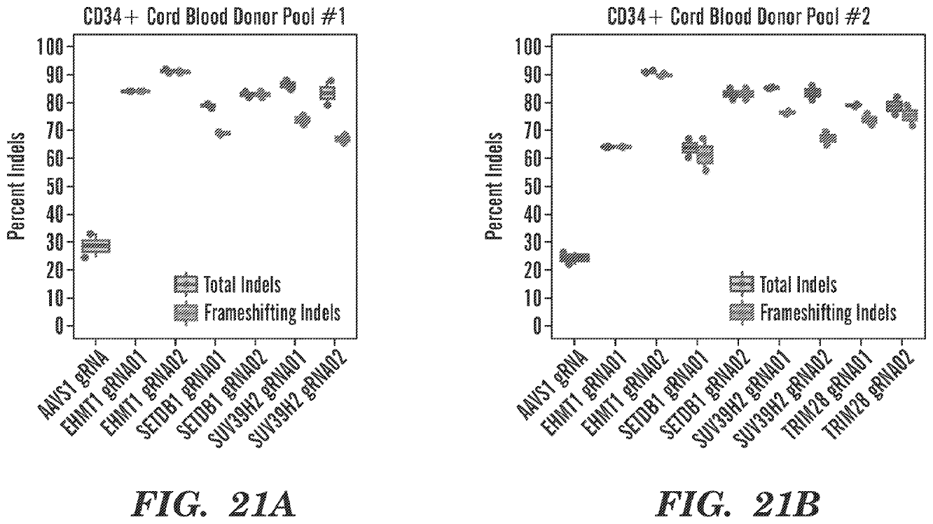

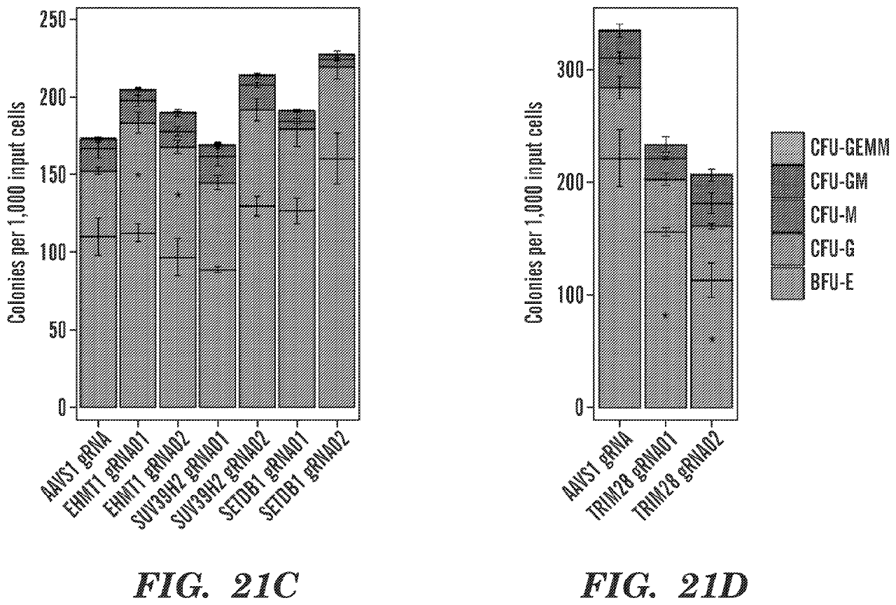

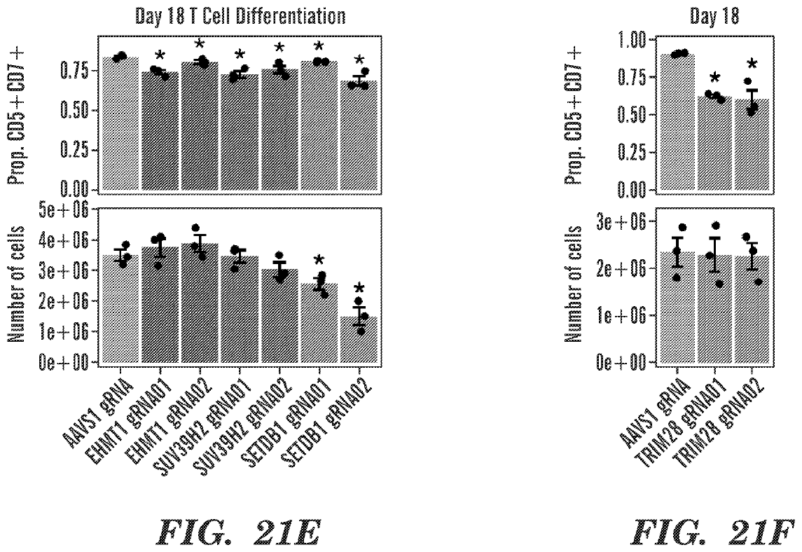

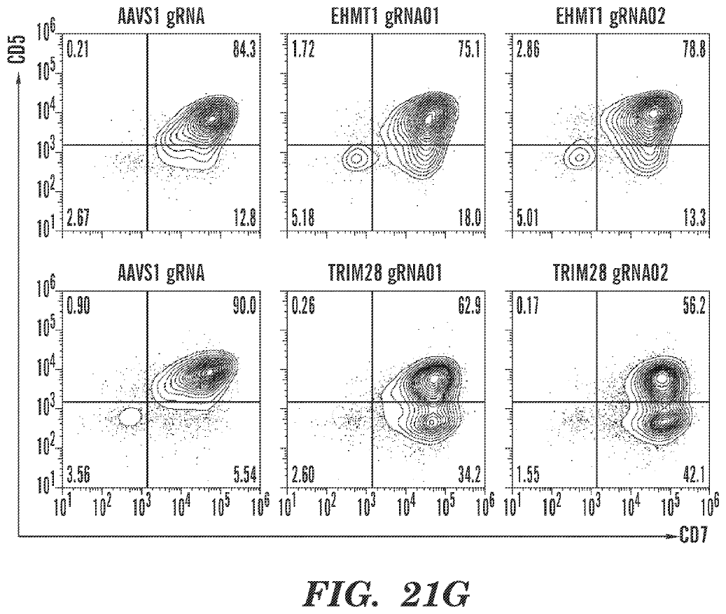

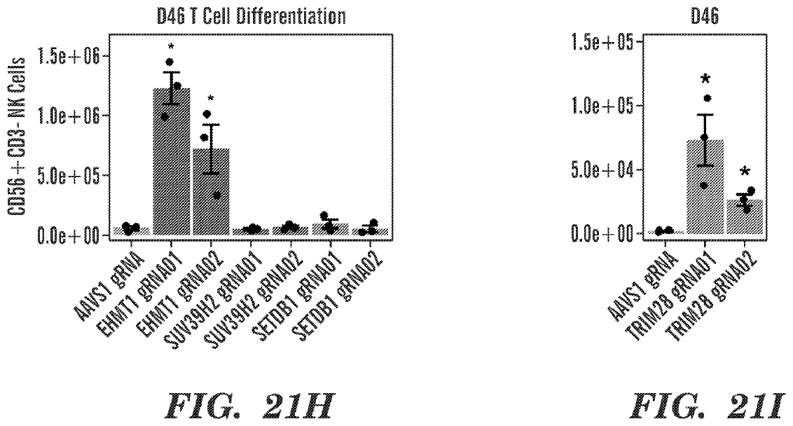

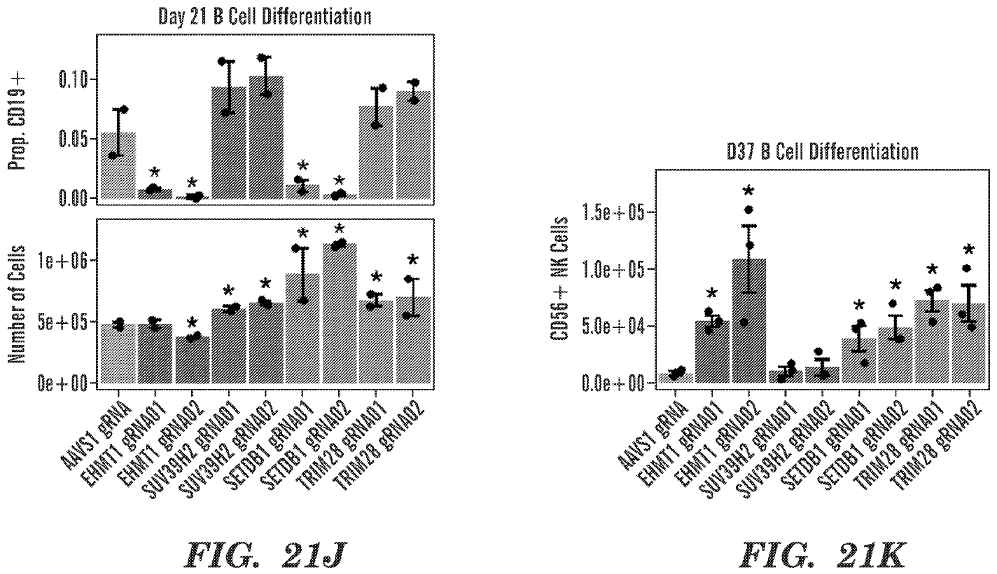

CRISPR/Cas9 was utilized to generate arrayed knockouts with two distinct gRNAs per gene in primary HSPCs, and differentiation to myeloid-erythroid, T cell and B cell fates was assessed (FIG. 19A). Delivery of Cas9 ribonucleoproteins (RNPs) resulted in highly efficient formation of frameshifting indels for each gRNA in CD34+ umbilical cord blood, enabling experiments directly on the knockout pool of cells (FIGS. 19A-29B). T cell differentiation was initiated on CD34+ HSPCs 48 hours post-delivery of Cas9 RNPs using a Notch-based, stroma-free assay. Following six weeks of in vitro differentiation, the inventors observed that knockout of EHMT1 resulted in a reproducibly pronounced reduction of CD56-CD3+ T cells (gRNA01: 13.9±1.8%, gRNA02: 6.6±0.69%) and a coupled increase of CD56+CD3−natural killer (NK) cells (gRNA01: 35.4±5.7%, gRNA02: 58.7±4.9%) compared to an AAVS1-targeting gRNA control (T cells: 38.7±12.5%, NK cells: 1.2±0.45%) (FIGS. 19C, 21H). To further support that this lineage shift is potentially mediated through TE regulatory machinery, the experiments to knockout TRIM28 were independently repeated. Knockout of TRIM28 phenocopied the T-to-NK cell lineage shift observed in EHMT1 knockouts (FIGS. 19D, 21I). Furthermore, knockout of EHMT1 or TRIM28 resulted in a reduction of T cell associated antigens, such as CD5, two weeks into in vitro differentiation without affecting overall cell viability, indicating an early manifestation of the T-to-NK fate choice within lymphoid progenitors (FIGS. 21E-21G). NK cells produced from EHMT1 (FIG. 19E) or TRIM28 (FIG. 19F) knockouts also expressed canonical NK markers such as CD7 (FIGS. 19G-19H) and CD8 (FIGS. 19I-19J), supportive of their NK identity. Interestingly, loss of EHMT1 or TRIM28 resulted in differing degrees of CD8 expression, likely indicating the generation of distinct NK subtypes.

To further evaluate perturbations to H3K9 machinery on other hematopoietic lineages, the inventors then replicated the genetic knockouts in CD34+ cord blood HSPCs and assessed B lymphoid and myeloid differentiation through an MS5-stroma co-culture assay. Consistent with observations from an in vitro T cell differentiation assay, knockout of EHMT1 resulted in an NK lineage shift at the attenuation of CD19+ B cells and CD14+ monocytes following five weeks of differentiation (FIGS. 19K-19M). While the inventors did not observe a statistically significant decrease in CD19+ B cells (FIG. 19L), knockout of TRIM28 also enhanced the proportion of NK cell fates at the expense of CD14+ monocytes (FIGS. 19K, 19M). Additionally, in this assay, knockout of SETDB1 phenocopied loss of EHMT1 (FIGS. 19K, 21J). Finally, knockout of EHMT1, TRIM28 or SETDB1 resulted in an overall increase in NK cell numbers (FIG. 21K), underscoring the role of heterochromatin regulation in NK cell fate commitment. Taken together, these data indicate that TE regulatory machinery can influence hematopoietic lineage decisions and that EHMT1 and TRIM28 regulate differentiation to NK cells.

### METHODS

### CRISPR/Cas9 Knockouts in CD34+ Umbilical Cord Blood

Genetic knockouts of select genes were generated in primary hematopoietic stem and progenitor cells (HSPCs) via nucleofection of CRISPR/Cas9 ribonucleoproteins (RNPs). Two independent knockouts were generated for each gene using separate gRNAs and knockouts were replicated across two different donor pools of human umbilical cord blood. First, CD34+ cord blood cells (AllCells) were thawed via dropwise addition of RPMI 1640+10% FBS. Cells were centrifuged at 200 g for 8 minutes and subsequently washed with FACS buffer (PBS+2% FBS). Cells were cultured for 48 hours prior to nucleofection at a density of 250,000 cells/mL in SFEM II™ media (StemCell Technologies) supplemented with 100 ng/mL SCF, TPO, FLT3L, and IL-6 (Peprotech) within a 5% O₂, 5% CO₂, 37° C. incubator. Next, the top two protospacer sequences for each desired gene were selected from the Brunello genome-wide knockout library (Broad Institute) based on the Rule Set 2 score. A control gRNA targeting the AAVS1 locus was designed in Benchling™. All gRNA sequences were synthesized by IDT as an Alt-R CRISPR/Cas9 sgRNA. RNPs were complexed by incubating 105 pmol of Cas9 protein (IDT) with 125 pmol of sgRNA in a 5 uL total volume for 10 minutes at room temperature. CD34+ cord blood HSPCs were washed with FACS buffer and 125,000 cells were resuspended in 20 μL of P3 Primary Cell Nucleofection Solution (Lonza) per nucleofection. RNPs were nucleofected into HSPCs with 3.85 uM electroporation enhancer (IDT) using a 16-reaction nucleovette on a Lonza 4D-Nucleofector (program DZ-100). Cells were cultured for 36-48 hours at 5% O₂, 5% CO₂ in 96-well U-bottom plates (Corning) then subsequently harvested for genomic DNA extraction and initiation of in vitro differentiation assays.

### Quantification of Indel Frequencies

Genomic DNA was extracted from 50,000 cord blood HSPCs using a custom extraction buffer (1 mM CaCl₂, 3 mM MgCl₂, 1 mM EDTA, 10 mM Tris-HCl, 1% Triton X-100, and 0.2 mg/ml Proteinase K) and subsequently incubated at 65° C. for 10 minutes then 95° C. for 15 minutes. Indel loci were PCR dialed-out from genomic DNA using NEBNext™ High Fidelity Master Mix (NEB) and primers that span ˜200 bps of the expected Cas9 cut site. PCR amplicons were gel extracted from a 2% agarose gel and submitted for Sanger Sequencing using forward and reverse PCR primers. Indel frequencies for each gRNA were quantified using TIDE analysis (tide.nki.nl) where the reference nucleotide sequence was from a mock-nucleofected control. The proportion of frameshifting indels was then quantified from the total TIDE indel frequency estimates.

### In Vitro T Cell Differentiation

Following 36-48 hours post-nucleofection of Cas9 RNPs, 5,000 cord blood HSPCs were differentiated to T cell lineages using the StemSpan™ T cell Generation Kit (Stem Cell Technologies) per the manufacturer's instructions with n=3 replicate differentiations per gRNA. Total viable cell counts were performed on Day 14, 28 and 42 of differentiation. T cell development was assessed by flow cytometry on days 14 and 42 of differentiation to assess lymphoid progenitors (CD45, CD5 and CD7) and mature T cells (CD45, CD4, CD8, CD3, CD56, CD5 and CD7), respectively. Cells were stained with propidium iodide as a viability marker and the following antibodies for flow cytometry: CD45 APC-Cy7 (557833, BD Biosciences), CD4 PE-Cy5 (IM2636U, Beckman Coulter Immunotech), CD8-BV421 (RPA-T8, BD Horizon), CD5-BV510 (UCHT2, BD Biosciences), CD3-PE-Cy7 (UCHT1, BD Pharmigen), CD7-PE (555361, BD Pharmigen), CD56 FITC (BD Pharmigen).

### In Vitro B Cell Differentiation

MS5 stromal cells were seeded onto gelatin-coated 24-well plates at a density of 10,000/well. Cells were allowed to grow for 48 hours in Myelocult™ H5100 (Stem Cell Technologies) supplemented with 100 ng/mL SCF (R&D Systems), 50 ng/mL TPO (Peprotech), 10 ng/mL FLT3L (R&D Systems), and 25 ng/mL IL-7 (R&D Systems) in order to condition the media prior to seeding hematopoietic cells. Following 36-48 hours post-nucleofection of Cas9 RNPs, 2,500 CD34+ cord blood HSPCs were seeded onto MS5 stroma with at least n=3 replicate differentiations per gRNA per time-point. Fresh medium with cytokines was supplied weekly over five weeks of differentiation. Wells were harvested at regular intervals throughout the differentiation by gentle trituration to obtain total cell counts and for flow cytometric assessment of hematopoietic differentiation. Cells were stained with propidium iodide and either a hematopoietic lineage antibody panel, comprising CD45 APC-Cy7 (BD Biosciences), CD19 PE (BioLegend), CD56 FITC (BioLegend), CD33 APC (BioLegend), CD14 PerCP (BD Biosciences), or a B-cell specific antibody panel, comprising CD45 APC-Cy7 (BD Biosciences), CD19 PE (BioLegend), CD20 PE-Cy5 (BD Biosciences), IgM BV510 (BD Biosciences).

### Hematopoietic Colony Assays

Following 36-48 hours post-nucleofection of Cas9 RNPs, 1,000 cord blood HSPCs were plated in 60-mm dishes containing 1.5 mL of methylcellulose SF H4636 (StemCell Technologies) with n=3 replicate assays per gRNA. After 14 days of culture in a humidified chamber, plates were scored in a gRNA-blinded manner for granulocyte, erythrocyte, monocyte/macrophage, and CFU-GM/GEMM colonies.

### Flow Cytometry

Cells were stained for flow cytometry at room temperature for 20 minutes in the dark, followed by a washing step with PBS+2% FBS. Data were collected on a Sony MA900 and analyzed using FlowJo™. The following antibodies/stains were utilized at a 1:100 dilution: Propidium Iodide, CD45-APC-Cy7 (BD, clone 2D1), CD19-PE (BioLegend, clone HIB19), CD10-BV510 (BioLegend, clone HI10a), CD56-FITC (BioLegend, clone 5.1H11), CD33-APC (BioLegend, clone WM53), CD14-PerCP (BD, clone MφP-9), hIgM-BV510 (BD, clone G20-127), CD20-PE-Cy5 (BD, clone 2H7), CD7-PE (BD, clone M-T701), CD5-BV510 (BD, clone UCHT2), CD3-PE-Cy7 (BD, clone SK7), CD4-PE-Cy5 (BD, clone RPA-T4), CD8-BV421 (BD, clone RPA-T8), NKG2D-APC (BD, clone 1D11).

### Example 2

### Introduction

The regulation of gene expression is fundamental for the establishment of cellular identity and function. The coordinated activities of transcription factors (TFs) and chromatin factors on a DNA template orchestrate transcriptional networks that drive cellular phenotypes and govern cell fate decisions. The hematopoietic system is an archetypical example of the necessity of these mechanisms to regulate the dynamic differentiation processes that generate diverse, mature blood cell types from multipotent hematopoietic stem and progenitor cells (HSPCs). Yet, the understanding of how TFs and chromatin factors elicit commitment toward distinct hematopoietic lineages is continuously evolving. Insights into the regulatory architecture governing hematopoietic differentiation can facilitate novel approaches to derive specific blood cells in vitro for therapeutic applications.

Transposable elements in the human genome have garnered increased attention due to mounting evidence that evolution has co-opted them as transcriptional regulators in specific cellular contexts. These ancestral elements, comprising retrotransposons and DNA transposons, can serve as binding sites for TFs and participate as transcriptional enhancers. While TE-derived regulatory activity can be observed across multiple human tissues, TEs comprise a higher proportion of enhancer states in the hematopoietic lineage, suggesting that TEs are particularly important for hematopoietic transcriptional regulatory networks.

The regulatory contributions of TEs are tightly controlled by chromatin machinery. TRIM28, a well-documented suppressor of TEs, is recruited to specific genomic sites via direct interactions with KRAB-zinc finger proteins (KZFPs). Together this complex achieves TE silencing through deposition of H3K9 methylation and removal of histone acetylation via the NuRD complex. Formation of heterochromatin more generally via H3K9 methyltransferases also functions to silence TE families. Importantly, these processes can contribute to the dynamic activity of TEs during cell state transitions, such as somatic cell reprogramming and early embryogenesis.

Increasing evidence suggests that certain cell types are prone to TE-mediated gene regulation, particularly immune cells. Genome-wide chromatin profiling studies in immune cells have revealed a significant fraction of TEs contribute to putative enhancers that modulate essential immune-related functions, such as interferon-inducible inflammasome activation. Furthermore, endogenous retrovirus (ERV) proteins have been co-opted in immune cells to serve a dominant negative function, interfering with viral infection. Speculation that TEs prompted the evolution of inflammatory gene regulatory networks, led us to ask whether TEs are involved in the dynamic differentiation processes that generate immune cells from HSPCs. There is a lack an understanding of the potential role of TEs in hematopoietic differentiation and lineage determination.

In this study, the contributions of TEs were systematically dissected to human hematopoiesis. A comprehensive cell type-resolved atlas of enhancer-gene regulation was built, comprising every major cell type in the human hematopoietic system. Lineage and cell type-specific enrichments of TE families were identified in putative enhancers. TEs were particularly wired to the regulatory landscape of lymphoid cells, serving as docking sites for critical TFs and exhibiting dynamic expression during lymphoid differentiation. TE derepression, achieved by modulation of regulators of heterochromatin formation within HSPCs, resulted in the surprising acquisition of natural killer (NK) cell fates in T or B cell supportive differentiation conditions. Specifically, knockout of the transcriptional co-repressor TRIM28 or the H3K9 methyltransferase EHMT1 generated distinct lymphoid progenitor populations with enriched activity for NK-relevant TFs. Further, NK cells derived as a result of knockout of TRIM28 or EHMT1 exhibited distinct classes of derepressed TEs and downstream effector properties. These findings deepen our understanding of the essential role of TE regulation during hematopoietic differentiation, and enable novel approaches to derive diverse sets of NK cells ex vivo for potential therapeutic applications.

### Results

### Genome-Wide Framework for Enhancer-Gene Inference in the Hematopoietic System

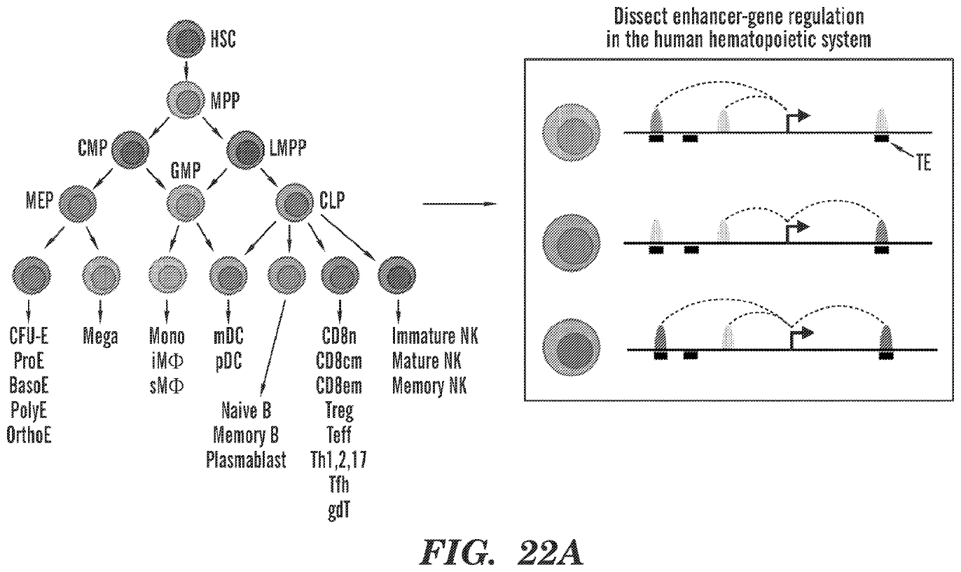

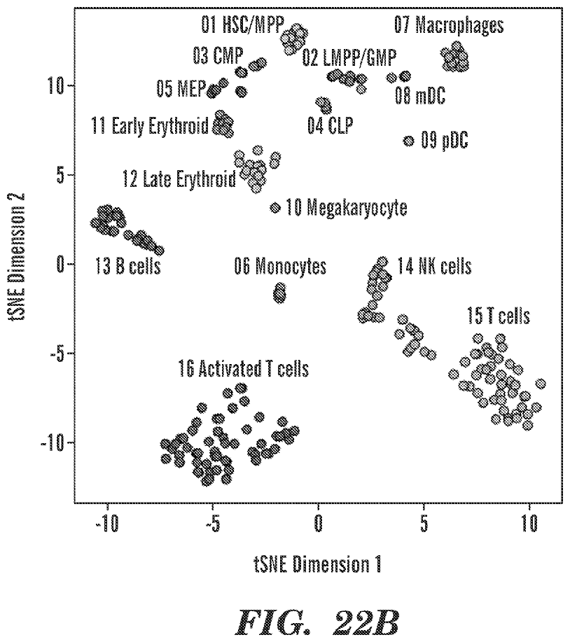

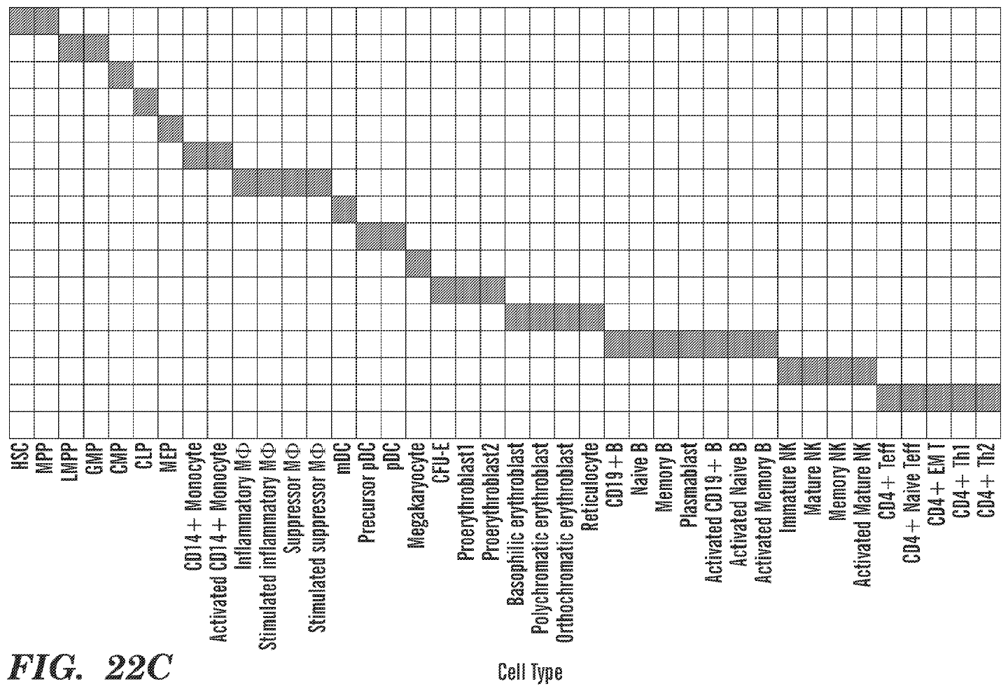

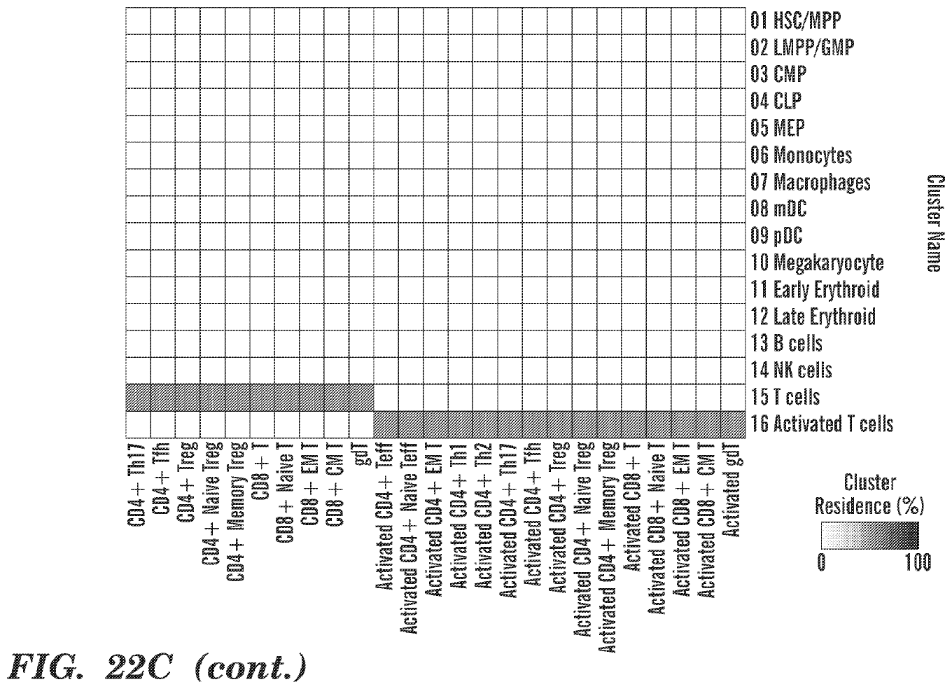

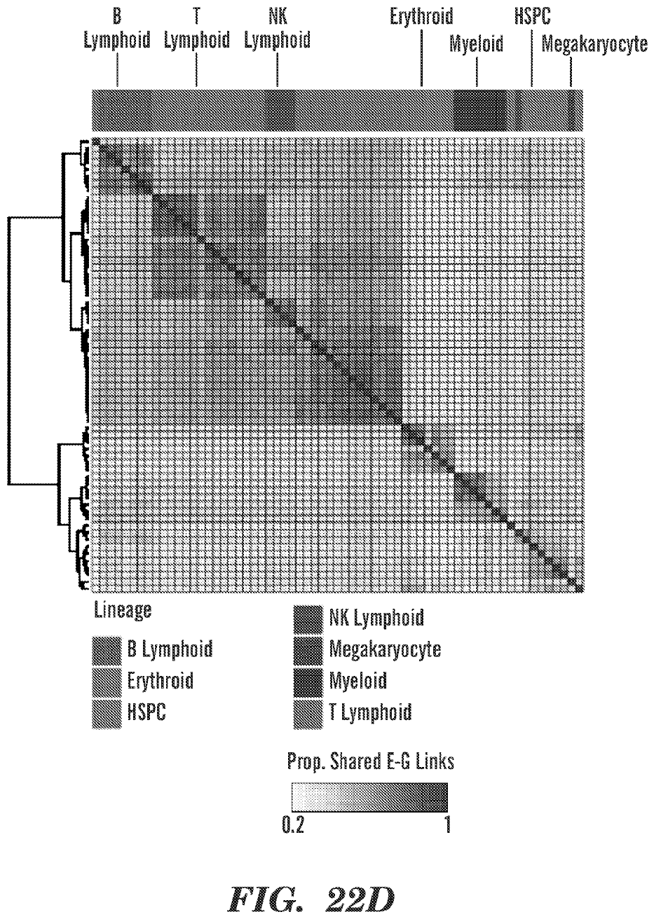

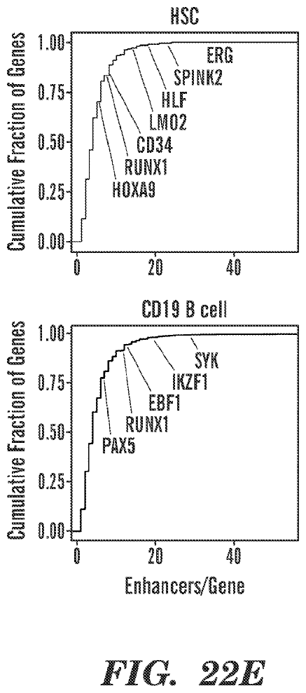

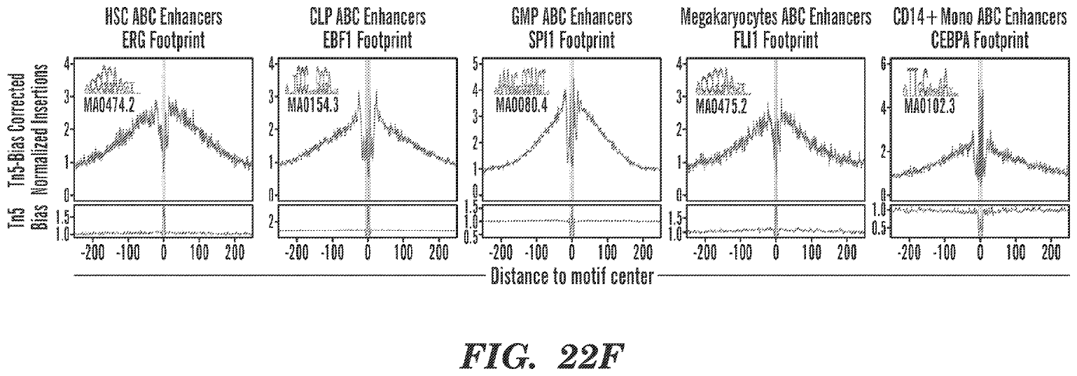

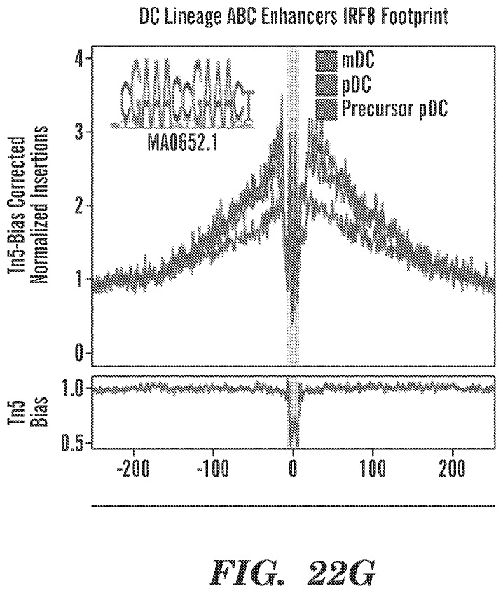

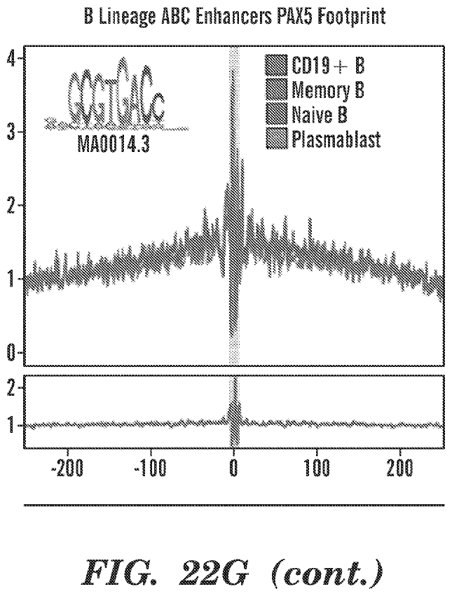

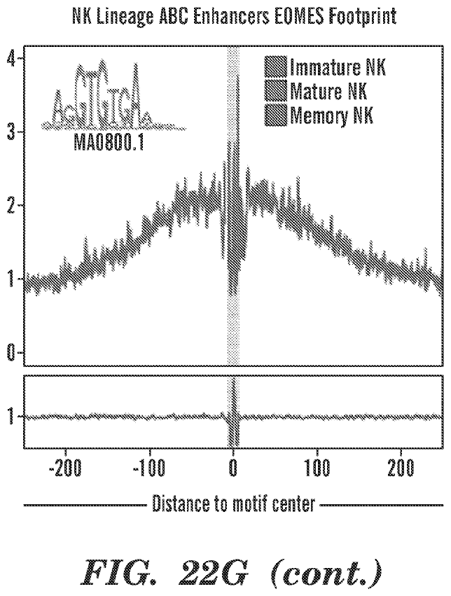

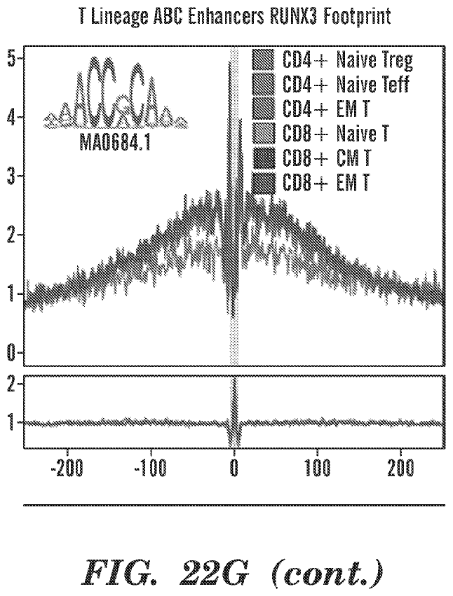

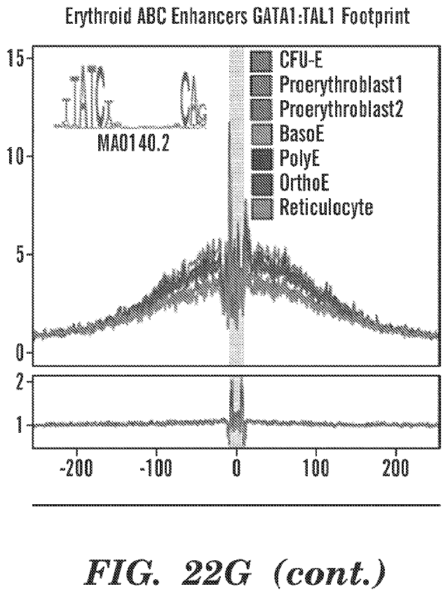

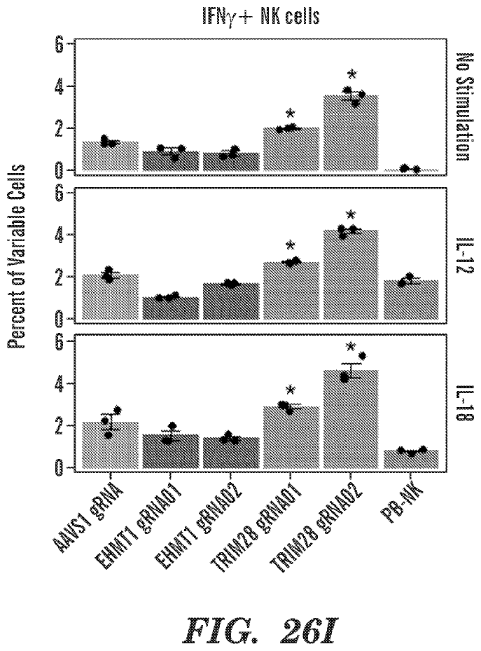

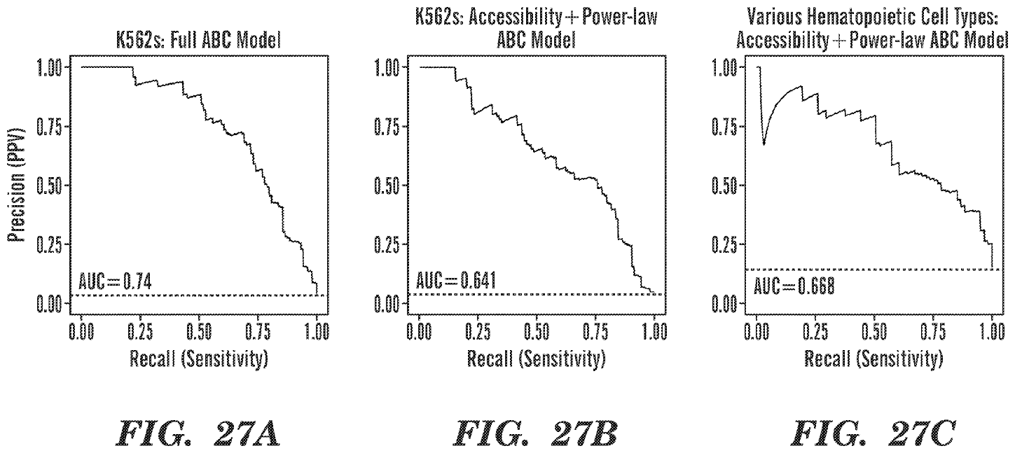

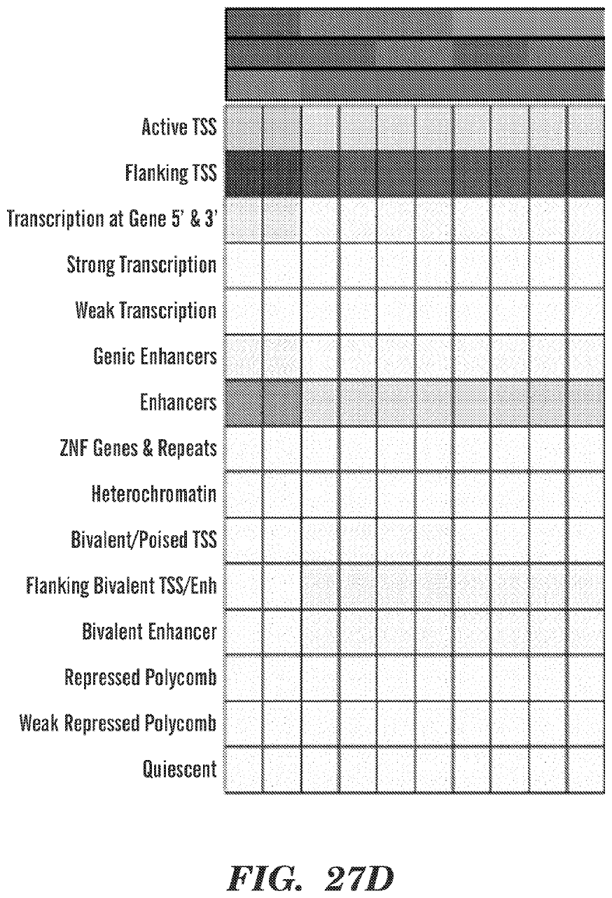

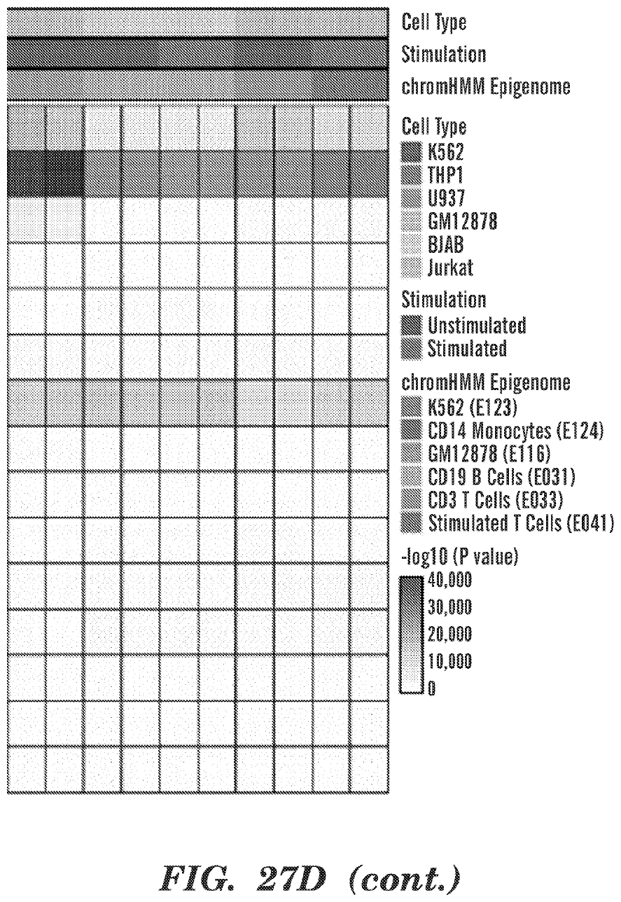

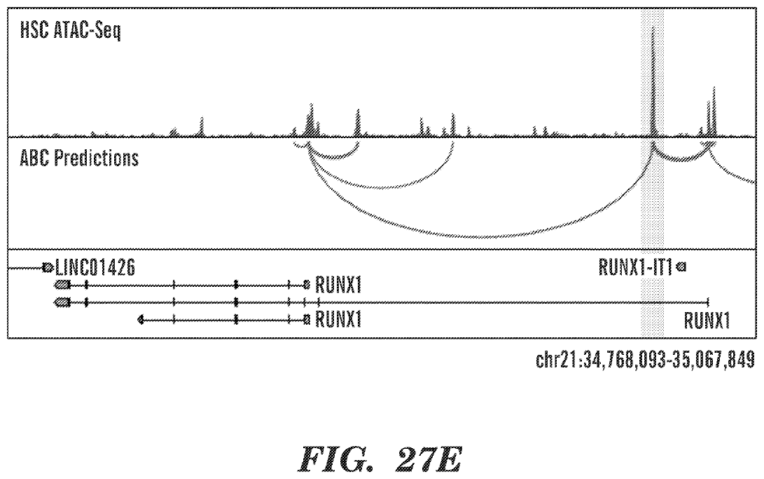

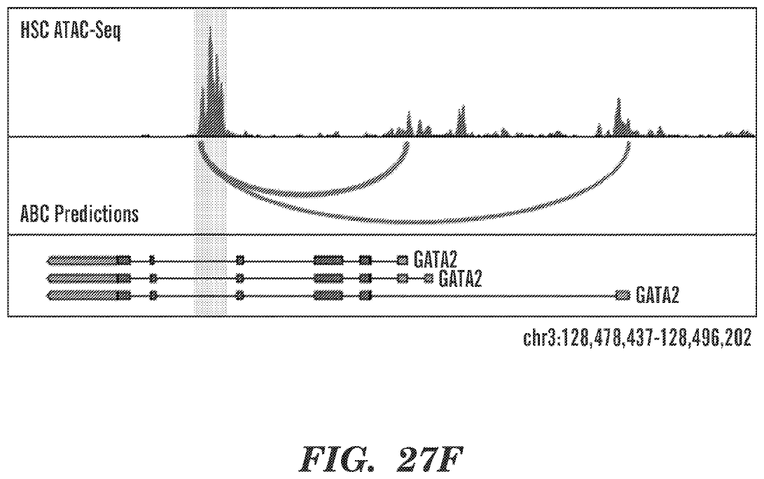

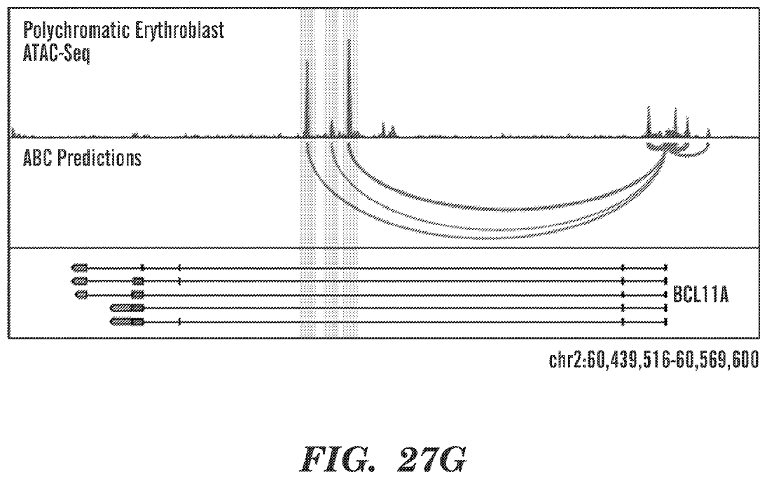

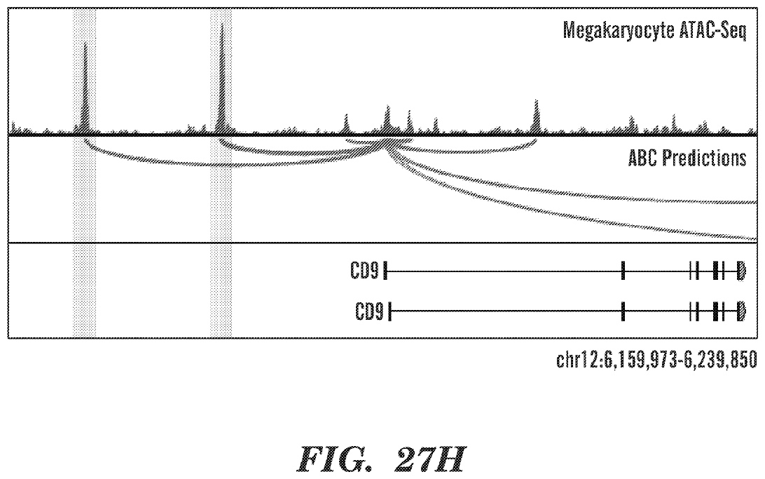

To systematically investigate the regulatory contributions of TEs to human hematopoietic differentiation (FIG. 22A), robust reference maps of enhancer-gene regulation were first built. Although hematopoiesis has been the subject of numerous profiling efforts over decades to map cis-regulatory elements, chromatin states and transcription, less attention has been paid to the enumeration of enhancers and the genes they regulate within primary cells spanning the entirety of the human hematopoietic system. The Activity-by-Contact (ABC) model developed to infer enhancer-gene regulation, robustly predicts CRISPRi perturbation experiments, and thus, effectively identifies functional enhancers. The model posits that an enhancer's relative contribution to gene transcription is dependent on its activity (measured by chromatin accessibility and H3K27ac ChIP-Seq) and contact frequency with the gene's promoter (measured by HiC or a power-law function of promoter-enhancer genomic distance). The applicability of the ABC model was extended by defining enhancer activity solely in terms of chromatin accessibility, which consequently enables regulatory predictions for low-abundance cell types samples, where H3K27ac ChIP-Seq is experimentally infeasible, such as hematopoietic stem cells (HSCs). When benchmarked against experimental CRISPRi-FlowFISH screens in various hematopoietic cell types, it was found that this modified model resulted in near comparable performance in identifying enhancer-gene pairs, as assessed by precision recall statistics (FIGS. 27A-27C). Additionally, enhancer predictions were compared from each of these hematopoietic cell lines to chromHMM-defined regulatory regions and observed that predicted enhancers were strongly enriched within active chromatin (FIG. 27D). The modified model predicted experimentally validated enhancer-gene interactions within the relevant cell type, including intronic enhancers regulating RUNX1 and GATA2 within HSCs (FIGS. 27E-27F), enhancers regulating BCL11A in erythroid precursors (FIG. 27G), and enhancers regulating CD9 in megakaryocytes (FIG. 27H). Finally, the ABC model accurately identified known examples of TE-derived enhancers, specifically the MER41G enhancer that regulates APOL1 within innate immune cells upon interferon-gamma stimulation (FIG. 27I). These results indicate that our modified ABC framework provides a tractable and scalable approach to identify putative enhancers in the human hematopoietic system and the potential contributions of TEs to cell-type specific gene regulation.

### A Comprehensive Atlas of Human Hematopoietic Enhancer-Gene Regulation

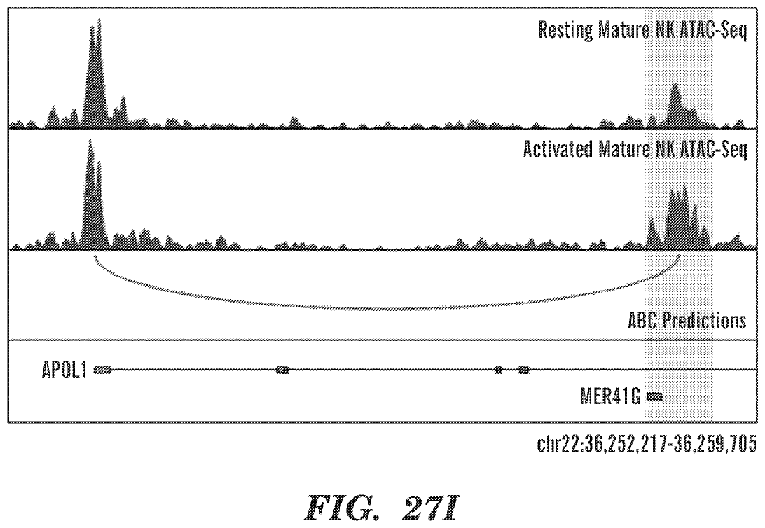

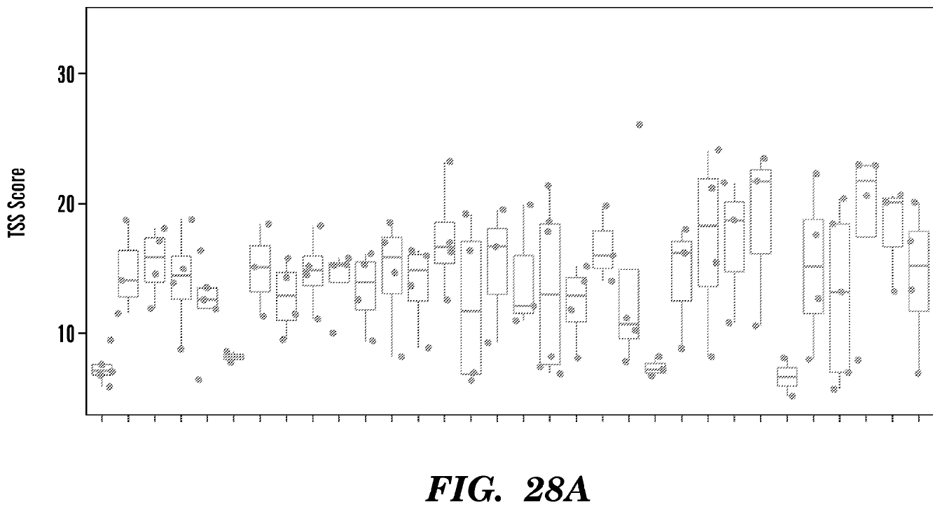

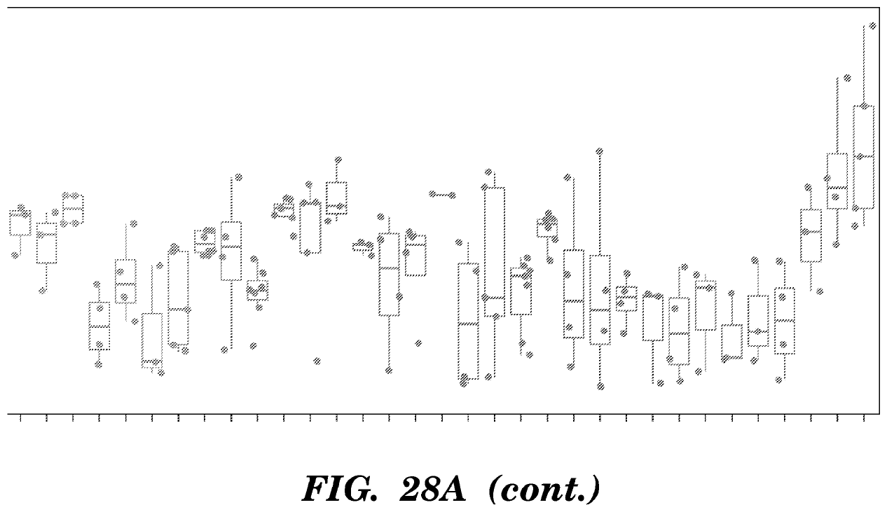

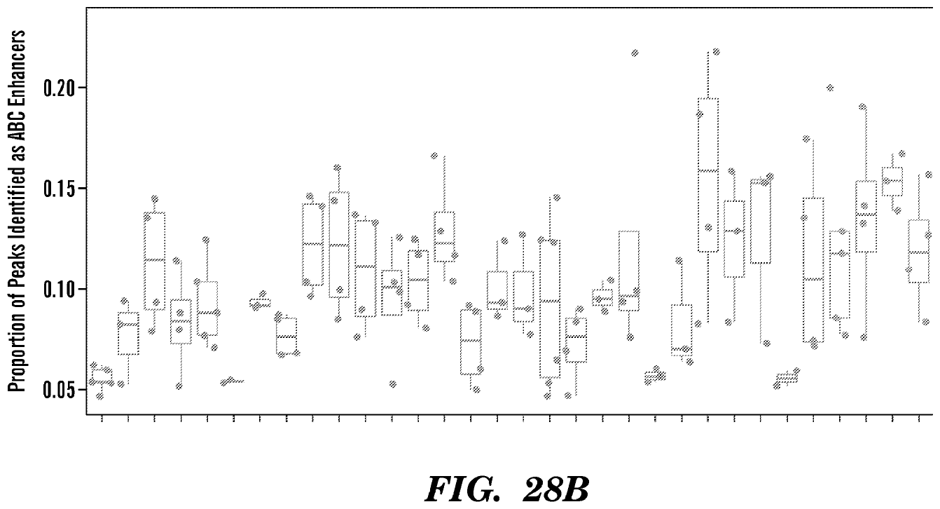

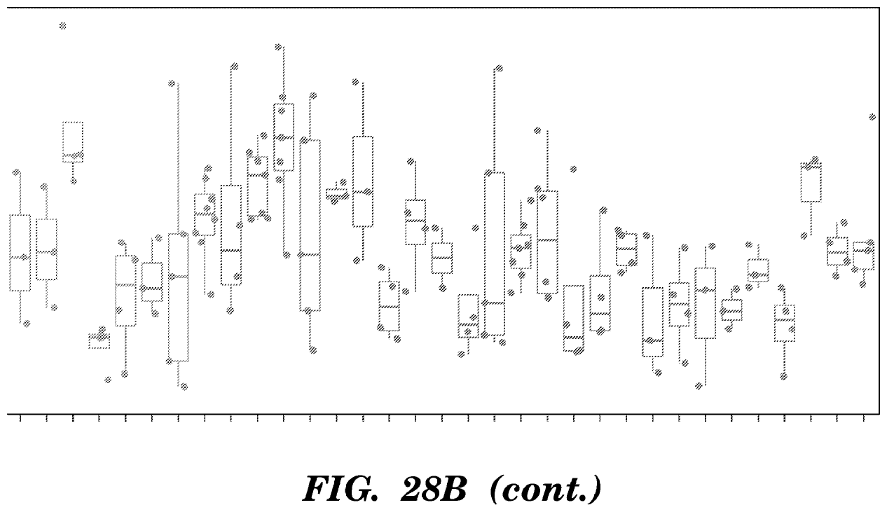

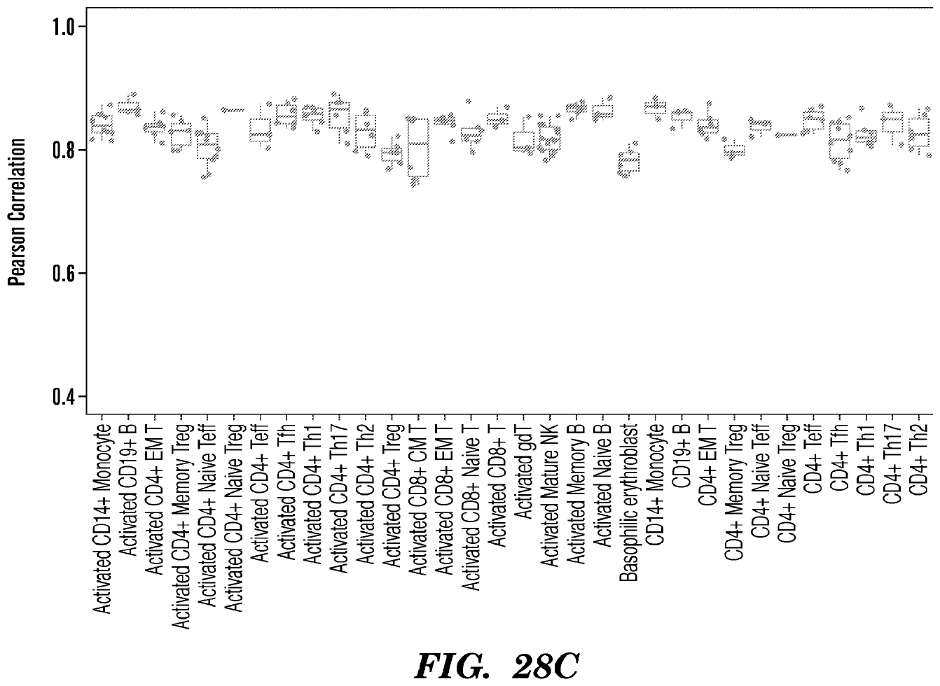

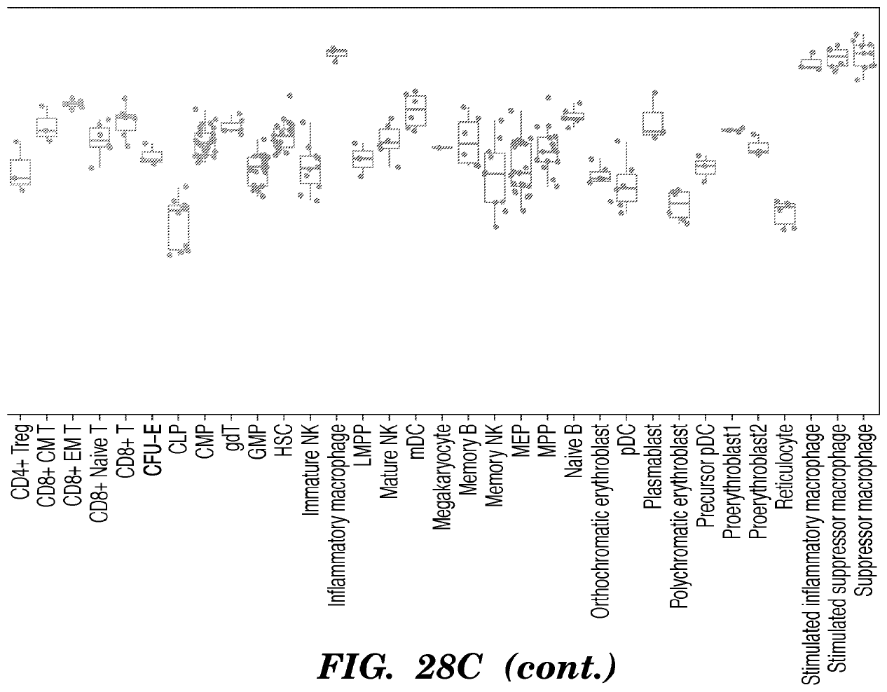

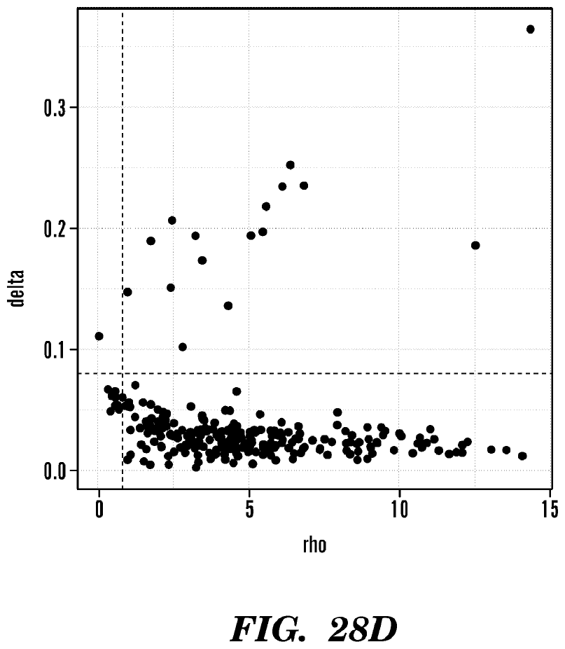

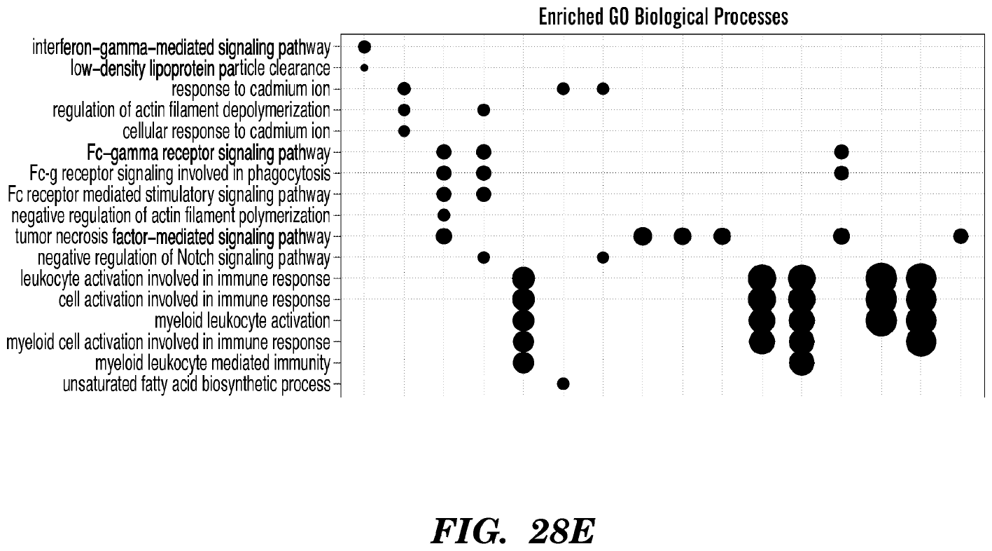

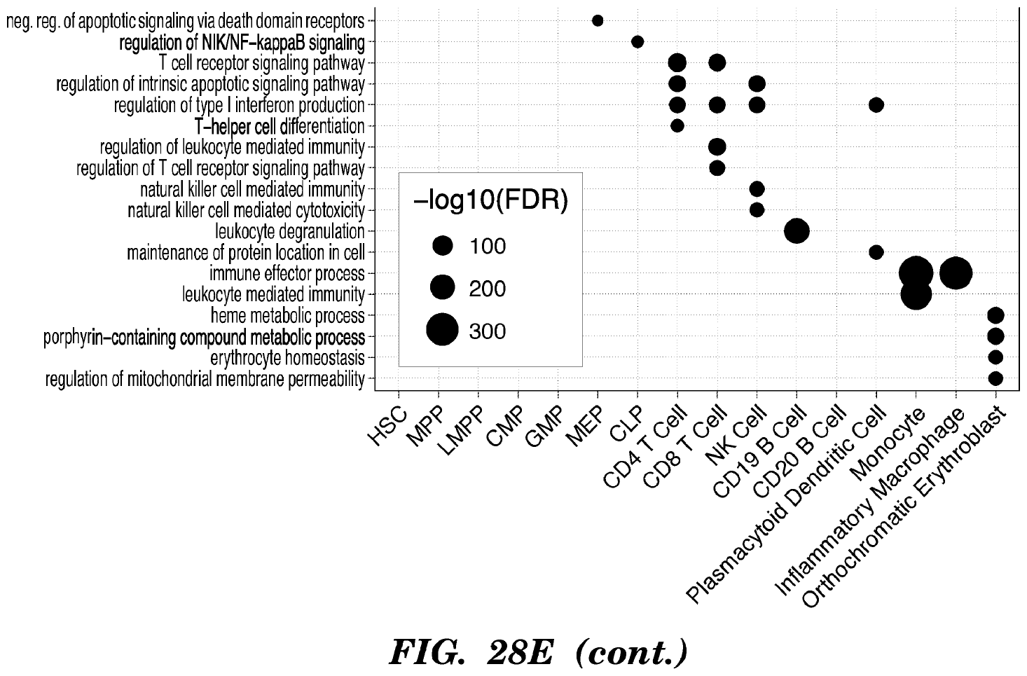

The modified ABC model was utilized to build a compendium of genome-wide enhancer-gene maps for all major cell types and states in the human hematopoietic system. Publicly available chromatin accessibility data was curated on primary human samples, spanning HSCs and hematopoietic progenitors to mature, differentiated progeny in the myeloid, erythroid and lymphoid lineages. In total, the dataset comprised 258 samples, encompassing 65 different hematopoietic cell types or states. Each hematopoietic cell type was represented by at least 3 different donors in nearly every instance. All the chromatin accessibility data was uniformly processed (see Methods), and all samples exhibited high enrichment of signal over background (FIG. 28A).

The modified ABC model was applied to generate genome-wide enhancer-gene maps for each of the 65 hematopoietic cell types in our dataset (see Methods). A total of 3,793,020 enhancer-gene links representing a comprehensive resource of regulatory logic governing the human hematopoietic system. Across all cell types, a gene was predicted to be regulated by 3.22±0.14 ABC enhancers and an ABC enhancer was predicted to regulate 2.35±0.12 genes. Within a given cell type, ABC enhancers constituted on average only 11.2% of accessible chromatin regions (FIG. 28B), underscoring the specific nature of these genome-wide predictions. Furthermore, this dataset amounted to 207,648 unique ABC enhancers across all hematopoietic cell types, which we refer to as a pan-hematopoiesis ABC enhancer peakset. To facilitate subsequent analysis, a matrix was built of the quantitative degree of chromatin accessibility over the pan-hematopoiesis ABC enhancer peakset for all 258 samples. Chromatin accessibility signal in ABC enhancers was highly reproducible across technical and donor replicates for each hematopoietic cell type (FIG. 28C).

To visualize global patterns from our ABC predictions, t-SNE was utilized (FIG. 22B) and density clustering to identify 16 distinct clusters based on chromatin accessibility within the pan-hematopoiesis ABC enhancer peakset (FIG. 28D). Accessibility of ABC enhancers alone was sufficient to delineate the broad set of hematopoietic cell types in our dataset, reflective of their cell-type specificity. Striking concordance between the FACS-sorted was also observed, immunophenotypic identity of the samples and the unbiased determined clusters (FIG. 22C). In further agreement with the cell type specificity of ABC enhancers, enhancer-gene links were distinct across cell types, yet shared a higher fraction of links within a given lineage (FIG. 22D). This observation suggests that lineage-level gene regulation is more finely tuned by ABC enhancer accessibility than by different sets of enhancer-gene connections. The predicted enhancer-gene links were further investigated and found that genes with complex enhancer landscapes were canonical regulators of cell identity, such as ERG, LMO2, HOXA9 and RUNX1 within HSCs and SYK, IKZF1, EBF1, and PAX5 in CD19+ B cells (FIG. 22E). More generally, ABC-linked genes were enriched for canonical biological processes for the given cell type (FIG. 28E). Finally, the ABC maps were used to infer potential activity of trans-factors in ABC enhancers. TF footprinting was performed specifically within a cell type's ABC enhancers using ATAC-Seq data and observed motif footprints for canonical TFs that regulate cellular identity (FIG. 22F). TFs also displayed dynamic activity within ABC enhancers across cell types belonging to the same hematopoietic lineage (FIG. 22G), further support that ABC predictions are reflective of transcriptional enhancers. Overall, the ABC maps provide a rich, informative and comprehensive resource to dissect enhancer-gene regulation in the human hematopoietic system.

### TE Families are Enriched in ABC Enhancers and Encode for Cell-Type Specific Transcriptional Regulators

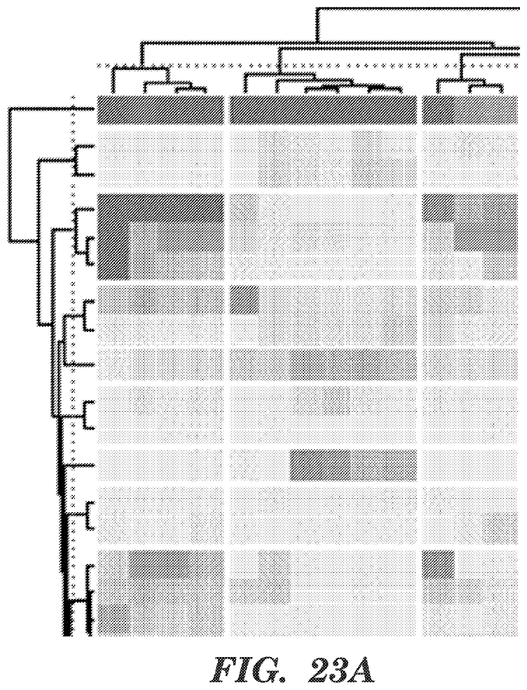

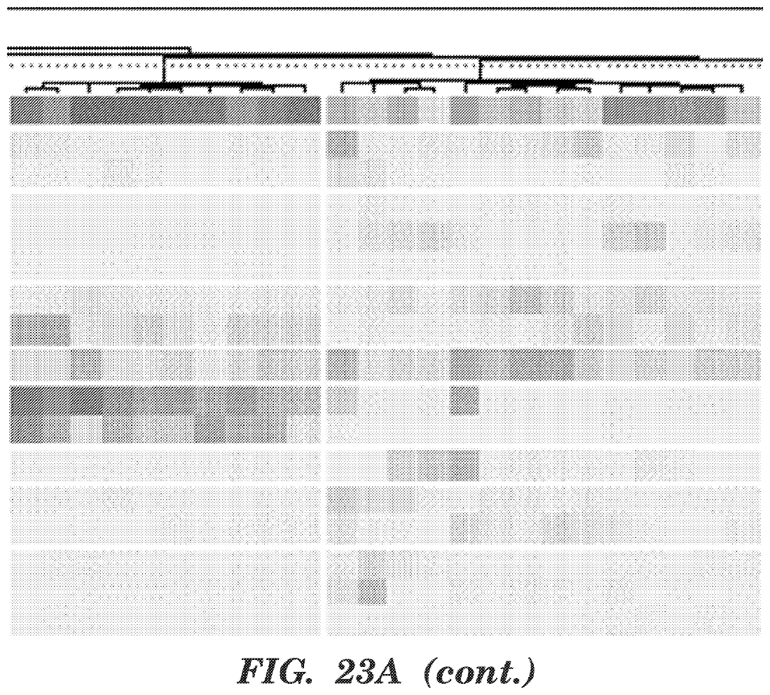

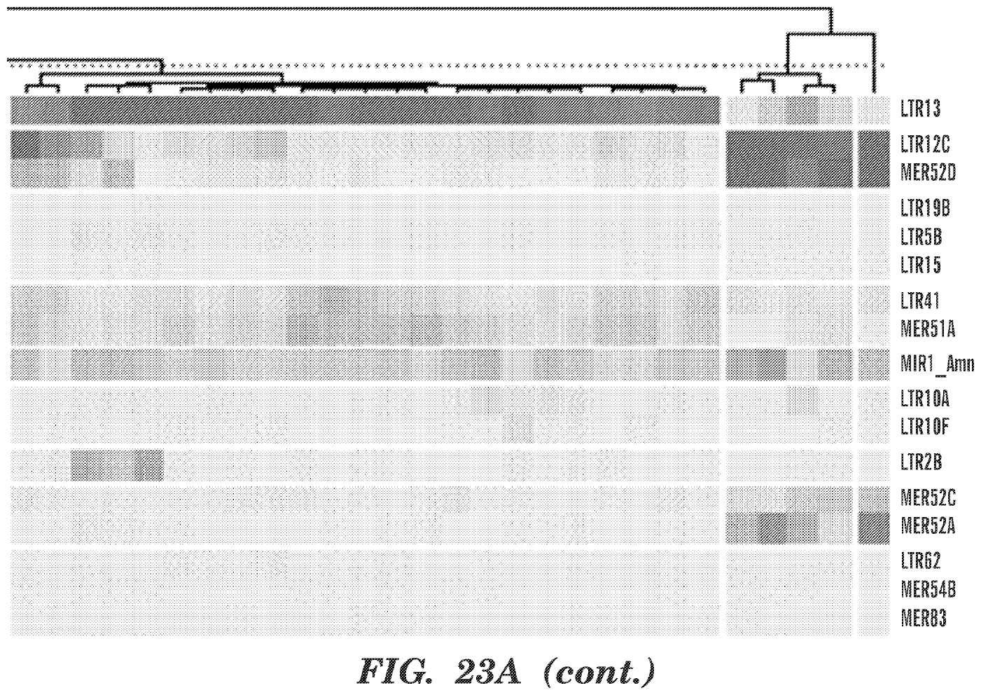

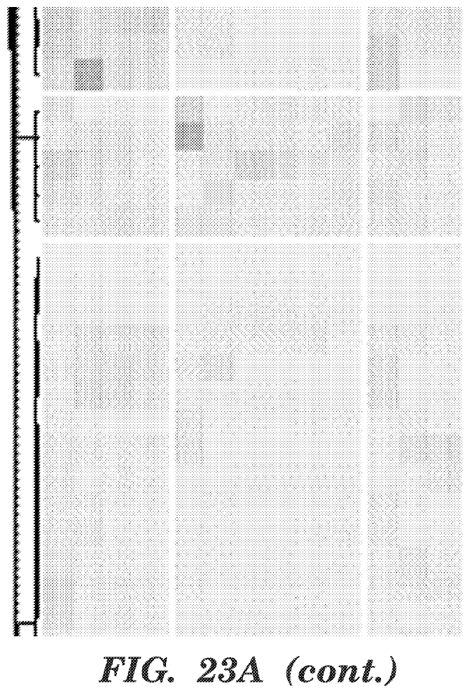

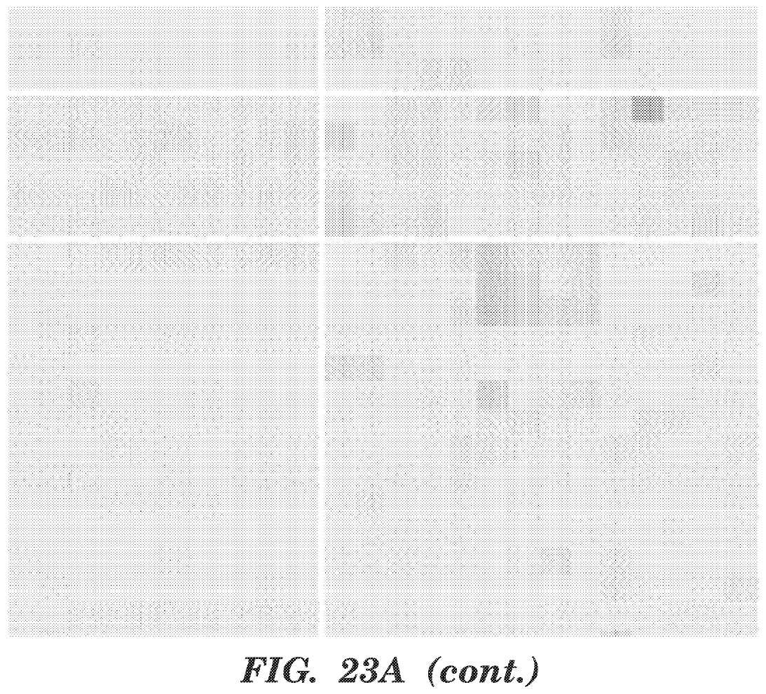

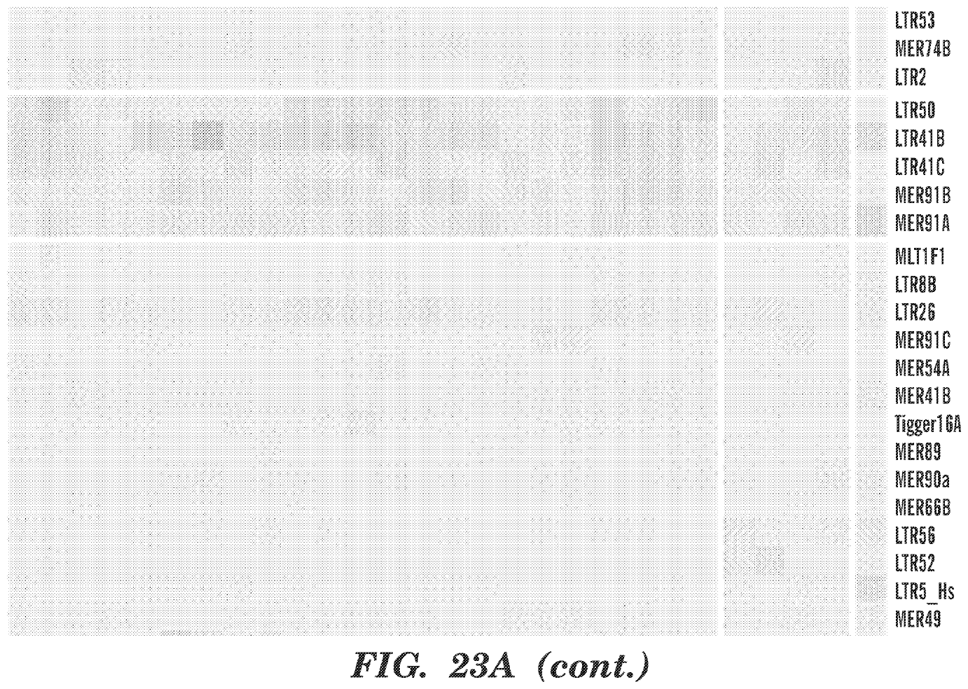

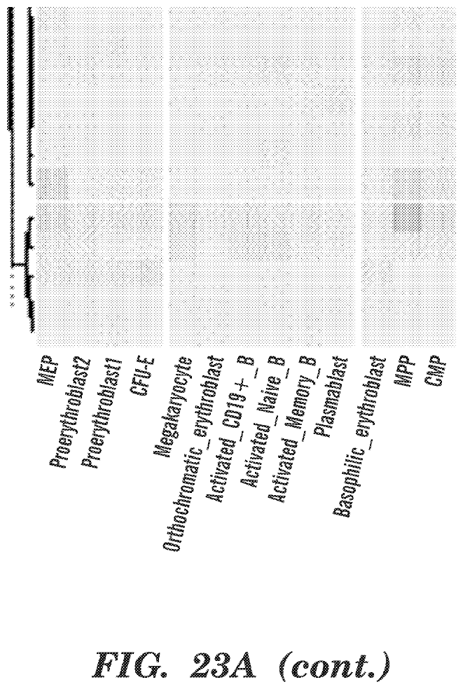

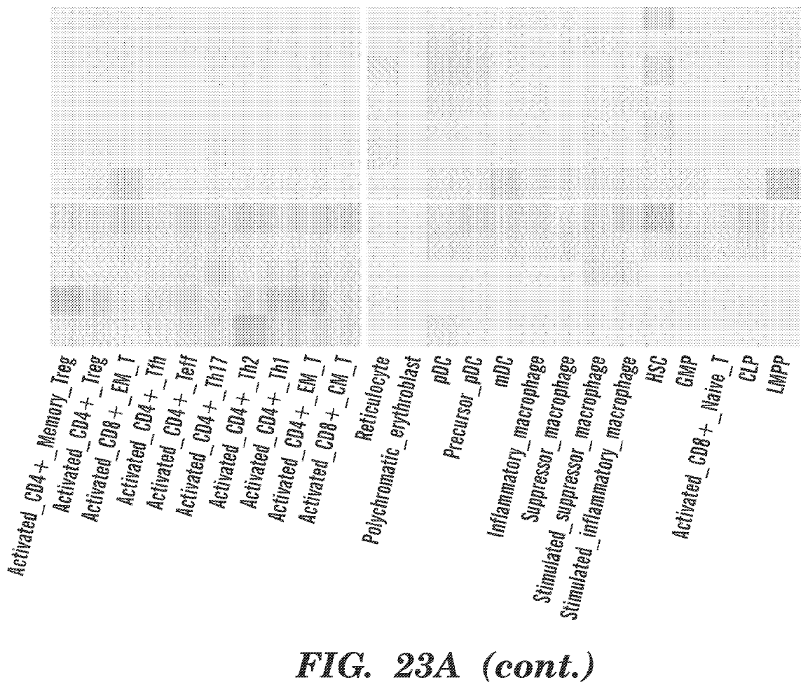

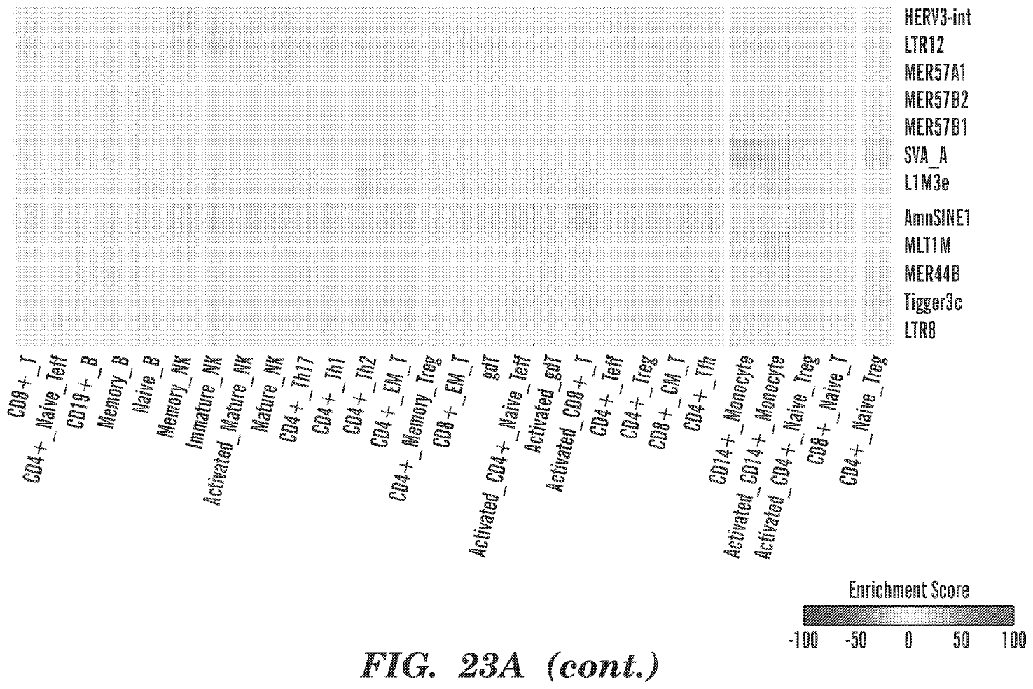

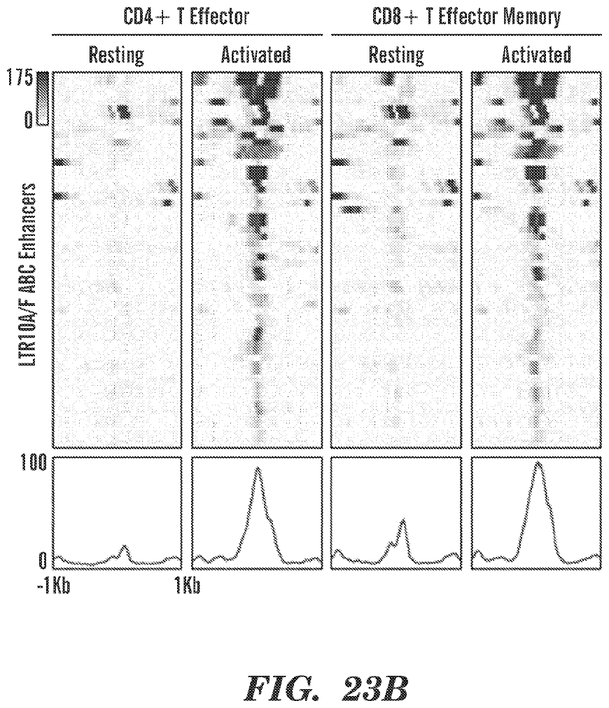

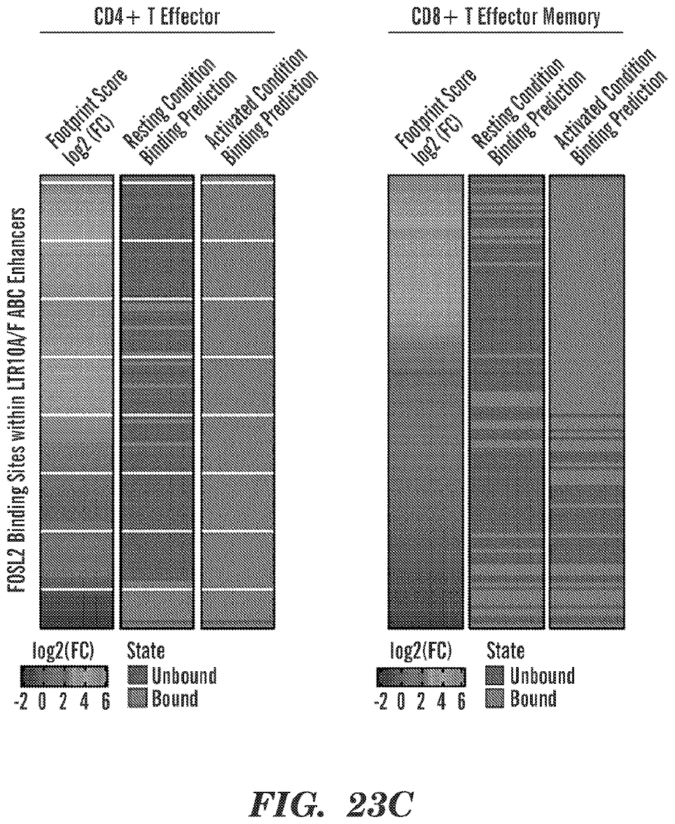

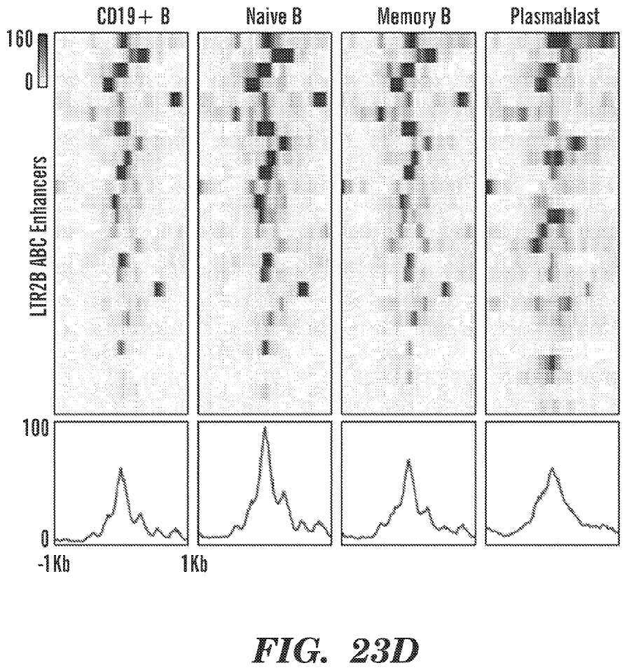

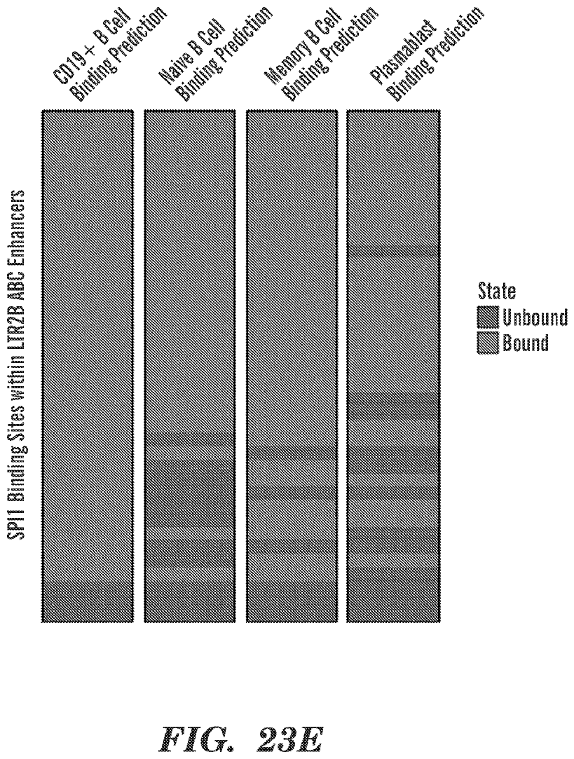

Having demonstrated the utility of the ABC maps, it was investigated how TEs contribute to hematopoietic gene regulation. The Repbase TE database was utilized and segregated the annotated TEs in the human genome by family-level classification. The genomic coordinates of these TE families were intersected with ABC enhancers from each of the hematopoietic cell types in our dataset and identified 51 TE families that were significantly enriched in ABC enhancers (FIG. 23A, Methods). An overwhelming fraction of the significantly enriched TE families consisted of MER and LTR elements (43/51). Notably, TE families exhibited cell type and state-specific enrichments. In particular, LTR10A/F elements were selectively enriched within activated CD4 and CD8 T cell subsets. The specificity and degree of these enrichments prompted further investigation into the basis of TE co-option in lymphoid cells. ATAC-Seq signal over LTR10A/F-containing ABC enhancers was pronounced only in activated states and generally low-to-inaccessible in resting states (FIG. 23B), confirming the prior enrichment result and suggesting that these elements may be involved in regulating T cell activation. The LTR10A-G family has previously been documented to contain transcription factor motifs for the AP-1 family of TFs, which is consistent with the role of AP-1 in T cell activation. It was asked whether these LTR10A/F ABC enhancers are regulated by AP-1 TFs. The ATAC-Seq data was leveraged on activated and resting T cells in our dataset and implemented the TOBIAS transcription factor occupancy framework to infer TF occupancy in ABC enhancers. Indeed, nearly all AP-1 motifs within LTR10A/F ABC enhancers were predicted to be bound by AP-1 TFs in activated T cells, whereas binding was largely absent in the resting state (FIG. 23C), further supporting the role of these elements as putative TE-derived enhancers. In addition, enrichment of LTR2B elements was also observed within ABC enhancers of B cells (FIG. 23B), and the enhancer elements were highly accessible across the B cell lineage (FIG. 23D). Furthermore, TOBIAS predicted binding of SPI1, an important TF regulating B cell identity in LTR2B ABC enhancers (FIG. 23E). This may reflect cell-type specific tuning of enhancer activity.

Overall, the analysis argues for a pervasive and cell type-specific contribution of TE families to hematopoietic ABC enhancers, and a particular role for TEs in transcriptional regulatory networks of lymphoid cells via TFs essential for cell identity and function.

TEs and their Epigenetic Regulatory Machinery are Dynamically Expressed During Hematopoietic Differentiation

Hematopoiesis involves the dynamic control of transcriptional regulatory networks to generate a diverse repertoire of hematopoietic and immune cell types. In this context, it was investigated whether TEs are dynamically regulated during hematopoietic differentiation, given their contribution to gene regulation within specific cell types. TE expression was quantified from RNA-Seq data on FACS sorted hematopoietic populations spanning the hematopoietic hierarchy (see Methods), which identified 1,295 expressed TE families. As expected, hierarchical clustering based on expressed genes revealed lineage relationships that have been well documented as distinct hematopoietic populations (FIG. 18A). Notably, clustering based solely on expressed TEs also largely recapitulated these lineage relationships (FIG. 18B), in concordance with the cell type specificity of TE expression within the hematopoietic system. This result is in line with observations of selective expression of particular TE families within pluripotent stem cells and mature somatic cells, as well as during dynamic processes such as embryonic development and cellular reprogramming. To dissect the dynamics of TE expression during hematopoiesis, differentially expressed TEs were identified in hematopoietic progenitors and differentiated populations relative to HSCs. Notably, expression of TEs was elevated in differentiated lymphoid cell types, initiating at common lymphoid progenitors (CLP). Conversely, a larger number of TEs exhibited decreased expression in various myeloid and erythroid progenitors (FIG. 18C).

Various cellular mechanisms exist to repress TEs within the human genome that largely depend on the formation of heterochromatin at TE loci. Thus, the dynamic expression of TEs observed during hematopoietic differentiation prompted investigation of whether regulators of heterochromatin formation exhibit dynamic expression during differentiation. The expression of gene modules involved in H3K9 methylation was quantified using a scRNA-Seq atlas of the human hematopoietic system. Mature cell types exhibited significantly reduced expression of genes associated with H3K9 methylation compared to HSPCs (FIG. 18I), consistent with the up-regulation of TE expression. The NuRD complex demonstrated similar expression dynamics as the H3K9 methylation gene program (FIG. 18J), further supporting the observations that the expression of TE regulatory machinery is inversely correlated with TE expression during lymphoid differentiation. In further agreement with these observations, H3K9 demethylation modules were elevated in mature hematopoietic cell types compared to HSPCs (FIG. 18K). The collective observations that TEs are upregulated during lymphoid differentiation with associated attenuation of TE regulatory machinery data establish that TEs are dynamically regulated during hematopoietic differentiation.

### Modulation of Chromatin Machinery Regulating TEs Directs Lymphoid Lineage Decisions

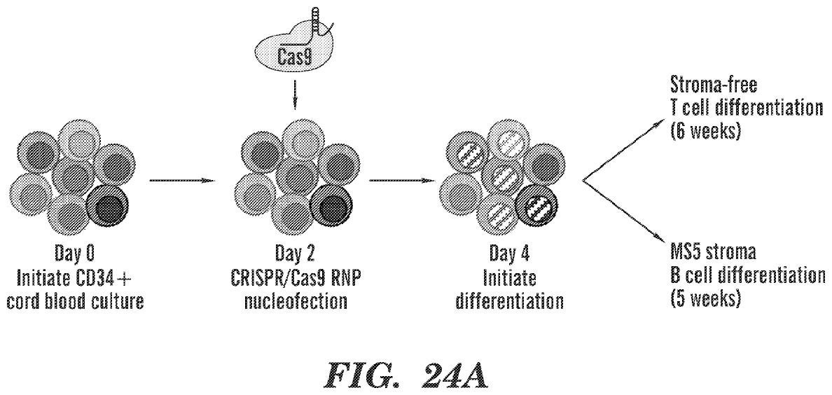

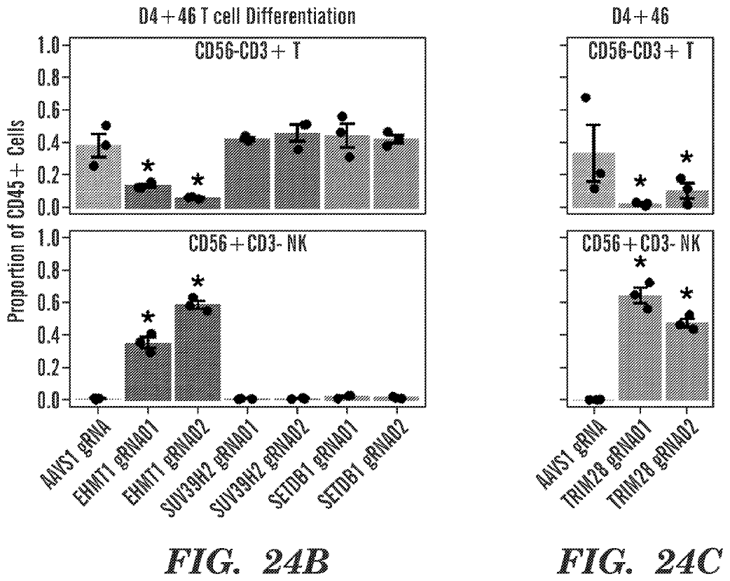

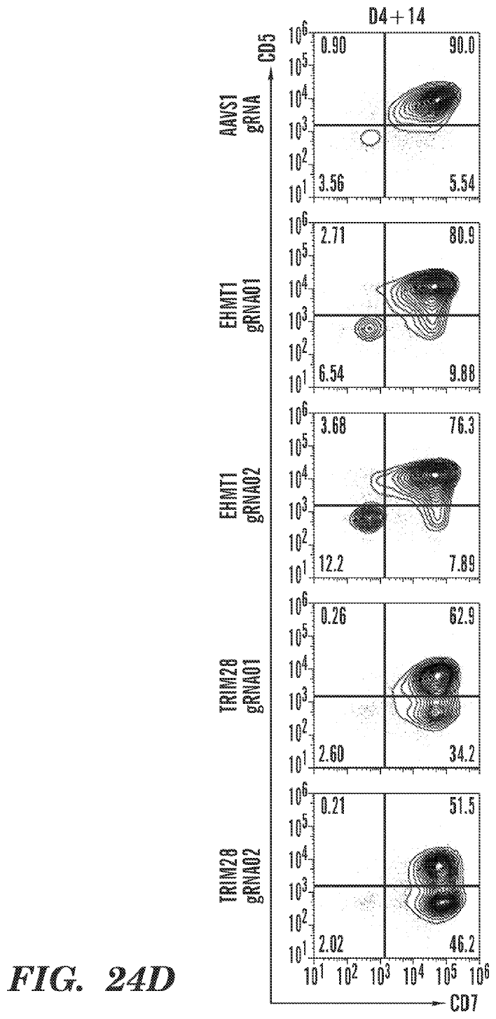

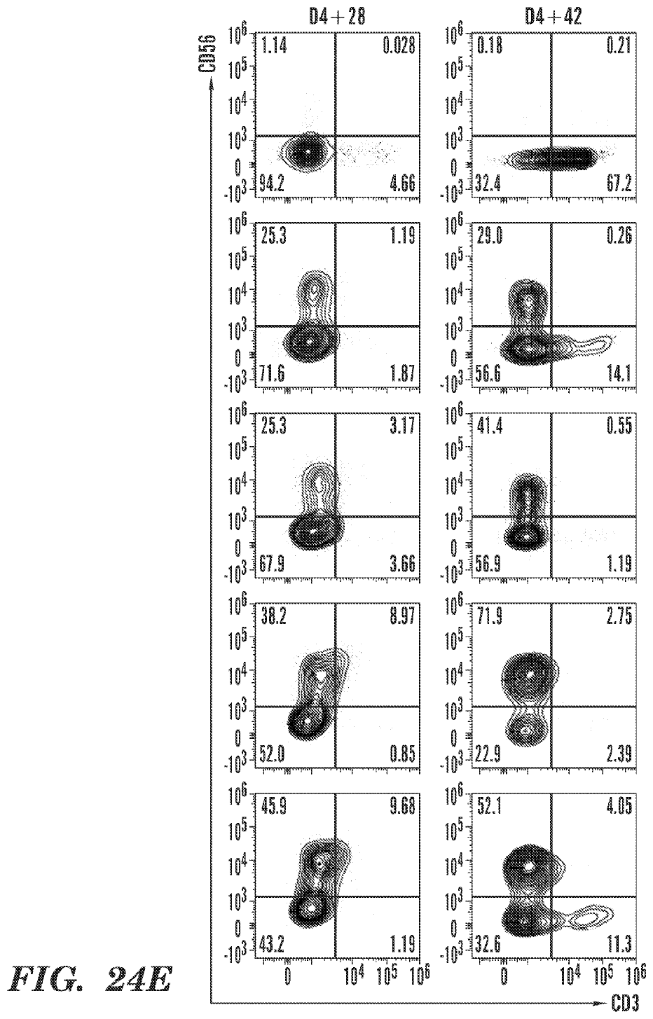

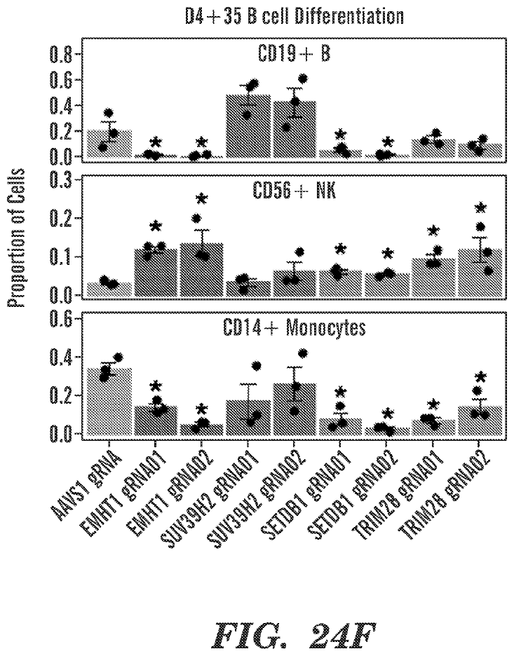

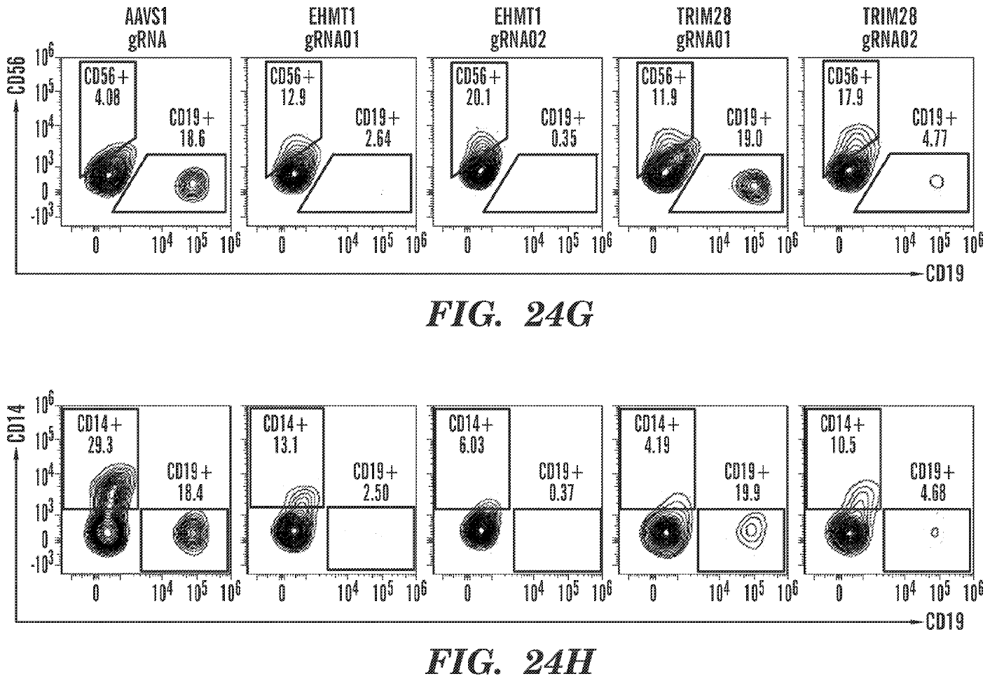

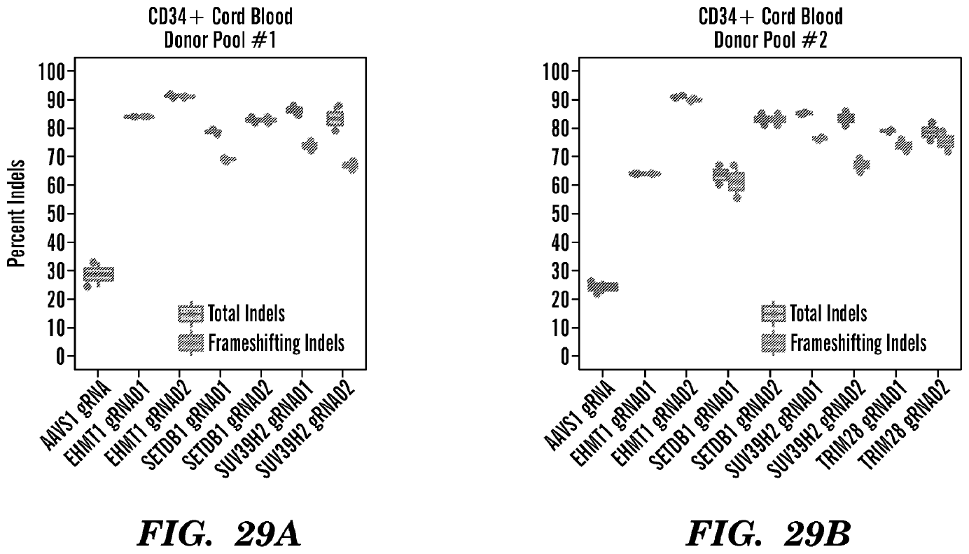

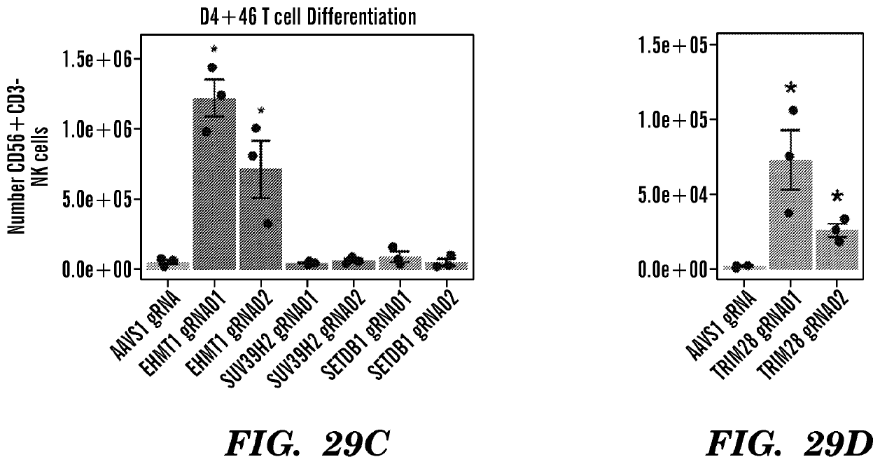

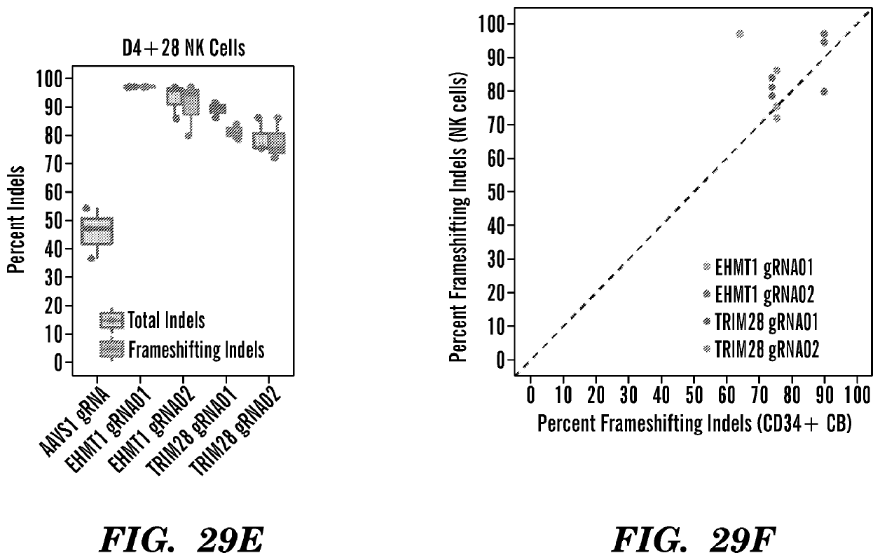

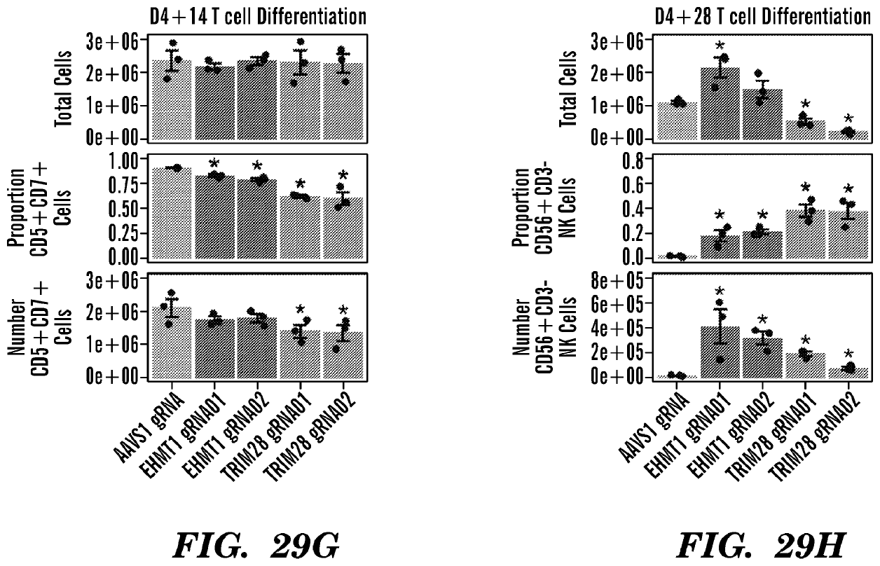

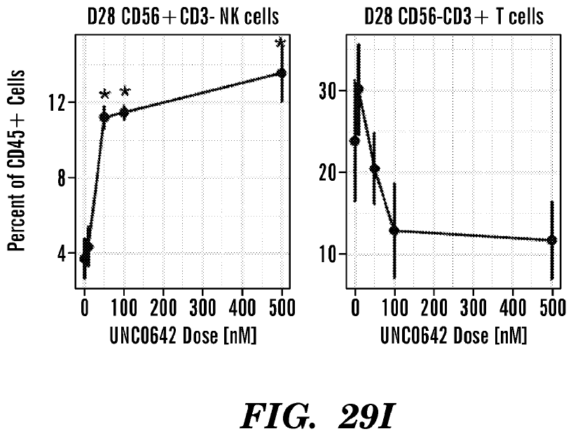

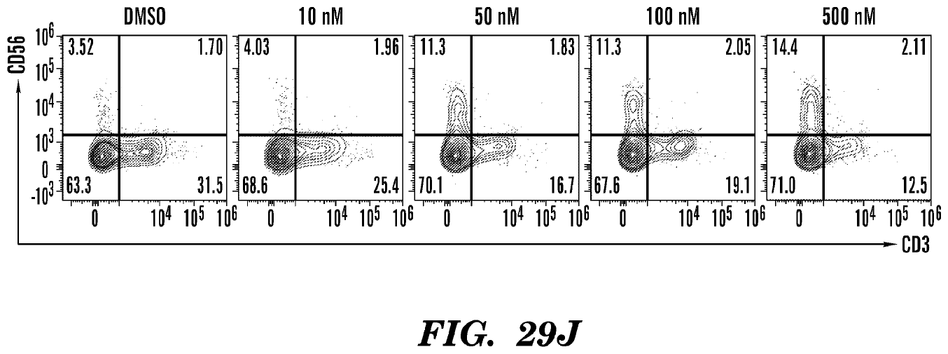

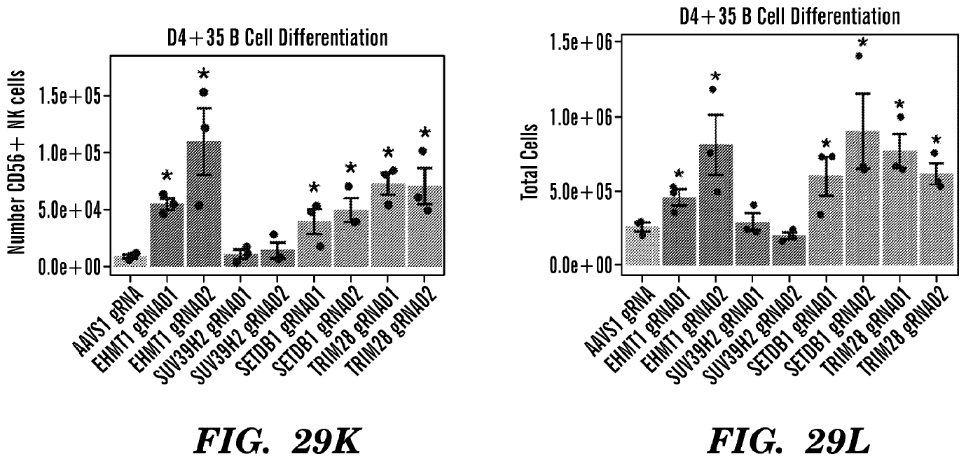

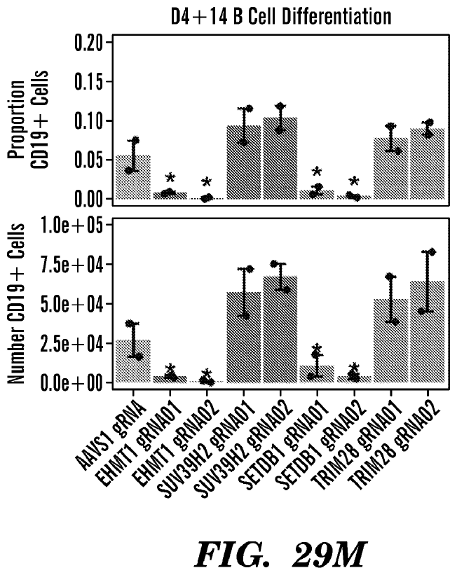

Given the observations that TEs are associated with lymphoid-specific enhancers and appear to be coordinately expressed during lymphoid differentiation, it was hypothesized that TEs directly influence lymphoid lineage decisions. To derepress TEs in HSPCs, a gene-centric loss-of-function approach was employed to knockout regulators of heterochromatin formation, specifically H3K9 lysine methyltransferases and the transcriptional co-repressor TRIM28, and then assessed lineage transitions in T and B cell-supportive differentiation conditions (FIG. 24A). CRISPR/Cas9 ribonucleoproteins (RNPs) were nucleofected into human CD34+ umbilical cord blood and generated replicate knockouts using two distinct gRNAs per gene, as well as a control targeting the AAVS1 locus. Overall, frameshifting indel formation was highly efficient for each gRNA across multiple donor pools, enabling differentiation experiments directly on the pool of nucleofected cells (FIGS. 29A-29B).

T cell differentiation was first initiated using a stroma-free assay (see Methods) following CRISPR/Cas9 RNP delivery. After six weeks of in vitro differentiation (noted as D4+46), it was observed that knockout of EHMT1 resulted in a reduction of CD56−CD3+ T cells and an associated increase of CD56+CD3− natural killer (NK) cells compared to an AAVS1 gRNA control (FIG. 29B). Furthermore, knockout of TRIM28, a key regulator of TE silencing, phenocopied that of EHMT1 (FIG. 29C), suggesting a central role of TE regulatory machinery in the determination of NK cell fate. The proportional increase in CD56+CD3− NK cells at D4+46 was also accompanied by increased absolute numbers of CD56+CD3− cells (FIGS. 29C-29D). Sequencing of isolated NK cells confirmed the presence of frameshifting indels (FIGS. 29E-29F), further supporting the conclusion that the knockouts generate NK cells. To dissect the kinetics of this T-to-NK lineage bias, time course profiling was performed every two weeks over the six weeks of differentiation (FIGS. 24D-24E). Notably, an altered distribution of lymphoid progenitors was observed at D4+14 characterized by an attenuated frequency of cells expressing the T cell-associated marker CD5 without affecting overall cell viability (FIG. 24D, FIG. 29G). These data imply that NK lineage bias manifests in early stages of lymphoid differentiation. NK cells were evident an additional two weeks later at D4+28 and proportionally further increased by D4+46 (FIG. 24E, FIG. 29H). Finally, sustained treatment with a potent enzymatic inhibitor of GLP/G9a, UNC0642 over the course of T cell differentiation resulted in a dose-dependent phenocopy of the EHMT1 CRISPR/Cas9 knockout (FIGS. 29I-29J), leading to the acquisition of CD56+CD3− NK cells and general attenuation of CD56−CD3+ T cells. These data indicate that modulation of H3K9 methyltransferase activity and regulation of heterochromatin facilitates NK lineage choice.

The data in T cell-supportive differentiation conditions underscore a role for TE regulation in lymphoid fate decisions. It was next asked whether similar mechanisms may be applicable during B-lymphoid differentiation. The CRISPR/Cas9 knockouts were replicated in CD34+ umbilical cord blood HSPCs and utilized an MS5-stroma co-culture assay, which is conducive for B cell development as well as myeloid differentiation. Consistent with the observations in T cell differentiation conditions, knockout of EHMT1 resulted in NK lineage skewing at the expense of CD19+ B cells and CD14+ monocytes following five weeks of differentiation on MS5 stroma (FIGS. 24F-24H). Knockout of TRIM28 also enhanced the proportion of CD56+ NK cells at the expense of CD14+ monocytes, and a trend towards a decrease in CD19+ B cells (FIG. 24F). In further support of the modulation of TE regulatory machinery to facilitate NK fates, knockout of SETDB1 also enhanced the generation of CD56+ NK cells (FIG. 24F, FIG. 29K). As observed in the T cell-supportive conditions, proportional increases in NK cells were accompanied by an increase in the absolute numbers of NK cells compared to the AAVS1 gRNA control (FIGS. 29K-29L). In further concordance with the T cell differentiation experiment, lineage alterations manifested early during differentiation since knockout of EHMT1 and SETDB1 resulted in the precipitous decrease in CD19+ progenitors at D4+14 (FIG. 29M). Taken together, the data demonstrate that the modulation of EHMT1 or TRIM28 has a pronounced influence on NK cell development during lymphoid differentiation, further establishing that manipulation of the chromatin machinery regulating TEs can direct hematopoietic lineage decisions.

Knockout of EHMT1 or TRIM28 Generates Hematopoietic Progenitors with Distinct NK Characteristics

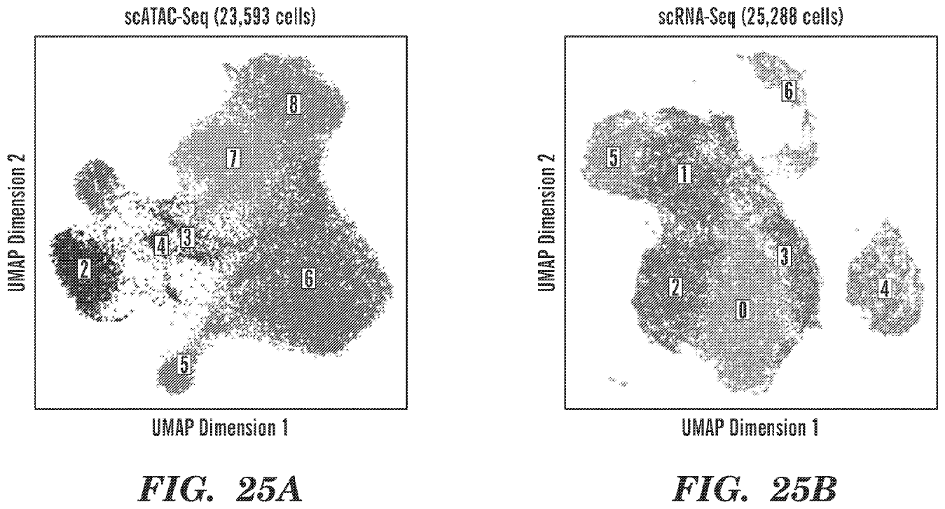

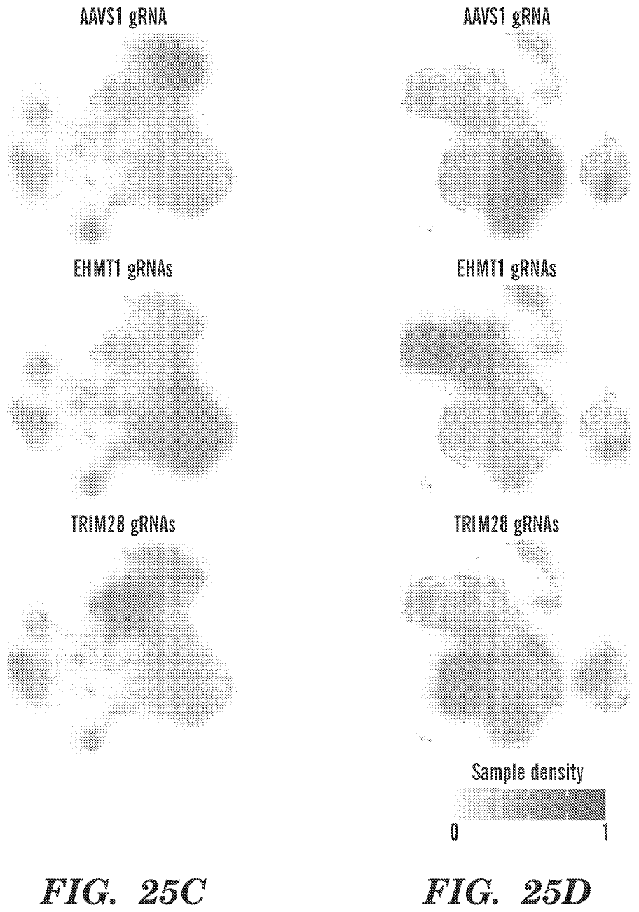

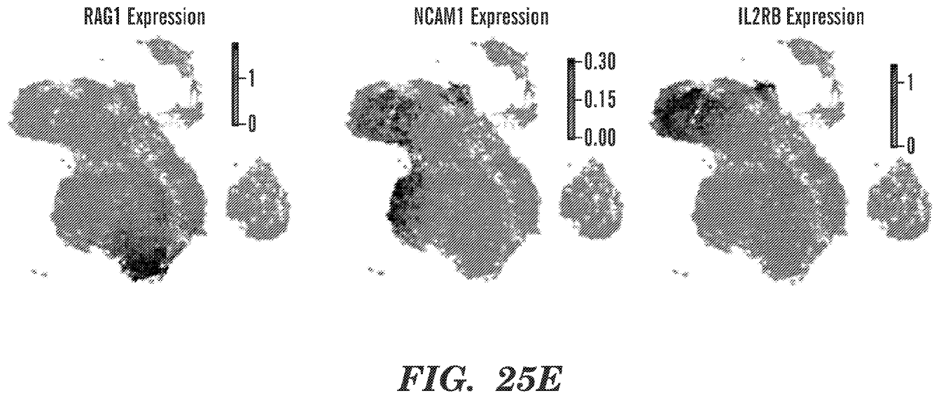

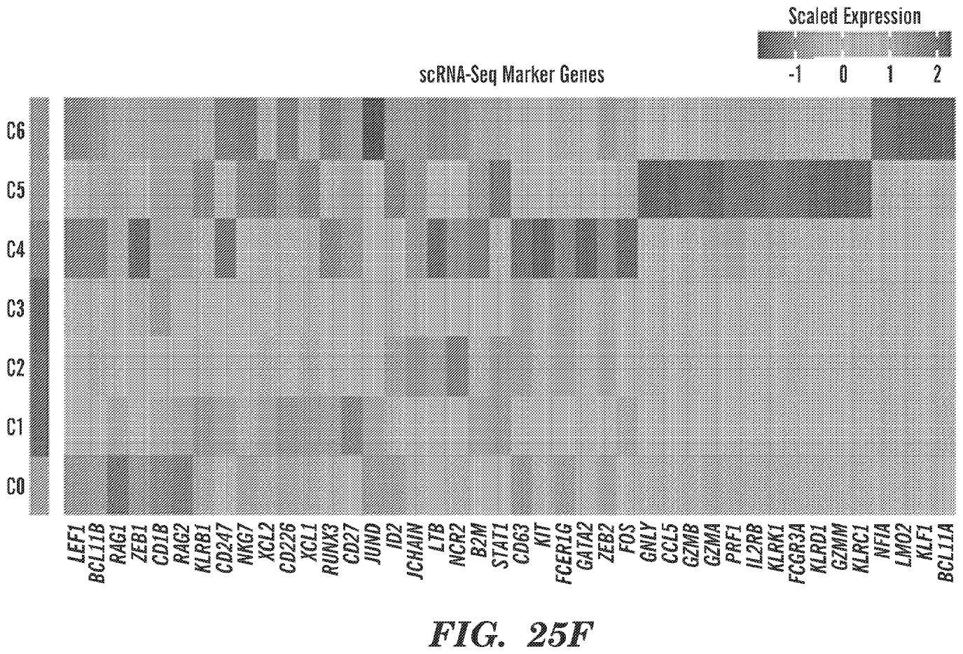

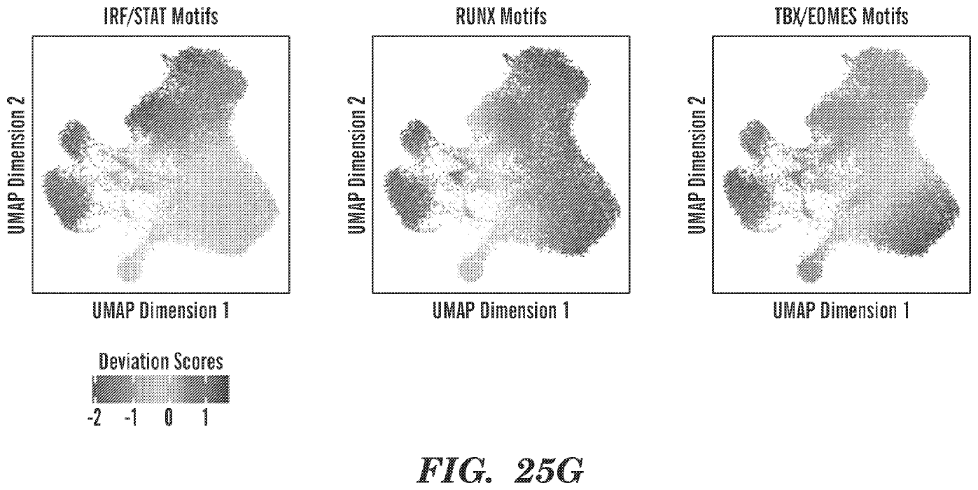

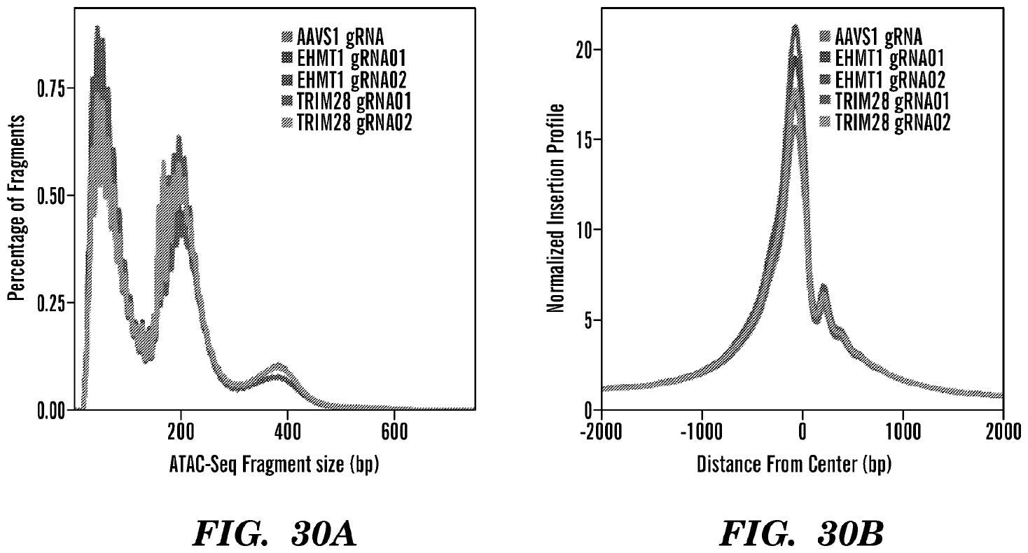

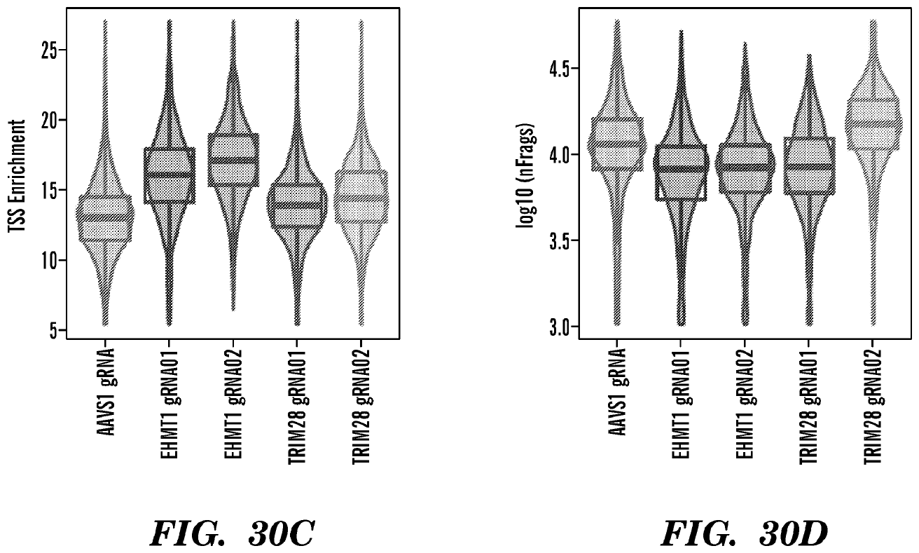

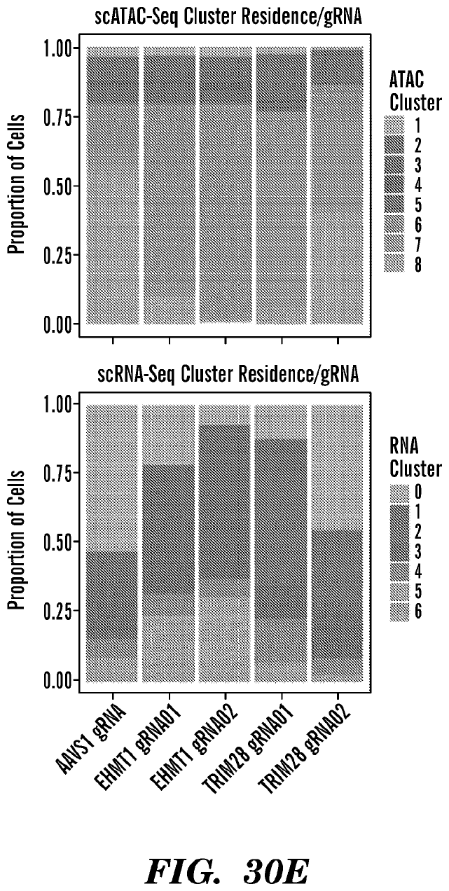

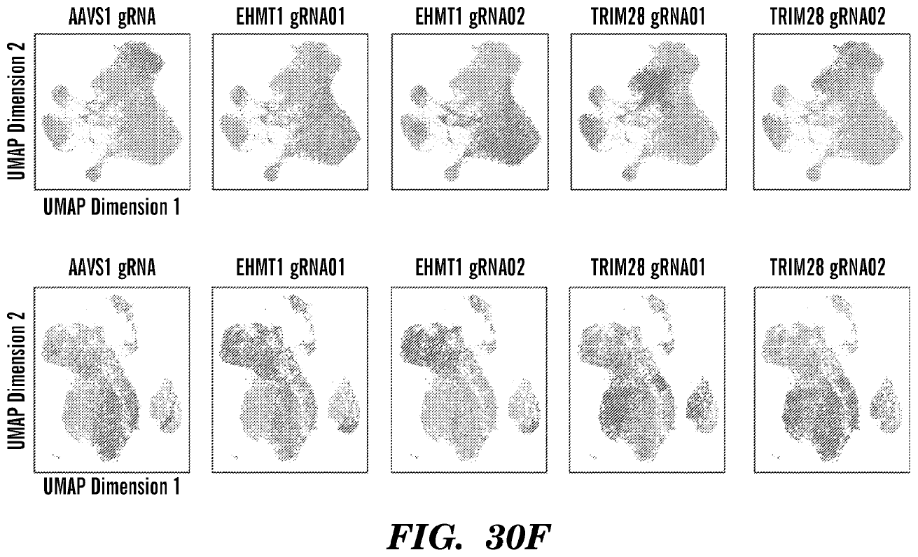

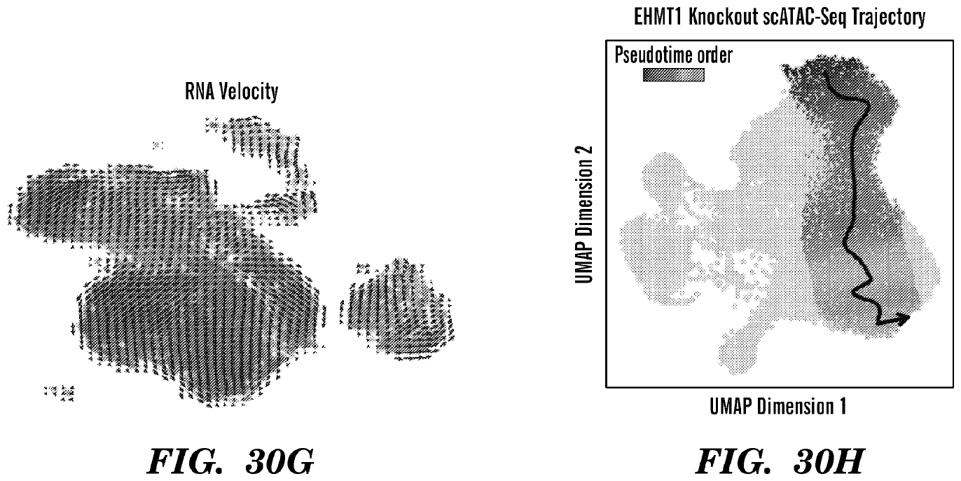

It was hypothesized that knockout of EHMT1 or TRIM28 influences NK lineage selection early during lymphoid differentiation. Motivated by the data demonstrating an altered distribution of progenitor populations within T cell-supportive conditions (FIG. 24E), single cell RNA-Seq and ATAC-Seq was utilized to dissect the molecular landscapes of hematopoietic progenitors across EHMT1 and TRIM28 knockout conditions at D4+14. Chromatin accessibility was measured across a total of 23,593 cells. All gRNA conditions exhibited a canonical fragment size distribution and high signal-to-noise (FIGS. 30A-30D). Chromatin states were first identified by clustering scATAC-Seq profiles from all cells, revealing 8 distinct clusters (FIG. 25A). These data were complemented by also capturing 25,288 single cell transcriptomes with scRNA-Seq across the same gRNA conditions. Clustering of all cells revealed 7 transcriptional states (FIG. 25B). For both assays, high concordance in clustering was observed across different gRNAs targeting the same gene (FIGS. 30E-30F), establishing that the clustering results were driven by biological rather than technical effects. Furthermore, EHMT1 gRNA and TRIM28 gRNA conditions clustered distinctly from the AAVS1 gRNA control in both assays, indicating that knockout of EHMT1 or TRIM28 are driving distinct chromatin and transcriptional states within hematopoietic progenitors (FIGS. 25C-25D). The single cell transcriptional information was utilized to annotate the identity of scRNA-Seq clusters. Notably, RAG1 expression was primarily restricted to cluster 0, which is enriched for AAVS1 gRNA cells and suggests a T cell-fated state (FIG. 25E). In further support of this, cluster 0 also selectively expressed genes linked to T cell development, such as LEF1, BCL11B, ZEB1, CD1B and RAG2 (FIG. 25F). Interestingly, the gene encoding for CD56 (NCAM1) was only expressed in clusters enriched for EHMT1 and TRIM28 knockout cells. Moreover, IL2RB, one of the earliest markers for NK-fated progenitors, already was expressed in EHMT1 knockout clusters (cluster 1 and 5) (FIG. 25E). EHMT1 knockout clusters were also positively enriched for NK genes, such as KLRK1 (encoding for NKG2D), FCGR3A (encoding for CD16), granzymes and perforin (GZMA, GZMB, GZMM, PRF1), CD247, CD226 (encoding DNAM-1) and ID2. This expression is consistent with prior reports of transcriptional markers of NK progenitors within human fetal and adult tissues. TRIM28 knockout cells within cluster 2 were characterized by high expression of LTB, NCR2, and B2M with low levels of RAG1 and RAG2, showing an earlier stage of progenitor development compared to the EHMT1 knockout cells.

Collectively, the transcriptional data underscore the distinct, NK-biased states arising from EHMT1 and TRIM28 knockouts. Transcription factor activity exhibited in hematopoietic progenitors as a result of loss of EHMT1 or TRIM28 was examined next. The scATAC-Seq data was leveraged to infer TF activity with chromVAR and identified key developmental TFs with cluster-specific activity (FIG. 25G). IRF and STAT TFs exhibited prominent activity within TRIM28 knockout cells, which is consistent with the transcriptional upregulation of STAT1 in TRIM28 knockout cells (scRNA-Seq cluster 2). Previous studies have documented inflammatory signatures associated with derepression of TEs via loss of TRIM28, as well as the relevance of STAT1 to NK cell function. RUNX3 is expressed in developing NK cells and increases with NK maturation. In line with this prior literature, RUNX TF activity was observed within AAVS1 and EHMT1 gRNA scATAC-Seq clusters (clusters 6 and 8) and specific upregulation of RUNX3 expression within EHMT1 gRNA scRNA-Seq clusters (clusters 1 and 5). Additionally, TBX21/EOMES, developmentally important TFs for NK development, also exhibited localized activity within EHMT1 gRNA scATAC-Seq clusters. In further support of the distinct genetic regulation, RNA velocity (FIG. 30G) and scATAC-Seq trajectory analysis (FIG. 30H) were both reflective of cellular trajectories diverging from the AAVS1 gRNA control population. Taken together, these data reflect the distinct transcriptional and chromatin states characterized by knockout of EHMT1 or TRIM28, and the resulting induction of NK fates within hematopoietic progenitors.

### EHMT1 and TRIM28 Knockout NK Cells Exhibit Distinct States and Effector Functions

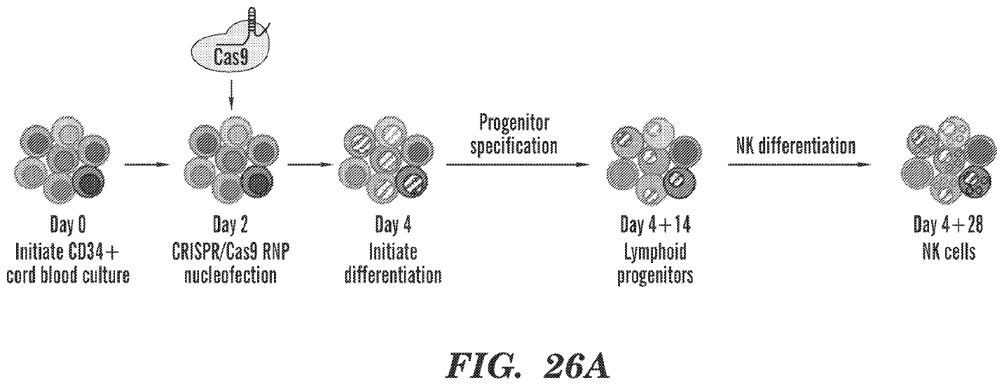

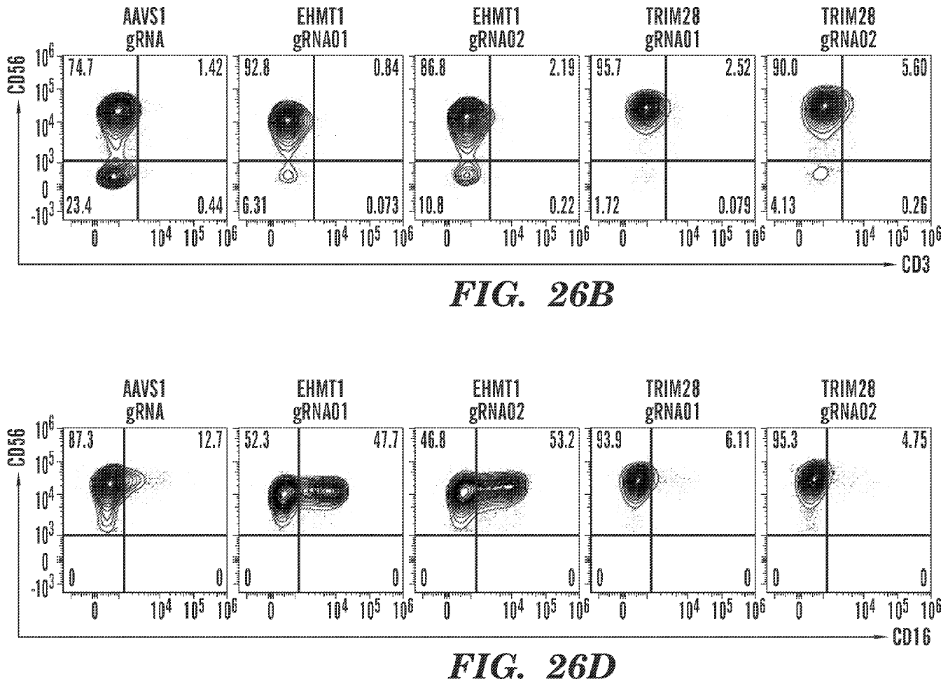

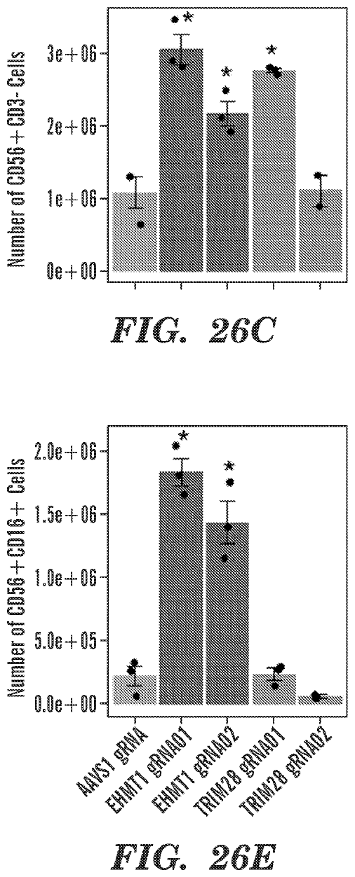

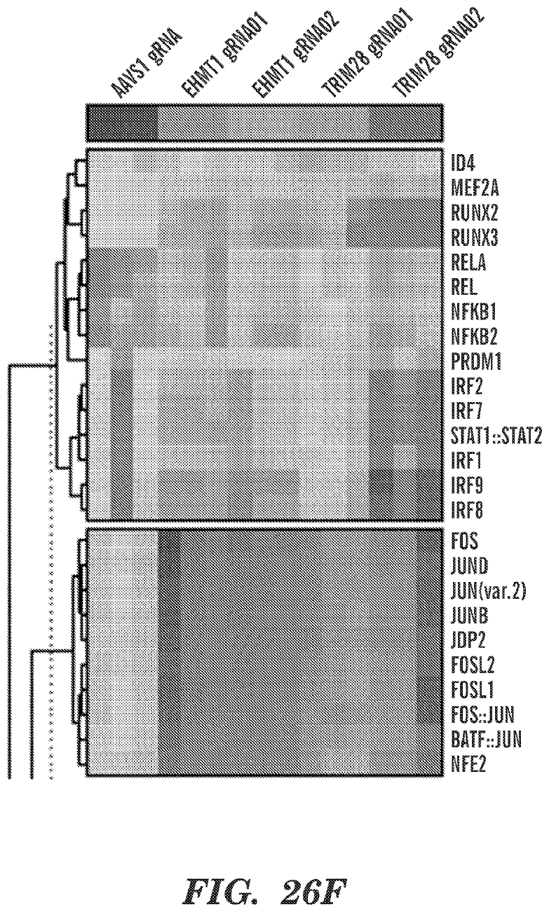

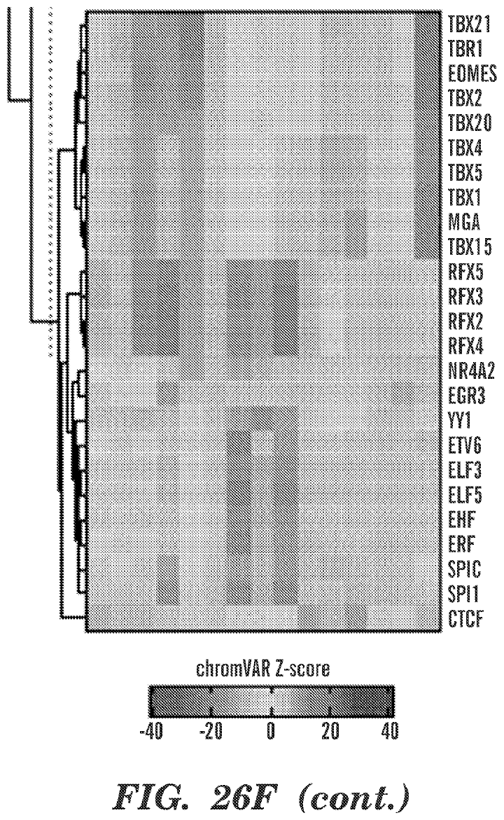

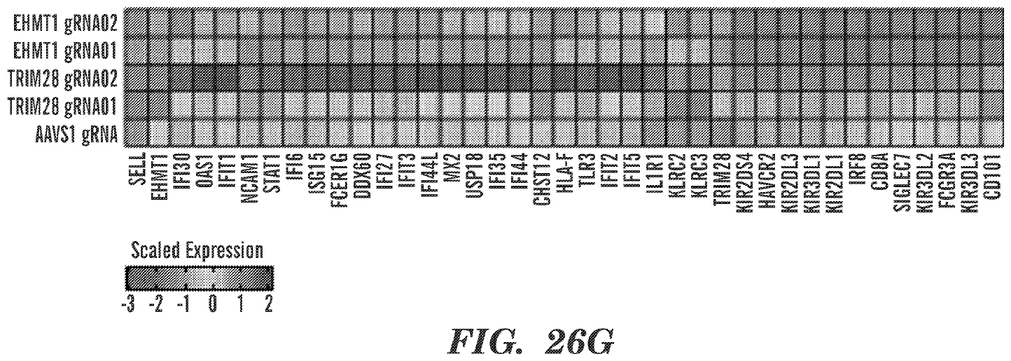

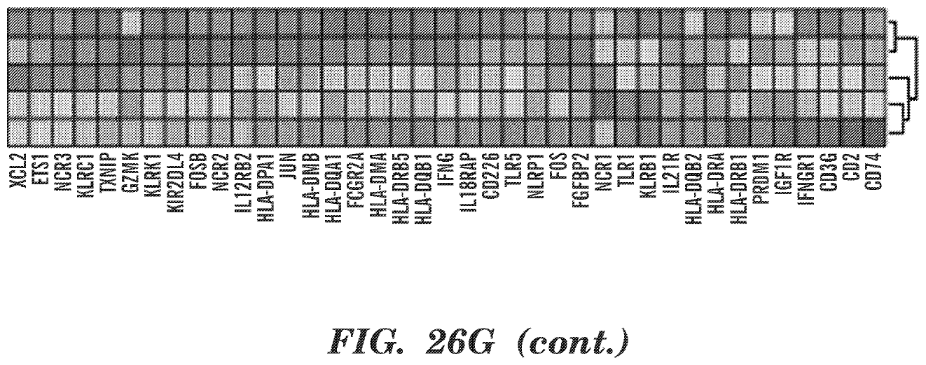

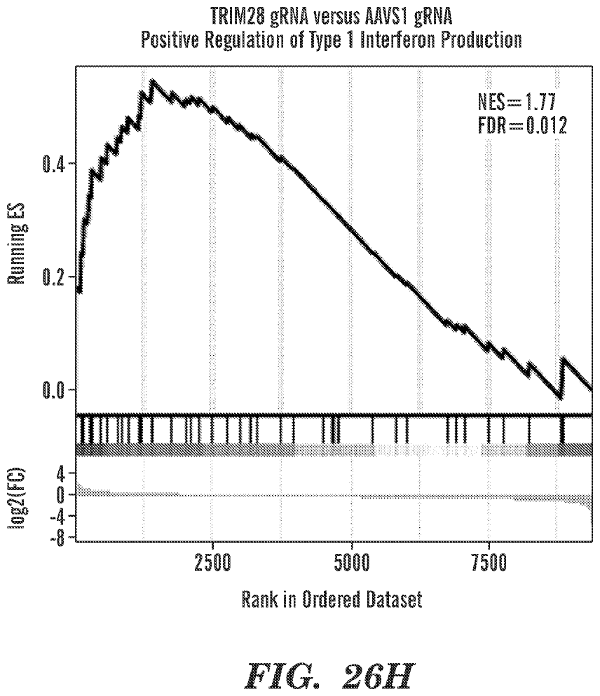

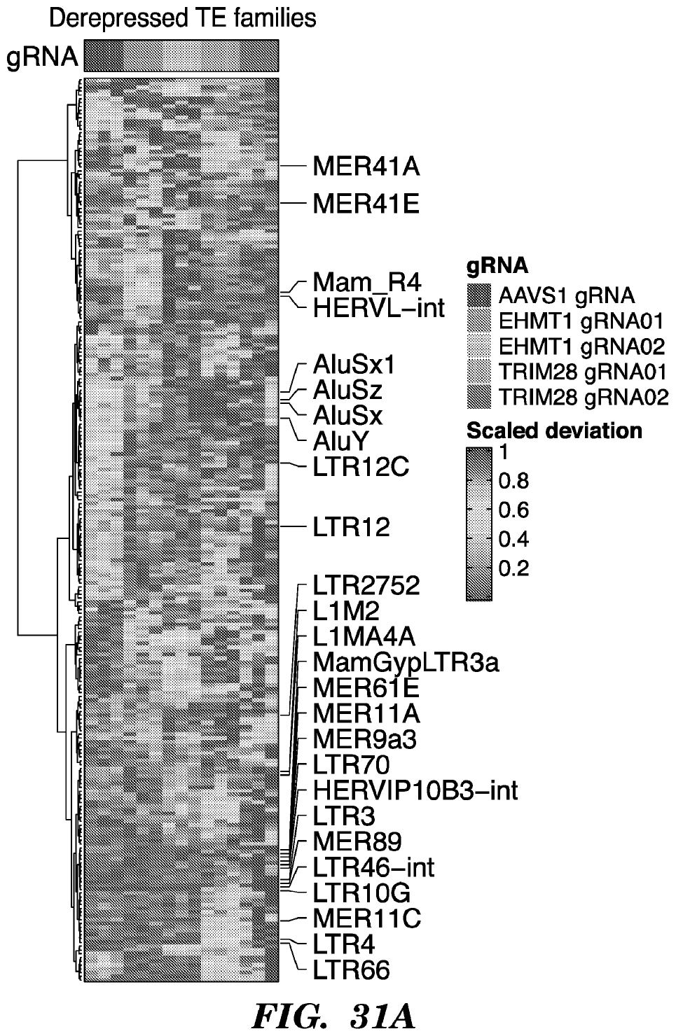

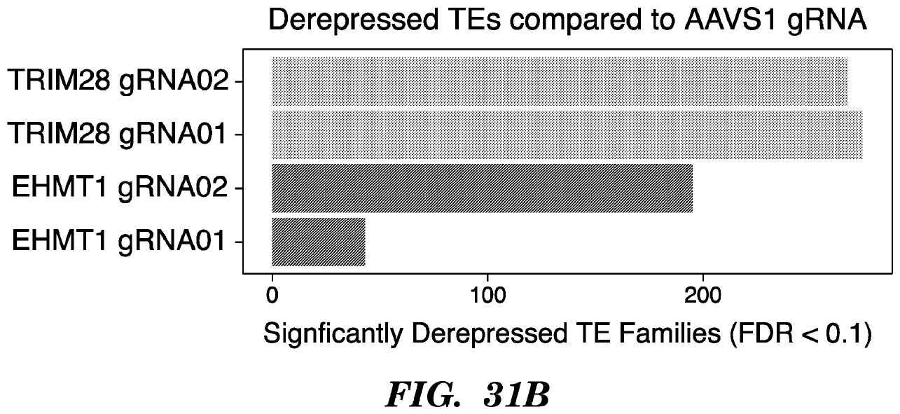

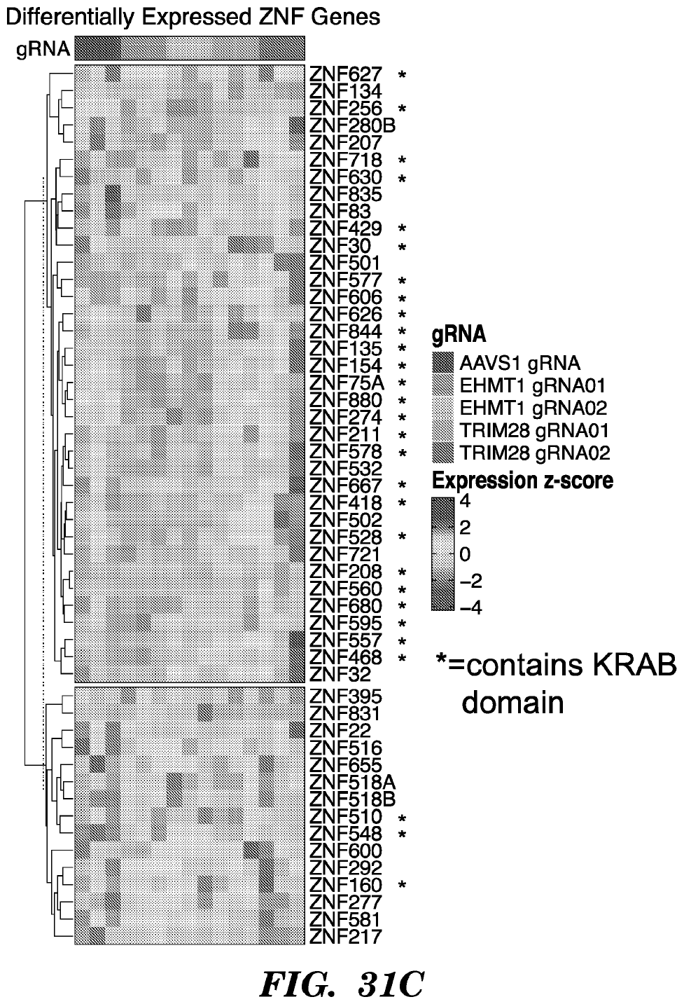

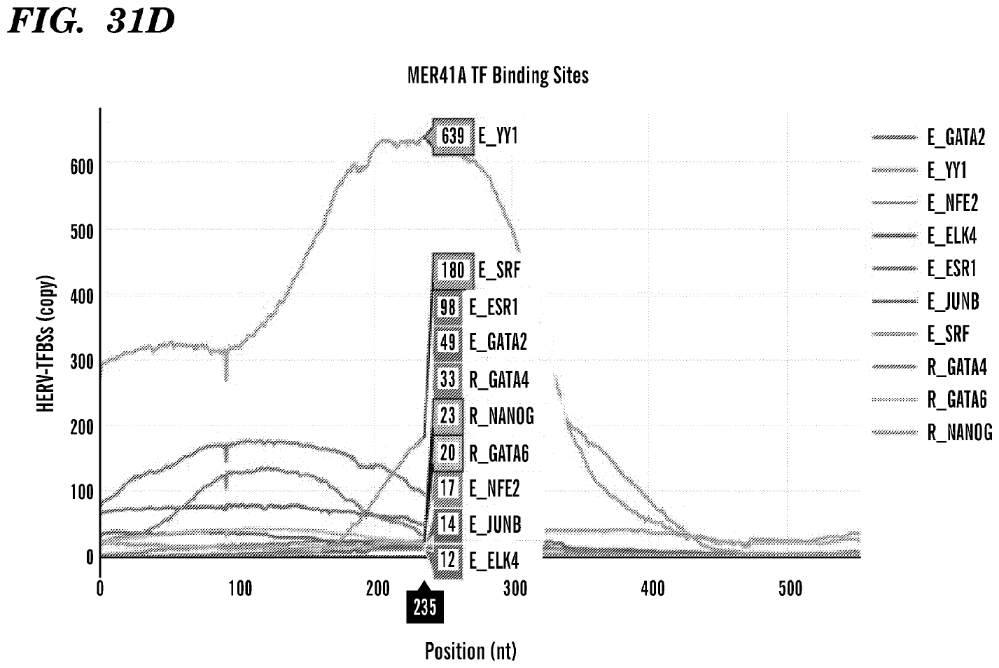

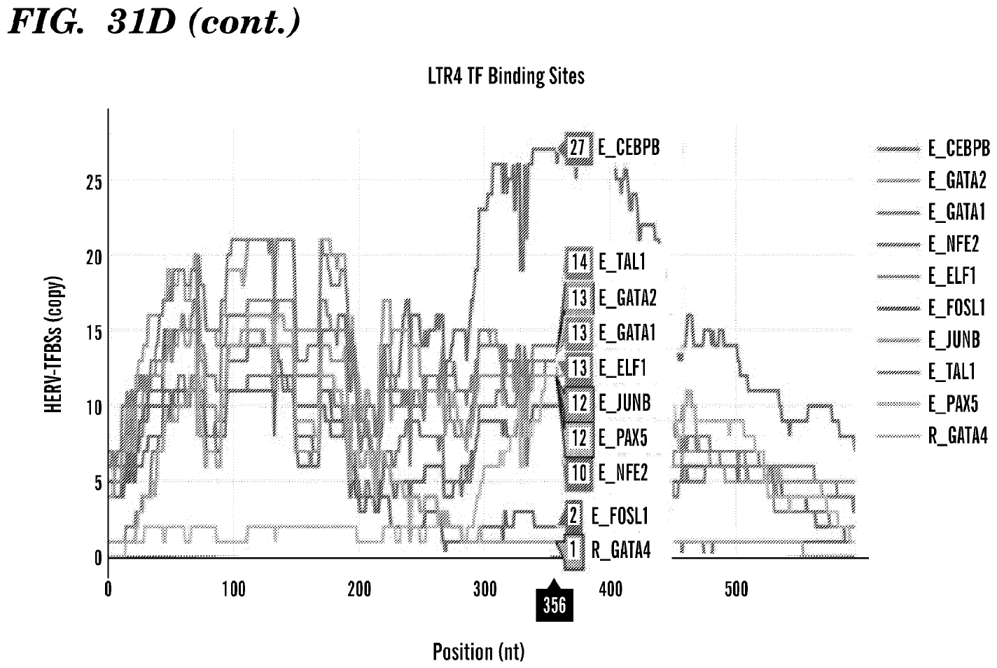

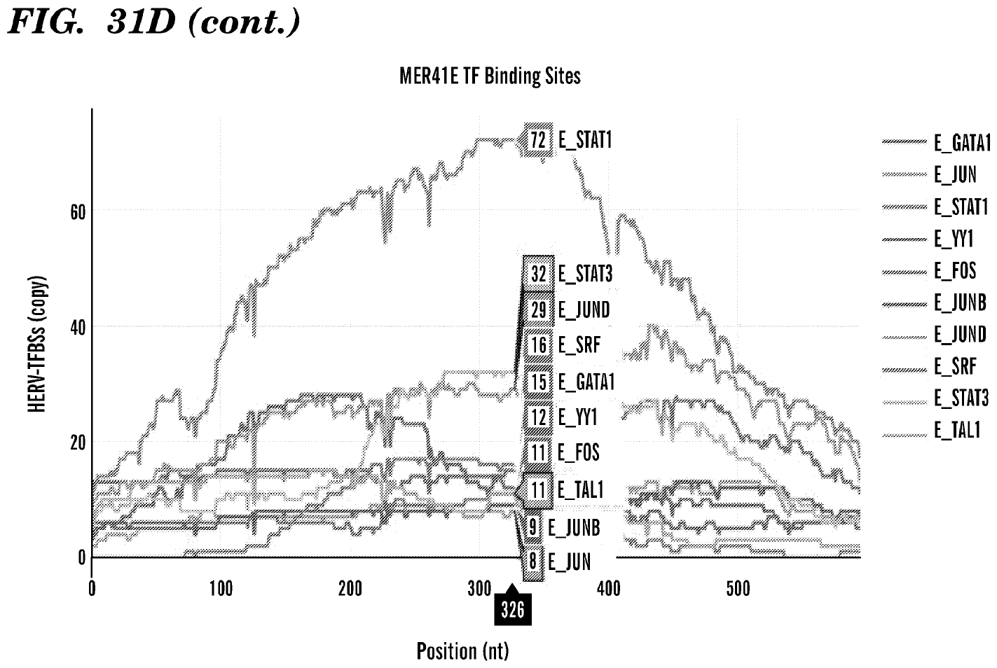

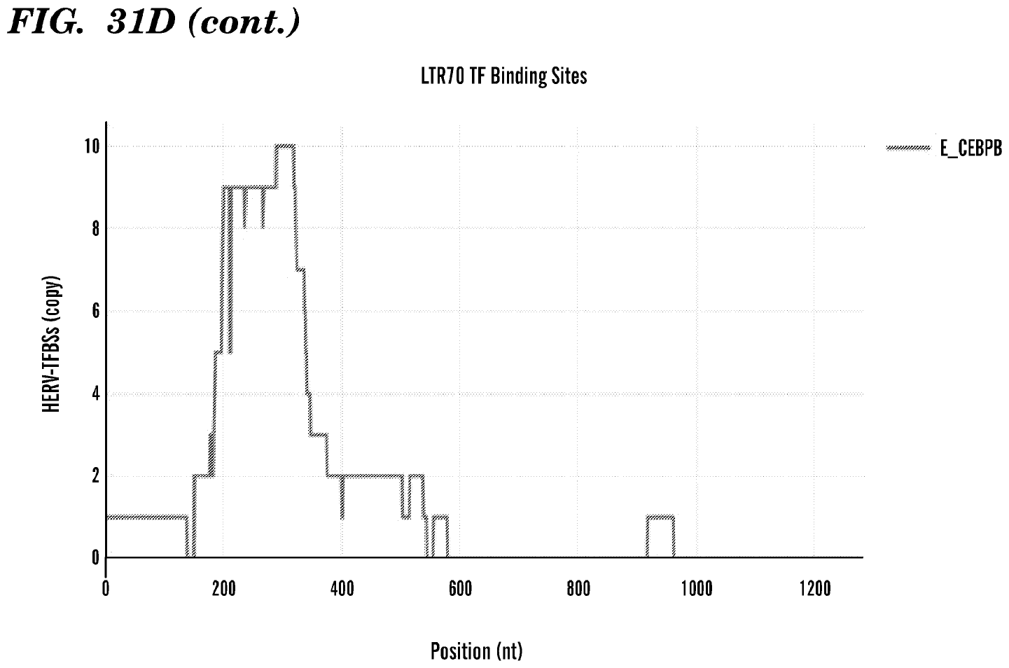

To characterize the NK cells generated as a result of loss of EHMT1 or TRIM28, lymphoid progenitors were differentiated in NK supportive differentiation conditions for an additional two weeks and tested the resulting NK cells in phenotypic and molecular assays (FIG. 26A). NK cells isolated on D4+28 exhibited derepression of several hundred TE families, including LTR, MER, L1 and Alu elements (FIGS. 31A-31B). The classes of derepressed TEs were largely distinct between TRIM28 and EHMT1 knockouts, indicating that EHMT1 and TRIM28 regulate separate classes of TEs within NK cells. In addition, 36 zinc finger proteins were transcriptionally upregulated in TRIM28 knockout NK cells and 26 are annotated to contain a KRAB domain (FIG. 31C), consistent with the known mechanism-of-action of TRIM28 in the KZFP TE silencing pathway. This is in contrast to EHMT1 knockout NK cells where only 3 KZFPs were transcriptionally upregulated, and in line with the non-overlapping sets of TEs derepressed between EHMT1 and TRIM28 knockouts. In either case, derepressed TEs contained TF binding sites relevant for NK cell differentiation and function (FIG. 31D), supportive of a molecular model whereby TE derepression enables TFs to bind chromatin to drive selection of NK cell fate.

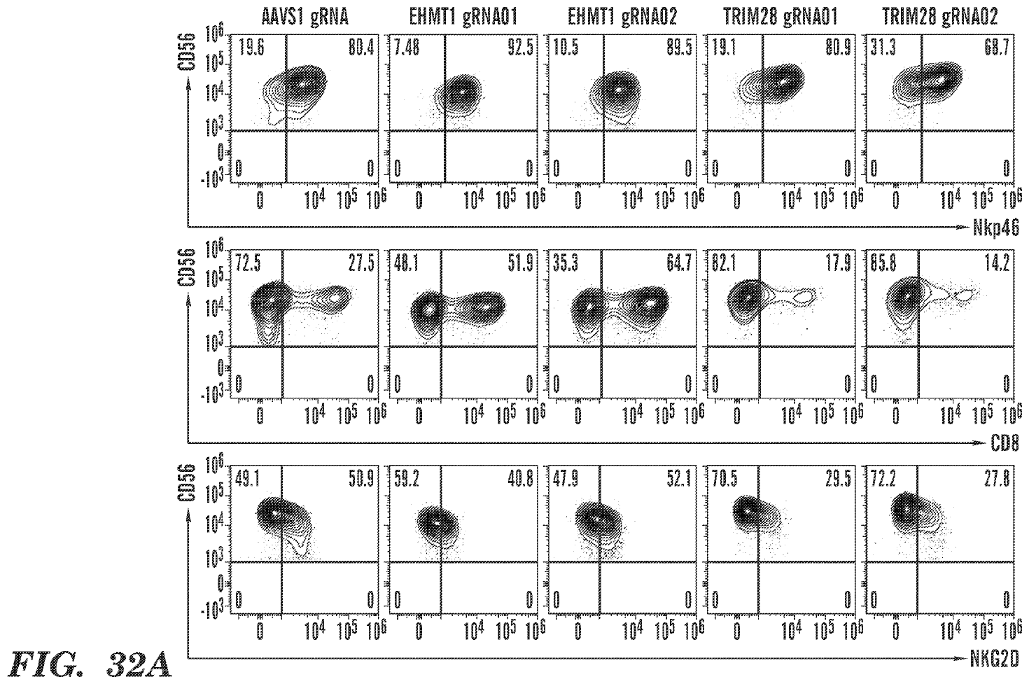

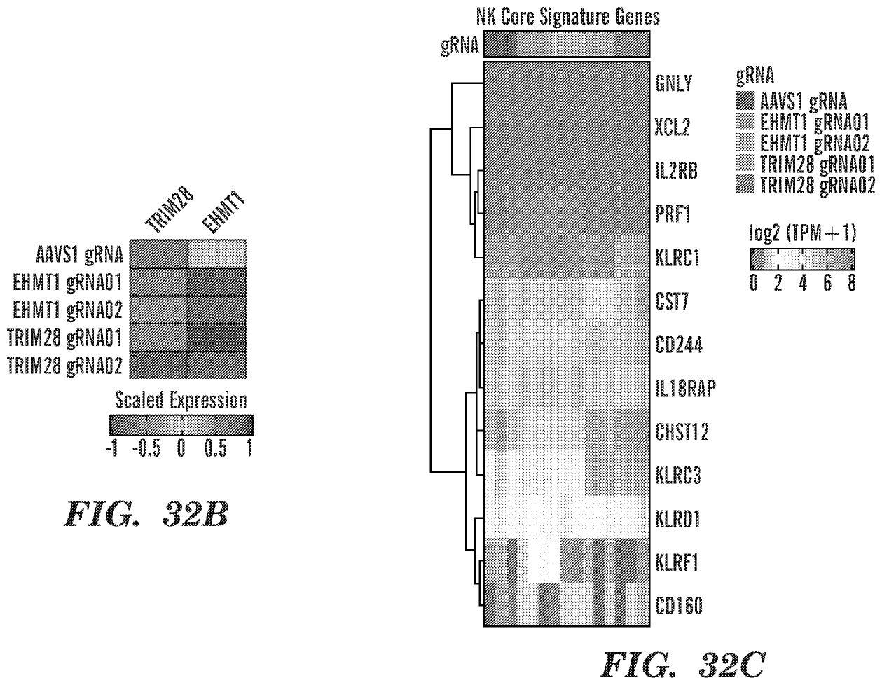

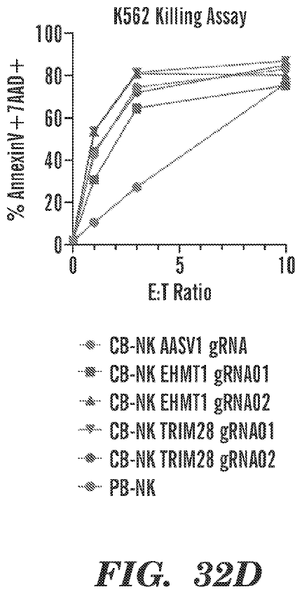

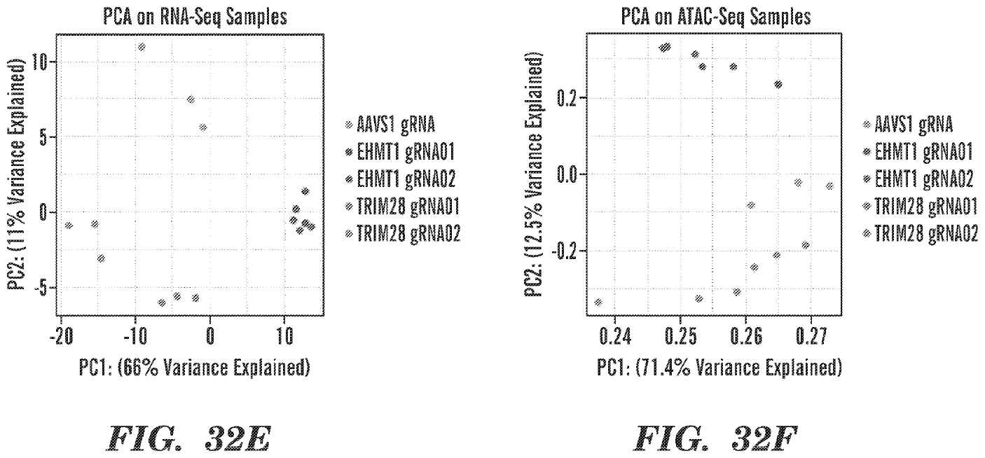

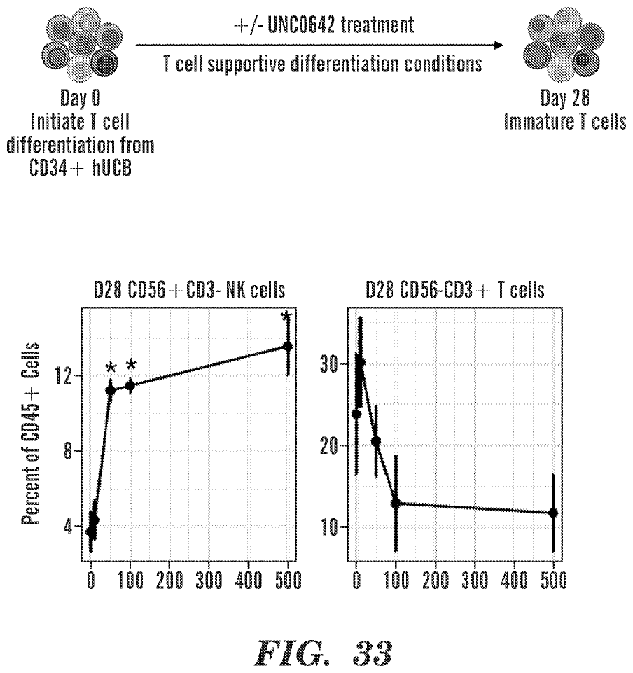

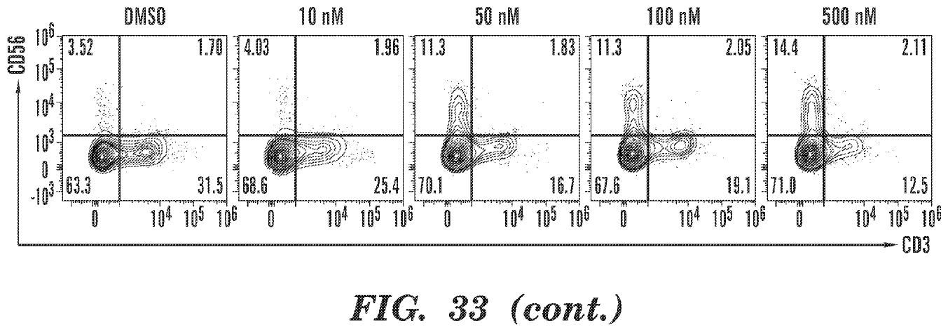

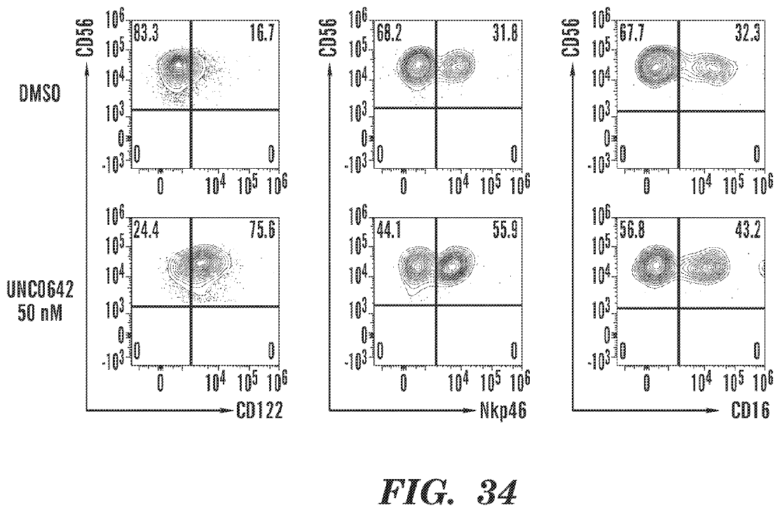

Based on these findings the phenotypic properties of EHMT1 and TRIM28 knockout NK cells were further investigated. NK cells were efficiently derived in all gRNA conditions and expressed canonical NK surface markers (FIG. 32A). RNA-Seq confirmed transcriptional downregulation of TRIM28 and EHMT1 within TRIM28 gRNA and EHMT1 gRNA conditions, respectively, and each condition expressed core NK signature genes (FIGS. 32B-32C). All knockout NK cells displayed cytotoxicity against K562s in co-culture, further supporting their NK identity (FIG. 32D). Notably, nearly three-fold more NK cells were generated in EHMT1 knockouts (and for one gRNA targeting TRIM28 compared to the AAVS1 gRNA control; FIGS. 26B-26C). Knockout of EHMT1 also resulted in a greater frequency of CD16+ NK cells (FIGS. 26D-26E), suggestive of a more mature state. AP-1 transcription factors exhibited enhanced activity with EHMT1 knockout NK cells, whereas inflammatory TFs (IRFs, STATs and NFKB) were more active in TRIM28 knockouts (FIG. 26F, FIGS. 32E-32F). TRIM28 knockout NK cells correspondingly exhibited transcriptional upregulation of various interferon related genes (FIGS. 26G-26H), and enhanced IFN-γ production (FIG. 26I). In further support of a more mature state, EHMT1 knockout NK cells upregulated various KIR and HLA-II genes in comparison to TRIM28 knockout and AAVS1 gRNA control populations. Overall, these data indicate that knockout of EHMT1 and TRIM28 generate NK cells with distinct molecular and phenotypic properties.

### DISCUSSION

In this study, analytical models, omics analysis and chemical/genetic perturbations were combined to implicate TEs as intrinsic to the regulatory logic of the human hematopoietic system, particularly during lymphoid differentiation. To understand the contribution of TEs to gene regulation, genome-wide maps of enhancer-gene regulation were built on an individual cell type basis within the human hematopoietic system. It was observed that TEs exhibited dynamic activity during lymphoid differentiation. By modulating regulators of heterochromatin formation, it was demonstrated that derepression of TEs during lymphoid differentiation resulted in the acquisition of NK cell fates, even in differentiation conditions designed to support T or B cells. In particular, it was documented that knockout of EHMT1 or TRIM28 within HSPCs generated distinct lymphoid progenitor states that diverged from T-fated progenitors, which coincided with derepression of distinct TE families within in vitro derived NK cells. It was noted that knockout of EHMT1 generated more CD16+ NK cells, whereas knockout of TRIM28 resulted in elevated IFN-γ production, reflective of distinct NK states.

The availability of epigenomic data from various cell types has fueled scientific inquiry and hypothesis generation on the role of TEs in regulating lineage-specific transcriptional networks. While abundant, these data often lack insights on putative functional enhancers, which hinders identifying true regulatory relationships. To address this challenge, the ABC model was employed—which performs robustly to predict CRISPR experiments of endogenous enhancer activity—to generate enhancer-gene predictions within the hematopoietic system. The experimental limitations of perturbing repetitive elements within rare primary cells often limits the degree to which individual elements can be functionally tested for regulatory activity. By utilizing state-of-the-art models to identify putative functional enhancers, the approach utilized in this study provides higher confidence in the regulatory potential of implicated TEs. Furthermore, to systematically study dynamic differentiation processes. ABC predictions were generated on a comprehensive set of hematopoietic cell types to capture stages of differentiation from hematopoietic stem cells to erythroid, myeloid and lymphoid lineages, as well as stimulation states within immune populations. Given the long-standing importance of hematopoiesis as a paradigm of stem cell biology, the methods represent a valuable strategy for understanding the role of TEs in gene regulation in other stem cell systems, and may inform the genetic bases of hematopoietic diseases.

A core insight from the study is that TEs are dynamically expressed during hematopoietic differentiation and progressively upregulated during lymphopoiesis with associated downregulation of regulators of heterochromatin formation. Indeed, this observation is consistent with reports of other dynamic processes, such as embryonic development, where widespread DNA-demethylation causes stage-specific TE expression. Importantly, the work extends beyond the observation that TE expression corresponds with stage and lineage-specific signposts of hematopoietic differentiation. Indeed, a diverse set of regulators of heterochromatin was systematically perturbed within HSPCs to derepress TEs during hematopoietic differentiation, and discovered that knockout of the H3K9 lysine methyltransferase, EHMT1 or the transcriptional co-repressor TRIM28 directly dictate NK lineage skewing at the expense of B and T cell fates. Distinct TE families were derepressed in EHMT1 knockouts compared to TRIM28 knockouts, which is consistent with prior observations in murine embryonic stem cells that invoked EHMT1/EHMT2 involvement in silencing MERVL elements with little involvement from TRIM28. These distinct sets of derepressed TEs appear to contribute to the different NK phenotypes observed between the two knockouts. Of note, although SUV39H2 was knocked out and did not observe a phenotype, this may be due to compensation by SUV39H1, as SUV39H1/2 double knockouts are needed to observe heterochromatin disruption in T cells.

Seminal studies have implicated endogenous retroviruses in regulating innate immune pathways. The study lends compelling support to the long-standing hypothesis that the innate immune system evolved through co-option of endogenous viruses. The study demonstrates that TE derepression facilitates development of NK cells—the very cells that are responsible for protection from viral pathogens. Upregulation of interferon-related genes was observed within TRIM28 knockout NK cells, which is consistent with innate immune sensing of TE derived transcripts. Furthermore, TRIM28 knockout NK cells also experienced enhanced IFN-γ production, a critical inflammatory mediator that these cells secrete for antiviral innate immunity.

NK cells have garnered increased attention as adoptive immunotherapy for cancer treatment. Accumulating evidence over the past decade has underscored the functional and molecular heterogeneity of human NK cells owing to transcription factor utilization, prior antigen exposure, developmental ontogeny, and tissue residence, among several factors. These variables ultimately influence the anti-tumor activity and phenotypic properties of NK cells. However, despite considerable interest and ongoing experimentation, clinical studies have not yet fully resolved the specific NK subtypes that are most efficacious against various forms of cancer. Therefore, strategies to engineer and derive targeted populations of NK cells in vitro with distinct effector functions will be essential to develop effective therapies and better understand of the cells responsible for cancer remission. The study reveals fundamental mechanisms by which TEs dictate hematopoietic differentiation, and illustrate the potential of leveraging TE regulatory machinery to modulate TEs for the in vitro generation of NK cells with diverse properties for translational applications.

### REFERENCES

Adoue, Véronique, Bénédicte Binet, Agathe Malbec, Joanna Fourquet, Paola Romagnoli, Joost P. M. van Meerwijk, Sebastian Amigorena, and Olivier P. Joffre. 2019. “The Histone Methyltransferase SETDB1 Controls T Helper Cell Lineage Integrity by Repressing Endogenous Retroviruses.” Immunity 50 (3): 629-644.e8. https://doi.org/10.1016/j.immuni.2019.01.003.
Bauer, Daniel E., Sophia C. Kamran, Samuel Lessard, Jian Xu, Yuko Fujiwara, Came Lin, Zhen Shao, et al. 2013. “An Erythroid Enhancer of BCL11A Subject to Genetic Variation Determines Fetal Hemoglobin Level.” Science (New York, N.Y) 342 (6155): 253-57. https://doi.org/10.1126/science.1242088.
Bentsen, Mette, Philipp Goymann, Hendrik Schultheis, Kathrin Klee, Anastasiia Petrova, René Wiegandt, Annika Fust, et al. 2020. “ATAC-Seq Footprinting Unravels Kinetics of Transcription Factor Binding during Zygotic Genome Activation.” Nature Communications 11 (1): 4267. https://doi.org/10.1038/s41467-020-18035-1.
Best, Steve, Paul Le Tissier, Greg Towers, and Jonathan P. Stoye. 1996. “Positional Cloning of the Mouse Retrovirus Restriction Gene Fvl.” Nature 382 (6594): 826-29. https://doi.org/10.1038/382826a0.
Bray, Nicolas L., Harold Pimentel, Páll Melsted, and Lior Pachter. 2016. “Near-Optimal Probabilistic RNA-Seq Quantification.” Nature Biotechnology 34 (5): 525-27. https://doi.org/10.1038/nbt.3519.
Buenrostro, Jason D., M. Ryan Corces, Caleb A. Lareau, Beijing Wu, Alicia N. Schep, Martin J. Aryee, Ravindra Majeti, Howard Y. Chang, and William J. Greenleaf. 2018. “Integrated Single-Cell Analysis Maps the Continuous Regulatory Landscape of Human Hematopoietic Differentiation.” Cell 0 (0). https://doi.org/10.1016/j.cell.2018.03.074.
Bulut-Karslioglu, Aydan, Inti A. De La Rosa-Velázquez, Fidel Ramirez, Maxim Barenboim, Megumi Onishi-Seebacher, Julia Arand, Carmen Galin, et al. 2014. “Suv39h-Dependent H3K9me3 Marks Intact Retrotransposons and Silences LINE Elements in Mouse Embryonic Stem Cells.” Molecular Cell 55 (2): 277-90. https://doi.org/10.1016/j.molcel.2014.05.029.
Calderon, Diego, Michelle L. T. Nguyen, Anja Mezger, Arwa Kathiria, Fabian Müller, Vinh Nguyen, Ninnia Lescano, et al. 2019. “Landscape of Stimulation-Responsive Chromatin across Diverse Human Immune Cells.” Nature Genetics 51 (10): 1494-1505. https://doi.org/10.1038/s41588-019-0505-9.
Chuong, Edward B., Nels C. Elde, and Cédric Feschotte. 2016. “Regulatory Evolution of Innate Immunity through Co-Option of Endogenous Retroviruses.” Science (New York, N. Y.) 351 (6277): 1083-87. https://doi.org/10.1126/science.aad5497.
Chuong, Edward B., Elde, Nels C., and Feschotte, Cedric.. 2017. “Regulatory Activities of Transposable Elements: From Conflicts to Benefits.” Nature Reviews Genetics 18 (2): 71-86. https://doi.org/10.1038/nrg.2016.139.
Chuong, Edward B., M. A. Karim Rumi, Michael J. Soares, and Julie C. Baker. 2013. “Endogenous Retroviruses Function as Species-Specific Enhancer Elements in the Placenta.” Nature Genetics 45 (3): 325-29. https://doi.org/10.1038/ng.2553.
Cichocki, Frank, Bartosz Grzywacz, and Jeffrey S. Miller. 2019. “Human NK Cell Development: One Road or Many?” Frontiers in Immunology 10: 2078. https://doi.org/10.3389/fimmu.2019.02078.
Consortium, The ENCODE Project. 2012. “An Integrated Encyclopedia of DNA Elements in the Human Genome.” Nature 489 (7414): 57-74. https://doi.org/10.1038/nature11247.
Corces, M. Ryan, Jason D. Buenrostro, Beijing Wu, Peyton G. Greenside, Steven M. Chan, Julie L. Koenig, Michael P. Snyder, et al. 2016. “Lineage-Specific and Single-Cell Chromatin Accessibility Charts Human Hematopoiesis and Leukemia Evolution.” Nature Genetics advance online publication (August). https://doi.org/10.1038/ng.3646.
Corces, M. Ryan, Jeffrey M. Granja, Shadi Shams, Bryan H. Louie, Jose A. Seoane, Wanding Zhou, Tiago C. Silva, et al. 2018. “The Chromatin Accessibility Landscape of Primary Human Cancers.” Science 362 (6413): eaav1898. https://doi.org/10.1126/science.aav1898.
Corces, M Ryan, Alexandro E Trevino, Emily G Hamilton, Peyton G Greenside, Nicholas A Sinnott-Armstrong, Sam Vesuna, Ansuman T Satpathy, et al. 2017. “An Improved ATAC-Seq Protocol Reduces Background and Enables Interrogation of Frozen Tissues.” Nature Methods 14 (10): 959-62. https://doi.org/10.1038/nmeth.4396.
Crinier, Adeline, Pierre Milpied, Bertrand Escalière, Christelle Piperoglou, Justine Galluso, Anais Balsamo, Lionel Spinelli, et al. 2018. “High-Dimensional Single-Cell Analysis Identifies Organ-Specific Signatures and Conserved NK Cell Subsets in Humans and Mice.” Immunity 49 (5): 971-986.e5. https://doi.org/10.1016/j.immuni.2018.09.009.
Dege, Carissa, Katherine H. Fegan, J. Philip Creamer, Melissa M. Berrien-Elliott, Stephanie A. Luff, Darren Kim, Julia A. Wagner, et al. 2020. “Potently Cytotoxic Natural Killer Cells Initially Emerge from Erythro-Myeloid Progenitors during Mammalian Development.” Developmental Cell 0 (0). https://doi.org/10.1016/j.devcel.2020.02.016.
Ebihara, Takashi, Christina Song, Stacy H. Ryu, Beatrice Plougastel-Douglas, Liping Yang, Ditsa Levanon, Yoram Groner, et al. 2015. “Runx3 Specifies Lineage Commitment of Innate Lymphoid Cells.” Nature Immunology 16 (11): 1124-33. https://doi.org/10.1038/ni.3272.
Ecco, Gabriela, Michael Imbeault, and Didier Trono. 2017. “KRAB Zinc Finger Proteins.” Development (Cambridge, England) 144 (15): 2719-29. https://doi.org/10.1242/dev.132605.
Feschotte, Cédric, and Clément Gilbert. 2012. “Endogenous Viruses: Insights into Viral Evolution and Impact on Host Biology.” Nature Reviews Genetics 13 (4): 283-96. https://doi.org/10.1038/nrg3199.
Freud, Aharon G., Bethany L. Mundy-Bosse, Jianhua Yu, and Michael A. Caligiuri. 2017. “The Broad Spectrum of Human Natural Killer Cell Diversity.” Immunity 47 (5): 820-33. https://doi.org/10.1016/j.immuni.2017.10.008.
Friedli, Marc, Priscilla Turelli, Adamandia Kapopoulou, Benjamin Rauwel, Nathaly Castro-Diaz, Helen M. Rowe, Gabriela Ecco, et al. 2014. “Loss of Transcriptional Control over Endogenous Retroelements during Reprogramming to Pluripotency.” Genome Research 24 (8): 1251-59. https://doi.org/10.1101/gr.172809.114.Friedman, J. R., W. J. Fredericks, D. E. Jensen, D. W. Speicher, X. P. Huang, E. G. Neilson, and F. J. Rauscher. 1996. “KAP-1, a Novel Corepressor for the Highly Conserved KRAB Repression Domain.” Genes & Development 10 (16): 2067-78. https://doi.org/10.1101/gad.10.16.2067.
Fuentes, Daniel R, Tomek Swigut, and Joanna Wysocka. 2018. “Systematic Perturbation of Retroviral LTRs Reveals Widespread Long-Range Effects on Human Gene Regulation.” Edited by Edith Heard and Detlef Weigel. ELife 7 (August): e35989. https://doi.org/10.7554/eLife.35989.
Fueyo, Raquel, Julius Judd, Cedric Feschotte, and Joanna Wysocka. 2022. “Roles of Transposable Elements in the Regulation of Mammalian Transcription.” Nature Reviews Molecular Cell Biology, February, 1-17. https://doi.org/10.1038/s41580-022-00457-y.
Fukuda, Kei, and Yoichi Shinkai. 2020. “SETDB1-Mediated Silencing of Retroelements.” Viruses 12 (6): 596. https://doi.org/10.3390/v12060596.
Fulco, Charles P., Joseph Nasser, Thouis R. Jones, Glen Munson, Drew T. Bergman, Vidya Subramanian, Sharon R. Grossman, et al. 2019. “Activity-by-Contact Model of Enhancer-Promoter Regulation from Thousands of CRISPR Perturbations.” Nature Genetics 51 (12): 1664-69. https://doi.org/10.1038/s41588-019-0538-0.
Gao, Xin, Kirby D. Johnson, Yuan-I. Chang, Meghan E. Boyer, Colin N. Dewey, Jing Zhang, and Emery H. Bresnick. 2013. “Gata2 Cis-Element Is Required for Hematopoietic Stem Cell Generation in the Mammalian Embryo.” Journal of Experimental Medicine 210 (13): 2833-42. https://doi.org/10.1084/jem.20130733.
Gasperini, Molly, Jacob M. Tome, and Jay Shendure. 2020. “Towards a Comprehensive Catalogue of Validated and Target-Linked Human Enhancers.” Nature Reviews Genetics 21 (5): 292-310. https://doi.org/10.1038/s41576-019-0209-0.
Gazquez-Gutierrez, Ana, Jeroen Witteveldt, Sara R Heras, and Sara Macias. 2021. “Sensing of Transposable Elements by the Antiviral Innate Immune System.” RNA (New York, N. Y.), April, rna.078721.121. https://doi.org/10.1261/ma.078721.121.
Granja, Jeffrey M., Sandy Klemm, Lisa M. McGinnis, Arwa S. Kathiria, Anja Mezger, M. Ryan Corces, Benjamin Parks, et al. 2019. “Single-Cell Multiomic Analysis Identifies Regulatory Programs in Mixed-Phenotype Acute Leukemia.” Nature Biotechnology, December, 1-8. https://doi.org/10.1038/s41587-019-0332-7.
Grow, Edward J., Ryan A. Flynn, Shawn L. Chavez, Nicholas L. Bayless, Mark Wossidlo, Daniel J. Wesche, Lance Martin, et al. 2015. “Intrinsic Retroviral Reactivation in Human Preimplantation Embryos and Pluripotent Cells.” Nature 522 (7555): 221-25. https://doi.org/10.1038/nature14308.
Guo, Yalin, Ivan Maillard, Sankhamala Chakraborti, Ellen V. Rothenberg, and Nancy A. Speck. 2008. “Core Binding Factors Are Necessary for Natural Killer Cell Development and Cooperate with Notch Signaling during T-Cell Specification.” Blood 112 (3): 480-92. https://doi.org/10.1182/blood-2007-10-120261.
He, Jiangping, Isaac A. Babarinde, Li Sun, Shuyang Xu, Ruhai Chen, Junjie Shi, Yuanjie Wei, et al. 2021. “Identifying Transposable Element Expression Dynamics and Heterogeneity during Development at the Single-Cell Level with a Processing Pipeline ScTE.” Nature Communications 12 (1): 1456. https://doi.org/10.1038/s41467-021-21808-x.
Heinz, Sven, Christopher Benner, Nathanael Spann, Eric Bertolino, Yin C. Lin, Peter Laslo, Jason X. Cheng, Cornelis Murre, Harinder Singh, and Christopher K. Glass. 2010. “Simple Combinations of Lineage-Determining Transcription Factors Prime Cis-Regulatory Elements Required for Macrophage and B Cell Identities.” Molecular Cell 38 (4): 576-89. https://doi.org/10.1016/j.molcel.2010.05.004.
Huang, Jialiang, Xin Liu, Dan Li, Zhen Shao, Hui Cao, Yuannyu Zhang, Eirini Trompouki, et al. 2016. “Dynamic Control of Enhancer Repertoires Drives Lineage and Stage-Specific Transcription during Hematopoiesis.” Developmental Cell 36 (1): 9-23. https://doi.org/10.1016/j.devcel.2015.12.014.
Imbeault, Michaël, Pierre-Yves Helleboid, and Didier Trono. 2017. “KRAB Zinc-Finger Proteins Contribute to the Evolution of Gene Regulatory Networks.” Nature 543 (7646): 550-54. https://doi.org/10.1038/nature21683.
Ivancevic, Atma, David M. Simpson, and Edward B. Chuong. 2021. “Endogenous Retroviruses Mediate Transcriptional Rewiring in Response to Oncogenic Signaling in Colorectal Cancer.” bioRxiv. https://doi.org/10.1101/2021.10.28.466196.
Jin, Ying, Oliver H. Tam, Eric Paniagua, and Molly Hammell. 2015. “TEtranscripts: A Package for Including Transposable Elements in Differential Expression Analysis of RNA-Seq Datasets.” Bioinformatics (Oxford, England) 31 (22): 3593-99. https://doi.org/10.1093/bioinformatics/btv422.
Karimi, Mohammad M., Preeti Goyal, Irma A. Maksakova, Misha Bilenky, Danny Leung, Jie Xin Tang, Yoichi Shinkai, et al. 2011. “DNA Methylation and SETDB1/H3K9me3 Regulate Predominantly Distinct Sets of Genes, Retroelements and Chimaeric Transcripts in Mouse ES Cells.” Cell Stem Cell 8 (6): 676-87. https://doi.org/10.1016/j.stem.2011.04.004.
Keenan, Christine R., Nadia Iannarella, Gaetano Naselli, Naiara G. Bediaga, Timothy M. Johanson, Leonard C. Harrison, and Rhys S. Allan. 2020. “Extreme Disruption of Heterochromatin Is Required for Accelerated Hematopoietic Aging.” Blood 135 (23): 2049-58. https://doi.org/10.1182/blood.2019002990.
Kunarso, Galih, Na-Yu Chia, Justin Jeyakani, Catalina Hwang, Xinyi Lu, Yun-Shen Chan, Huck-Hui Ng, and Guillaume Bourque. 2010. “Transposable Elements Have Rewired the Core Regulatory Network of Human Embryonic Stem Cells.” Nature Genetics 42 (7): 631-34. https://doi.org/10.1038/ng.600.
Lara-Astiaso, David, Assaf Weiner, Erika Lorenzo-Vivas, Irina Zaretsky, Diego Adhemar Jaitin, Eyal David, Hadas Keren-Shaul, et al. 2014. “Chromatin State Dynamics during Blood Formation.” Science 345 (6199): 943-49. https://doi.org/10.1126/science.1256271.
Laurenti, Elisa, and Berthold Göttgens. 2018. “From Haematopoietic Stem Cells to Complex Differentiation Landscapes.” Nature 553 (7689): 418-26. https://doi.org/10.1038/nature25022.
Layer, Ryan M., Brent S. Pedersen, Tonya DiSera, Gabor T. Marth, Jason Gertz, and Aaron R. Quinlan. 2018. “GIGGLE: A Search Engine for Large-Scale Integrated Genome Analysis.” Nature Methods 15 (2): 123-26. https://doi.org/10.1038/nmeth.4556.
Le Coz, Carole, David N. Nguyen, Chun Su, Brian E. Nolan, Amanda V. Albrecht, Suela Xhani, Di Sun, et al. 2021. “Constrained Chromatin Accessibility in PU.1-Mutated Agammaglobulinemia Patients.” Journal of Experimental Medicine 218 (7): e20201750. https://doi.org/10.1084/jem.20201750.
Levanon, Ditsa, Varda Negreanu, Joseph Lotem, Karen Rae Bone, On Brenner, Dena Leshkowitz, and Yoram Groner. 2014. “Transcription Factor Runx3 Regulates Interleukin-15-Dependent Natural Killer Cell Activation.” Molecular and Cellular Biology 34 (6): 1158-69. https://doi.org/10.1128/MCB.01202-13.
Liu, Enli, David Marin, Pinaki Banerjee, Homer A. Macapinlac, Philip Thompson, Rafet Basar, Lucila Nassif Kerbauy, et al. 2020. “Use of CAR-Transduced Natural Killer Cells in CD19-Positive Lymphoid Tumors.” New England Journal of Medicine 382 (6): 545-53. https://doi.org/10.1056/NEJMoa1910607.
Liu, Feng, Dalia Barsyte-Lovejoy, Fengling Li, Yan Xiong, Victoria Korboukh, Xi-Ping Huang, Abdellah Allali-Hassani, et al. 2013. “Discovery of an in Vivo Chemical Probe of the Lysine Methyltransferases G9a and GLP.” Journal of Medicinal Chemistry 56 (21): 8931-42. https://doi.org/10.1021/jm401480r.
Love, Michael I., Wolfgang Huber, and Simon Anders. 2014. “Moderated Estimation of Fold Change and Dispersion for RNA-Seq Data with DESeq2.” Genome Biology 15 (12): 550. https://doi.org/10.1186/s13059-014-0550-8.
Ludwig, Leif S., Caleb A. Lareau, Erik L. Bao, Satish K. Nandakumar, Christoph Muus, Jacob C. Ulirsch, Kaitavjeet Chowdhary, et al. 2019. “Transcriptional States and Chromatin Accessibility Underlying Human Erythropoiesis.” Cell Reports 27 (11): 3228-3240.e7. https://doi.org/10.1016/j.celrep.2019.05.046.
Maksakova, Irina A., Peter J. Thompson, Preeti Goyal, Steven Jm Jones, Prim B. Singh, Mohammad M. Karimi, and Matthew C. Lorincz. 2013. “Distinct Roles of KAP1, HP1 and G9a/GLP in Silencing of the Two-Cell-Specific Retrotransposon MERVL in Mouse ES Cells.” Epigenetics & Chromatin 6 (1): 15. https://doi.org/10.1186/1756-8935-6-15.
Matsui, Toshiyuki, Danny Leung, Hiroki Miyashita, Irma A. Maksakova, Hitoshi Miyachi, Hiroshi Kimura, Makoto Tachibana, Matthew C. Lorincz, and Yoichi Shinkai. 2010. “Proviral Silencing in Embryonic Stem Cells Requires the Histone Methyltransferase ESET.” Nature 464 (7290): 927-31. https://doi.org/10.1038/nature08858.
Miller, Jeffrey S., Yvette Soignier, Angela Panoskaltsis-Mortari, Sarah A. McNeamey, Gong H. Yun, Susan K. Fautsch, David McKenna, et al. 2005. “Successful Adoptive Transfer and in Vivo Expansion of Human Haploidentical NK Cells in Patients with Cancer.” Blood 105 (8): 3051-57. https://doi.org/10.1182/blood-2004-07-2974.
Nasser, Joseph, Drew T. Bergman, Charles P. Fulco, Philine Guckelberger, Benjamin R. Doughty, Tejal A. Patwardhan, Thouis R. Jones, et al. 2021. “Genome-Wide Enhancer Maps Link Risk Variants to Disease Genes.” Nature, April, 1-6. https://doi.org/10.1038/s41586-021-03446-x.
Nottingham, Wade T., Andrew Jarratt, Matthew Burgess, Caroline L. Speck, Jan-Fang Cheng, Shyam Prabhakar, Eddy M. Rubin, et al. 2007. “Runx1-Mediated Hematopoietic Stem-Cell Emergence Is Controlled by a Gata/Ets/SCL-Regulated Enhancer.” Blood 110 (13): 4188-97. https://doi.org/10.1182/blood-2007-07-100883.
Ohnuki, Mari, Koji Tanabe, Kenta Sutou, Ito Teramoto, Yuka Sawamura, Megumi Narita, Michiko Nakamura, et al. 2014. “Dynamic Regulation of Human Endogenous Retroviruses Mediates Factor-Induced Reprogramming and Differentiation Potential.” Proceedings of the National Academy of Sciences of the United States of America 111 (34): 12426-31. https://doi.org/10.1073/pnas.1413299111.
Pehrsson, Erica C., Mayank N. K. Choudhary, Vasavi Sundaram, and Ting Wang. 2019. “The Epigenomic Landscape of Transposable Elements across Normal Human Development and Anatomy.” Nature Communications 10 (1): 5640. https://doi.org/10.1038/s41467-019-13555-x.
Petersen, Romina, John J. Lambourne, Biola M. Javierre, Luigi Grassi, Roman Kreuzhuber, Dace Ruklisa, Isabel M. Rosa, et al. 2017. “Platelet Function Is Modified by Common Sequence Variation in Megakaryocyte Super Enhancers.” Nature Communications 8 (1): 16058. https://doi.org/10.1038/ncomms16058.
Picelli, Simone, Omid R Faridani, Asa K Björklund, Gösta Winberg, Sven Sagasser, and Rickard Sandberg. 2014. “Full-Length RNA-Seq from Single Cells Using Smart-Seq2.” Nature Protocols 9 (1): 171-81. https://doi.org/10.1038/nprot.2014.006.
Rawat, Priyanka, and Asmita Das. 2022. “Differential Expression of Disparate Transcription Factor Regime Holds the Key for NK Cell Development and Function Modulation.” Life Sciences 297 (May): 120471. https://doi.org/10.1016/j.lfs.2022.120471.
Renoux, Virginie M., Alya Zriwil, Claudia Peitzsch, Jakob Michaëlsson, Danielle Friberg, Shamit Soneji, and Ewa Sitnicka. 2015. “Identification of a Human Natural Killer Cell Lineage-Restricted Progenitor in Fetal and Adult Tissues.” Immunity 43 (2): 394-407. https://doi.org/10.1016/j.immuni.2015.07.011.
Roadmap Epigenomics Consortium, Anshul Kundaje, Wouter Meuleman, Jason Ernst, Misha Bilenky, Angela Yen, Alireza Heravi-Moussavi, et al. 2015. “Integrative Analysis of 111 Reference Human Epigenomes.” Nature 518 (7539): 317-30. https://doi.org/10.1038/nature14248.
Romee, Rizwan, Maximillian Rosario, Melissa M. Berrien-Elliott, Julia A. Wagner, Brea A. Jewell, Timothy Schappe, Jeffrey W. Leong, et al. 2016. “Cytokine-Induced Memory-like Natural Killer Cells Exhibit Enhanced Responses against Myeloid Leukemia.” Science Translational Medicine 8 (357): 357ra123. https://doi.org/10.1126/scitranslmed.aaf2341.
Romee, Rizwan, Stephanie E. Schneider, Jeffrey W. Leong, Julie M. Chase, Catherine R. Keppel, Ryan P. Sullivan, Megan A. Cooper, and Todd A. Fehniger. 2012. “Cytokine Activation Induces Human Memory-like NK Cells.” Blood 120 (24): 4751-60. https://doi.org/10.1182/blood-2012-04-419283.
Rowe, Helen M., Johan Jakobsson, Daniel Mesnard, Jacques Rougemont, Séverine Reynard, Tugce Aktas, Pierre V. Maillard, et al. 2010. “KAP1 Controls Endogenous Retroviruses in Embryonic Stem Cells.” Nature 463 (7278): 237-40. https://doi.org/10.1038/nature08674.
Saito, Takaya, and Marc Rehmsmeier. 2017. “Precrec: Fast and Accurate Precision-Recall and ROC Curve Calculations in R.” Bioinformatics 33 (1): 145-47. https://doi.org/10.1093/bioinformatics/btw570.
Schep, Alicia N., Beijing Wu, Jason D. Buenrostro, and William J. Greenleaf 2017. “ChromVAR: Inferring Transcription-Factor-Associated Accessibility from Single-Cell Epigenomic Data.” Nature Methods 14 (10): 975-78. https://doi.org/10.1038/nmeth.4401.
Schmidt, Dominic, Petra C. Schwalie, Michael D. Wilson, Benoit Ballester, Angela Gongalves, Claudia Kutter, Gordon D. Brown, Aileen Marshall, Paul Flicek, and Duncan T. Odom. 2012. “Waves of Retrotransposon Expansion Remodel Genome Organization and CTCF Binding in Multiple Mammalian Lineages.” Cell 148 (1-2): 335-48. https://doi.org/10.1016/j.cell.2011.11.058.
Smith, Jason P., M. Ryan Corces, Jin Xu, Vincent P. Reuter, Howard Y. Chang, and Nathan C. Sheffield. 2021. “PEPATAC: An Optimized Pipeline for ATAC-Seq Data Analysis with Serial Alignments.” NAR Genomics and Bioinformatics 3 (4): lqab101. https://doi.org/10.1093/nargab/lqab101.
Srinivasachar Badarinarayan, Smitha, and Daniel Sauter. 2021. “Switching Sides: How Endogenous Retroviruses Protect Us from Viral Infections.” Journal of Virology 95 (12): e02299-20. https://doi.org/10.1128/JVI.02299-20.
Stunnenberg, Hendrik G., International Human Epigenome Consortium, and Martin Hirst. 2016. “The International Human Epigenome Consortium: A Blueprint for Scientific Collaboration and Discovery.” Cell 167 (5): 1145-49. https://doi.org/10.1016/j.cell.2016.11.007.
Sundaram, Vasavi, Yong Cheng, Zhihai Ma, Daofeng Li, Xiaoyun Xing, Peter Edge, Michael P. Snyder, and Ting Wang. 2014. “Widespread Contribution of Transposable Elements to the Innovation of Gene Regulatory Networks.” Genome Research 24 (12): 1963-76. https://doi.org/10.1101/gr.168872.113.
Tirosh, Itay, Benjamin Izar, Sanjay M. Prakadan, Marc H. Wadsworth, Daniel Treacy, John J. Trombetta, Asaf Rotem, et al. 2016. “Dissecting the Multicellular Ecosystem of Metastatic Melanoma by Single-Cell RNA-Seq.” Science 352 (6282): 189-96. https://doi.org/10.1126/science.aad0501.
Ulirsch, Jacob C., Caleb A. Lareau, Erik L. Bao, Leif S. Ludwig, Michael H. Guo, Christian Benner, Ansuman T. Satpathy, et al. 2019. “Interrogation of Human Hematopoiesis at Single-Cell and Single-Variant Resolution.” Nature Genetics, March, 1. https://doi.org/10.1038/s41588-019-0362-6.
Vierstra, Jeff, John Lazar, Richard Sandstrom, Jessica Halow, Kristen Lee, Daniel Bates, Morgan Diegel, et al. 2020. “Global Reference Mapping of Human Transcription Factor Footprints.” Nature 583 (7818): 729-36. https://doi.org/10.1038/s41586-020-2528-x.
Wagner, Julia A., Maximillian Rosario, Rizwan Romee, Melissa M. Berrien-Elliott, Stephanie E. Schneider, Jeffrey W. Leong, Ryan P. Sullivan, et al. 2017. “CD56bright NK Cells Exhibit Potent Antitumor Responses Following IL-15 Priming.” The Journal of Clinical Investigation 127 (11): 4042-58. https://doi.org/10.1172/JCI90387.
Wang, Jianrong, Cristina Vicente-Garcia, Davide Seruggia, Eduardo Moltó, Ana Fernandez-Miñán, Ana Neto, Elbert Lee, et al. 2015. “MIR Retrotransposon Sequences Provide Insulators to the Human Genome.” Proceedings of the National Academy of Sciences of the United States of America 112 (32): E4428-4437. https://doi.org/10.1073/pnas.1507253112.
Yamamoto, Ryo, Adam C. Wilkinson, and Hiromitsu Nakauchi. 2018. “Changing Concepts in Hematopoietic Stem Cells.” Science 362 (6417): 895-96. https://doi.org/10.1126/science.aat7873.
Ye, Mengliang, Christel Goudot, Thomas Hoyler, Benjamin Lemoine, Sebastian Amigorena, and Elina Zueva. 2020. “Specific Subfamilies of Transposable Elements Contribute to Different Domains of T Lymphocyte Enhancers.” Proceedings of the National Academy of Sciences 117 (14): 7905-16. https://doi.org/10.1073/pnas.1912008117.

### Data and Code Availability

All -omics data generated to support conclusions for this study is available on the Gene Expression Omnibus under accession: GSEXXXXXXXX.

Processed data and analysis code is available on GitHub: https://github.com/mnajia/hemeTEs

### Experimental Model and Subject Details

### Cell Culture

### Statistics

Investigators were not blind to the experimental samples and conditions, unless otherwise stated. Relevant statistics are mentioned in the figure legends or documented in the methods section. P-value <0.05 was considered significant for all data, unless otherwise stated, and relevant p-values and FDRs are mentioned in associated figures and/or legends.

### Experimental Methods

### CRISPR/Cas9 Knockouts in CD34+ Umbilical Cord Blood

Genetic knockouts of select genes were generated in primary hematopoietic stem and progenitor cells (HSPCs) via nucleofection of CRISPR/Cas9 ribonucleoproteins (RNPs). Two independent knockouts were generated for each gene using separate gRNAs and knockouts were replicated across two different donor pools of human umbilical cord blood. First, CD34+ umbilical cord blood cells (AllCells) were thawed from liquid nitrogen storage via dropwise addition of RPMI 1640+10% FBS. Cells were centrifuged at 200 g for 8 minutes and subsequently washed with FACS buffer (PBS+2% FBS). Cells were cultured for 48 hours prior to nucleofection at a density of 250,000 cells/mL in SFEM II media (StemCell Technologies, 09605) supplemented with 100 ng/mL SCF, TPO, FLT3L, and IL-6 (Peprotech) within a 5% O2, 5% CO2, 37C incubator. Next, the top two protospacer sequences for each desired gene were selected from the Brunello genome-wide knockout library (Broad Institute) based on the Rule Set 2 score. A control gRNA targeting the AAVS1 locus was designed in Benchling. All gRNA sequences are available (SEQ ID NOs: 4-12) and were synthesized by IDT as an Alt-R CRISPR/Cas9 sgRNA. RNPs were complexed by incubating 105 pmol of Cas9 protein (IDT, 1081058) with 125 pmol of sgRNA in a 5 uL total volume for 10 minutes at room temperature. CD34+ cord blood HSPCs were washed with FACS buffer and 125,000 cells were resuspended in 20 uL of P3 Primary Cell Nucleofection Solution (Lonza, V4XP-3032) per nucleofection. RNPs were nucleofected into HSPCs with 3.85 uM electroporation enhancer (IDT, 1075915) using a 16-reaction nucleovette and pulse code DZ-100 on a Lonza 4D-Nucleofector. Cells were cultured for 36-48 hours at 5% O2, 5% CO2 in 96-well U-bottom plates (Corning, 351177) then subsequently harvested for genomic DNA extraction and initiation of in vitro differentiation assays.

### Quantification of Indel Frequencies

Genomic DNA was extracted from at least 50,000 cells using a custom extraction buffer (1 mM CaCl₂), 3 mM MgCl2, 1 mM EDTA, 10 mM Tris-HCl, 1% Triton X-100, and 0.2 mg/ml Proteinase K) and then subjected to the following thermal program: 65 C for 10 minutes then 95 C for 15 minutes. gRNA-targeted genomic loci were PCR dialed-out from genomic DNA using NEBNext High Fidelity Master Mix (NEB, M0541S) and primers that flank ˜200 bps of the expected Cas9 cut site (PCR primers are listed in SEQ ID NOs: 13-30). PCR amplicons were gel extracted from a 2% agarose gel and submitted for Sanger Sequencing using forward and reverse PCR primers. Indel frequencies for each gRNA were quantified using TIDE analysis (http://tide.nki.nl) where the reference nucleotide sequence was derived from a mock-nucleofected control. The proportion of frameshifting indels was then determined from the total TIDE indel frequency estimates.

### In Vitro T Cell Differentiation

Following 36-48 hours post-nucleofection of Cas9 RNPs, 5,000 cord blood HSPCs were differentiated to T cells using the StemSpan T cell Generation Kit (Stem Cell Technologies, 09940) per the manufacturer's instructions with n=3 replicate differentiations per gRNA in 24-well plates. After 14 days of differentiation, cells were collected and 50,000 cells/sample were replated into new 24-well plates. Similarly, after 28 days of differentiation, cells were collected and 250,000 cells were replated into new 24-well plates for the final two weeks of differentiation. Total viable cell counts were performed on Days 14, 28 and 42 of differentiation using a TrypanBlue exclusion dye and a hemocytometer. Aliquots of cells were collected for flow cytometry on days 14, 28 and 42 of differentiation to assess lymphoid progenitors (CD45, CD5 and CD7) and mature lymphoid populations (CD45, CD4, CD8, CD3, CD56, CD5 and CD7). Cells were stained with either DAPI or propidium iodide as a viability marker and the following antibodies for flow cytometry: CD45 APC-Cy7 (BD Biosciences, 557833), CD4 PE-Cy5 (Beckman Coulter Immunotech, IM2636U), CD8 BV421 (BD Horizon, RPA-T8), CD5 BV510 (BD Biosciences, UCHT2), CD3 PE-Cy7 (BD Pharmigen, UCHT1), CD7 PE (BD Pharmigen, 555361), CD56 FITC (BD Pharmigen, 362545).

### In Vitro B Cell Differentiation

MS5 stromal cells were seeded onto gelatin-coated 24-well plates at a density of 10,000/well. Cells were allowed to grow for 48 hours in Myelocult H5100 (Stem Cell Technologies, 05150) supplemented with 100 ng/mL SCF (R&D Systems), 50 ng/mL TPO (Peprotech), 10 ng/mL FLT3L (R&D Systems), and 25 ng/mL IL-7 (R&D Systems) in order to condition the media prior to seeding hematopoietic cells. Following 36-48 hours post-nucleofection of Cas9 RNPs, 2,500 CD34+ cord blood HSPCs were seeded onto MS5 stroma with at least n=3 replicate differentiations per gRNA per time-point. Fresh medium with cytokines was supplied weekly over five weeks of differentiation. Wells were harvested at regular intervals throughout the differentiation by gentle trituration to obtain total cell counts and for flow cytometric assessment of hematopoietic differentiation. Cells were stained with propidium iodide and either a hematopoietic lineage antibody panel, comprising CD45 APC-Cy7 (BD Biosciences, 557833), CD19 PE (BioLegend), CD56 FITC (BioLegend, 362545), CD33 APC (BioLegend), CD14 PerCP (BD Biosciences), or a B-cell specific antibody panel, comprising CD45 APC-Cy7 (BD Biosciences, 557833), CD19 PE (BioLegend), CD20 PE-Cy5 (BD Biosciences), IgM BV510 (BD Biosciences, 563113).

### In Vitro NK Cell Differentiation

CRISPR/Cas9 RNPs were delivered to CD34+ umbilical cord blood cells, as described above. NK differentiation was initiated 48 hours post-RNP nucleofection using a StemSpan NK cell Generation Kit (Stem Cell Technologies, 09960), per the manufacturer's instructions with n=3 replicate differentiations per gRNA in 24-well plates. Cells were replated into new 24-well plates after 14 days of differentiation and harvested for flow cytometry profiling, functional assays, and molecular assays after 28 days of differentiation.

### Flow Cytometry

Cells were stained with antibodies and a viability dye (DAPI or propidium iodide) at room temperature for 20 minutes in the dark for flow cytometric profiling, followed by a wash step with FACS buffer (PBS+2% FBS). Data was collected on a Sony MA900 and analyzed using FlowJo. Antibodies for flow cytometry were used at 1:100 dilution, unless otherwise stated.

### UNC0642 Treatment During T Cell Differentiation

CD34+ umbilical cord blood cells (AllCells) were thawed from liquid nitrogen storage and T cell differentiation was immediately initiated with a StemSpan T cell Generation Kit (Stem Cell Technologies, 09940), as described above. Cells were treated with UNC0642 (Cayman Chemical, 14604) over the entire duration of the differentiation and fresh compound was supplemented every 3-4 days to the media. A range of doses were tested (10 nM, 50 nM, 100 nM, and 500 nM) alongside a 0.5% DMSO vehicle control with n=3 replicate differentiations per condition. Cells were collected after 28 days of differentiation to assess NK and T cell populations by flow cytometry.

### Single Cell-Omics Assays on CRISPR-Perturbed Lymphoid Progenitors

Umbilical cord blood HSPCs were genome edited with Cas9 RNPs targeting EHMT1, TRIM28, and the AAVS1 locus, as described previously. The CRISPR-edited HSPCs were then differentiated to lymphoid progenitors over 14 days using the StemSpan T cell Generation Kit (Stem Cell Technologies cat. no. 09940) with n=3 replicate differentiations per gRNA. The cells were harvested, washed with FACS buffer (PBS+2% FBS), stained with DAPI for 10 minutes at room temperature, and sorted with a Sony MA900 FACS to isolate viable, single cells. FACS sorted cells were centrifuged at 300 g for 5 minutes at 4 C, resuspended in FACS buffer and the concentration was determined with a hemocytometer. scRNA-Seq and scATAC-Seq targeting 5,000 cells per gRNA was performed using the Chromium NextGEM Single Cell 3′ Reagent Kit v3.1 (10× Genomics) and Chromium NextGEM Single Cell ATAC Reagent Kit v1.1 (10× Genomics), respectively. scRNA-Seq libraries were barcoded with dual Illumina indices, whereas scATAC-Seq libraries were barcoded with single indices. The concentration of each final library was quantified using a High Sensitivity DNA Bioanalyzer Assay (Agilent). scRNA-Seq libraries were equimolar pooled and shallow sequenced on an Illumina MiniSeq to determine the number of high-confidence cell barcodes per library. The scRNA-Seq libraries were then renormalized and deep sequenced on a NextSeq 550 in a 28-10-10-44 read configuration. scATAC-Seq libraries were equimolar pooled and sequenced on a NextSeq 550 in a 34-8-16-34 read configuration.

### Bulk RNA-Sequencing

RNA from at least 5,000 cells was extracted using a 2×TCL lysis buffer (Qiagen cat. no. 1070498). At least two technical replicates were prepared per sample using the SMART-Seq2 protocol (Picelli et al. 2014), with some modifications. Briefly, RNA was purified from cellular lysate using 2.2×RNA SPRI beads (Beckman Coulter cat. no. A63987) then immediately reverse transcribed in the presence of a template switching oligo (Exiqon) with Maxima RNase H-minus RT (Thermo Fisher Scientific cat. no. EP0751) using a polyT primer containing the ISPCR sequence. Whole transcriptome amplification proceeded with KAPA HiFi HotStart ReadyMix using an ISPCR primer according to the following thermal program: 98 C for 3 minutes, 21 cycles of 98 C for 15 seconds, 67 C for 20 seconds, and 72 C for 6 minutes, and a final extension step of 72 C for 5 minutes. The amplified cDNA was cleaned with 0.8×DNA SPRI beads (Beckman Coulter cat. no. B23318). Ten nanograms of amplified cDNA was tagmented at 58 C for 10 minutes in a 10 uL reaction containing 2 μL of 5× tagmentation buffer (50 mM Tris-HCl, 25 mM MgCl2 pH 8.0), 2 μL of Tris Buffer (10 mM Tris-HCl, 1% Tween-20 pH 8), and 4 uL Nextera Tn5 transposase (Illumina). The reaction was stopped with 1% SDS and incubated at 72 C for 10 minutes, then 4 C for 3 minutes. The tagmented library was cleaned with 1×DNA SPRI beads followed by an indexing PCR with NEBNext High Fidelity polymerase to incorporate sample index barcodes and Illumina flow cell handles. The index PCR proceeded with the following thermal program: 72 C for 3 minutes, 98 C for 30 seconds, 12 cycles of 98 C for 10 seconds, 60 C for 30 seconds, 72 C for 30 seconds, and a final extension step of 72 C for 5 minutes. The final libraries were pooled, diluted and sequenced on a MiniSeq with a 75-cycle High Output Kit with the following read configuration: 36-8-8-38.

### Bulk ATAC-Sequencing

NK cells were FACS sorted following in vitro differentiation from CD34+ umbilical cord blood and at least 50,000 cells per sample were used as input for ATAC-Seq. Nuclei isolation, tagmentation and library construction were followed as described in the Omni-ATAC-Seq protocol (Corces et al. 2017). The concentration of the final ATAC-Seq libraries was quantified using a High Sensitivity DNA Bioanalyzer Assay (Agilent) in the size range of 100-1000 bp. The libraries were then equimolar pooled and shallow sequenced on a MiniSeq to determine the quality of the libraries. The ATAC-Seq libraries were processed with the PEPATAC pipeline (Smith et al. 2021). to determine the number of deduplicated, aligned reads to the hg38 genome and pertinent QC metrics, such as TSS enrichment and fragment length distributions. The libraries were then renormalized, pooled and sequenced on a NextSeq 500 with a 150-cycle High Output Kit v2 with the following read configuration: 76-8-8-75.

### NK Functional Assays

In vitro derived NK cells were collected on D4+28, washed with FACS buffer, and then cultured overnight in RP-10 medium (RPMI-1640 supplemented with 10% FBS, 1×penicillin/streptomycin, 2 mM L-glutamine, and 7.5 mmol HEPES) with 1 ng/mL recombinant human IL-15 (Miltenyi). K562 cells were cultured in RP-10 medium and labeled with 5 μM of CellTrace Violet (Thermo Fisher Scientific) in PBS for 20 minutes at 37° C. K562 were washed twice with RP-10 and co-cultured at various effector:target ratios. To measure NK cell cytotoxicity, NK cells and target cells were co-cultured for 4 hours, then stained with 2 μL of PE-Annexin V (Biolegend) and 2 μL of 7-AAD (BD Biosciences) in 50 μL Annexin V binding buffer (Biolegend) for 15 minutes at room temperature. To measure intracellular IFNγ and degranulation, NK cells were stimulated with recombinant human IL-12 (R&D), recombinant human IL-18 (R&D), or were co-cultured target cells for 1 hour, followed by the addition of 0.2 μL BD GolgiPlug (BD Biosciences), 0.13 μL BD GolgiStop (BD Biosciences) and 1 μL of APC-CD107a (Biolegend). After an additional 5 hours of co-culture, cells were stained for intracellular IFNγ using BD Cytofix/Cytoperm (BD Biosciences). Cells were acquired using BD LSRFortessa and analyzed using FlowJo.

### Analytical Methods

### Comparison of ABC Model Modifications to Genetic Perturbation Data

Various modifications to the ABC model were investigated and evaluated the enhancer-gene predictions against the results of genetic perturbation experiments on the candidate enhancer elements. First, genetic perturbation data was utilized in K562s and defined true-positive enhancer-gene pairs as previously described. Next, K562 epigenomic data was utilized and a 10-cell type averaged HiC dataset to generate enhancer-gene predictions using the ABC model on tissue-specific, non-ubiquitously expressed genes. Different definitions of enhancer “Activity” were evaluated in the model by only using chromatin accessibility data, as well as approximating HiC data with a power-law estimate. Each experimentally tested enhancer-gene pair was intersected with the predictions from the modified ABC models and calculated precision-recall statistics using the precrec package in R. Furthermore, the predictive performance of a chromatin accessibility-only ABC model was evaluated against an expanded compendium of genetic perturbations in other hematopoietic cell types. ATAC-Seq data was utilized on GM12878, BJAB, Jurkat, and THP1 cell lines+/−stimulation to generate ABC maps and calculated precision-recall statistics using genetic perturbation data.

### Curation of Public Chromatin Accessibility Data for ABC Model Predictions

Published chromatin accessibility data was curated on primary, FACS sorted cells to capture all major cell types and states within the human hematopoietic system. To be consistent across different studies, ATAC-Seq was utilized to measure chromatin accessibility. Raw fastq files were downloaded for HSPC populations from (Corces et al. 2016), erythroid subpopulations corresponding to temporal stages of differentiation from (Ludwig et al. 2019), myeloid and plasmacytoid dendritic cells from (Ulirsch et al. 2019), precursor dendritic cells and megakaryocytes from (Buenrostro et al. 2018), macrophages from ENCODE, and immune cells +/−stimulation from (Calderon et al. 2019). Each hematopoietic cell type, designated by a FACS immunophenotype was represented by at least 3 different human donors within our final curated dataset. In the case of the Calderon, et al. dataset, ATAC-Seq samples were excluded with a reported TSS score <6 (9/175 samples).

### Uniform Processing of Chromatin Accessibility Data

The PEPATAC pipeline was implemented to uniformly process and align raw ATAC-Seq data to the hg38 genome. All fastq files were first trimmed to remove Illumina adapter sequences using Skewer with command line options “-f sanger -t 20 -m pe -x”. Pre-alignments with Bowtie2 were performed to remove reads mapping to the mitochondrial genome, alpha satellite repeats, Alu repeats, and ribosomal DNA repeats using “-k 1 -D 20 -R 3 -N 1 -L 20 -i S,1,0.50 -X 2000-rg-id” options. Bowtie2 was used to align the remaining reads to the hg38 genome using “—very-sensitive X 2000—rg-id” options. Uniquely mapped reads was isolated with samtools and options “-f 2 -q 10 -b -@20”. Samblaster was used to mark duplicate reads, resulting in the final aligned, deduplicated BAM file that was used in all downstream analyses. For DNase-Seq data, hg38-aligned BAM files were downloaded from the ENCODE data portal.

### Generation of Enhancer-Gene Predictions Using the ABC Model

Enhancer-gene maps were generated for each hematopoietic cell type in our dataset using the ABC model https://github.com/broadinstitute/ABC-Enhancer-Gene-Prediction as previously described by Nasser, et al. 2021 with some modifications. First, peaks with MACS2 were called using the ATAC-Seq sample with the highest TSS score amongst all donor samples of a hematopoietic cell type. To enable comparisons between ATAC-Seq and DNase-Seq data, nucleosome-free ATAC-Seq reads were used (outputted from the PEPATAC pipeline) for defining candidate enhancers and quantifying enhancer activity. Candidate enhancer elements overlapping the ENCODE hg38 blacklist (https://www.encodeproject.org/files/ENCFF356LFX) were excluded. Gene annotations were downloaded from the UCSC genome browser http://hgdownload.soe.ucsc.edu/goldenPath/hg38/database/refGene.txt.gz to define TSSs and gene bounds for the model. Next, chromatin accessibility data was only used to quantify enhancer “Activity” (supported by our previous analysis) and samples from different donors of the same cell type were included as replicate experiments in the model. Chromatin accessibility in each candidate enhancer element was quantile-normalized to the distribution observed in K562s. A power-law estimate was used as the input for the “Contact” term in the model. ABC scores were then computed for each candidate enhancer-gene pair within a 5 Mb window of a gene's TSS and we retained enhancer-gene predictions with an ABC score >0.02. Enhancer-gene predictions were used for non-ubiquitously expressed genes, as defined in (Fulco et al. 2019).

Overlap of ABC Enhancers with chromHMM Regulatory States

The predictions were verified from the modified ABC model by intersecting the predicted ABC enhancers from GM12878, BJAB, Jurkat, and THP1 cell lines+/−stimulation with matched ChIP-Seq defined chromHMM regulatory states from the Roadmap Epigenomics Project. ChromHMM 15-state models were downloaded and determined the number of ABC enhancers that overlapped each chromHMM state for each cell type. The expected background was calculated as the coverage of a chromHMM state in the human genome and determined the statistical significance of ABC enhancers overlapping a state using a binomial test in R.

### Construction of a Pan-Hematopoiesis ABC Enhancer Peakset and Chromatin Accessibility Counts Matrix

To facilitate global analyses, the unique, non-redundant set of ABC enhancers was determined across all ABC predictions in the dataset and constructed a chromatin accessibility counts matrix over these regions for all samples. To do this, the ABC enhancer regions were shrunk by 150 bps on either side using bedtools slop and then determined the unique set of ABC enhancers per hematopoietic cell type. All unique ABC enhancers were concatenated from each cell type into one bed file and used bedtools merge to create a non-overlapping list of ABC enhancer regions. This list represents the unique set of ABC enhancers across all cell types in our dataset, which is termed a pan-hematopoiesis ABC enhancer peakset. Next, hg38-aligned, deduplicated BAM files and bedtools multicov were used to count chromatin accessibility reads within this peakset for all samples in our dataset. An ABC enhancers x samples counts matrix within R were constructed.

### Clustering and Visualization of Pan-Hematopoiesis ABC Enhancers

Prior to clustering, CPM normalized the ABC enhancer counts matrix using the edgeR package: cpm(matrix, log=TRUE, prior.count=5). To correct for technical covariates, differences in data quality were regressed out by using the TSS score as a covariate with the removeBatchEffect( ) function in the limma package. Finally, quantile normalization were performed using normalize.quantiles( ) from the preprocessCore package. The top 120,000 ABC enhancer by row variance using rowVars( ) in the matrixStats package for clustering were used. PCA was then performed using prcomp_irlba( ) on the first 50 PCs without scaling. The Euclidean distance was calculated with dist( ) across samples within the first 9 PCs (since the first 9 PCs explained approximately 80% of the variance). Density clustering was performed using densityClust( ) to get a decision plot (Figure S2D). Cluster assignments were extracted for rho=0.8 and delta=0.08 with findClusters( ). The cluster assignments were visualized using t-SNE: Rtsne(perplexity=40, theta=0, max_iter=1000, pca=TRUE). Finally, a confusion matrix was created to understand which samples reside in which clusters and visualized the matrix with the pheatmap package in R.

Transcription Factor Footprinting within ABC Enhancers

The ChrAccR R package (v.0.9.17) was used to perform transcription factor footprinting analysis using ATAC-Seq data. DsATAC object was created using hg38-aligned, deduplicated BAM files from the PEPATAC pipeline and ABC enhancers as the input peakset. Biological replicates for each cell type (e.g. different donor samples) were merged with mergeSamples(dsa, mergeGroups=“bio_group”, countAggrFun=“sum”). Footprinting analysis was performed specifically within ABC enhancer peaks using getMotifFootprints( ) and PWMs from the JASPAR motif database.

### Visualization of ABC Enhancer-Gene Predictions

ATAC-Seq sequencing tracks was visualized with pyGenomeTracks (https://github.com/deeptools/pyGenomeTracks). Bigwig files were utilized, outputted from the PEPATAC pipeline, containing Tn5 offset-corrected insertion sites. Reads-in-peaks normalization were performed as described in (Corces et al. 2018) when comparing multiple samples. ABC enhancer-gene links were visualized as arcs weighted by the ABC score and centered on the predicted gene's TSS and the midpoint of the ABC enhancer.

### Processing of the RepeatMasker Transposable Element Reference Database

RepeatMasker TE annotations were downloaded within the hg38 human reference genome from the UCSC Genome Browser (http://hgdownload.soe.ucsc.edu/goldenPath/hg38/database/rmsk.txt.gz). TEs belonging to DNA, LINE, LTR, RC, Retroposon, and SINE classes were considered and residing within standard chromosomes. Furthermore, ambiguous TE annotations were filtered out and all tRNA related families. A GRanges object was constructed in R of genomic intervals for each TE family and used it for all downstream analyses.

### Enrichment of Transposable Elements in ABC Enhancers

The GIGGLE framework was utilized to generate indices of genomic intervals for TE families. The non-redundant set of ABC enhancers for each cell type were queried against the TE family database using the GIGGLE search function with a genome size of 3099922541 bp. Significantly enriched TE families were defined as those TE families with at least 20 overlaps with ABC enhancer regions, an odds ratio >2.5 and a −log 10(p-value)<0.01 in at least one cell type.

Transcription Factor Occupancy Analysis within ABC Enhancers

TF occupancy was predicted within TE-containing ABC enhancers using the TOBIAS transcription factor occupancy framework (https://github.com/loosolab/TOBIAS), as described in (Bentsen et al. 2020). In brief, ATAC-Seq BAM files were merged from various donors belonging to the same cell type and performed bias correction of the ATAC-Seq signal with ATACorrect across all peaks in the pan-hematopoiesis ABC enhancer peakset. Footprint scores on the bias-corrected ATAC-Seq signal with FootprintScores and estimated transcription factor occupancy using BINDetect and the position frequency matrix of the TF motif. In the case of predicting AP-1 occupancy, differential binding was identified with BINDetect between resting and activated T cell states within TE-containing ABC enhancers.

TE Quantification from RNA-Seq and Differential Expression Analysis

RNA-Seq data on FACS-sorted hematopoietic populations from (Corces et al. 2016) was downloaded and reanalyzed. First, fastq files were mapped to the hg38 reference transcriptome with kallisto. The resulting BAM files were used with TEtranscripts (Jin et al. 2015) (https://github.com/mhammell-laboratory/TEtranscripts) to quantify the expression of TEs. Curated GTF files on TE annotations were downloaded from the Hammell Lab website for TEtranscript quantification. Differential expression was subsequently performed with DESeq2 1.10.1 (Love, Huber, and Anders 2014).

Calculation of Gene Module Scores for Regulators of Heterochromatin from Single Cell RNA-Seq Data

scRNA-Seq data was reanalyzed on healthy human hematopoiesis previously reported in (Granja et al. 2019). The processed scRNA-Seq data was processed from the publication's GitHub repository (https://github.com/GreenleafLab/MPAL-Single-Cell-2019) and imported it as a Seurat object in R. Gene sets were scored to generate a new aggregate expression level corrected for the background expression of each cell, as described previously (Tirosh et al. 2016). Gene sets were downloaded from the Gene Ontology database and scored using the AddModuleScore function in Seurat. Gene module scores were visualized across the transcriptional clusters originally annotated by (Granja et al. 2019).

Processing scRNA-Seq Data on In Vitro Derived Lymphoid Progenitors

scRNA-Seq libraries were demultiplexed and fastqs were generated with cellranger mkfastq (10× Genomics, version 7.0.1). Reads were aligned to the hg38 reference genome and quantified using cellranger count (10× Genomics, version 7.0.1). Downstream analysis was performed using Seurat (version 3.2.3) within R. Count matrices from the cellranger output were preprocessed by filtering for cells and genes (percent mitochondrial reads <20%, at least 1000 genes detected/cell, and less than 20,000 UMIs/cell). Normalization and variance stabilization of the count data was performed using sctransform and regressing out the proportion of mitochondrial reads and cell cycle phase. PCA was performed and used the first 20 PCs to run UMAP analysis. Transcriptional clusters were identified with FindClusters( ) at a resolution of 0.2. Differentially expressed genes were determined for each cluster by a Wilcoxon rank-sum test with an FDR cutoff of 0.01 and log 2(fold-change) of at least 0.25.

Processing scATAC-Seq Data on In Vitro Derived Lymphoid Progenitors

Sequencing libraries were demultiplexed and fastqs were generated with cellranger atac mkfastq (10× Genomics, version 1.2.0). Reads were aligned to the hg38 reference genome and quantified using cellranger count (10× Genomics, version 1.2.0). All downstream analysis was performed with ArchR (version 1.0.2) in R. Quality control filtering was performed and excluded cells that had a TSS enrichment less than 5 and fewer than 1000 aligned fragments. Putative doublets were excluded with filterDoublets( ). Dimensionality was reduced with two rounds of latent semantic indexing (LSI) with a cluster resolution of 0.2. LSI dimensions were clustered on the iterative using Seurat's SNN graph clustering function at a resolution of 0.3. A UMAP was subsequently added with addUMAP( ) and the default parameters. To help annotate scATAC-Seq clusters, marker genes were identified with Gene Scores using getMarkerFeatures( ) and accounting for biases in TSS enrichment and the number of aligned fragments. Pseudobulk replicates were created per gRNA condition per cluster followed by peak calling with MACS2. A union peakset was created using an iterative overlap removal method implemented in addReproduciblePeakSet( ). TF activity was inferred with chromVAR using the clustered TF motif archetype collection from (Virestra et al. 2020).

### Processing and Analysis of Bulk ATAC-Seq on In Vitro Derived NK Cells

ATAC-Seq libraries were demultiplexed with bcl2fastq and processed with the PEPATAC pipeline, as described above. A consensus peakset across all samples was generated using the PEPATACr package in R and a count matrix was constructed using bedtools multicov to count ATAC-Seq reads from hg38-aligned, deduplicated BAM files within the consensus peakset. Using the ATAC-Seq count matrix as an input, transcription factor activity was inferred with chromVAR and CIS-BP motif matches within these peaks from motifmatchr. GC bias-corrected deviations were computed using the chromVAR deviations function. A comparable analysis was performed using TE families rather than motifs to determine derepressed TEs within knockout conditions compared the AAVS1 gRNA control. Statistical significance of TE accessibility deviations was computed with the differentialDeviations function from chromVAR and FDR-corrected p-values <0.01 were considered significant. Differentially accessible peaks were determined with DESeq2 using the raw ATAC-Seq count matrix and statistically significant peaks were identified at an independent hypothesis weighting (IHW) value of 0.01. Differentially accessible peaks were then clustered with k-means clustering and used as the input to GREAT for annotation of biological processes.

### Processing and Analysis of Bulk RNA-Seq on In Vitro Derived NK Cells

RNA-Seq paired end reads were pseudo-aligned with kallisto (Bray et al. 2016) to the hg19 reference transcriptome. Transcript-level abundance estimates were imported into R with the tximport package and constructed into a gene-summarized count and abundance matrices for all samples. Differential gene expression analysis was performed using DESeq2 (Love, Huber, and Anders 2014) on the estimated count matrix. Statistically significant genes varying between a knockout population and the AAVS1 gRNA control population were identified at an FDR<0.05.

### Sequences:

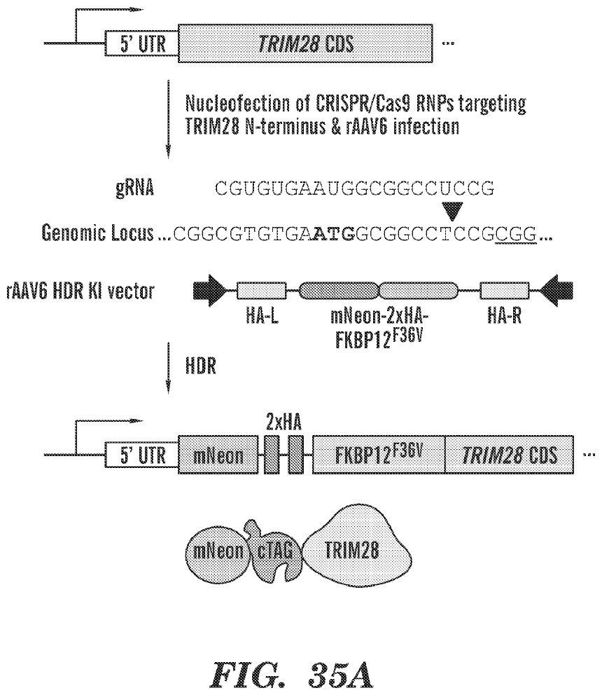

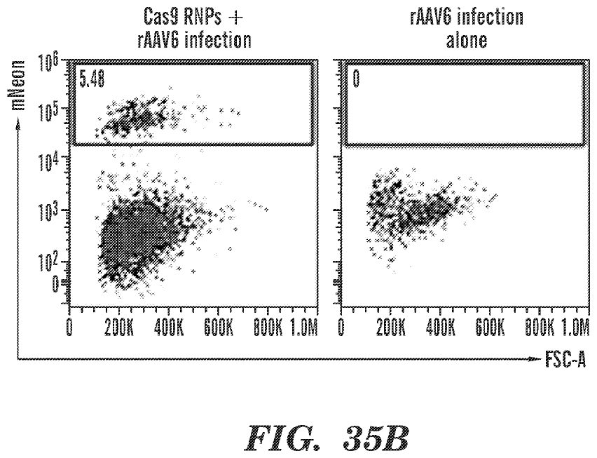

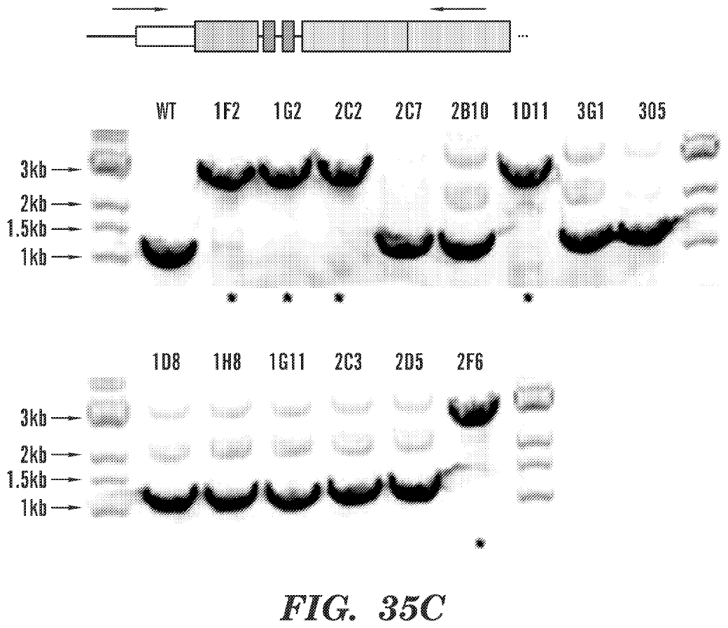

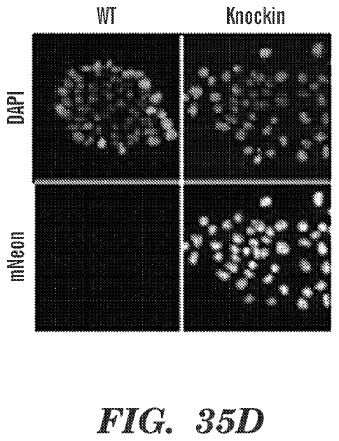

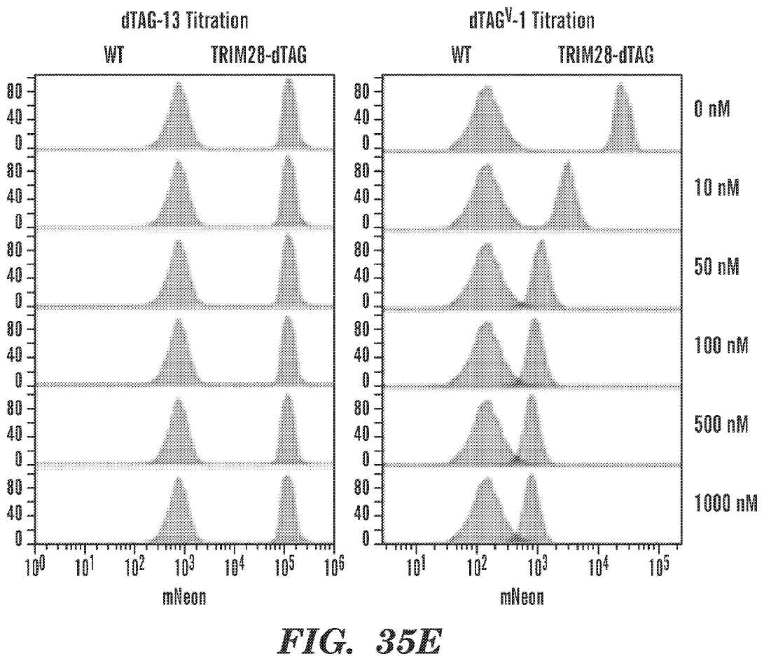

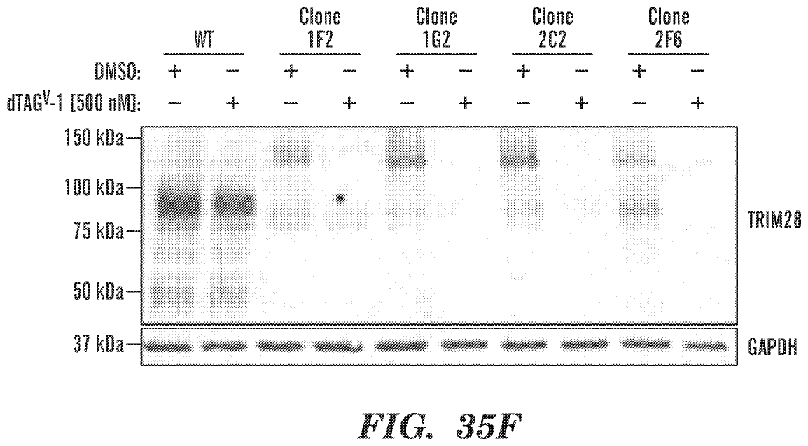

SEQ ID NO. 1: human TRIM28 genomic DNA, NG_046945.1
GTGACGCAGAGGCTGGAGACGACTCTACGGCGGCGAAGAGACGC
GGGTTGAGGAAGAGGGACGGATTGCCCATGCGCTTGGGCGCACAGCGGCCCGCT
TCTGTGTGGTCTGGAGGTGGAGCTGAGAGGGGAATCACACTCTATAAAGGTTCG
CATACCCCACTGGCGGATTCAATTGCGGCAGTGACGTCACAGAGGCCCCGCCCC
GCCCCCACAAGAGCCCCACCGACGTGGGGTTGGCGGTGGTGGAAGGACTAGGAG
TTGGCGCGTGCGTACTGGCGGCCTCTCCCGCACCGACCGGCCTGGGCCCCGCCCC
CGGGCGTGAGGCGCCCAATGCGCGTGCGCGGCGGCGTCGGCGCCAGTTATTTCT
GTCCCGCCCCCCGGCCTCGGCTCTTTCTGCGAGCGGGCGCGCGGGCGAGCGGTTG
TGCTTGTGCTTGTGGCGCGTGGTGCGGGTTTCGGCGGCGGCTGAGGAAGAAGCG
CGGGCGGCGCCTTCGGGAGGCGAGCAGGCAGCAGTTGGCCGTGCCGTAGCAGCG
TCCCGCGCGCGGCGGGCAGCGGCCCAGGAGGCGCGTGGCGGCGCTCGGCCTCGC
GGCGGCGGCGGCGGCAGCGGCCCAGCAGTTGGCGGCGAGCGCGTCTGCGCCTGC
GCGGCGGGCCCCGCGCCCCTCCTCCCCCCCTGGGCGCCCCCGGCGGCGTGTGAAT
GGCGGCCTCCGCGGCGGCAGCCTCGGCAGCAGCGGCCTCGGCCGCCTCTGGCAG
CCCGGGCCCGGGCGAGGGCTCCGCTGGCGGCGAAAAGCGCTCCACCGCCCCTTC
GGCCGCAGCCTCGGCCTCTGCCTCAGCCGCGGCGTCGTCGCCCGCGGGGGGCGG
CGCCGAGGCGCTGGAGCTGCTGGAGCACTGCGGCGTGTGCAGAGAGCGCCTGCG
ACCCGAGAGGGAGCCCCGCCTGCTGCCCTGTTTGCACTCGGCCTGTAGTGCCTGC
TTAGGGCCCGCGGCCCCCGCCGCCGCCAACAGCTCGGGGGACGGCGGGGCGGCG
GGCGACGGCACCGGTAAGTACGAAGTGATCGGTGCCACCCCTCCCCCTACTCTCT
GCCTTTGATTCCGACTGGGTGCAGAGATGAGGATGCCACCTGGGCGAGAGGATG
GGGGCCCGGACAGGGCACGGGAAATACTTTCTGGGTCCTGCATACGAACGTGGG
TTTGTGCTGGCCGCTGAGATGGGACATCTGACTAAAGTTGGAGAAAAGAAGGCT
CGGGGAGGGGAGGGGCTGGTTCGCTGCGGGATAATGGTCGGGGGCCCACCCAGC
AGGGGAATGGTGGGGGCCATAACCTGGGTGGGAACTTGTAACAGTCTCCCACAT
CCCTGCTTCTCGAAGTGGTGGACTGTCCCGTGTGCAAGCAACAGTGCTTCTCCAA
AGACATCGTGGAGAATTATTTCATGCGTGATAGTGGCAGCAAGGCTGCCACCGA
CGCCCAGGATGCGAACCAGGTGCGTCCTATCTCAGCAACCACAAGGAGGTTTCT
GGGGAGGGGGCATCTGCGCAGGAGGAGCTTGGCACCAGCTCCAGGCTGTTACTC
CACTTTCCCAAGGCTCTGGGTGGGCTGCCTAGGTTGGGTCAAGGGACCAATCTTA
AATCTCCGGTTGTATTTTCTGGGATGTAAACGTGGATCTATCAAGTTGTCTTGCCT
TCTCTGACCCTGCCTTTGTCTGGCAGTGCTGCACTAGCTGTGAGGATAATGCCCC
AGCCACCAGCTACTGTGTGGAGTGCTCGGAGCCTCTGTGTGAGACCTGTGTAGAG
GCGCACCAGCGGGTGAAGTACACCAAGGACCATACTGTGCGCTCTACTGGTACA
TGAGGCTGAGGGGGGCTGTTGGAGTTGTTCTCCCATGTGTGCCCTCAGTTGCTTTT
ATGATGTTGGTTGCATCTGGTGGATGGGTCCTAGAGTTCTCTAGGGGGTGCCGCC
CGAAGGGCCCGAGGGCAGAACTCCAGAAGCAGAAAAACTGGGGTTGTGGTGTGT
AGTCTCTAGGGCCTAGGTGGGAGAGGGTGGGAAGGGGAAATAAGGGAGACCTT
AATGTGCTGCAGGGAGGTACAAAGGGTTTGAGAAGGCTTATCAGGGAGTIGTCA
GACCTGGTGGTGAAGGGCTCAGCATATGCAAACAGGGAAAGGCATGGTGTGAAG
GGCTTTCTGGGTTTGTGGTTCCCTGGCACACATCTGGTATAAGATGCTGCTAGGG
AAAGTAGACTGTGGGTCCATGGATTATAAGTGATGATGATTTTTGGAAGTGTTTT
AGCTTTTGAGTTGGCAGAGACTTACCCAAAGTTTGGTTTGGCATTTCCAGCTTTTC
ACAGACACAGCTAGCTTTGGTTTGGAAGGAGCTTGAGAGTCTTTTGAGTCTAGCC
AAGTTTTGGGGCCAGAATCCTCCCATGAGTGGCCTTTTGGTAGCCATACTGTGAT
TCTTCCCATCCCAGAGGACAGCATTGGTGGAAGATAGCGGGAAGGTGGTGGCTG
TGGGGCCTGAGCAGCCAGGAGGGATACGCAGGGGCTGCAGGGTGTCCTACATTC
CAGACGTCCCAGGCAAAGGTCAGGGTGCAGGAATGAGTGGCATTGGGGAAGTCA
GTTTCCCCAATGAGCCCTGATGCTACATATTTTGGATTTGGTTGGTATGTGGGACC
AGTGTGTTTGATCAGTTTTCTAATGGGGAGACAGGCTTTGGAGGTTATTAATGGG
TTGGTCTTTGGGTGGGATTATGCTGAGCACCAAGATTCTGCTCTAGAGGGGGTGT
GACCACCATTCTGAGGGTCCTGGTTTGACATTTCATCTCTCCCAGTATGAGAACC
TCTACAAGGTTTCCTCTGGGCCCTTGGATTGTAAGAGACCCTGGTGTTCTCTTGTC
AGGGAAGAGGGAGGAAGTTAACCATGAAAGGAGAAGAAAAGGAATCCAGGGTG
GTGTGTAGGCAGAATTCTGAGTATGTCCACTTGGAGAAGTGTTCAGTAAAGGGA
ACAGTGTGGGGAAGGGTCAGCGGGGGTGGGGGGTGGGGTGGGGTGGTCCTCTTT
TTCTAGACGCTGTTATCCTTACCCTTACATTGTTGTTATCTCTAGAAGCTAGAAGA
AAGGGATGTGTTTCTCAGCTATGTTGGGGCAGAGGATTCTGGGAGCAAGGAGTT
GGGCTTGCAGGCCTGTGTTCTTCCCTGGCCACACCCTCTACATCTTCCCAATAAAT
GGCCCAGTGTCACAGAACAGGCCGGAAATGAGGCCCCAACTCATTCTGCTTCCT
GAGTTGGGGGTGGTGAAGGGCAAGGTCCAGCCTTATGATTCCCACTCCCCAGGG
CCAGCCAAGTCTCGGGATGGTGAACGTACTGTCTATTGCAACGTACACAAGCAT
GAACCCCTTGTGCTGTTTTGTGAGAGCTGTGATACTCTCACCTGCCGAGACTGCC
AGCTCAATGCCCACAAGGACCACCAGTGAGTCCCAAGGCATAGTGGTTGGGTGG
GTGGGTGCCACCCCTTCCGTAGCTTAGTGCTCAGGAACACATCTGTCTGCTCTCA
GGTACCAGTTCTTAGAGGATGCAGTGAGGAACCAGCGCAAGCTCCTGGCCTCAC
TGGTGAAGCGCCTTGGGGACAAACATGCAACATTGCAGAAGAGCACCAAGGAG
GTTCGCAGCTCGTAAGTGTGGGTTCTGGGGCTGTGGGGGTGGCCCAGGGCAGCA
AGACCCTACCTAGCCTGACCTGCTGTGTCCCCTAGAATCCGCCAGGTGTCTGACG
TACAGAAGCGTGTGCAAGTGGATGTCAAGATGGCCATCCTGCAGATCATGAAGG
AGCTGAATAAGCGGGGCCGTGTGCTGGTCAATGATGCCCAGGTAAGCCTTGTGC
CGGTGAGAAGGGTCCCTGAGCCCCCTCTGCTGATTGATGATGCTGTCTGGGGTGA
GGAGTGATCCTAGTATTCTTCTGCCTTCTCTGCTTACCTCATACACCTTCTATCTG
CAGAAGGTGACTGAGGGGCAGCAGGAGCGCCTGGAGCGGCAGCACTGGACCAT
GACCAAGATCCAGAAGCACCAGGAGCACATTCTGCGCTTTGCCTCTTGGGCTCTG
GAGAGTGACAACAACACAGCCCTTTTGCTTTCTAAGAAGTTGGTGTGTACTGGTG
GGCTCCTGGCTGGTGGGTTCCAGGCAGGTGGTTCCCAATACCTCAAATCCCTATT
TGTTGTTGGTGTGCTCATTCTTTCCTCCCTTTCACTCCCAACCAGATCTACTTCCA
GCTGCACCGGGCCCTCAAGATGATTGTGGATCCCGTGGAGCCACATGGCGAGAT
GAAGTTTCAGTGGGACCTCAATGCCTGGACCAAGAGTGCCGAGGCCTTTGGTGG
GTCCCCAGCTTTACCTCACTCTGTTATTACCCCACGTGCTGCACCTACTTACGTTT
CTCTCTTCTTTTTGCAGGCAAGATTGTGGCAGAGCGTCCTGGCACTAACTCAACA
GGCCCTGCACCCATGGCCCCTCCAAGAGCCCCAGGGCCCCTGAGCAAGCAGGGC
TCTGGCAGCAGCCAGGTGAGCAGGAGAGAGGACCCAGGAAGGGGTGGGCAGGG
AATGGGGTCCAGTAGCAGGAGAGAGGACCCAGGCAGGGGGGGTGGGCAGGGAA
TGGGGTCCAGTAGGGTTCCTGTCCCACTGAGGCAGAGGGTTCTGCTTTGTTCACA
GCCCATGGAGGTGCAGGAAGGCTATGGCTTTGGGTCAGGTGAGTGGGTCTGCCT
AGTGGGTGGGGAAGGGCCCCAGTGCTGTTTCTACCCCAACCTGCCAGTCTTCTTT
CTCCATTTTCTAAGGAGATGATCCCTACTCAAGTGCAGAGCCCCATGTGTCAGGT
GTGAAACGGTAAGTATGGCACCTCCCCTGGGGGTGAGGTGGATGGAGGGTGGGG
GTGTACTCATGCCAGGTTGCTGCATCTACAGGTCCCGCTCAGGTGAGGGCGAGGT
GAGCGGCCTTATGCGCAAGGTGCCACGAGTGAGCCTTGAACGCCTGGACCTGGA
CCTCACAGCTGACAGCCAGCCACCCGTCTTCAAGGTCTTCCCAGGCAGTACCACT
GAGGACTACAACCTTATTGTTATTGAACGTGGCGCTGCCGCTGCAGCTACCGGCC
AGCCAGGGACTGCGCCTGCAGGAACCCCTGGTGCCCCACCCCTGGCTGGCATGG
CCATTGTCAAGGTAAGCCTGTCCCAAGGAACTATAGCTGTAGGATGAAGCCTGT
AGTCCAGGTCTGGACCCTGTTGAACACCCCTCATCACCACCTTGCAGGAGGAGG
AGACGGAGGCTGCCATTGGAGCCCCTCCTACTGCCACTGAGGGCCCTGAGACCA
AACCTGTGCTTATGGCTCTTGCGGAGGGTCCTGGTGCTGAGGGTCCCCGCCTGGC
CTCACCTAGTGGCAGCACCAGCTCAGGGCTGGAGGTGGTGGCTCCTGAGGGTAC
CTCAGCCCCAGGTGGTGGCCCGGGAACCCTGGATGACAGTGCCACCATTTGCCGT
GTCTGCCAGAAGCCAGGCGATCTGGTTATGTGCAACCAGTGTGAGTTTTGTTTCC
ACCTGGACTGTCACCTGCCGGCCCTGCAGGATGTACCAGGGTGAGTGTGAGGCT
GGTGGGGGTCAAGTCTGGGTGTTGGGCTGTCTGGACAGGATCATGTGCAGACCC
TTATTTTCTTCACCCTAGGGAGGAGTGGAGCTGCTCACTCTGCCATGTGCTCCCTG
ACCTGAAGGAGGAGGATGGCAGCCTCAGCCTGGATGGTGCAGACAGCACTGGCG
TGGTGGCCAAGCTCTCACCAGCCAACCAGCGGGTGAGGGCTGGGGTTACTTAGG
TGGGGTTGCCCAGAGAGGCTTTATAGGTGCTGCCCAGAGCTGTGACATCCCTTAC
AATGTTTGTAGAAATGTGAGCGTGTACTGCTGGCCCTATTCTGTCACGAACCCTG
CCGCCCCCTGCATCAGCTGGCTACCGACTCCACCTTCTCCCTGGTGAGTCCTAGG
ATGGGAAAGGGGAAGGGGGTGGTGGCTGCTGGGTCTCGCCCTCAACCTGTGCAT
GTATATGTGTGTCTTTGTGTGTGTATGTGTGATCTCTGCCTGCAGGACCAGCCCGG
TGGCACCCTGGATCTGACCCTGATCCGTGCCCGCCTCCAGGAGAAGTTGTCACCT
CCCTACAGCTCCCCACAGGAGTTTGCCCAGGATGTGGGCCGCATGTTCAAGCAAT
TCAACAAGTTAACTGAGGTGAGCCAGTGGAATGGAGAGGCTGTGGGCAGGGGG
AGATGTGAAGGAAAGAACTAGGACCCATTCATCCACTGCATTCCTGCTTGGCCCA
GGACAAGGCAGACGTGCAGTCCATCATCGGCCTGCAGCGCTTCTTCGAGACGCG
CATGAACGAGGCCTTCGGTGACACCAAGTTCTCTGCTGTGCTGGTGGAGCCCCCG
CCGATGAGCCTGCCTGGTGCTGGCCTGAGTTCCCAGGAGCTGTCTGGTGGCCCTG
GTGATGGCCCCTGAGGCTGGAGCCCCCATGGCCAGCCCAGCCTGGCTCTGTTCTC
TGTCCTGTCACCCCATCCCCACTCCCCTGGTGGCCTGACTCCCACTCCCTGGTGGC
CCCATCCCCCAGTTCCTCACGATATGGTTTTTACTTCTGTGGATTTAATAAAAACT
TCACCAGTT
SEQ ID NO. 2: human TRIM28 mRNA NM_005762.3
AGTGACGCAGAGGCTGGAGACGACTCTACGGCGGCGAAGAGACGC
GGGTTGAGGAAGAGGGACGGATTGCCCATGCGCTTGGGCGCACAGCGGCCCGCT
TCTGTGTGGTCTGGAGGTGGAGCTGAGAGGGGAATCACACTCTATAAAGGTTCG
CATACCCCACTGGCGGATTCAATTGCGGCAGTGACGTCACAGAGGCCCCGCCCC
GCCCCCACAAGAGCCCCACCGACGTGGGGTTGGCGGTGGTGGAAGGACTAGGAG
TTGGCGCGTGCGTACTGGCGGCCTCTCCCGCACCGACCGGCCTGGGCCCCGCCCC
CGGGCGTGAGGCGCCCAATGCGCGTGCGCGGCGGCGTCGGCGCCAGTTATTTCT
GTCCCGCCCCCCGGCCTCGGCTCTTTCTGCGAGCGGGCGCGCGGGCGAGCGGTTG
TGCTTGTGCTTGTGGCGCGTGGTGCGGGTTTCGGCGGCGGCTGAGGAAGAAGCG
CGGGCGGCGCCTTCGGGAGGCGAGCAGGCAGCAGTTGGCCGTGCCGTAGCAGCG
TCCCGCGCGCGGCGGGCAGCGGCCCAGGAGGCGCGTGGCGGCGCTCGGCCTCGC
GGCGGCGGCGGCGGCAGCGGCCCAGCAGTTGGCGGCGAGCGCGTCTGCGCCTGC
GCGGCGGGCCCCGCGCCCCTCCTCCCCCCCTGGGCGCCCCCGGCGGCGTGTGAAT
GGCGGCCTCCGCGGCGGCAGCCTCGGCAGCAGCGGCCTCGGCCGCCTCTGGCAG
CCCGGGCCCGGGCGAGGGCTCCGCTGGCGGCGAAAAGCGCTCCACCGCCCCTTC
GGCCGCAGCCTCGGCCTCTGCCTCAGCCGCGGCGTCGTCGCCCGCGGGGGGCGG
CGCCGAGGCGCTGGAGCTGCTGGAGCACTGCGGCGTGTGCAGAGAGCGCCTGCG
ACCCGAGAGGGAGCCCCGCCTGCTGCCCTGTTTGCACTCGGCCTGTAGTGCCTGC
TTAGGGCCCGCGGCCCCCGCCGCCGCCAACAGCTCGGGGGACGGCGGGGCGGCG
GGCGACGGCACCGTGGTGGACTGTCCCGTGTGCAAGCAACAGTGCTTCTCCAAA
GACATCGTGGAGAATTATTTCATGCGTGATAGTGGCAGCAAGGCTGCCACCGAC
GCCCAGGATGCGAACCAGTGCTGCACTAGCTGTGAGGATAATGCCCCAGCCACC
AGCTACTGTGTGGAGTGCTCGGAGCCTCTGTGTGAGACCTGTGTAGAGGCGCACC
AGCGGGTGAAGTACACCAAGGACCATACTGTGCGCTCTACTGGGCCAGCCAAGT
CTCGGGATGGTGAACGTACTGTCTATTGCAACGTACACAAGCATGAACCCCTTGT
GCTGTTTTGTGAGAGCTGTGATACTCTCACCTGCCGAGACTGCCAGCTCAATGCC
CACAAGGACCACCAGTACCAGTTCTTAGAGGATGCAGTGAGGAACCAGCGCAAG
CTCCTGGCCTCACTGGTGAAGCGCCTTGGGGACAAACATGCAACATTGCAGAAG
AGCACCAAGGAGGTTCGCAGCTCAATCCGCCAGGTGTCTGACGTACAGAAGCGT
GTGCAAGTGGATGTCAAGATGGCCATCCTGCAGATCATGAAGGAGCTGAATAAG
CGGGGCCGTGTGCTGGTCAATGATGCCCAGAAGGTGACTGAGGGGCAGCAGGAG
CGCCTGGAGCGGCAGCACTGGACCATGACCAAGATCCAGAAGCACCAGGAGCAC
ATTCTGCGCTTTGCCTCTTGGGCTCTGGAGAGTGACAACAACACAGCCCTTTTGC
TTTCTAAGAAGTTGATCTACTTCCAGCTGCACCGGGCCCTCAAGATGATTGTGGA
TCCCGTGGAGCCACATGGCGAGATGAAGTTTCAGTGGGACCTCAATGCCTGGAC
CAAGAGTGCCGAGGCCTTTGGCAAGATTGTGGCAGAGCGTCCTGGCACTAACTC
AACAGGCCCTGCACCCATGGCCCCTCCAAGAGCCCCAGGGCCCCTGAGCAAGCA
GGGCTCTGGCAGCAGCCAGCCCATGGAGGTGCAGGAAGGCTATGGCTTTGGGTC
AGGAGATGATCCCTACTCAAGTGCAGAGCCCCATGTGTCAGGTGTGAAACGGTC
CCGCTCAGGTGAGGGCGAGGTGAGCGGCCTTATGCGCAAGGTGCCACGAGTGAG
CCTTGAACGCCTGGACCTGGACCTCACAGCTGACAGCCAGCCACCCGTCTTCAAG
GTCTTCCCAGGCAGTACCACTGAGGACTACAACCTTATTGTTATTGAACGTGGCG
CTGCCGCTGCAGCTACCGGCCAGCCAGGGACTGCGCCTGCAGGAACCCCTGGTG
CCCCACCCCTGGCTGGCATGGCCATTGTCAAGGAGGAGGAGACGGAGGCTGCCA
TTGGAGCCCCTCCTACTGCCACTGAGGGCCCTGAGACCAAACCTGTGCTTATGGC
TCTTGCGGAGGGTCCTGGTGCTGAGGGTCCCCGCCTGGCCTCACCTAGTGGCAGC
ACCAGCTCAGGGCTGGAGGTGGTGGCTCCTGAGGGTACCTCAGCCCCAGGTGGT
GGCCCGGGAACCCTGGATGACAGTGCCACCATTTGCCGTGTCTGCCAGAAGCCA
GGCGATCTGGTTATGTGCAACCAGTGTGAGTTTTGTTTCCACCTGGACTGTCACC
TGCCGGCCCTGCAGGATGTACCAGGGGAGGAGTGGAGCTGCTCACTCTGCCATG
TGCTCCCTGACCTGAAGGAGGAGGATGGCAGCCTCAGCCTGGATGGTGCAGACA
GCACTGGCGTGGTGGCCAAGCTCTCACCAGCCAACCAGCGGAAATGTGAGCGTG
TACTGCTGGCCCTATTCTGTCACGAACCCTGCCGCCCCCTGCATCAGCTGGCTAC
CGACTCCACCTTCTCCCTGGACCAGCCCGGTGGCACCCTGGATCTGACCCTGATC
CGTGCCCGCCTCCAGGAGAAGTTGTCACCTCCCTACAGCTCCCCACAGGAGTTTG
CCCAGGATGTGGGCCGCATGTTCAAGCAATTCAACAAGTTAACTGAGGACAAGG
CAGACGTGCAGTCCATCATCGGCCTGCAGCGCTTCTTCGAGACGCGCATGAACG
AGGCCTTCGGTGACACCAAGTTCTCTGCTGTGCTGGTGGAGCCCCCGCCGATGAG
CCTGCCTGGTGCTGGCCTGAGTTCCCAGGAGCTGTCTGGTGGCCCTGGTGATGGC
CCCTGAGGCTGGAGCCCCCATGGCCAGCCCAGCCTGGCTCTGTTCTCTGTCCTGT
CACCCCATCCCCACTCCCCTGGTGGCCTGACTCCCACTCCCTGGTGGCCCCATCC
CCCAGTTCCTCACGATATGGTTTTTACTTCTGTGGATTTAATAAAAACTTCACCAG
TT
SEQ ID NO. 3: human TRIM28 protein NP_005753.1
MAASAAAASAAAASAASGSPGPGEGSAGGEKRSTAPSAAASASASAA
ASSPAGGGAEALELLEHCGVCRERLRPEREPRLLPCLHSACSACLGPAAPAAANSSG
DGGAAGDGTVVDCPVCKQQCFSKDIVENYFMRDSGSKAATDAQDANQCCTSCEDN
APATSYCVECSEPLCETCVEAHQRVKYTKDHTVRSTGPAKSRDGERTVYCNVHKHE
PLVLFCESCDTLTCRDCQLNAHKDHQYQFLEDAVRNQRKLLASLVKRLGDKHATLQ
KSTKEVRSSIRQVSDVQKRVQVDVKMAILQIMKELNKRGRVLVNDAQKVTEGQQE
RLERQHWTMTKIQKHQEHILRFASWALESDNNTALLLSKKLIYFQLHRALKMIVDPV
EPHGEMKFQWDLNAWTKSAEAFGKIVAERPGTNSTGPAPMAPPRAPGPLSKOGSGS
SQPMEVQEGYGFGSGDDPYSSAEPHVSGVKRSRSGEGEVSGLMRKVPRVSLERLDL
DLTADSQPPVFKVFPGSTTEDYNLIVIERGAAAAATGQPGTAPAGTPGAPPLAGMAI
VKEEETEAAIGAPPTATEGPETKPVLMALAEGPGAEGPRLASPSGSTSSGLEVVAPEG
TSAPGGGPGTLDDSATICRVCQKPGDLVMCNQCEFCFHLDCHLPALQDVPGEEWSC
SLCHVLPDLKEEDGSLSLDGADSTGVVAKLSPANQRKCERVLLALFCHEPCRPLHQL
ATDSTFSLDQPGGTLDLTLIRARLQEKLSPPYSSPQEFAQDVGRMFKQFNKLTEDKA
DVQSIIGLQRFFETRMNEAFGDTKFSAVLVEPPPMSLPGAGLSSQELSGGPGDGP
SEQ ID NO: 4: AAVS gRNA: GGACGCACCATTCTCACAAA
SEQ ID NO: 5: EHMT1 gRNA01: GGGCCGGTGCACAAACAGCG
SEQ ID NO: 6: EHMT1 gRNA02: TTCGGCTGCTTCCATCAACG
SEQ ID NO: 7: SUV39H2 gRNA01: TTTCGAACTAGCAATGGACG
SEQ ID NO: 8: SUV39H2 gRNA02: GAATCTAAACAATTATGAGG
SEQ ID NO: 9: SETDB1 gRNA01: CCTTACCTGAATCAATACTG
SEQ ID NO: 10: SETDB1 gRNA02: GTTATCTATAAGACACCTTG
SEQ ID NO: 11: TRIM28 gRNA01: CCAGCGGGTGAAGTACACCA
SEQ ID NO: 12: TRIM28 gRNA02: CTTCCCAGGCAGTACCACTG
AAVS1 gRNAs:
gRNA 01 Forward (SEQ ID NO: 13): GCTTCCTTACACTTCCCAAGAGGA
gRNA 01 Reverse (SEQ ID NO: 14): TCGTGGGGTCCAGGCCAAGTAG
EHMT gRNAs:
gRNA 01 Forward (SEQ ID NO: 15): TTGTGCAGATGATGGGCGTTTC
gRNA 01 Reverse (SEQ ID NO: 16): CAACCCTCACTTCCCTCCTCTG
gRNA 02 Forward (SEQ ID NO: 17): CTCCCTCCCCCTTCTTTGTCTG
gRNA 02 Reverse (SEQ ID NO: 18): TCTTGCCTAATTGCTCTGGGCT
SUV39H2 gRNAs:
gRNA 01 Forward (SEQ ID NO: 19): GGATAGCTCTGCAGAGATGGCA
gRNA 01 Reverse (SEQ ID NO: 20): AGCAGCATGTGTTACATCTGAGT
gRNA 02 Forward (SEQ ID NO: 21): TTGCCAGTTGAAAGATGGGGAA
gRNA 02 Reverse (SEQ ID NO: 22): TGGCAACACAGATGATAGGACA
SETDB1 gRNAs:
gRNA 01 Forward (SEQ ID NO: 23): ACTGGCTTTGACCTTTTCTGCA
gRNA 01 Reverse (SEQ ID NO: 24): CAGGGTGTAGCACATAGGGAACA
gRNA 02 Forward (SEQ ID NO: 25): CCTCCTACCGTGCTCCCATG
gRNA 02 Reverse (SEQ ID NO: 26): CCGGGATACGTTCCTTGCTGTA
TRIM28 gRNAs
gRNA 01 Forward (SEQ ID NO: 27): GCCTAGGTTGGGTCAAGGGAC
gRNA 01 Reverse (SEQ ID NO: 28): AGACTACACACCACAACCCCAG
gRNA 02 Forward (SEQ ID NO: 29): AACGGTAAGTATGGCACCTCCC
gRNA 02 Reverse (SEQ ID NO: 30): TGGTGATGAGGGGTGTTCAACA
TRIM28 N-Term in FIG. 35A (SEQ ID NO: 31)
CGGCGTGTGAATGGCGGCCTCCGCGG . . .
TRIM28 gRNA (SEQ ID NO: 32)
CGUGUGAAUGGCGGCCUCCG

1. A method for generating a natural killer (NK) cell comprising: differentiating a pluripotent stem cell engineered to lack TRIM28 expression and/or activity for a sufficient time to promote differentiation to a CD56⁺ NK cell.

2. The method of claim 1, wherein the pluripotent stem cell comprises an induced pluripotent stem (iPS) cell, an embryonic stem cell, a cord blood cell, and/or a bone marrow cell.

3. The method of claim 2, wherein the cord blood cell and/or the bone marrow cell comprises a CD34+ hemogenic endothelial cell.

4. The method of claim 1, wherein the pluripotent stem cell engineered to lack TRIM28 expression and/or activity is generated using a CRISPR-Cas9 system.

5. The method of claim 2, wherein the pluripotent stem cell is engineered to delete or mutate a gene and/or protein encoding TRIM28, thereby reducing expression and/or activity of TRIM28.

6. The method of claim 4, further comprising treatment with at least one additional inhibitor of EHMT1 and/or SETDB1.

7. The method of claim 1, wherein the NK cell generated is a CD56+, CD56+CD3−, CD56+CD3−CD8+, or CD56CD3−CD8− NK cell.

8. The method of claim 1, wherein the cell engineered to lack TRIM28 expression comprises a pattern of transposable elements that is substantially similar to the pattern of transposable elements in a cell committed to the lymphoid lineage.

9. A method for generating an NK cell comprising contacting a pluripotent stem cell with an inhibitor of TRIM28 expression and/or activity and culturing under conditions and for a sufficient time to promote differentiation to an NK cell.

10. The method of claim 9, wherein the NK cell is CD56+, CD56+CD3−, CD56+CD3−CD8+, or CD56CD3−CD8− NK cell, etc.

11. The method of claim 9, wherein the inhibitor of TRIM28 expression and/or activity comprises an inhibitory nucleic acid, a small molecule, or a peptide.

12. The method of claim 11, wherein the inhibitory nucleic acid is selected from the group consisting of an siRNA, an shRNA, a miRNA, an antisense oligonucleotide, an aptamer, a ribozyme, and a triplex forming oligonucleotide.

13. The method of claim 12, further comprising a step of administering or contacting with at least one inhibitor that modulates methylation of DNA.

14. The method of claim 13, wherein at least one inhibitor that inhibits methylation of DNA inhibits the expression and/or activity of one or more of: DNMT; MBD; DNA demethylase; HMT; methyl-histone binding protein; histone demethylase; HAT; acetyl-binding protein; or HDAC.

15. The method of claim 11, wherein further comprising administering or contacting with at least one inhibitor that targets sumoylation.

16. The method of claim 15, wherein at least one inhibitor that targets sumoylation is an E3 ligase inhibitor.

17. A method for generating an NK cell, the method comprising: contacting a pluripotent stem cell treated with an inhibitor that disrupts TRIM28 binding with one or more binding partners.

18. The method of claim 17 wherein the one or more binding partners is selected from the group consisting of KRAB-ZNF transcription factors, MDM2, p53, the NuRD complex (comprising of NuRD, Mi2α, and an HDAC), SETDB1, CBF-A, and HP1.

19. The method of claim 17, wherein the cell engineered to lack TRIM28 expression comprises a pattern of transposable elements that is substantially similar to the pattern of transposable elements in a cell committed to the lymphoid lineage.

20. The method of claim 1, wherein the NK cell is a CD56+, CD56+CD3−, CD56+CD3−CD8+, or CD56CD3−CD8− NK cell.

21. An engineered NK cell generated using the method of any one of claims 1-20, wherein the NK cell lacks TRIM28 expression or activity.

22. An engineered NK cell generated using the method of any one of claims 17-21.

23. A therapeutic cell composition comprising an NK cell of claim 22 or a population thereof, and a pharmaceutically acceptable carrier.

24. The therapeutic composition of claim 23, for use in cellular replacement therapy in a patient.

25. A therapeutic CAR-NK cell composition comprising an NK cell that lacks TRIM28 expression and/or activity, wherein the NK cell expresses a chimeric antigen receptor (CAR).

26. The therapeutic CAR-NK cell of claim 25, wherein the NK cell is an CD56+, CD56+CD3−, CD56+CD3−CD8+, or CD56CD3−CD8− NK cell.

27. The therapeutic CAR-NK cell of claim 25, wherein the NK cell is generated by in vitro differentiation of a pluripotent stem cell engineered to lack TRIM28 expression and/or activity.

28. The therapeutic CAR-NK cell composition of claim 25, wherein the composition further comprises a pharmaceutically acceptable carrier.

29. The therapeutic CAR-NK cell composition of claim 25, wherein the cell is autologous to the subject to be treated.

30. The therapeutic CAR-NK composition of claim 29, further comprising a pharmaceutically acceptable carrier.

31. A method of treating a subject in need thereof, the method comprising: administering an NK cell of claim 21 in combination with a NK cell engager (NKCE), bispecific killer cell engager (BiKE), or trispecific killer cell engager (TRiKE) to a subject in need thereof.

32. A method of treating a subject in need thereof, comprising administering a therapeutic cell composition of claim 25 or 31 to a subject in need thereof.

33. The method of claim 32, wherein the subject in need thereof has or is at risk of having cancer.

34. The method of claim 31 or 32, wherein the subject in need thereof has or is undergoing chemotherapy and/or irradiation.

35. The method of claim 33, wherein the cancer comprises a leukemia or a lymphoma.

36. The method of claim 35, wherein the cancer is of a B-cell lymphoma; a low grade/follicular non-Hodgkin's lymphoma (NHL); a small lymphocytic (SL) NHL; an intermediate grade/follicular NHL; an intermediate grade diffuse NHL; a high grade immunoblastic NHL; a high grade lymphoblastic NHL; a high grade small non-cleaved cell NHL; a bulky disease NHL; a mantle cell lymphoma; an AIDS-related lymphoma; a Waldenstrom's Macroglobulinemia); a chronic lymphocytic leukemia (CLL); an acute lymphoblastic leukemia (ALL); a Hairy cell leukemia; or a chronic myeloblastic leukemia.

37. The method of claims 32-34, wherein the subject in need thereof is human.

38. A method for comparing the pattern of transposable elements in a progenitor cell to the pattern of transposable elements in a progenitor cell committed to the lymphoid progenitor cell, and wherein the presence of a substantially similar pattern of transposable elements as compared to a lymphoid progenitor cell is detected, the cell is identified as a lymphoid progenitor cell.

39. The method of claim 38, wherein the reference comprises a reference cell or population or a reference value.

40. The method of claim 39, wherein the reference cell or population comprises a hematopoietic stem cell or a myeloid progenitor cell.

41. The method of claim 40, further comprising a step of isolating the lymphoid progenitor cell.

42. The method of claim 38, wherein the transposable elements are selected from the group comprising: endogenous retroviruses (ERVs), long interspersed elements (LINEs), and short interspersed elements (SINEs).


SEQUENCE LISTING (32 sequences)

SEQ ID NO: 1 - DNA, 6652 bp, Homo sapiens
  source 1..6652: mol_type = genomic DNA
agtgacgcagaggctggagacgactctacggcggcgaagagacgcgggttgaggaagagggacggattgcccatgcgcttgggcgcacagcggcccgcttctgtgtggtctggaggtggagctgagaggggaatcacactctataaaggttcgcataccccactggcggattcaattgcggcagtgacgtcacagaggccccgccccgcccccacaagagccccaccgacgtggggttggcggtggtggaaggactaggagttggcgcgtgcgtactggcggcctctcccgcaccgaccggcctgggccccgcccccgggcgtgaggcgcccaatgcgcgtgcgcggcggcgtcggcgccagttatttctgtcccgccccccggcctcggctctttctgcgagcgggcgcgcgggcgagcggttgtgcttgtgcttgtggcgcgtggtgcgggtttcggcggcggctgaggaagaagcgcgggcggcgccttcgggaggcgagcaggcagcagttggccgtgccgtagcagcgtcccgcgcgcggcgggcagcggcccaggaggcgcgtggcggcgctcggcctcgcggcggcggcggcggcagcggcccagcagttggcggcgagcgcgtctgcgcctgcgcggcgggccccgcgcccctcctccccccctgggcgcccccggcggcgtgtgaatggcggcctccgcggcggcagcctcggcagcagcggcctcggccgcctctggcagcccgggcccgggcgagggctccgctggcggcgaaaagcgctccaccgccccttcggccgcagcctcggcctctgcctcagccgcggcgtcgtcgcccgcggggggcggcgccgaggcgctggagctgctggagcactgcggcgtgtgcagagagcgcctgcgacccgagagggagccccgcctgctgccctgtttgcactcggcctgtagtgcctgcttagggcccgcggcccccgccgccgccaacagctcgggggacggcggggcggcgggcgacggcaccggtaagtacgaagtgatcggtgccacccctccccctactctctgcctttgattccgactgggtgcagagatgaggatgccacctgggcgagaggatgggggcccggacagggcacgggaaatactttctgggtcctgcatacgaacgtgggtttgtgctggccgctgagatgggacatctgactaaagttggagaaaagaaggctcggggaggggaggggctggttcgctgcgggataatggtcgggggcccacccagcaggggaatggtgggggccataacctgggtgggaacttgtaacagtctcccacatccctgcttctcgaagtggtggactgtcccgtgtgcaagcaacagtgcttctccaaagacatcgtggagaattatttcatgcgtgatagtggcagcaaggctgccaccgacgcccaggatgcgaaccaggtgcgtcctatctcagcaaccacaaggaggtttctggggagggggcatctgcgcaggaggagcttggcaccagctccaggctgttactccactttcccaaggctctgggtgggctgcctaggttgggtcaagggaccaatcttaaatctccggttgtattttctgggatgtaaacgtggatctatcaagttgtcttgccttctctgaccctgcctttgtctggcagtgctgcactagctgtgaggataatgccccagccaccagctactgtgtggagtgctcggagcctctgtgtgagacctgtgtagaggcgcaccagcgggtgaagtacaccaaggaccatactgtgcgctctactggtacatgaggctgaggggggctgttggagttgttctcccatgtgtgccctcagttgcttttatgatgttggttgcatctggtggatgggtcctagagttctctagggggtgccgcccgaagggcccgagggcagaactccagaagcagaaaaactggggttgtggtgtgtagtctctagggcctaggtgggagagggtgggaaggggaaataagggagaccttaatgtgctgcagggaggtacaaagggtttgagaaggcttatcagggagttgtcagacctggtggtgaagggctcagcatatgcaaacagggaaaggcatggtgtgaagggctttctgggtttgtggttccctggcacacatctggtataagatgctgctagggaaagtagactgtgggtccatggattataagtgatgatgatttttggaagtgttttagcttttgagttggcagagacttacccaaagtttggtttggcatttccagcttttcacagacacagctagctttggtttggaaggagcttgagagtcttttgagtctagccaagttttggggccagaatcctcccatgagtggccttttggtagccatactgtgattcttcccatcccagaggacagcattggtggaagatagcgggaaggtggtggctgtggggcctgagcagccaggagggatacgcaggggctgcagggtgtcctacattccagacgtcccaggcaaaggtcagggtgcaggaatgagtggcattggggaagtcagtttccccaatgagccctgatgctacatattttggatttggttggtatgtgggaccagtgtgtttgatcagttttctaatggggagacaggctttggaggttattaatgggttggtctttgggtgggattatgctgagcaccaagattctgctctagagggggtgtgaccaccattctgagggtcctggtttgacatttcatctctcccagtatgagaacctctacaaggtttcctctgggcccttggattgtaagagaccctggtgttctcttgtcagggaagagggaggaagttaaccatgaaaggagaagaaaaggaatccagggtggtgtgtaggcagaattctgagtatgtccacttggagaagtgttcagtaaagggaacagtgtggggaagggtcagcgggggtggggggtggggtggggtggtcctctttttctagacgctgttatccttacccttacattgttgttatctctagaagctagaagaaagggatgtgtttctcagctatgttggggcagaggattctgggagcaaggagttgggcttgcaggcctgtgttcttccctggccacaccctctacatcttcccaataaatggcccagtgtcacagaacaggccggaaatgaggccccaactcattctgcttcctgagttgggggtggtgaagggcaaggtccagccttatgattcccactccccagggccagccaagtctcgggatggtgaacgtactgtctattgcaacgtacacaagcatgaaccccttgtgctgttttgtgagagctgtgatactctcacctgccgagactgccagctcaatgcccacaaggaccaccagtgagtcccaaggcatagtggttgggtgggtgggtgccaccccttccgtagcttagtgctcaggaacacatctgtctgctctcaggtaccagttcttagaggatgcagtgaggaaccagcgcaagctcctggcctcactggtgaagcgccttggggacaaacatgcaacattgcagaagagcaccaaggaggttcgcagctcgtaagtgtgggttctggggctgtgggggtggcccagggcagcaagaccctacctagcctgacctgctgtgtcccctagaatccgccaggtgtctgacgtacagaagcgtgtgcaagtggatgtcaagatggccatcctgcagatcatgaaggagctgaataagcggggccgtgtgctggtcaatgatgcccaggtaagccttgtgccggtgagaagggtccctgagccccctctgctgattgatgatgctgtctggggtgaggagtgatcctagtattcttctgccttctctgcttacctcatacaccttctatctgcagaaggtgactgaggggcagcaggagcgcctggagcggcagcactggaccatgaccaagatccagaagcaccaggagcacattctgcgctttgcctcttgggctctggagagtgacaacaacacagcccttttgctttctaagaagttggtgtgtactggtgggctcctggctggtgggttccaggcaggtggttcccaatacctcaaatccctatttgttgttggtgtgctcattctttcctccctttcactcccaaccagatctacttccagctgcaccgggccctcaagatgattgtggatcccgtggagccacatggcgagatgaagtttcagtgggacctcaatgcctggaccaagagtgccgaggcctttggtgggtccccagctttacctcactctgttattaccccacgtgctgcacctacttacgtttctctcttctttttgcaggcaagattgtggcagagcgtcctggcactaactcaacaggccctgcacccatggcccctccaagagccccagggcccctgagcaagcagggctctggcagcagccaggtgagcaggagagaggacccaggaaggggtgggcagggaatggggtccagtagcaggagagaggacccaggcagggggggtgggcagggaatggggtccagtagggttcctgtcccactgaggcagagggttctgctttgttcacagcccatggaggtgcaggaaggctatggctttgggtcaggtgagtgggtctgcctagtgggtggggaagggccccagtgctgtttctaccccaacctgccagtcttctttctccattttctaaggagatgatccctactcaagtgcagagccccatgtgtcaggtgtgaaacggtaagtatggcacctcccctgggggtgaggtggatggagggtgggggtgtactcatgccaggttgctgcatctacaggtcccgctcaggtgagggcgaggtgagcggccttatgcgcaaggtgccacgagtgagccttgaacgcctggacctggacctcacagctgacagccagccacccgtcttcaaggtcttcccaggcagtaccactgaggactacaaccttattgttattgaacgtggcgctgccgctgcagctaccggccagccagggactgcgcctgcaggaacccctggtgccccacccctggctggcatggccattgtcaaggtaagcctgtcccaaggaactatagctgtaggatgaagcctgtagtccaggtctggaccctgttgaacacccctcatcaccaccttgcaggaggaggagacggaggctgccattggagcccctcctactgccactgagggccctgagaccaaacctgtgcttatggctcttgcggagggtcctggtgctgagggtccccgcctggcctcacctagtggcagcaccagctcagggctggaggtggtggctcctgagggtacctcagccccaggtggtggcccgggaaccctggatgacagtgccaccatttgccgtgtctgccagaagccaggcgatctggttatgtgcaaccagtgtgagttttgtttccacctggactgtcacctgccggccctgcaggatgtaccagggtgagtgtgaggctggtgggggtcaagtctgggtgttgggctgtctggacaggatcatgtgcagacccttattttcttcaccctagggaggagtggagctgctcactctgccatgtgctccctgacctgaaggaggaggatggcagcctcagcctggatggtgcagacagcactggcgtggtggccaagctctcaccagccaaccagcgggtgagggctggggttacttaggtggggttgcccagagaggctttataggtgctgcccagagctgtgacatcccttacaatgtttgtagaaatgtgagcgtgtactgctggccctattctgtcacgaaccctgccgccccctgcatcagctggctaccgactccaccttctccctggtgagtcctaggatgggaaaggggaagggggtggtggctgctgggtctcgccctcaacctgtgcatgtatatgtgtgtctttgtgtgtgtatgtgtgatctctgcctgcaggaccagcccggtggcaccctggatctgaccctgatccgtgcccgcctccaggagaagttgtcacctccctacagctccccacaggagtttgcccaggatgtgggccgcatgttcaagcaattcaacaagttaactgaggtgagccagtggaatggagaggctgtgggcagggggagatgtgaaggaaagaactaggacccattcatccactgcattcctgcttggcccaggacaaggcagacgtgcagtccatcatcggcctgcagcgcttcttcgagacgcgcatgaacgaggccttcggtgacaccaagttctctgctgtgctggtggagcccccgccgatgagcctgcctggtgctggcctgagttcccaggagctgtctggtggccctggtgatggcccctgaggctggagcccccatggccagcccagcctggctctgttctctgtcctgtcaccccatccccactcccctggtggcctgactcccactccctggtggccccatcccccagttcctcacgatatggtttttacttctgtggatttaataaaaacttcaccagtt

SEQ ID NO: 2 - RNA, 3364 nt, Homo sapiens
  source 1..3364: mol_type = mRNA
agtgacgcagaggctggagacgactctacggcggcgaagagacgcgggttgaggaagagggacggattgcccatgcgcttgggcgcacagcggcccgcttctgtgtggtctggaggtggagctgagaggggaatcacactctataaaggttcgcataccccactggcggattcaattgcggcagtgacgtcacagaggccccgccccgcccccacaagagccccaccgacgtggggttggcggtggtggaaggactaggagttggcgcgtgcgtactggcggcctctcccgcaccgaccggcctgggccccgcccccgggcgtgaggcgcccaatgcgcgtgcgcggcggcgtcggcgccagttatttctgtcccgccccccggcctcggctctttctgcgagcgggcgcgcgggcgagcggttgtgcttgtgcttgtggcgcgtggtgcgggtttcggcggcggctgaggaagaagcgcgggcggcgccttcgggaggcgagcaggcagcagttggccgtgccgtagcagcgtcccgcgcgcggcgggcagcggcccaggaggcgcgtggcggcgctcggcctcgcggcggcggcggcggcagcggcccagcagttggcggcgagcgcgtctgcgcctgcgcggcgggccccgcgcccctcctccccccctgggcgcccccggcggcgtgtgaatggcggcctccgcggcggcagcctcggcagcagcggcctcggccgcctctggcagcccgggcccgggcgagggctccgctggcggcgaaaagcgctccaccgccccttcggccgcagcctcggcctctgcctcagccgcggcgtcgtcgcccgcggggggcggcgccgaggcgctggagctgctggagcactgcggcgtgtgcagagagcgcctgcgacccgagagggagccccgcctgctgccctgtttgcactcggcctgtagtgcctgcttagggcccgcggcccccgccgccgccaacagctcgggggacggcggggcggcgggcgacggcaccgtggtggactgtcccgtgtgcaagcaacagtgcttctccaaagacatcgtggagaattatttcatgcgtgatagtggcagcaaggctgccaccgacgcccaggatgcgaaccagtgctgcactagctgtgaggataatgccccagccaccagctactgtgtggagtgctcggagcctctgtgtgagacctgtgtagaggcgcaccagcgggtgaagtacaccaaggaccatactgtgcgctctactgggccagccaagtctcgggatggtgaacgtactgtctattgcaacgtacacaagcatgaaccccttgtgctgttttgtgagagctgtgatactctcacctgccgagactgccagctcaatgcccacaaggaccaccagtaccagttcttagaggatgcagtgaggaaccagcgcaagctcctggcctcactggtgaagcgccttggggacaaacatgcaacattgcagaagagcaccaaggaggttcgcagctcaatccgccaggtgtctgacgtacagaagcgtgtgcaagtggatgtcaagatggccatcctgcagatcatgaaggagctgaataagcggggccgtgtgctggtcaatgatgcccagaaggtgactgaggggcagcaggagcgcctggagcggcagcactggaccatgaccaagatccagaagcaccaggagcacattctgcgctttgcctcttgggctctggagagtgacaacaacacagcccttttgctttctaagaagttgatctacttccagctgcaccgggccctcaagatgattgtggatcccgtggagccacatggcgagatgaagtttcagtgggacctcaatgcctggaccaagagtgccgaggcctttggcaagattgtggcagagcgtcctggcactaactcaacaggccctgcacccatggcccctccaagagccccagggcccctgagcaagcagggctctggcagcagccagcccatggaggtgcaggaaggctatggctttgggtcaggagatgatccctactcaagtgcagagccccatgtgtcaggtgtgaaacggtcccgctcaggtgagggcgaggtgagcggccttatgcgcaaggtgccacgagtgagccttgaacgcctggacctggacctcacagctgacagccagccacccgtcttcaaggtcttcccaggcagtaccactgaggactacaaccttattgttattgaacgtggcgctgccgctgcagctaccggccagccagggactgcgcctgcaggaacccctggtgccccacccctggctggcatggccattgtcaaggaggaggagacggaggctgccattggagcccctcctactgccactgagggccctgagaccaaacctgtgcttatggctcttgcggagggtcctggtgctgagggtccccgcctggcctcacctagtggcagcaccagctcagggctggaggtggtggctcctgagggtacctcagccccaggtggtggcccgggaaccctggatgacagtgccaccatttgccgtgtctgccagaagccaggcgatctggttatgtgcaaccagtgtgagttttgtttccacctggactgtcacctgccggccctgcaggatgtaccaggggaggagtggagctgctcactctgccatgtgctccctgacctgaaggaggaggatggcagcctcagcctggatggtgcagacagcactggcgtggtggccaagctctcaccagccaaccagcggaaatgtgagcgtgtactgctggccctattctgtcacgaaccctgccgccccctgcatcagctggctaccgactccaccttctccctggaccagcccggtggcaccctggatctgaccctgatccgtgcccgcctccaggagaagttgtcacctccctacagctccccacaggagtttgcccaggatgtgggccgcatgttcaagcaattcaacaagttaactgaggacaaggcagacgtgcagtccatcatcggcctgcagcgcttcttcgagacgcgcatgaacgaggccttcggtgacaccaagttctctgctgtgctggtggagcccccgccgatgagcctgcctggtgctggcctgagttcccaggagctgtctggtggccctggtgatggcccctgaggctggagcccccatggccagcccagcctggctctgttctctgtcctgtcaccccatccccactcccctggtggcctgactcccactccctggtggccccatcccccagttcctcacgatatggtttttacttctgtggatttaataaaaacttcaccagtt

SEQ ID NO: 3 - protein, 835 aa, Homo sapiens
MAASAAAASAAAASAASGSPGPGEGSAGGEKRSTAPSAAASASASAAASSPAGGGAEALELLEHCGVCRERLRPEREPRLLPCLHSACSACLGPAAPAAANSSGDGGAAGDGTVVDCPVCKQQCFSKDIVENYFMRDSGSKAATDAQDANQCCTSCEDNAPATSYCVECSEPLCETCVEAHQRVKYTKDHTVRSTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPAPMAPPRAPGPLSKQGSGSSQPMEVQEGYGFGSGDDPYSSAEPHVSGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 4 - RNA, 20 nt, synthetic construct
  source 1..20: mol_type = other RNA
ggacgcaccattctcacaaa

SEQ ID NO: 5 - RNA, 20 nt, synthetic construct
  source 1..20: mol_type = other RNA
gggccggtgcacaaacagcg

SEQ ID NO: 6 - RNA, 20 nt, synthetic construct
  source 1..20: mol_type = other RNA
ttcggctgcttccatcaacg

SEQ ID NO: 7 - RNA, 20 nt, synthetic construct
  source 1..20: mol_type = other RNA
tttcgaactagcaatggacg

SEQ ID NO: 8 - RNA, 20 nt, synthetic construct
  source 1..20: mol_type = other RNA
gaatctaaacaattatgagg

SEQ ID NO: 9 - RNA, 20 nt, synthetic construct
  source 1..20: mol_type = other RNA
ccttacctgaatcaatactg

SEQ ID NO: 10 - RNA, 20 nt, synthetic construct
  source 1..20: mol_type = other RNA
gttatctataagacaccttg

SEQ ID NO: 11 - RNA, 20 nt, synthetic construct
  source 1..20: mol_type = other RNA
ccagcgggtgaagtacacca

SEQ ID NO: 12 - RNA, 20 nt, synthetic construct
  source 1..20: mol_type = other RNA
cttcccaggcagtaccactg

SEQ ID NO: 13 - RNA, 24 nt, synthetic construct
  source 1..24: mol_type = other RNA
gcttccttacacttcccaagagga

SEQ ID NO: 14 - RNA, 22 nt, synthetic construct
  source 1..22: mol_type = other RNA
tcgtggggtccaggccaagtag

SEQ ID NO: 15 - RNA, 22 nt, synthetic construct
  source 1..22: mol_type = other RNA
ttgtgcagatgatgggcgtttc

SEQ ID NO: 16 - RNA, 22 nt, synthetic construct
  source 1..22: mol_type = other RNA
caaccctcacttccctcctctg

SEQ ID NO: 17 - RNA, 22 nt, synthetic construct
  source 1..22: mol_type = other RNA
ctccctcccccttctttgtctg

SEQ ID NO: 18 - RNA, 22 nt, synthetic construct
  source 1..22: mol_type = other RNA
tcttgcctaattgctctgggct

SEQ ID NO: 19 - RNA, 22 nt, synthetic construct
  source 1..22: mol_type = other RNA
ggatagctctgcagagatggca

SEQ ID NO: 20 - RNA, 23 nt, synthetic construct
  source 1..23: mol_type = other RNA
agcagcatgtgttacatctgagt

SEQ ID NO: 21 - RNA, 22 nt, synthetic construct
  source 1..22: mol_type = other RNA
ttgccagttgaaagatggggaa

SEQ ID NO: 22 - RNA, 22 nt, synthetic construct
  source 1..22: mol_type = other RNA
tggcaacacagatgataggaca

SEQ ID NO: 23 - RNA, 22 nt, synthetic construct
  source 1..22: mol_type = other RNA
actggctttgaccttttctgca

SEQ ID NO: 24 - RNA, 23 nt, synthetic construct
  source 1..23: mol_type = other RNA
cagggtgtagcacatagggaaca

SEQ ID NO: 25 - RNA, 20 nt, synthetic construct
  source 1..20: mol_type = other RNA
cctcctaccgtgctcccatg

SEQ ID NO: 26 - RNA, 22 nt, synthetic construct
  source 1..22: mol_type = other RNA
ccgggatacgttccttgctgta

SEQ ID NO: 27 - RNA, 21 nt, synthetic construct
  source 1..21: mol_type = other RNA
gcctaggttgggtcaagggac

SEQ ID NO: 28 - RNA, 22 nt, synthetic construct
  source 1..22: mol_type = other RNA
agactacacaccacaaccccag

SEQ ID NO: 29 - RNA, 22 nt, synthetic construct
  source 1..22: mol_type = other RNA
aacggtaagtatggcacctccc

SEQ ID NO: 30 - RNA, 22 nt, synthetic construct
  source 1..22: mol_type = other RNA
tggtgatgaggggtgttcaaca

SEQ ID NO: 31 - DNA, 26 bp, unidentified
  source 1..26: mol_type = unassigned DNA
cggcgtgtgaatggcggcctccgcgg

SEQ ID NO: 32 - RNA, 20 nt, synthetic construct
  source 1..20: mol_type = other RNA
cgtgtgaatggcggcctccg